(19) United States
(12) Patent Application Publication
(10) Pub. No.: US 2024/0336611 A1
(43) Pub. Date: Oct. 10, 2024

(54) ANALOGS FOR THE TREATMENT OF DISEASE

(71) Applicant: Insilico Medicine IP Limited, Hong Kong (CN)

(72) Inventors: Aleksandrs ZAVORONKOVS, Hong Kong (CN); Aleksandr ALIPER, Ramenskoye (RU); Vladimir ALADINSKIY, Kaliningrad (RU); Andrey KUKHARENKO, Moscow (RU); Luoheng QUIN, Shanghai (CN); Xin CHENG, Shanghai (CN)

(21) Appl. No.: 18/547,190

(22) PCT Filed: Feb. 23, 2022
(86) PCT No.: PCT/CN2022/077478

§ 371 Date: Aug. 21, 2023

(30) Foreign Application Priority Data
Feb. 24, 2021 (WO) PCT/CN2021/077670

(51) Int. Cl. C07D 471/08; A61K 31/4178; A61K 31/422; A61K 31/427; A61K 31/4375; A61K 31/438; A61K 31/4439; A61K 31/496; A61K 31/4995; C07D 401/14; C07D 403/14; C07D 413/14; C07D 417/14; C07D 471/04; C07D 471/10; C07D 487/10

(52) U.S. Cl. CPC C07D 471/08; A61K 31/4178; A61K 31/422; A61K 31/427; A61K 31/4375; A61K 31/438; A61K 31/4439; A61K 31/496; A61K 31/4995; C07D 401/14; C07D 403/14; C07D 413/14; C07D 417/14; C07D 471/04; C07D 471/10; C07D 487/10

(57) ABSTRACT

Provided are TNIK and/or MAP4K4 kinases inhibitors for the treatment of disease. In one aspect, disclosed herein are kinase inhibitors having a structure of Formulas (A), (A*), (I), (II), (AA), (B), (C), (D), (B*), (C*) and (D*). Further described herein are pharmaceutical composition comprising these compounds and methods of using these compounds. In one aspect, disclosed herein are methods of treating a disease or condition by administering the kinases inhibitors described herein.

### CROSS REFERENCE

This application claims the benefit of International Application No. PCT/CN2021/077670, filed Feb. 24, 2021, which is hereby incorporated by reference in its entirety.

### BACKGROUND OF THE INVENTION

A biologically active enzyme known as Traf2- and Nck-interacting protein kinase is an enzyme commonly known as the TNIK in humans, and which is encoded by the TNIK gene. TNIK as a serine/threonine kinase is involved in various biological processes. There is a need for new drug candidates that can target TNIK.

### SUMMARY OF THE INVENTION

A biologically active enzyme known as Traf2- and Nck-interacting protein kinase is an enzyme commonly known as the TNIK in humans, and which is encoded by the TNIK gene. TNIK as a serine/threonine kinase is involved ii various biological processes including acting as an essential regulatory component of the Wnt signaling pathway. TNIK directly binds TCF4 and b-catenin and phosphorylates TCF4. TNIK plays an activator of Wnt target gene expressions. TNIK also modulates the actin cytoskeleton and activates the c-Jun N-terminal kinase pathway, which is responsive to environmental stress. It, is also part of a signaling complex composed of NEDD4, RAP2A and TNIK, which regulates neuronal dendrite extension and arborization during development. More generally, TNIK may play a role in cytoskeletal rearrangements and regulate cell spreading. TNIK also caused weak Smad1 T322 phosphorylation, involved in TGF-b1 signaling transduction.

TNIK is also considered to be a germinal center kinase (GCK), which can be characterized by an N-terminal kinase domain and a C-terminal GCK domain that serves a regulatory function.

TNIK activation of Wnt signaling plays important roles in carcinogenesis and embryonic development. Mutations in this gene are associated with an autosomal recessive form of cognitive disability.

Additionally, TNIK is linked to colorectal cancer, and possibly other cancers. As such, TNIK has been identified as an attractive candidate for drug targeting in colorectal cancer.

The current data imply TNIK is a potential target for the generation of small molecule inhibitors to specifically block the Wnt pathway in disease states such as colorectal cancer or the autosomal recessive form of cognitive disability.

Also, it is known that TGF-β-activated EMT can be inhibited through the attenuation of Smad aid non-Smad signaling pathways, including the Wnt, NF-kB, FAK-Src-paxillin-related focal adhesion, and MAP kinases (ERK and JNK) signaling pathways. As such, therapeutic targets associated with EMT, such as TNIK being a target for inhibition, can be used for therapies for treating and/or preventing EMT-based disorders, such as cancer metastasis and fibrosis.

Accordingly, it would be advantageous to have a TNIK inhibitor that can inhibit the kinase activity of TNIK. It would also be advantageous to have a specific TNIK inhibitor that selectively inhibits TNIK, as a member of Ste20 family of MAP kinase kinase kinase kinases (MAP4K).

In an aspect, the present disclosure provides a compound represented by Formula (A):

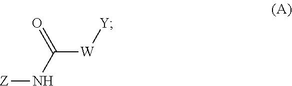

or a pharmaceutically acceptable salt thereof, wherein:
Z is selected from optionally substituted 3- to 12-membered heterocycle and optionally substituted C₃-C₁₂ carbocycle, wherein each is optionally substituted with one or more substituents independently selected at each occurrence from —N(R¹⁰)₂, halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₁-C₁₀ alkyl, C₃₋₁₂ carbocycle, 3- to 12-membered heterocycle;
   wherein the C₁-C₁₀ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R¹⁰ is selected from optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
W is selected from optionally substituted 5- to 8-membered heterocycle and optionally substituted C₃-C₈ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; and
Y is selected from optionally substituted 5- to 8-membered heterocycle and optionally substituted C₃-C₈ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.

In certain embodiments, the disclosure provides a compound represented by Formula (I):

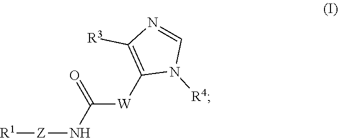

or a pharmaceutically acceptable salt thereof, wherein:
R¹ is selected from:
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   substituted C₁-C₆ alkyl, wherein the C₁-C₆ alkyl is substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   optionally substituted 3 to 8-membered heterocycle; wherein the 3- to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each are optionally substituted with one or more substituents independently selected at each occurrence from one or more halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from one or more halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   optionally substituted C₃₋₁₀ carbocycle, wherein the C₃₋₁₀ carbocycle is optionally substituted with one or more substituents independently selected at each occurrence from one or more halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl; and
W is selected from optionally substituted thiazole, wherein the thiazole is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

In certain embodiments, the present disclosure provides a compound represented by Formula (II):

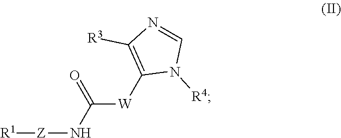

or a pharmaceutically acceptable salt thereof, wherein:
R¹ is selected from:
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   substituted C₁-C₆ alkyl, wherein the C₁-C₆ alkyl is substituted with one or more substituents independently selected at each occurrence from one or more halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   optionally substituted 3 to 10-membered heterocycle; wherein the 3- to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
   R³ is selected from optionally substituted C₁-C₆ alkyl, optionally substituted 3- to 10-membered heterocycle and optionally substituted C₃₋₁₀ carbocycle, wherein each is optionally substituted with one or more substituents independently selected at each occurrence from one or more halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
Z is selected from optionally substituted 3- to 12-membered heterocycle and optionally substituted C₃-C₁₂ carbocycle, wherein each is optionally substituted with one or more substituents are independently selected at each occurrence from halogen, —OH, —CN, —NO₂, —NH₂, oxo, ═S, —C₁₋₁₀ haloalkyl, —O—C₁₋₁₀ alkyl; and
W is selected from optionally substituted 5- to 8-membered heteroaryl, wherein the 5- to 8-membered heteroaryl is optionally substituted with one or more substituents independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle.

In certain embodiments, the present disclosure provides a compound represented by Formula (AA):

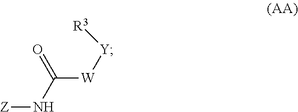

or a pharmaceutically acceptable salt thereof, wherein:
Z is selected from optionally substituted 3- to 12-membered heteroaryl and optionally substituted C₆-C₁₂ carbocycle, wherein each is optionally substituted with one or more substituents independently selected at each occurrence from R¹;
R¹ is selected from
   —N(R¹⁰)₂, halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₁-C₁₀ alkyl, C₃₋₁₂ carbocycle, and 3- to 12-membered heterocycle;
   wherein the C₁-C₁₀ alkyl is optionally substituted with one or more substituents independently selected at each occurrence from halogen, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂;
R¹⁰ is selected from optionally substituted C₁-C₆ alkyl, wherein the C₁-C₆ alkyl is optionally substituted with one or more substituents independently selected at each occurrence from halogen, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
W is selected from optionally substituted 5- to 8-membered heterocycle, wherein the 5- to 8-membered heterocycle has at least 2 heteroatoms; and wherein the 5- to 8-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; wherein when W is imidazole and Z is phenyl and R¹ is piperidine, piperidine is substituted;
and
Y is selected from optionally substituted 5- to 10-membered heterocycle and optionally substituted C₃-C₈ carbocycle, wherein each is optionally substituted with one or more substituents independently selected at each occurrence from R⁴;
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.
each R⁴ is independently selected from halogen, —OH, —CN, —NO₂, —NH₂, —N(H)C₁-C₆ alkyl, —N(C₁-C₆ alkyl)₂, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; and
wherein when W is imidazole, Y is imidazole, and Z is phenyl substituted with piperazine, the piperazine is substituted.

In certain embodiments, the present disclosure provides a compound represented by Formula (A*):

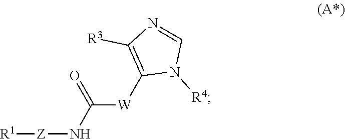

or a pharmaceutically acceptable salt thereof, wherein:
R¹ is selected from:
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
   optionally substituted 3 to 10-membered heterocycle; wherein the 3- to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
R³ is selected from optionally substituted C₁-C₆ alkyl, optionally substituted 3- to 10-membered heterocycle and optionally substituted C₃₋₁₀ carbocycle, wherein each is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted 3- to 8-membered heterocycle, wherein the 3- to 8-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
W is selected from optionally substituted thiazole, wherein the thiazole is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

In certain embodiments, the present disclosure provides a compound represented by Formula (B*):

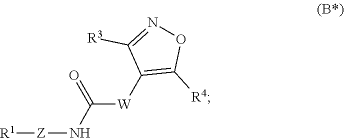

or a pharmaceutically acceptable salt thereof, wherein:
R¹ is selected from:
   optionally substituted 3 to 10-membered heterocycle; wherein the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
W is selected from optionally substituted 3- to 12-membered heterocycle, wherein the 3- to 12-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

In certain embodiments, the present disclosure provides a compound represented by Formula (C*):

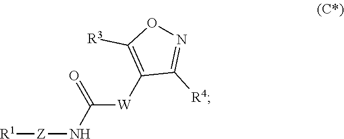

or a pharmaceutically acceptable salt thereof, wherein:
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
R¹ is selected from:
   optionally substituted 3 to 10-membered heterocycle; wherein the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
W is selected from optionally substituted 3- to 12-membered heterocycle, wherein the 3- to 12-membered is optionally substituted with one or more substituents selected at each occurrence from one or more halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

In certain embodiments, the present disclosure provides a compound represented by Formula (D*):

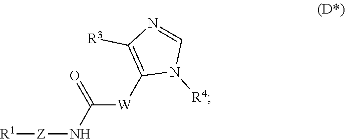

or a pharmaceutically acceptable salt thereof, wherein:
R¹ is selected from:
   optionally substituted 3 to 10-membered heterocycle; wherein the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from halogen, oxo, —C₁₋₁₀ haloalkyl, —NH₂, —CN, —O—C₁₋₁₀ alkyl, and —NO₂;
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein the each is optionally substituted with one or more substituents independently selected at each occurrence from one or more halogen, —OH, —CN, —NO₂, —NH₂, oxo, ═S, —C₁₋₁₀ haloalkyl, —O—C₁₋₁₀ alkyl;
W is selected from optionally substituted 3- to 12-membered heterocycle having at least two heteroatoms, wherein the 3- to 12-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle; and
wherein when W is imidazole, Y is imidazole, Z is phenyl, and R¹ is piperazine, the piperazine is substituted.

In some aspects, the disclosure provides a pharmaceutical composition comprising a compound or salt of Formula (A), and a pharmaceutically acceptable excipient.

In some aspects, the disclosure provides a pharmaceutical composition comprising a compound or salt of Formula (I), and a pharmaceutically acceptable excipient.

In some aspects, the disclosure provides a pharmaceutical composition comprising a compound or salt of Formula (II), and a pharmaceutically acceptable excipient.

In some aspects, the disclosure provides a method of treating or preventing disease comprising administering a compound or salt of Formula (I) or a pharmaceutical composition comprising a compound or salt of Formula (I) and a pharmaceutically acceptable excipient to a subject in need thereof. In some aspects, the disease is a cancer. In some cases, the cancer is selected from colorectal cancer, gastric cancer, breast cancer, lung cancer, pancreatic cancer, prostate cancer, multiple myeloma, chronic myelogenous leukemia, cancer metastasis, fibrosis and psychiatric disorders.

In some aspects, the disclosure provides a method of treating or preventing disease comprising administering a compound or salt of Formula (II) or a pharmaceutical composition comprising a compound or salt of Formula (II) and a pharmaceutically acceptable excipient to a subject in need thereof. In some aspects, the disease is a cancer. In some cases, the cancer is selected from colorectal cancer, gastric cancer, breast cancer, lung cancer, pancreatic cancer, prostate cancer, multiple myeloma, chronic myelogenous leukemia, cancer metastasis, fibrosis and psychiatric disorders.

In some aspects, the disclosure provides a method of treating or preventing disease comprising administering a compound or salt of Formula (A) or a pharmaceutical composition comprising a compound or salt of Formula (A) and a pharmaceutically acceptable excipient to a subject in need thereof. In some aspects, the disease is a cancer. In some cases, the cancer is selected from colorectal cancer, gastric cancer, breast cancer, lung cancer, pancreatic cancer, prostate cancer, multiple myeloma, chronic myelogenous leukemia, cancer metastasis, fibrosis and psychiatric disorders.

In some aspects, the disclosure provides a method of inhibiting TNIK kinase comprising administering a compound or salt of Formula (A), Formula (I), or Formula (II) or a pharmaceutical composition comprising a compound or salt of Formula (A), Formula (I), or Formula (II) and a pharmaceutically acceptable excipient to a subject in need thereof.

In some aspects, the disclosure provides a method of inhibiting MAP4K4 kinase comprising administering a compound or salt of Formula (A), Formula (I), or Formula (II) or a pharmaceutical composition comprising a compound or salt of Formula (A), Formula (I), or Formula (II) and a pharmaceutically acceptable excipient to a subject in need thereof.

In some aspects, the disclosure provides a pharmaceutical composition comprising Formulas (I), Formula (II), Formula (IIA), Formula (AA), Formula (B), Formula (C), Formula (D), Formula (A*), Formula (B*), Formula (C*), or Formula (D*) or salt of any one thereof and a pharmaceutically acceptable excipient.

In some aspects, the present disclosure provides a method of treating or preventing disease, comprising administering a compound or salt of Formulas (I), Formula (II), Formula (IIA), Formula (AA), Formula (B), Formula (C), Formula (D), Formula (A*), Formula (B*), Formula (C*), or Formula (D*) or a pharmaceutical composition of Formulas (I), Formula (II), Formula (IIA), Formula (AA), Formula (B), Formula (C), Formula (D), Formula (A*), Formula (B*), Formula (C*), or Formula (D*) to a subject in need thereof. In some cases, the disease is cancer. In some cases the cancer is selected from colorectal cancer, gastric cancer, breast cancer, lung cancer, pancreatic cancer, prostate cancer, multiple myeloma, chronic myelogenous leukemia, cancer metastasis, fibrosis and psychiatric disorders. In some cases, the disease is a fibrotic disease or condition selected from pulmonary fibrosis, cystic fibrosis, liver fibrosis, myocardial fibrosis, kidney fibrosis, brain fibrosis, arterial fibrosis, arthrofibrosis, intestinal fibrosis, Dupytren's contracture fibrosis, keloid fibrosis, mediastinal fibrosis, myelofibrosis, peyronie's disease fibrosis, progressive massive fibrosis, retroperitoneal fibrosis, scleroderma sclerosis fibrosis, and adhesive capsulitis fibrosis. In some cases, the disease is a fibrotic disease or condition selected from liver cirrhosis, pulmonary fibrosis, renal interstitial fibrosis, myocardial infarction, systemic sclerosis (SSc), and graft-versus-host disease (GVHD). In some cases, the disease is kidney fibrosis. In some cases, the disease is skin fibrosis. In some cases, the disease is idiopathic pulmonary fibrosis (IPF). In some cases, the disease is associated with TNIK kinase.

In some aspects, the present disclosure provides a method of treating or preventing a disease comprising inhibiting TNIK kinase by administering a compound or salt of any one of Formulas (I), Formula (II), Formula (IIA), Formula (AA), Formula (B), Formula (C), Formula (D), Formula (A*), Formula (B*), Formula (C*), or Formula (D*) or a pharmaceutical composition of Formulas (I), Formula (II), Formula (IIA), Formula (AA), Formula (B), Formula (C), Formula (D), Formula (A*), Formula (B*), Formula (C*), or Formula (D*) to a subject in need thereof.

In some aspects, the present disclosure provides a method of treating or presenting disease comprising inhibiting MAP4K4 kinase by administering a compound or salt of Formulas (I), Formula (II), Formula (IIA), Formula (AA), Formula (B), Formula (C), Formula (D), Formula (A*), Formula (B*), Formula (C*), or Formula (D*) or a pharmaceutical composition of Formulas (I), Formula (II), Formula (IIA), Formula (AA), Formula (B), Formula (C), Formula (D), Formula (A*), Formula (B*), Formula (C*), or Formula (D*) to a subject in need thereof.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### DETAILED DESCRIPTION OF THE INVENTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

### A. Definitions

Unless defined otherwise, all technical and scientific terms used herein have the same meaning as is commonly understood by one of skill in the art to which this invention belongs. All patents and publications referred to herein are incorporated by reference.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond. Unless stated otherwise specifically in the specification, an alkyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkyl is optionally substituted with oxo, halogen, —CN, —COOH, —COOMe, —OH, —OMe, —NH₂, or —NO₂. In some embodiments, the alkyl is optionally substituted with halogen, —CN, —OH, or —OMe. In some embodiments, the alkyl is optionally substituted with halogen.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to an alkyl group that may consist of 1 carbon atom, 2 carbon atoms, 3 carbon atoms, 4 carbon atoms, 5 carbon atoms or 6 carbon atoms, including straight-chain alkyl and branched-chain alkyl groups.

“Alkoxy” refers to a radical bonded through an oxygen atom of the formula —O-alkyl, where alkyl is an alkyl chain as defined above. Unless stated otherwise specifically in the specification, an alkoxy group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkoxy is optionally substituted with halogen, —CN, —COOH, COOMe, —OH, —OMe, —NH₂, or —NO₂. In some embodiments, the alkoxy is optionally substituted with halogen, —CN, —OH, or —OMe. In some embodiments, the alkoxy is optionally substituted with halogen.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like. Unless stated otherwise specifically in the specification, an alkenyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkenyl is optionally substituted with oxo, halogen, —CN, —COOH, —COOMe, —OH, —OMe, —NH₂, or —NO₂. In some embodiments, the alkenyl is optionally substituted with halogen, —CN, —OH, or —OMe. In some embodiments, the alkenyl is optionally substituted with halogen.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like. Unless stated otherwise specifically in the specification, an alkynyl group may be optionally substituted, for example, with oxo, halogen, amino, nitrile, nitro, hydroxyl, haloalkyl, alkoxy, carboxyl, carboxylate, aryl, cycloalkyl, heterocycloalkyl, heteroaryl, and the like. In some embodiments, the alkynyl is optionally substituted with oxo, halogen, —CN, —COOH, COOMe, —OH, —OMe, —NH₂, or —NO₂. In some embodiments, the alkynyl is optionally substituted with halogen, —CN, —OH, or —OMe. In some embodiments, the alkynyl is optionally substituted with halogen.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene). The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

“Aryl” refers to a radical derived from an aromatic monocyclic or aromatic multicyclic hydrocarbon ring system by removing a hydrogen atom from a ring carbon atom, wherein the ring system contains at least one aromatic ring. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene. The aryl radical may be a monocyclic, bicyclic, tricyclic or tetracyclic ring system, which may include fused or bridged ring systems. In some embodiments, the aryl is a 6- to 10-membered aryl. In some embodiments, the aryl is a 6-membered aryl (phenyl). Aryl radicals include, but are not limited to, aryl radicals derived from the hydrocarbon ring systems of anthrylene, naphthylene, phenanthrylene, anthracene, azulene, benzene, chrysene, fluoranthene, fluorene, as-indacene, s-indacene, indane, indene, naphthalene, phenalene, phenanthrene, pleiadene, pyrene, and triphenylene. Unless stated otherwise specifically in the specification, an aryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the aryl is optionally substituted with halogen, methyl, ethyl, —CN, —COOH, COOMe, —CF₃, —OH, —OMe, —NH₂, or —NO₂. In some embodiments, the aryl is optionally substituted with halogen, methyl, ethyl, —CN, —CF₃, —OH, or —OMe. In some embodiments, the aryl is optionally substituted with halogen.

“Heteroalkyl” refers to an alkyl group in which one or more skeletal atoms of the alkyl are selected from an atom other than carbon, e.g., oxygen, nitrogen (e.g., —NH—, —N(alkyl)-), sulfur, phosphorus, or combinations thereof. A heteroalkyl is attached to the rest of the molecule at a carbon atom of the heteroalkyl. In one aspect, a heteroalkyl is a C₁-C₆ heteroalkyl wherein the heteroalkyl is comprised of 1 to 6 carbon atoms and one or more atoms other than carbon, e.g., oxygen, nitrogen (e.g. —NH—, —N(alkyl)-), sulfur, phosphorus, or combinations thereof wherein the heteroalkyl is attached to the rest of the molecule at a carbon atom of the heteroalkyl. Examples of such heteroalkyl are, for example, —CH₂OCH₃, —CH₂CH₂OCH₃, —CH₂CH₂OCH₂CH₂OCH₃, —CH(CH₃)OCH₃, —CH₂NHCH₃, —CH₂N(CH₃)₂, —CH₂CH₂NHCH₃, or —CH₂CH₂N(CH₃)₂. Unless stated otherwise specifically in the specification, a heteroalkyl is optionally substituted for example, with oxo, halogen, amino, nitrile, nitro, hydroxyl, alkyl, alkenyl, alkynyl, haloalkyl, alkoxy, aryl, cycloalkyl, heterocycloalkyl, heteroaryl, and the like. In some embodiments, a heteroalkyl is optionally substituted with oxo, halogen, methyl, ethyl, —CN, —CF₃, —OH, —OMe, —NH₂, or —NO₂. In some embodiments, a heteroalkyl is optionally substituted with oxo, halogen, methyl, ethyl, —CN, —CF₃, —OH, or —OMe. In some embodiments, the heteroalkyl is optionally substituted with halogen.

“Aralkyl” refers to a radical of the formula —R‘-aryl where R’ is an alkylene chain as defined above, for example, methylene, ethylene, and the like.

“Aralkenyl” refers to a radical of the formula —R^{d}-aryl where R^{d} is an alkenylene chain as defined above. “Aralkynyl” refers to a radical of the formula —R^{e}-aryl, where R^{e} is an alkynylene chain as defined above.

“Carbocycle” refers to a saturated, unsaturated or aromatic ring system in which each ring atom of the ring system is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. In some embodiments, the carbocycle is an aryl. In some embodiments, the carbocycle is a cycloalkyl. In some embodiments, the carbocycle is a cycloalkenyl. In some embodiments, the carbocycle contains a triple bond. Unless stated otherwise specifically in the specification, a carbocycle can be optionally substituted.

“Cycloalkyl” refers to a fully saturated monocyclic or polycyclic hydrocarbon radical consisting solely of carbon and hydrogen atoms, which includes fused or bridged ring systems, and preferably having from three to twelve carbon atoms. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, norbornyl (i.e., bicyclo[2.2.1]heptanyl), norbornenyl, decalinyl, 7,7-dimethyl-bicyclo[2.2.1]heptanyl, and the like. Unless stated otherwise specifically in the specification, a cycloalkyl is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, a cycloalkyl is optionally substituted with oxo, halogen, methyl, ethyl, —CN, —COOH, COOMe, —CF₃, —OH, —OMe, —NH₂, or —NO₂. In some embodiments, a cycloalkyl is optionally substituted with oxo, halogen, methyl, ethyl, —CN, —CF₃, —OH, or —OMe. In some embodiments, the cycloalkyl is optionally substituted with halogen.

“Heterocycloalkyl” refers to a cycloalkyl group, as defined above, wherein one or more ring carbons are replaced with one or more heteroatoms, such as N, O, P, and S. A heterocycloalkyl may be optionally substituted.

“Cycloalkenyl” refers to an unsaturated non-aromatic monocyclic or polycyclic hydrocarbon radical consisting solely of carbon and hydrogen atoms, which includes fused or bridged ring systems, preferably having from three to twelve carbon atoms and comprising at least one double bond. In certain embodiments, a cycloalkenyl comprises one double bond. In certain embodiments, a cycloalkenyl comprises more than one double bond. In certain embodiments, a cycloalkenyl comprises three to ten carbon atoms. In other embodiments, a cycloalkenyl comprises five to seven carbon atoms. The cycloalkenyl may be attached to the rest of the molecule by a single bond. Examples of monocyclic cycloalkenyls includes, e.g., cyclopentenyl, cyclohexenyl, cycloheptenyl, and cyclooctenyl.

“Heterocycloalkenyl” refers to a cycloalkenyl group, as defined above, wherein one or more ring carbons are replaced with one or more heteroatoms, such as N, O, P, and S. A heterocycloalkenyl may be optionally substituted.

“Cycloalkylalkyl” refers to a radical of the formula —R^{c}-cycloalkyl where R^{c} is an alkylene chain as described above.

“Cycloalkylalkoxy” refers to a radical bonded through an oxygen atom of the formula —O—R^{c}-cycloalkyl where R^{c} is an alkylene chain as described above.

“Halo” or “halogen” refers to halogen substituents such as bromo, chloro, fluoro and iodo substituents.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

“Fluoroalkyl” refers to an alkyl radical, as defined above, that is substituted by one or more fluoro radicals, for example, trifluoromethyl, difluoromethyl, fluoromethyl, 2,2,2-trifluoroethyl, 1-fluoromethyl-2-fluoroethyl, and the like.

“Heterocycle” refers to a saturated, unsaturated or aromatic ring comprising one or more heteroatoms. Exemplary heteroatoms include N, O, Si, P, B, and S atoms. Heterocycles include e.g., 3- to 10-membered monocyclic rings, 6- to 12-membered bicyclic rings, and 6- to 12-membered bridged rings. Each ring of a bicyclic heterocycle may be selected from saturated, unsaturated, and aromatic rings. “Heterocyclene” refers to a divalent heterocycle linking the rest of the molecule to a radical group. Unless stated otherwise specifically in the specification, a heterocycle is optionally substituted, for example, with oxo, halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heterocycle is optionally substituted with halogen, methyl, ethyl, —CN, —CF₃, —OH, or —OMe. In some embodiments, the heterocycloalkyl is optionally substituted with halogen. In some embodiments, a heterocycle is a heteroaryl. In some embodiments, a heterocycle is a heterocycloalkyl. In some embodiments, a heterocycle is a heterocycloalkenyl. In some embodiments, a heterocycle contains one or more triple bonds.

In some embodiments, the heterocycle comprises one to three heteroatoms selected from the group consisting of nitrogen, oxygen, and sulfur. In some embodiments, the heterocycle comprises one to three heteroatoms selected from the group consisting of nitrogen and oxygen. In some embodiments, the heterocycle comprises one to three nitrogens. In some embodiments, the heterocycle comprises one or two nitrogens. In some embodiments, the heterocycle comprises one nitrogen. In some embodiments, the heterocycle comprises one nitrogen and one oxygen. Unless stated otherwise specifically in the specification, the heterocycle radical may be a monocyclic, bicyclic, tricyclic or tetracyclic ring system, which may include fused, spiro, or bridged ring systems; and the nitrogen, carbon, or sulfur atoms in the heterocycle radical may be optionally oxidized; the nitrogen atom may be optionally quaternized. Representative heterocycle include, the heteroaryl groups described below. Representative heterocycle also include, but are not limited to, heterocycles having from two to fifteen carbon atoms (C₂-C₁₅ heterocycloalkyl or C₂-C₁₅ heterocycloalkenyl), from two to ten carbon atoms (C₂-C₁₀ heterocycloalkyl or C₂-C₁₀ heterocycloalkenyl), from two to eight carbon atoms (C₂-C₅ heterocycloalkyl or C₂-C₈ heterocycloalkenyl), from two to seven carbon atoms (C₂-C₇ heterocycloalkyl or C₂-C₇ heterocycloalkenyl), from two to six carbon atoms (C₂-C₆ heterocycloalkyl or C₂-C₇ heterocycloalkenyl), from two to five carbon atoms (C₂-C₅ heterocycloalkyl or C₂-C₅ heterocycloalkenyl), or two to four carbon atoms (C₂-C₄ heterocycloalkyl or C₂-C₄ heterocycloalkenyl). Examples of such heterocycle radicals include, but are not limited to, aziridinyl, azetidinyl, oxetanyl, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 1,1-dioxo-thiomorpholinyl, 1,3-dihydroisobenzofuran-1-yl, 3-oxo-1,3-dihydroisobenzofuran-1-yl, methyl-2-oxo-1,3-dioxol-4-yl, and 2-oxo-1,3-dioxol-4-yl. The term heterocycle also includes all ring forms of the carbohydrates, including but not limited to the monosaccharides, the disaccharides and the oligosaccharides. In some embodiments, heterocycles have from 2 to 10 carbons in the ring. It is understood that when referring to the number of carbon atoms in a heterocycle, the number of carbon atoms in the heterocycle is not the same as the total number of atoms (including the heteroatoms) that make up the heterocycle (i.e. skeletal atoms of the heterocycle ring). In some embodiments, the heterocycle is a 3- to 8-membered. In some embodiments, the heterocycle is a 3- to 7-membered. In some embodiments, the heterocycle is a 3- to 6-membered. In some embodiments, the heterocycle is a 4- to 6-membered. In some embodiments, the heterocycle is a 5- to 6-membered.

“Heteroaryl” or “aromatic heterocycle” refers to a radical derived from a heteroaromatic ring radical that comprises one to thirteen carbon atoms, at least one heteroatom wherein each heteroatom may be selected from N, O, and S, and at least one aromatic ring. As used herein, the heteroaryl ring may be selected from monocyclic or bicyclic and fused or bridged ring systems rings wherein at least one of the rings in the ring system is aromatic, i.e., it contains a cyclic, delocalized (4n+2) π-electron system in accordance with the Hückel theory. The heteroatom(s) in the heteroaryl radical may be optionally oxidized. One or more nitrogen atoms, if present, are optionally quaternized. The heteroaryl may be attached to the rest of the molecule through any atom of the heteroaryl, valence permitting, such as a carbon or nitrogen atom of the heteroaryl. Examples of heteroaryls include, but are not limited to, pyridine, pyrimidine, oxazole, furan, thiophene, benzthiazole, and imdazopyridine. An “X-membered heteroaryl” refers to the number of endocylic atoms, i.e., X, in the ring. For example, a 5-membered heteroaryl ring or 5-membered aromatic heterocycle has 5 endocyclic atoms, e.g., triazole, oxazole, thiophene, etc. In some embodiments, the heteroaryl is a 5- to 10-membered heteroaryl. In some embodiments, the heteroaryl is a 5- to 6-membered heteroaryl. In some embodiments, the heteroaryl is a 6-membered heteroaryl. In some embodiments, the heteroaryl is a 5-membered heteroaryl. Examples include, but are not limited to, azepinyl, acridinyl, benzimidazolyl, benzothiazolyl, benzindolyl, benzodioxolyl, benzofuranyl, benzooxazolyl, benzothiazolyl, benzothiadiazolyl, benzo[b][1,4]dioxepinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzotriazolyl, benzo[4,6]imidazo[1,2-a]pyridinyl, carbazolyl, cinnolinyl, dibenzofuranyl, dibenzothiophenyl, furanyl, furanonyl, isothiazolyl, imidazolyl, indazolyl, indolyl, indazolyl, isoindolyl, indolinyl, isoindolinyl, isoquinolyl, indolizinyl, isoxazolyl, naphthyridinyl, oxadiazolyl, 2-oxoazepinyl, oxazolyl, oxiranyl, 1-oxidopyridinyl, 1-oxidopyrimidinyl, 1-oxidopyrazinyl, 1-oxidopyridazinyl, 1-phenyl-1H-pyrrolyl, phenazinyl, phenothiazinyl, phenoxazinyl, phthalazinyl, pteridinyl, purinyl, pyrrolyl, pyrazolyl, pyridinyl, pyrazinyl, pyrimidinyl, pyridazinyl, quinazolinyl, quinoxalinyl, quinolinyl, quinuclidinyl, isoquinolinyl, tetrahydroquinolinyl, thiazolyl, thiadiazolyl, triazolyl, tetrazolyl, triazinyl, and thiophenyl (i.e., thienyl). Unless stated otherwise specifically in the specification, a heteroaryl may be optionally substituted, for example, with halogen, amino, nitrile, nitro, hydroxyl, alkyl, alkenyl, alkynyl, haloalkyl, alkoxy, carboxyl, carboxylate, aryl, cycloalkyl, heterocycloalkyl, heteroaryl, and the like. In some embodiments, the heteroaryl is optionally substituted with halogen, methyl, ethyl, —CN, —COOH, COOMe, —CF₃, —OH, —OMe, —NH₂, or —NO₂. In some embodiments, the heteroaryl is optionally substituted with halogen, methyl, ethyl, —CN, —CF₃, —OH, or —OMe. In some embodiments, the heteroaryl is optionally substituted with halogen.

The term “substituted” refers to moieties having substituents replacing a hydrogen on one or more carbons or substitutable heteroatoms, e.g., NH, of the structure. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds. For purposes of this disclosure, the heteroatoms such as nitrogen may have hydrogen substituents and/or any permissible substituents of organic compounds described herein which satisfy the valences of the heteroatoms.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, and heterocycle, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, and heterocycle, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R b-C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

As used in the specification and claims, the singular form “a”, “an” and “the” includes plural references unless the context clearly dictates otherwise.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material, Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a disease or condition symptoms, preventing additional symptoms, ameliorating or preventing the underlying causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### B. Compounds of the Disclosure

In an aspect, the present disclosure provides a compound represented by Formula (A):

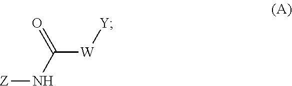

or a pharmaceutically acceptable salt thereof, wherein:
Z is selected from optionally substituted 3- to 12-membered heterocycle and optionally substituted C₃-C₁₂ carbocycle, wherein each is optionally substituted with one or more substituents independently selected at each occurrence from —N(R¹⁰)₂, halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₁-C₁₀ alkyl, C₃₋₁₂ carbocycle, 3- to 12-membered heterocycle;
   wherein the C₁-C₁₀ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R¹⁰ is selected from optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
W is selected from optionally substituted 5- to 8-membered heterocycle and optionally substituted C₃-C₈ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; and
Y is selected from optionally substituted 5- to 8-membered heterocycle and optionally substituted C₃-C₈ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.

In certain embodiments, the compound or salt of Formula (A) is represented by Formula (I).

In certain embodiments, the disclosure provides a compound represented by Formula (I):

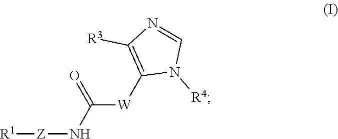

or a pharmaceutically acceptable salt thereof, wherein:
R¹ is selected from:
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from one or more halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   substituted C₁-C₆ alkyl, wherein the C₁-C₆ alkyl is substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   optionally substituted 3 to 8-membered heterocycle; wherein the 3- to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
Z is selected from optionally substituted 3- to 12-membered heterocycle and optionally substituted C₃-C₁₂ carbocycle, wherein the each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl; and
W is selected from optionally substituted thiazole, wherein the thiazole is optionally substituted with one or more substituents independently selected at each occurrence from one or more halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, and 3- to 12-membered heterocycle.

In certain embodiments, for a compound or salt of Formula (A) or Formula (I), Z is selected from optionally substituted phenyl and optionally substituted pyridine.

In certain embodiments, for a compound or salt of Formula (A) or Formula (I), the optional substituents of the optionally substituted phenyl of Z are selected from one or more halogen, —OH, —CN, —NO₂, —NH₂, oxo, ═S, C₁₋₁₀ alkyl, —C₁₋₁₀ haloalkyl, and —O—C₁₋₁₀ alkyl.

In certain embodiments, for a compound or salt of Formula (A) or Formula (I), the optional substituents of the optionally substituted phenyl of Z are selected from one or more halogens.

In certain embodiments, for a compound or salt of Formula (A) or Formula (I), Z is selected from

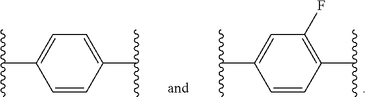 [SMILES: *c1ccc(*)cc1 ; *c1ccc(*)c(F)c1]

In certain embodiments, for a compound or salt of Formula (A) or Formula (I), Z is selected from pyridine,

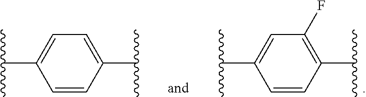 [SMILES: *c1ccc(*)cc1 ; *c1ccc(*)c(F)c1]

In certain embodiments, for a compound or salt of Formula (A) or Formula (I), W is unsubstituted thiazole.

In certain embodiments, for a compound or salt of Formula (A) or Formula (I), W is

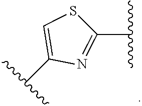 [SMILES: *c1csc(*)n1]

In certain embodiments, a compound represented by Formula (I) has a structure illustrated as:

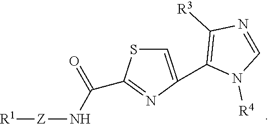

In certain embodiments, a compound represented by Formula (I) has a structure illustrated as:

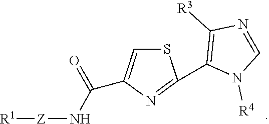

In certain embodiments, for a compound or salt of Formula (A) or Formula (I), R⁴ is selected from optionally substituted C₁-C₆ alkyl and optionally substituted C₃₋₆ carbocycle.

In certain embodiments, for a compound or salt of Formula (A) or Formula (I), R⁴ is selected from unsubstituted C₁-C₆ alkyl and unsubstituted C₃₋₆ carbocycle.

In certain embodiments, for a compound or salt of Formula (A) or Formula (I), R⁴ is selected from unsubstituted C₁-C₆ alkyl and unsubstituted C₃₋₆ cycloalkyl.

In certain embodiments, for a compound or salt of Formula (A) or Formula (I), R⁴ is selected from

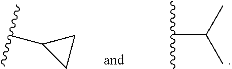

In certain embodiments, for a compound or salt of Formula (A) or Formula (I), R³ is selected from optionally substituted C₃₋₆ carbocycle.

In certain embodiments, for a compound or salt of Formula (A) or Formula (I), R³ is optionally substituted phenyl.

In certain embodiments, for a compound or salt of Formula (A) or Formula (I), the optional substituents of phenyl of R³ are selected from halogen and —C₁₋₁₀ haloalkyl.

In certain embodiments, for a compound or salt of Formula (A) or Formula (I), R³ is

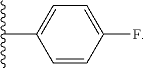 [SMILES: *c1ccc(F)cc1]

In certain embodiments, for a compound or salt of Formula (A) or Formula (I), R¹ is an optionally substituted 6-membered heterocycle.

In certain embodiments, for a compound or salt of Formula (A) or Formula (I), R¹ is an optionally substituted piperazine.

In certain embodiments, for a compound or salt of Formula (A) or Formula (I), the optional substituents of the optionally substituted piperazine for R¹ are selected from C₁₋₆ alkyl.

In certain embodiments, for a compound or salt of Formula (A) or Formula (I), R¹ is

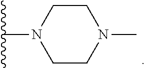 [SMILES: *N1CCN(C)CC1]

In some embodiments, Formula (A) or Formula (I), R¹ is monocyclic. In some embodiments, R¹ is bicyclic. In some embodiments, R¹ is a fused bicyclic group. In some embodiments, R¹ is a bridged bicyclic group. In some embodiments, R¹ is optionally substituted 5 membered heterocycle. In some embodiments, R¹ is optionally substituted heteroaryl. In some embodiments, R¹ is optionally substituted heterocycloalkyl. In some embodiments, R¹ contains 0-3 nitrogen and 0-1 oxygen atoms on the ring. In some embodiments, R¹ contains 1-2 nitrogen and 0-1 oxygen atoms on the ring. In some embodiments, R¹ contains 1-2 ring nitrogen atoms. In some embodiments, R¹ contains 2 ring nitrogen atoms. In some embodiments, R¹ contains 1 ring nitrogen atom.

In certain embodiments, the compound or salt of Formula (A) is represented by Formula (II).

In another aspect, the present disclosure provides a compound represented by Formula (II):

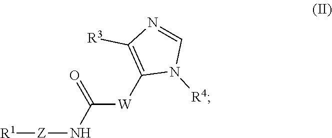

or a pharmaceutically acceptable salt thereof, wherein:
R¹ is selected from:
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   substituted C₁-C₆ alkyl, wherein the C₁-C₆ alkyl is substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   optionally substituted 3 to 10-membered heterocycle; wherein the 3- to 8-membered heterocycle is optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, and —NO₂;
R³ is selected from optionally substituted C₁-C₆ alkyl, optionally substituted 3- to 10-membered heterocycle and optionally substituted C₃₋₁₀ carbocycle, wherein each is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl; and
W is selected from optionally substituted 5- to 8-membered heteroaryl, wherein the optionally substituted 5- to 8-membered heteroaryl is optionally substituted with one or more substituents independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle.

In certain embodiments, for a compound or salt of Formula (A) or Formula (II), R⁴ is selected from optionally substituted C₁-C₆ alkyl and optionally substituted C₃₋₆ carbocycle.

In certain embodiments, for a compound or salt of Formula (A) or Formula (II), the optional substituents of optionally substituted C₁-C₆ alkyl and optionally substituted C₃₋₆ carbocycle are independently selected at each occurrence from one or more halogen, —OH, —CN, —NO₂, —NH₂, oxo, ═S, —O—C₁₋₁₀ alkyl, —C₁₋₁₀ haloalkyl, —O—C₁₋₁₀ alkyl.

In certain embodiments, for a compound or salt of Formula (A) or Formula (II), R⁴ is selected from unsubstituted C₁-C₆ alkyl and unsubstituted C₃₋₆ carbocycle.

In certain embodiments, for a compound or salt of Formula (A) or Formula (II), R⁴ is selected from unsubstituted C₁-C₆ alkyl and unsubstituted C₃₋₆ cycloalkyl.

In certain embodiments, for a compound or salt of Formula (II), Formula (II) is represented by Formula (IIA):

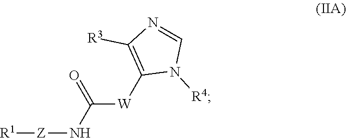

wherein R⁴ is selected from

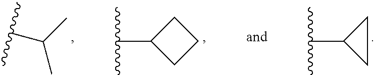

or a pharmaceutically acceptable salt thereof.

In certain embodiments, for a compound or salt of Formula (A), Formula (II) or Formula (IIA), R³ is selected from optionally substituted C₃₋₆ carbocycle.

In certain embodiments, for a compound or salt of Formula (A), Formula (II) or Formula (IIA), R³ is optionally substituted phenyl.

In certain embodiments, for a compound or salt of Formula (A), Formula (II) or Formula (IIA), the optional substituents of phenyl of R³ are selected from halogen and —C₁₋₁₀ haloalkyl.

In certain embodiments, for a compound or salt of Formula (A), Formula (II) or Formula (IIA), R³ is

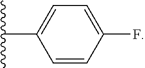 [SMILES: *c1ccc(F)cc1]

In certain embodiments, for a compound or salt of Formula (A), Formula (II) or Formula (IIA), W is selected from imidazole and oxazole.

In certain embodiments, for a compound or salt of Formula (A), Formula (II) or Formula (IIA), W is selected from

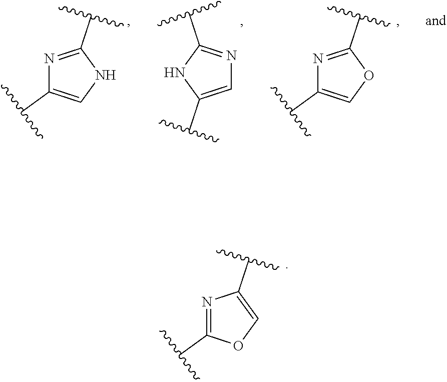 [SMILES: *c1c[nH]c(*)n1 ; *c1cnc(*)[nH]1 ; *c1coc(*)n1 ; *c1coc(*)n1]

In certain embodiments, for a compound or salt of Formula (A), Formula (II) or Formula (IIA), W is

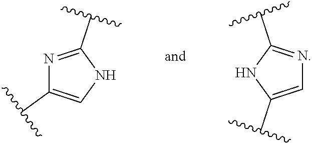 [SMILES: *c1c[nH]c(*)n1 ; "and" ; *c1cnc(*)[nH]1]

In certain embodiments, for a compound or salt of Formula (A), Formula (II) or Formula (IIA), W is

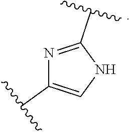 [SMILES: *c1c[nH]c(*)n1]

In certain embodiments, for a compound or salt of Formula (A), Formula (II) or Formula (IIA), W is

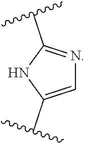 [SMILES: *c1cnc(*)[nH]1]

In certain embodiments, for a compound or salt of Formula (A), Formula (II) or Formula (IIA), Z is selected from optionally substituted phenyl and optionally substituted pyridine.

In certain embodiments, for a compound or salt of Formula (A), Formula (II) or Formula (IIA), the optional substituents of the optionally substituted phenyl of Z are selected from one or more halogen, —OH, —CN, —NO₂, —NH₂, oxo, ═S, C₁₋₁₀ alkyl, —C₁₋₁₀ haloalkyl, and —O—C₁₋₁₀ alkyl.

In certain embodiments, for a compound or salt of Formula (A), Formula (II) or Formula (IIA), the optional substituents of the optionally substituted phenyl of Z are selected from one or more halogens and C₁₋₁₀ alkyl.

In certain embodiments, for a compound or salt of Formula (A), Formula (II) or Formula (IIA), Z is selected from

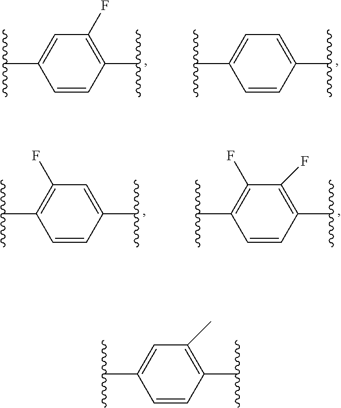

and pyridine.

In certain embodiments, for a compound or salt of Formula (A), Formula (II) or Formula (IIA), Z is selected from

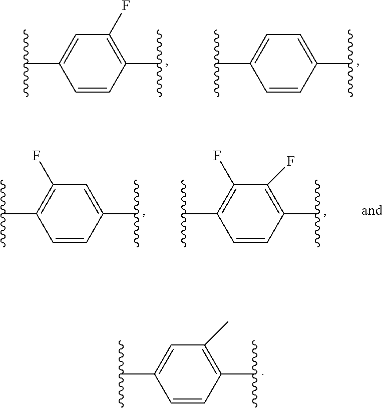

In certain embodiments, for a compound or salt of Formula (A), Formula (II) or Formula (IIA), the optional substituents of the optionally substituted phenyl of Z are selected from one or more halogens.

In certain embodiments, for a compound or salt of Formula (A), Formula (II) or Formula (IIA), Z is selected from

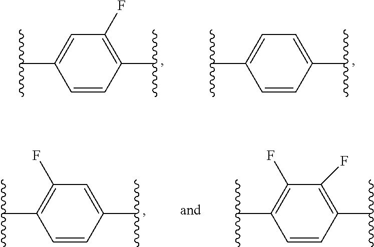 [SMILES: *c1ccc(*)c(F)c1 ; *c1ccc(*)cc1 ; *c1ccc(*)c(F)c1 ; *c1ccc(*)c(F)c1F]

In certain embodiments, for a compound or salt of Formula (A), Formula (II) or Formula (IIA), R¹ is an optionally substituted 6- to 10-membered heterocycle.

In certain embodiments, for a compound or salt of Formula (A), Formula (II) or Formula (IIA), R¹ is an optionally substituted 6- to 10-membered heterocycloalkyl.

In certain embodiments, for a compound or salt of Formula (A), Formula (II) or Formula (IIA), the optional substituents of the optionally substituted 6- to 9-membered heterocycloalkyl for R¹ are selected from C₁₋₆ alkyl.

In certain embodiments, for a compound or salt of Formula (A), Formula (II) or Formula (IIA), the 6- to 9-membered heterocycloalkyl is a spiro heterocycloalkyl.

In certain embodiments, for a compound or salt of Formula (A), Formula (II) or Formula (IIA), R¹ is selected from optionally substituted piperazine, optionally substituted diazabicyclo [3.2.1]octane, optionally substituted diazabicyclo[3.1.1]heptane, optionally substituted diazaspiro[3.5]nonane, and optionally substituted diazaspiro[3.3]heptane.

In certain embodiments, for a compound or salt of Formula (A), Formula (II) or Formula (IIA), when Z is phenyl, R¹ is substituted piperazine.

In some embodiments, R¹ is substituted with one or more substituents selected from halogen, —OH, —CN, —NO₂, —NH₂, oxo, ═S, —C₁₋₁₀ haloalkyl, —C₁₋₁₀ heteroalkyl, —O—C₁₋₁₀ alkyl, C₂₋₁₀ alkenyl, C₂₋₁₀ alkynyl, C₃₋₁₂ carbocycle, and 3- to 12-membered heterocycle.

In certain embodiments, for a compound or salt of Formula (A), Formula (II) or Formula (IIA), R¹ is substituted piperazine.

In certain embodiments, for a compound or salt of Formula (A), Formula (II) or Formula (IIA), R¹ is

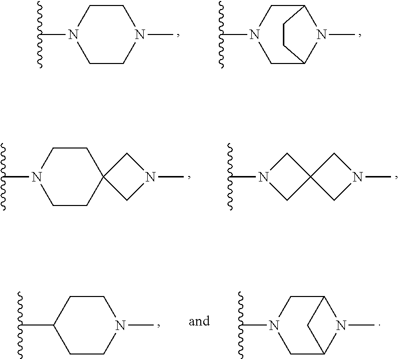

In certain embodiments, for a compound or salt of Formula (A), Formula (II) or Formula (IIA), W is selected from 5-membered heteroaryl. In some cases, W is selected from unsubstituted 5-membered heteroaryl. In some cases, W is selected from imidazole, oxazole, and thiazole. In some cases, W is selected from imidazole, oxazole, and thiazole, each of which are unsubstituted. In some cases, W is selected from

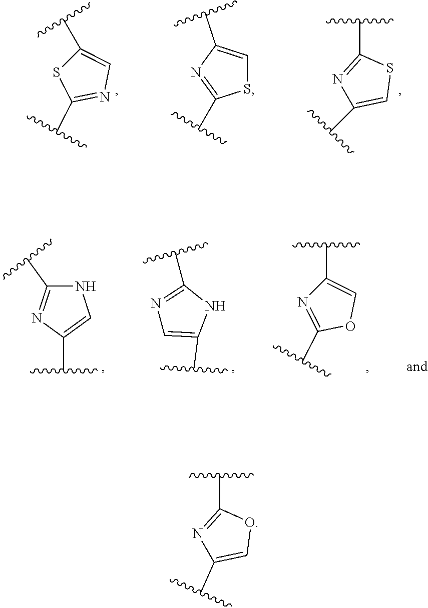

In some cases, W is selected from

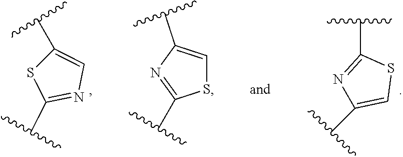 [SMILES: *c1cnc(*)s1 ; *c1csc(*)n1 ; "and" ; *c1csc(*)n1]

In some cases, W is selected from

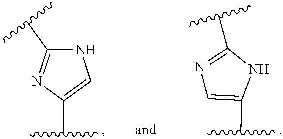 [SMILES: *c1c[nH]c(*)n1 ; *c1cnc(*)[nH]1]

In some cases, W is selected from

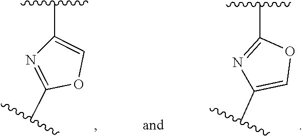 [SMILES: *c1coc(*)n1 ; *c1coc(*)n1]

In certain embodiments, for a compound or salt of Formula (A), Formula (II) or Formula (IIA), for R¹, the heterocycle has at least one nitrogen atom, phosphorous atom, or oxygen atom. In some cases, for R¹, the heterocycle has at least one nitrogen atom. In some cases, for R¹, the heterocycle has at least two nitrogen atoms. In some cases, for R¹, the heterocycle has at most two nitrogen atoms. In some cases, for R¹, the heterocycle has at most one nitrogen atom. In some cases, for R¹, the heterocycle has at one oxygen atom. In some cases, for R¹, the heterocycle is a spirocycle. In some cases, for R¹, the heterocycle is a bridged heterocycle. In some cases, for R¹, the heterocycle is unsaturated. In some cases, R¹, the heterocycle is saturated. In some cases, R¹ is selected from

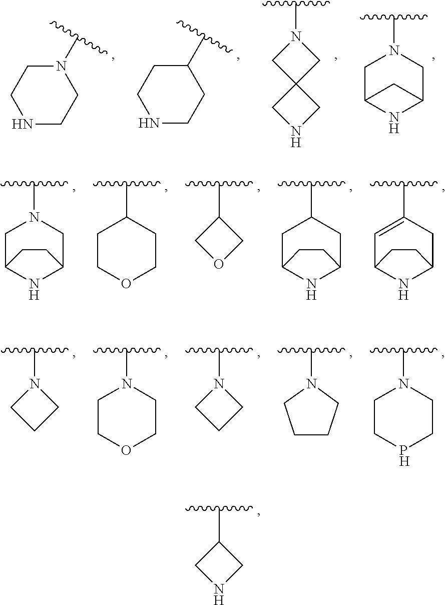

any of which are optionally substituted. In some cases, the optional substituents are independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂. In some cases, the optional substituents are independently selected at each occurrence from one or more halogen, —OH, —CN, —NO₂, —NH₂, oxo, ═S, —S(O₂)NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂.

In certain embodiments, for a compound or salt of Formula (A), Formula (II) or Formula (IIA), the compound is selected from

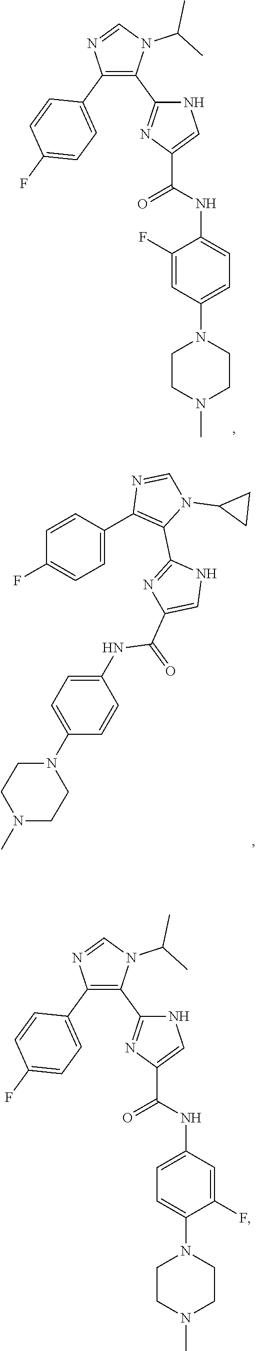 [SMILES: CC(C)n1cnc(-c2ccc(F)cc2)c1-c1nc(C(=O)Nc2ccc(N3CCN(C)CC3)cc2F)c[nH]1 ; CN1CCN(c2ccc(NC(=O)c3c[nH]c(-c4c(-c5ccc(F)cc5)ncn4C4CC4)n3)cc2)CC1 ; CC(C)n1cnc(-c2ccc(F)cc2)c1-c1nc(C(=O)Nc2ccc(N3CCN(C)CC3)c(F)c2)c[nH]1]

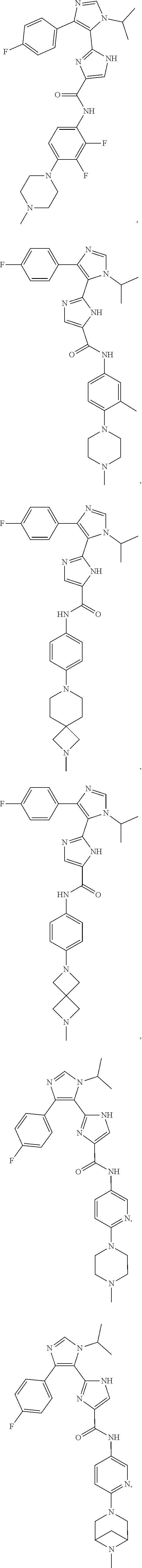

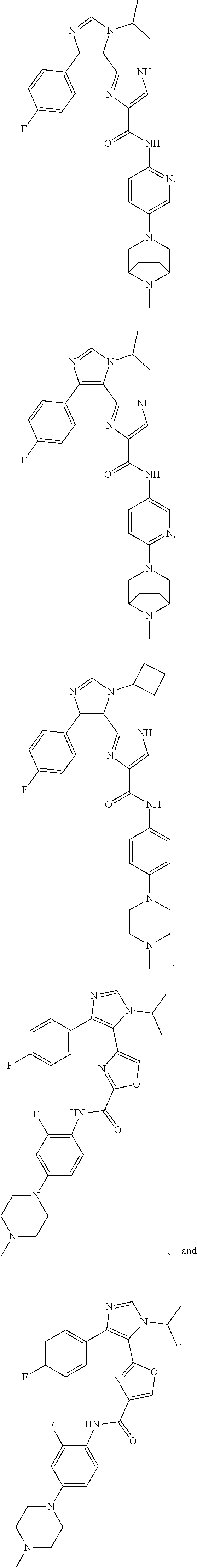

In another aspect, the present disclosure provides a compound represented by Formula (AA):

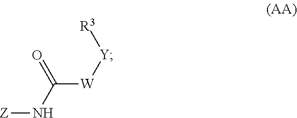

or a pharmaceutically acceptable salt thereof, wherein:
Z is selected from optionally substituted 3- to 12-membered heteroaryl and optionally substituted C₆-C₁₂ carbocycle, wherein each is optionally substituted with one or more substituents independently selected at each occurrence from R¹;
R¹ is selected from
   —N(R¹⁰)₂, halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₁-C₁₀ alkyl, C₃₋₁₂ carbocycle, and 3- to 12-membered heterocycle;
   wherein the C₁-C₁₀ alkyl is optionally substituted with one or more substituents independently selected at each occurrence from halogen, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, 3- to 12-membered heterocycle;
   wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from one or more hydroxy, halogen, oxo, —C₁₋₁₀ haloalkyl, —NH₂, —CN, and —NO₂;
R¹⁰ is selected from optionally substituted C₁-C₆ alkyl, wherein the C₁-C₆ alkyl is optionally substituted with one or more substituents independently selected at each occurrence from halogen, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
W is selected from optionally substituted 5- to 8-membered heterocycle, wherein the 5- to 8-membered heterocycle has at least 2 heteroatoms; and wherein the 5- to 8-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, wherein the C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; wherein when W is imidazole and Z is phenyl and R¹ is piperidine, piperidine is substituted;
Y is selected from optionally substituted 5- to 10-membered heterocycle and optionally substituted C₃-C₈ carbocycle, wherein each is optionally substituted with one or more substituents independently selected at each occurrence from R⁴;
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.
each R⁴ is independently selected from halogen, —OH, —CN, —NO₂, —NH₂, —N(H)C₁-C₆ alkyl, —N(C₁-C₆ alkyl)₂, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl; and
wherein when W is imidazole, Y is imidazole, and Z is phenyl substituted with piperazine, the piperazine is substituted.

In some embodiments, for a compound or salt of Formula (AA) or a pharmaceutically acceptable salt thereof,
Z is selected from optionally substituted 3- to 12-membered heteroaryl and optionally substituted C₆-C₁₂ carbocycle, wherein each is optionally substituted with one or more substituents independently selected at each occurrence from R¹;
R¹ is selected from
   —N(R¹⁰)₂, halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₁-C₁₀ alkyl, C₃₋₁₂ carbocycle, and 3- to 12-membered heterocycle, wherein the C₁-C₁₀ alkyl, C₃₋₁₂ carbocycle, and 3- to 12-membered heterocycle are each optionally substituted;
      R¹⁰ is selected from optionally substituted C₁-C₆ alkyl;
W is selected from optionally substituted 5- to 8-membered heterocycle, wherein the 5- to 8-membered heterocycle has at least 2 heteroatoms; wherein when W is imidazole and Z is phenyl and R¹ is piperidine, piperidine is substituted;
Y is selected from optionally substituted 5- to 10-membered heterocycle and optionally substituted C₃-C₈ carbocycle, wherein each is optionally substituted with one or more substituents independently selected at each occurrence from R⁴;
R³ is selected from an optionally substituted C₃₋₁₂ carbocycle;
each R⁴ is independently selected from halogen, —OH, —CN, —NO₂, —NH₂, —N(H)C₁-C₆ alkyl, —N(C₁-C₆ alkyl)₂, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted; and
wherein when W is imidazole, Y is imidazole, and Z is phenyl substituted with piperazine, the piperazine is substituted.

In some embodiments, for a compound or salt of Formula (AA), Y is selected from an optionally 5-membered heterocycle. In some cases, Y is selected from an optionally 5-membered heteroaryl. In some cases, the heterocycle of Y has at least two heteroatoms. In some cases, the heterocycle of Y has at most two heteroatoms. In some cases, the heterocycle of Y has at least one nitrogen atom. In some cases, the heterocycle of Y has at least one oxygen atom. In some cases, the heterocycle of Y has at one oxygen atom and one nitrogen atom. In some cases, the heterocycle of Y has two nitrogen atoms. In some cases, Y is selected from imidazole and isoxazole, each of which are optionally substituted. In some cases, Y is substituted with one R⁴. In some cases, Y and W are bound together through a carbon-carbon bond. In some cases, R³ of Y is ortho position with respect to W. In some cases, R⁴ of Y is ortho position with respect to W. In some cases, R³ is bound to a carbon atom of the heterocycle of Y. In some cases, R⁴ is bound to a carbon atom of the heterocycle of Y. In some cases, R⁴ is bound to a heteroatom of the heterocycle of Y. In some cases, R⁴ is bound to a nitrogen atom of the heterocycle of Y. In some cases, R³ is bound to a carbon atom and R⁴ is bond to a carbon atom on the heterocycle of Y, and the carbon atoms are separated by one or more carbon atoms. In some cases, R³ is bound to a carbon atom and R⁴ is bond to a nitrogen atom on the heterocycle of Y, and the carbon and nitrogen atoms are separated by one or more carbon atoms.

In some embodiments, for a compound or salt of Formula (AA), In some cases, Y is selected from an 8- to 10-membered heterocycle. In some cases, the heterocycle includes at least three heteroatoms. In some cases, the heterocycle includes at least three heteroatoms selected from nitrogen and oxygen. In some cases, the heterocycle is unsaturated. In some cases, Y is bicyclic. In some cases, Y is monocyclic. In some cases, Y is

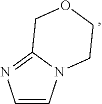

which is optionally substituted. In some cases, Y is

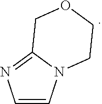

In some embodiments, for a compound or salt of Formula (AA), Formula (AA) is represented by Formula (B)

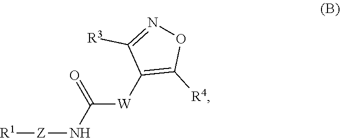

### Formula (C)

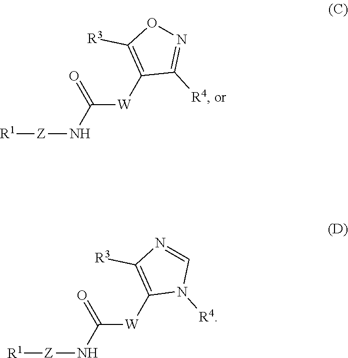

In some embodiments, for a compound or salt of Formula (AA), Formula (AA) is represented by Formula (B):

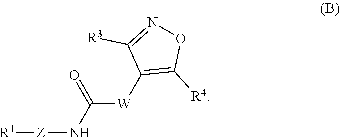

In some embodiments, for a compound or salt of Formula (AA), is represented by Formula (C):

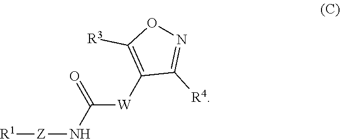

In some embodiments, for a compound or salt of Formula (AA), Formula (AA) is represented by Formula (D):

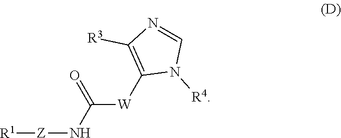

In some embodiments, for a compound or salt of Formula (AA), Formula (B), Formula (C), or Formula (D), R¹ is optionally substituted C₁-C₁₀ alkyl. In some cases, R¹ is substituted C₁-C₆ alkyl. In some cases, R¹ is C₁-C₁₀ alkyl is optionally substituted with one or more substituents independently selected at each occurrence from halogen, —CN, —NO₂, —NH₂, —C₁₋₁₀ haloalkyl, —O—C₁₋₁₀ alkyl, C₃₋₁₂ carbocycle, and 3- to 12-membered heterocycle.

In some embodiments, for a compound or salt of Formula (AA), Formula (B), Formula (C), or Formula (D), R¹ is an optionally substituted 3 to 10-membered heterocycle. In some cases, R¹ is an optionally substituted 4- to 8-membered heterocycle. In some cases, R¹ is an optionally substituted 4-membered heterocycle. In some cases, R¹ is an optionally substituted 6-membered heterocycle. In some cases, when R¹ is piperazine, the piperazine is substituted. In some cases, R¹ is not unsubstituted piperazine. In some cases, R¹ is a substituted 3 to 10-membered heterocycle.

In some embodiments, for a compound or salt of Formula (AA), Formula (B), Formula (C), or Formula (D), for R¹, the heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, —O—C₁₋₁₀ alkyl, and —NO₂.

In some embodiments, for a compound or salt of Formula (AA), Formula (B), Formula (C), or Formula (D), for R¹, the 3 to 10-membered heterocycle is optionally substituted with one or more substituents independently selected at each occurrence from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (AA), Formula (B), Formula (C), or Formula (D), for R¹, the 3 to 10-membered heterocycle is substituted with one or more substituents independently selected at each occurrence from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (AA), Formula (B), Formula (C), or Formula (D), for R¹, the heterocycle has at least one nitrogen atom, phosphorous atom, or oxygen atom. In some cases, for R¹, the heterocycle has at least one nitrogen atom. In some cases, for R¹, the heterocycle has at least two nitrogen atoms. In some cases, for R¹, the heterocycle has at most two nitrogen atoms. In some cases, for R¹, the heterocycle has at most one nitrogen atom. In some cases, for R¹, the heterocycle has two nitrogen atoms. In some cases, for R¹, the heterocycle is a spiro-heterocycle. In some cases, for R¹, the heterocycle is a bridged heterocycle. In some cases, for R¹, the heterocycle is unsaturated. In some cases, for R¹, the heterocycle is saturated.

In some embodiments, for a compound or salt of Formula (AA), Formula (B), Formula (C), or Formula (D), R¹ is selected from

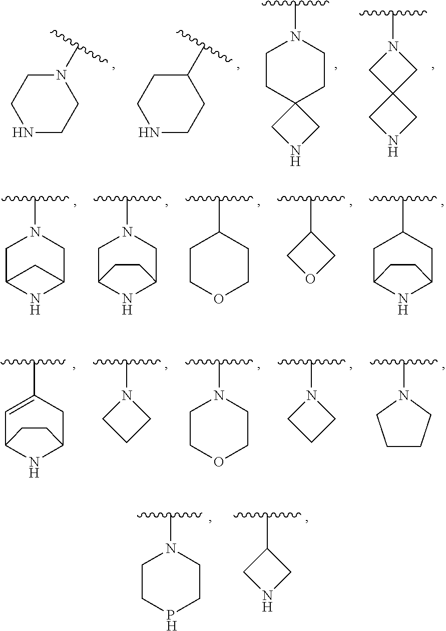

any of which are optionally substituted.

In some embodiments, for a compound or salt of Formula (AA), Formula (B), Formula (C), or Formula (D), R¹ is selected from

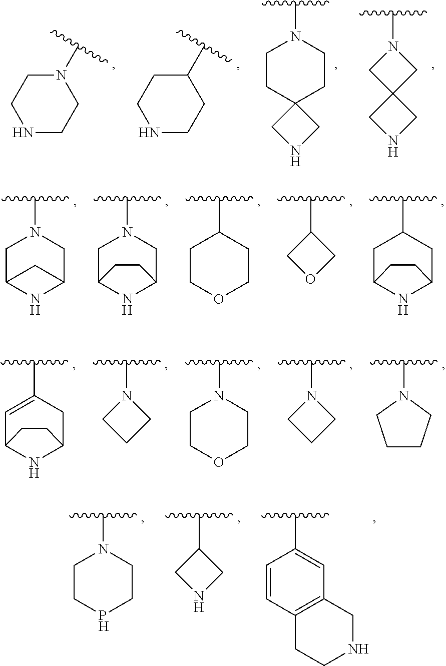

any of which are optionally substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (AA), Formula (B), Formula (C), or Formula (D), R¹ is selected from

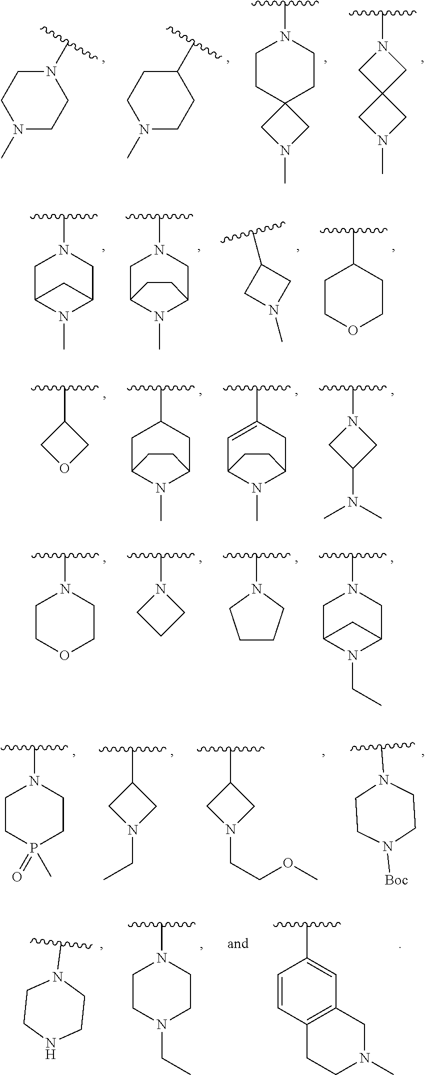

In some embodiments, for a compound or salt of Formula (AA), Formula (B), Formula (C), or Formula (D), R¹ is selected from

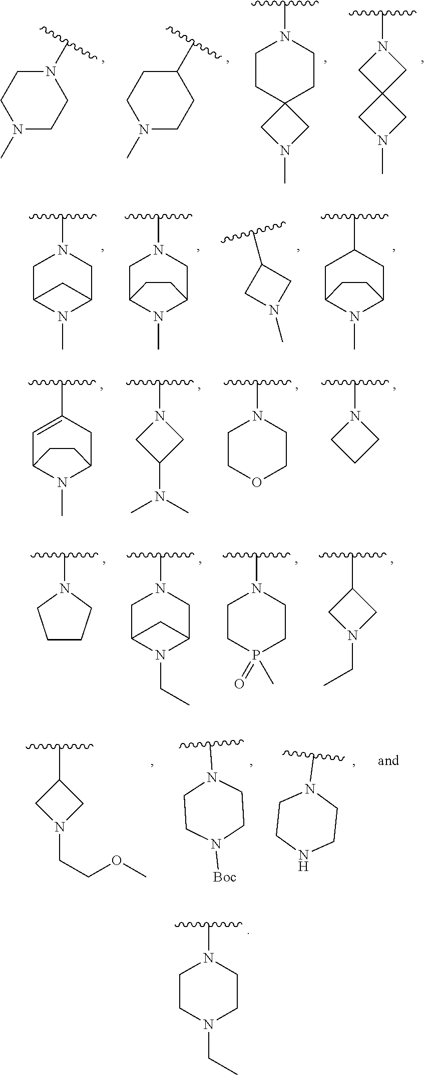

In some embodiments, for a compound or salt of Formula (AA), Formula (B), Formula (C), or Formula (D), R¹ is selected from

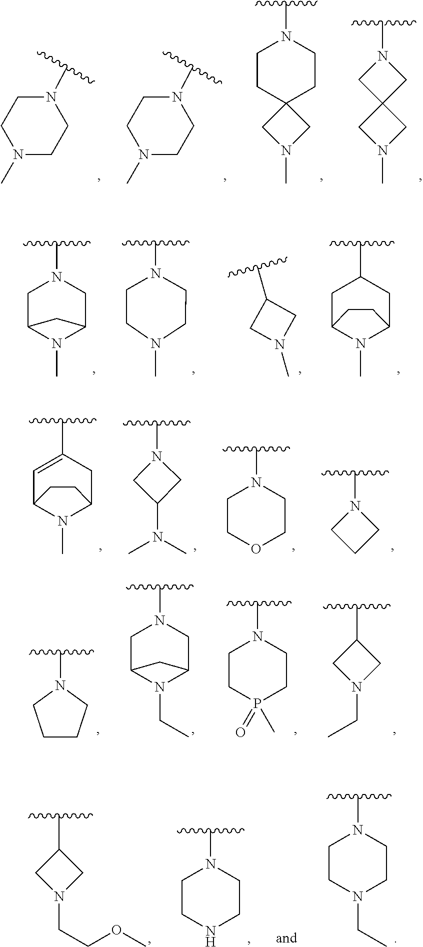

In some embodiments, for a compound or salt of Formula (AA), Formula (B), Formula (C), or Formula (D), R¹ is selected from

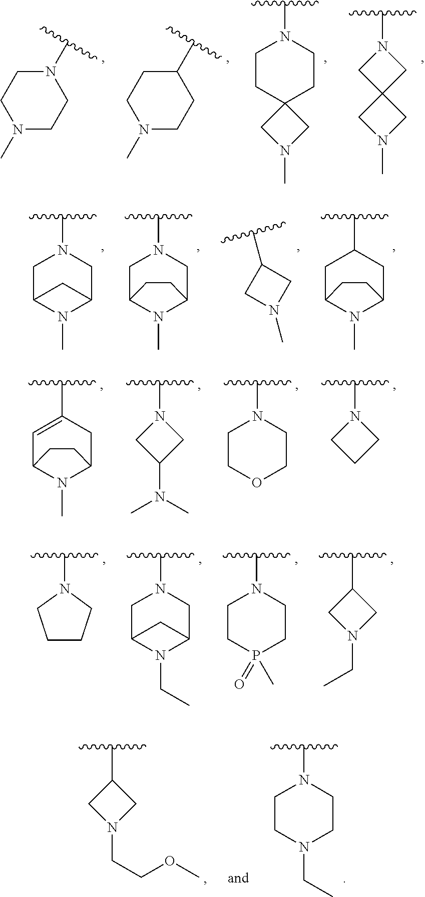

In some embodiments, for a compound or salt of Formula (AA), Formula (B), Formula (C), or Formula (D), R¹ is selected from

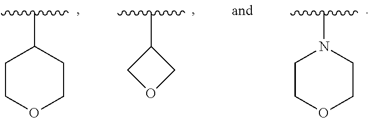 [SMILES: *C1CCOCC1 ; *C1COC1 ; *N1CCOCC1]

In some embodiments, for a compound or salt of Formula (AA), Formula (B), or Formula (C), each R⁴ is selected at each occurrence from halogen, —OH, —CN, —NO₂, —NH₂, —N(H)C₁-C₆ alkyl, —N(C₁-C₆ alkyl)₂, oxo, —C₁₋₁₀ haloalkyl, —O—C₁₋₁₀ alkyl, C₁₋₁₀ alkyl, C₃₋₁₂ carbocycle, and 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl. In some cases, each R⁴ is selected at each occurrence from unsubstituted C₁₋₁₀ alkyl, unsubstituted 3- to 6-membered heterocycle, and optionally substituted C₃-C₆ carbocycle, wherein the optional substituents are independently selected from one or more halogen —C₁₋₁₀ haloalkyl.

In some embodiments, for a compound or salt of Formula (D), each R⁴ is selected at each occurrence from —O—C₁₋₁₀ alkyl, C₁₋₁₀ alkyl, C₃₋₁₂ carbocycle, and 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl.

In some embodiments, for a compound or salt of Formula (AA), Formula (B), Formula (C), or Formula (D), each R⁴ is selected at each occurrence from unsubstituted C₁₋₁₀ alkyl, unsubstituted 3- to 6-membered heterocycle, and optionally substituted C₃-C₆ carbocycle, wherein the optional substituents are independently selected from one or more halogen —C₁₋₁₀ haloalkyl.

In some embodiments, for a compound or salt of Formula (AA), Formula (B), Formula (C), or Formula (D), each R⁴ is selected at each occurrence from C₁₋₁₀ alkyl, unsubstituted 4-membered heterocycle, and optionally substituted C₃-C₅ carbocycle, wherein the optional substituents are independently selected from one or more halogen —C₁₋₁₀ haloalkyl. In some cases, R⁴ is selected from a C₁₋₁₀ alkyl. In some cases, R⁴ is selected from a 4-membered heterocycle. In some cases, R⁴ is a 4-membered heterocycle. In some cases, R⁴ is a saturated 4-membered heterocycle.

In some embodiments, for a compound or salt of Formula (AA), Formula (B), Formula (C), or Formula (D), R⁴ is selected from

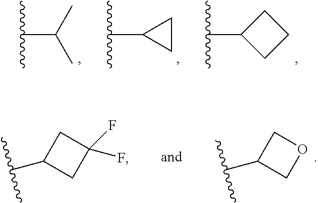

In some cases, R⁴ is selected from

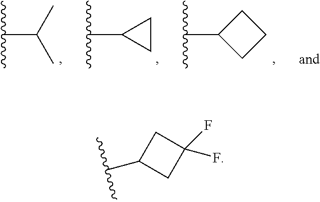

In some cases, R⁴ is selected from

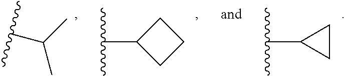

In some embodiments, for a compound or salt of Formula (AA), Formula (B), Formula (C), or Formula (D), R³ is an optionally substituted phenyl. In some cases, the optional substituents of phenyl of R³ are selected from halogen and —C₁₋₁₀ haloalkyl. In some cases, R³ is substituted phenyl. In some cases, R³ is selected from

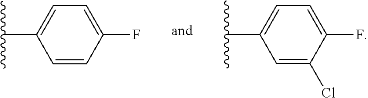 [SMILES: *c1ccc(F)cc1 ; *c1ccc(F)c(Cl)c1]

In some cases, R³ is

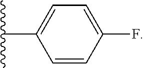 [SMILES: *c1ccc(F)cc1]

In some embodiments, or a compound or salt of Formula (AA), Formula (B), Formula (C), or Formula (D), the heterocycle of W has at least two heteroatoms. In some cases, the heterocycle of W has at least two heteroatoms selected from nitrogen, oxygen, and sulfur. In some cases, W is selected from 5-membered heteroaryl. In some cases, W is selected from unsubstituted 5-membered heteroaryl. In some cases, W is selected from imidazole, oxazole, and thiazole. In some cases, W is selected from imidazole, oxazole, and thiazole, each of which are unsubstituted. In some cases, W is selected from

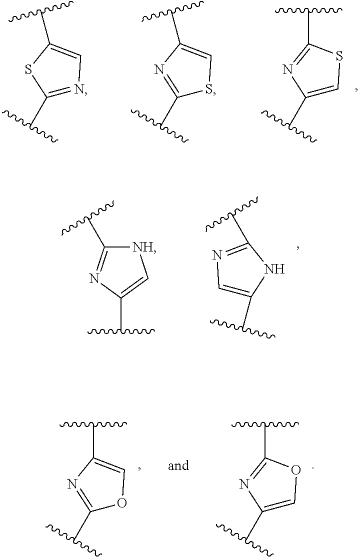

In some cases, W is selected from

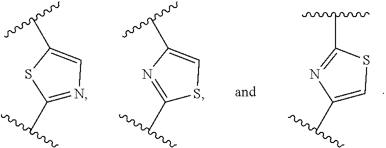 [SMILES: *c1cnc(*)s1 ; *c1csc(*)n1 ; *c1csc(*)n1 ; "."]

In some cases, W is selected from

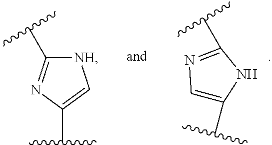 [SMILES: *c1c[nH]c(*)n1 ; *c1cnc(*)[nH]1]

In some cases, W is selected from

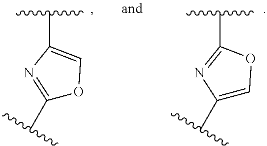 [SMILES: *c1coc(*)n1 ; *c1coc(*)n1]

In some embodiments, for a compound or salt of Formula (AA), Formula (B), Formula (C), or Formula (D), R¹ is selected from

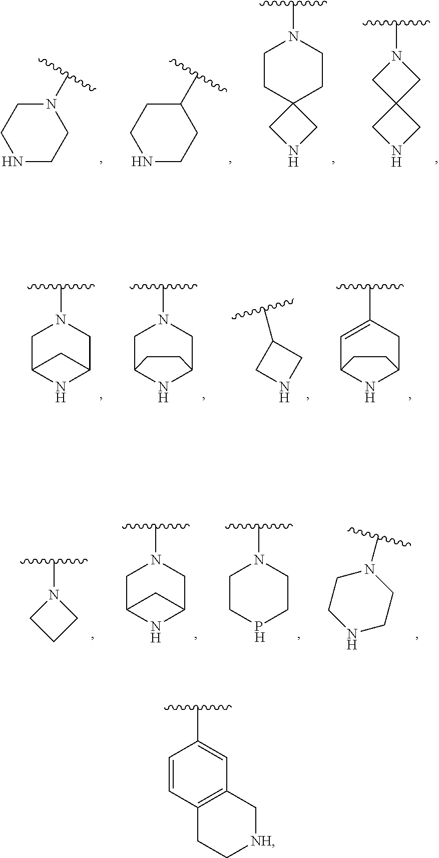

each of which are substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (AA), Formula (B), Formula (C), or Formula (D), R¹ is monocyclic. In some embodiments, R¹ is bicyclic. In some embodiments, R¹ is a fused bicyclic group. In some embodiments, R¹ is a bridged bicyclic group. In some embodiments, R¹ is optionally substituted 5 membered heterocycle. In some embodiments, R¹ is optionally substituted heteroaryl. In some embodiments, R¹ is optionally substituted heterocycloalkyl. In some embodiments, R¹ contains 0-3 nitrogen and 0-1 oxygen atoms on the ring. In some embodiments, R¹ contains 1-2 nitrogen and 0-1 oxygen atoms on the ring. In some embodiments, R¹ contains 1-2 ring nitrogen atoms. In some embodiments, R¹ contains 2 ring nitrogen atoms. In some embodiments, R¹ contains 1 ring nitrogen atom.

In some cases, for a compound or salt of Formula (AA), Formula (B), Formula (C), or Formula (D), R¹⁰ is selected from C₁-C₆ alkyl.

In some cases, for a compound or salt of Formula (AA), Formula (B), Formula (C), or Formula (D), when Z is phenyl substituted with piperazine, the piperazine is substituted. In some cases, when Z is phenyl substituted with piperazine and the phenyl is substituted with at least one more R¹, the piperazine is substituted.

In some cases, for a compound or salt of Formula (AA), Formula (B), Formula (C), or Formula (D), R¹ is substituted piperazine.

In some cases, for a compound or salt of Formula (AA), Formula (B), Formula (C), or Formula (D), R¹ is an optionally substituted 6- to 10-membered heterocycloalkyl. In some cases, the optional substituents of the optionally substituted 6- to 10-membered heterocycloalkyl for R¹ are selected from C₁₋₆ alkyl. In some cases, the 6- to 10-membered heterocycloalkyl is a spiro heterocycloalkyl. In some cases, R¹ is selected from optionally substituted piperazine, optionally substituted diazabicyclo [3.2.1]octane, optionally substituted diazabicyclo[3.1.1]heptane, optionally substituted diazaspiro[3.5]nonane, and optionally substituted diazaspiro[3.3]heptane. In some cases, the optional are selected from C₁₋₆ alkyl.

In another aspect, the present disclosure provides a compound represented by Formula (A*):

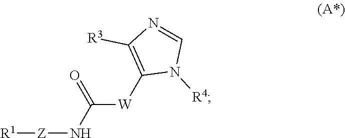

or a pharmaceutically acceptable salt thereof, wherein:
R¹ is selected from:
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
   optionally substituted 3 to 10-membered heterocycle; wherein the 3- to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
R³ is selected from optionally substituted C₁-C₆ alkyl, optionally substituted 3- to 10-membered heterocycle and optionally substituted C₃₋₁₀ carbocycle, wherein each is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
   hydrogen;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted 3- to 8-membered heterocycle, wherein the 3- to 8-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
W is selected from optionally substituted thiazole, wherein the thiazole is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

In some embodiments, for a compound or salt of Formula (A*),

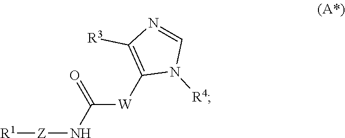

or a pharmaceutically acceptable salt thereof, wherein:
R¹ is selected from:
   —N(R⁵)₂, wherein R⁵ is selected from hydrogen, and optionally substituted C₁-C₆ alkyl;
   optionally substituted C₁-C₆ alkyl;
   optionally substituted 3 to 10-membered heterocycle;
R³ is selected from optionally substituted C₁-C₆ alkyl, optionally substituted 3- to 10-membered heterocycle and optionally substituted C₃_1₀ carbocycle;
R⁴ is selected from:
   hydrogen;
   optionally substituted C₁-C₆ alkyl;
   optionally substituted C₃_1₀ carbocycle;
   optionally substituted 3- to 8-membered heterocycle;
Z is selected from optionally substituted 3- to 12-membered heterocycle and optionally substituted C₃-C₁₂ carbocycle;
W is selected from optionally substituted thiazole.

In some embodiments, for a compound or salt of Formula (A*),
R¹ is selected from:
   optionally substituted 3 to 10-membered heterocycle;
R³ is selected from optionally substituted C₃_1₀ carbocycle;
R⁴ is selected from:
   optionally substituted C₁-C₆ alkyl;
   optionally substituted C₃_1₀ carbocycle;
   optionally substituted 3- to 8-membered heterocycle;
Z is selected from optionally substituted 3- to 12-membered heterocycle; and
W is selected from optionally substituted thiazole.

In some embodiments, Formula (A*) is represented by Formula (I).

In some embodiments, Formula (A*) is represented by Formula (II).

In some embodiments, for a compound or salt of Formula (A*), Z is selected from optionally substituted 3- to 12-membered heterocycle and optionally substituted C₃-C₁₂ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl. In some cases, for Z, the heterocycle includes at least one nitrogen atom. In some cases, Z is selected from optionally substituted phenyl and optionally substituted pyridine. In some cases, Z is selected from substituted phenyl and unsubstituted pyridine. In some cases, the phenyl of Z is optionally substituted with one or more substituents independently selected from halogen, —OH, —CN, —NO₂, —NH₂, oxo, ═S, C₁₋₁₀ alkyl, —C₁₋₁₀ haloalkyl, and —O—C₁₋₁₀ alkyl. In some cases, the phenyl of Z is optionally substituted with one or more substituents independently selected from halogen and C₁₋₁₀ alkyl. In some cases, Z is selected from

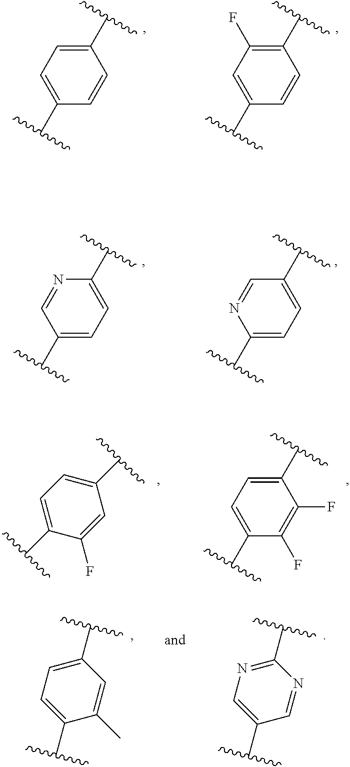

In some cases, Z is selected from

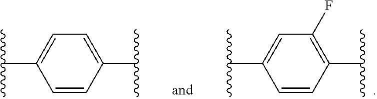 [SMILES: *c1ccc(*)cc1 ; *c1ccc(*)c(F)c1]

In some cases, Z is substituted phenyl. In some cases, Z is phenyl substituted with halogen.

In some embodiments, for a compound or salt of Formula (A*), R⁴ is selected from optionally substituted C₁-C₆ alkyl, optionally substituted C₃₋₆ carbocycle and optionally substituted 3- to 8-membered heterocycle. In some cases, R⁴ is selected from optionally substituted C₁-C₆ alkyl. In some cases, R⁴ is selected from optionally substituted C₃₋₆ carbocycle and optionally substituted 3- to 8-membered heterocycle. In some cases, R⁴ is selected from optionally substituted 3- to 8-membered heterocycle. In some cases, for R⁴, the optional substituents of the optionally substituted C₁-C₆ alkyl are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, for R⁴, the optional substituents of the optionally substituted C₃₋₆ carbocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, for R⁴, the optional substituents of the optionally substituted C₃₋₆ carbocycle are independently selected from halogen and —C₁₋₁₀ haloalkyl. In some cases, for R⁴, the optional substituents of the optionally substituted 3- to 8-membered heterocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, R⁴ is selected from unsubstituted C₁-C₆ alkyl, unsubstituted 3- to 6-membered heterocycle, and C₃₋₆ carbocycle optionally substituted with one or more halogens. In some cases, R⁴ is selected from

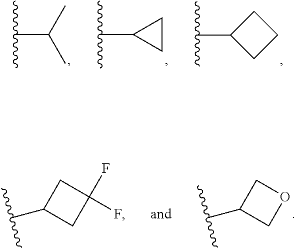

In some cases, R⁴ is selected from

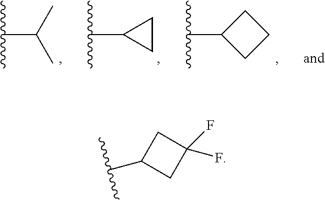

In some cases, R⁴ is selected from

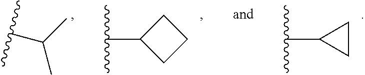

In some embodiments, for a compound or salt of Formula (A*), In some cases, R³ is selected from optionally substituted C₃₋₆ carbocycle. In some cases, R³ is selected from substituted C₃₋₆ carbocycle. In some cases, R³ is an optionally substituted phenyl. In some cases, R³ is selected from phenyl substituted with one more substituents selected from halogen and —C₁₋₁₀ haloalkyl. In some cases, the optional substituents of phenyl of R³ are selected from halogen and —C₁₋₁₀ haloalkyl. In some cases, R³ is selected from

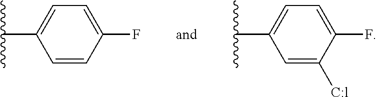

In some cases, R³ is

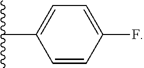 [SMILES: *c1ccc(F)cc1]

In some embodiments, for a compound or salt of Formula (A*), R¹ is substituted C₁-C₆alkyl.

In some embodiments, for a compound or salt of Formula (A*), R¹ is an optionally substituted 3- to 10-membered heterocycle. In some cases, R¹ is an optionally substituted 4-membered heterocycle. In some cases, for R¹, the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, —O—C₁₋₁₀ alkyl, and —NO₂. In some cases, for R¹, the 3 to 10-membered heterocycle is optionally substituted with one or more substituents independently selected at each occurrence from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (A*), R¹ is monocyclic. In some embodiments, R¹ is bicyclic. In some embodiments, R¹ is a fused bicyclic group. In some embodiments, R¹ is a bridged bicyclic group. In some embodiments, R¹ is optionally substituted 5 membered heterocycle. In some embodiments, R¹ is optionally substituted heteroaryl. In some embodiments, R¹ is optionally substituted heterocycloalkyl. In some embodiments, R¹ contains 0-3 nitrogen and 0-1 oxygen atoms on the ring. In some embodiments, R¹ contains 1-2 nitrogen and 0-1 oxygen atoms on the ring. In some embodiments, R¹ contains 1-2 ring nitrogen atoms. In some embodiments, R¹ contains 2 ring nitrogen atoms. In some embodiments, R¹ contains 1 ring nitrogen atom.

In some embodiments, for a compound or salt of Formula (A*), for R¹, the heterocycle has at least one nitrogen atom, phosphorous atom, or oxygen atom. In some cases, for R¹, the heterocycle has at least one nitrogen atom. In some cases, for R¹, the heterocycle has at least two nitrogen atoms. In some cases, for R¹, the heterocycle has at most two nitrogen atoms. In some cases, for R¹, the heterocycle has at most one nitrogen atom. In some cases, for R¹, the heterocycle is a spirocycle. In some cases, for R¹, the heterocycle is a bridged heterocycle. In some cases, for R¹, the heterocycle is unsaturated. In some cases, for R¹, the heterocycle is saturated. In some cases, R¹ is selected from

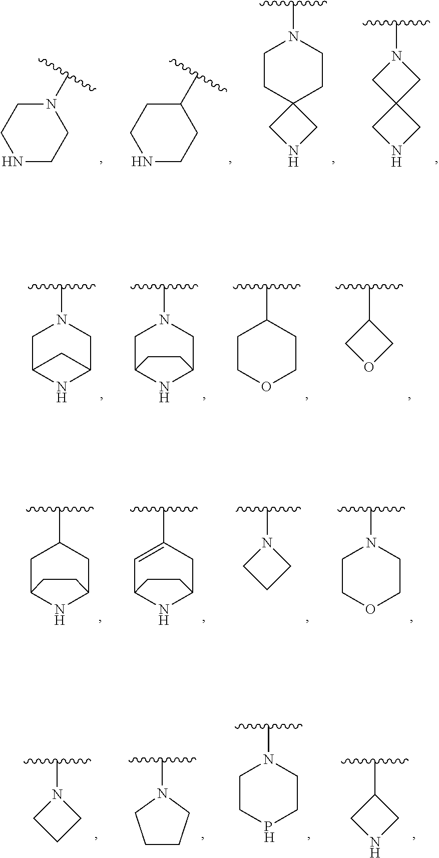

any of which are optionally substituted. In some cases, R¹ is selected from

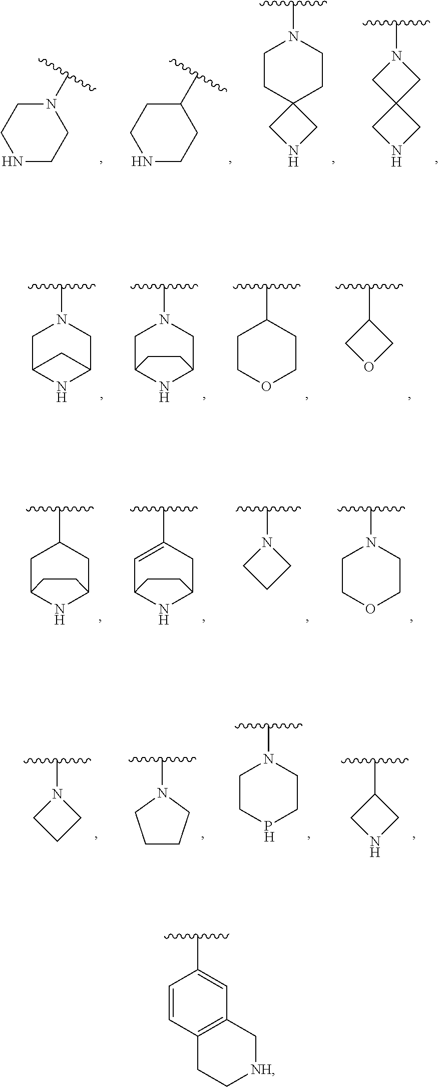

any of which are optionally substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl. In some cases, R¹ is selected from

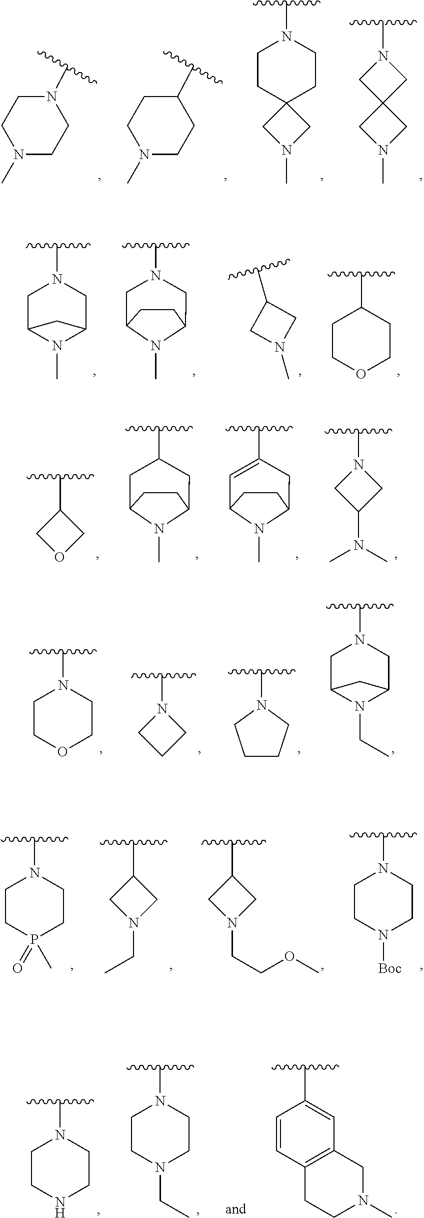

In some cases, R¹ is selected from

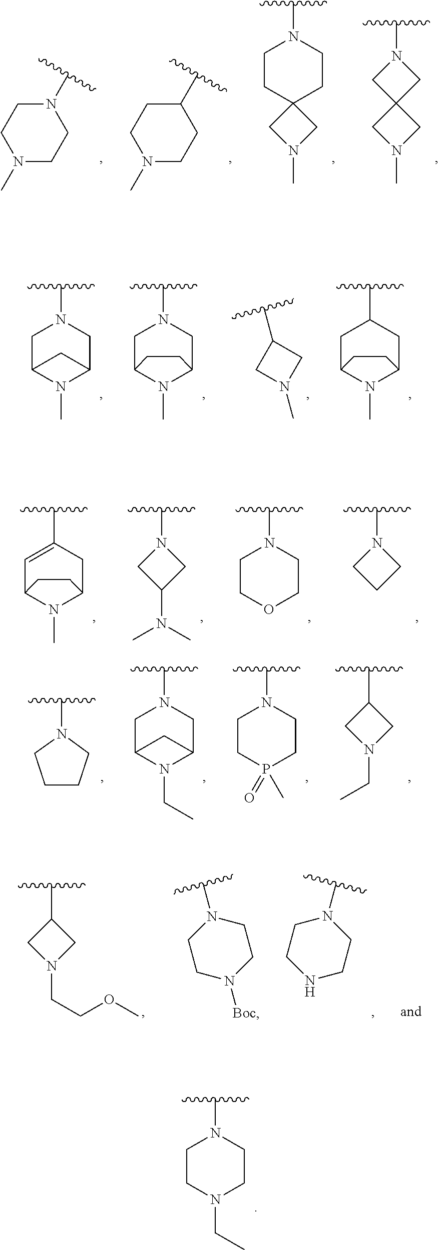

In some cases, R¹ is selected from

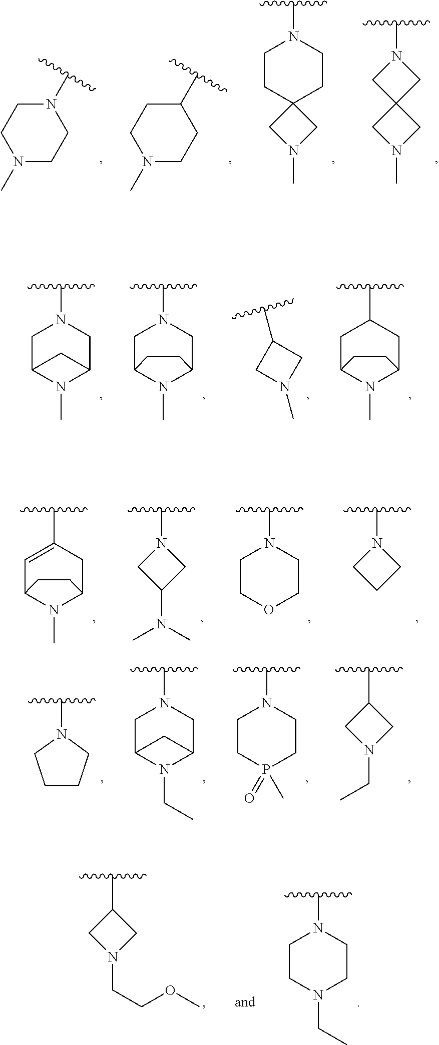

In some cases, R¹ is selected from

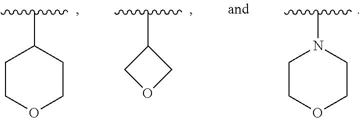 [SMILES: *C1CCOCC1 ; *C1COC1 ; *N1CCOCC1]

In some cases, R¹ is selected from

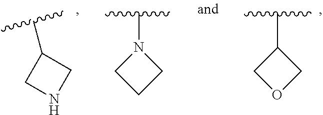

each of which is optionally substituted. In some cases, the optional substituents are selected form —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl

In some cases, W is unsubstituted thiazole. In some cases, W is

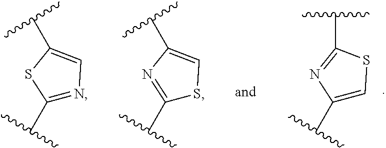 [SMILES: *c1cnc(*)s1 ; *c1csc(*)n1 ; *c1csc(*)n1 ; "."]

In some cases, W is

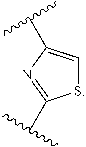 [SMILES: *c1csc(*)n1]

In some embodiments, for a compound or salt of Formula (A*), R¹ is selected from

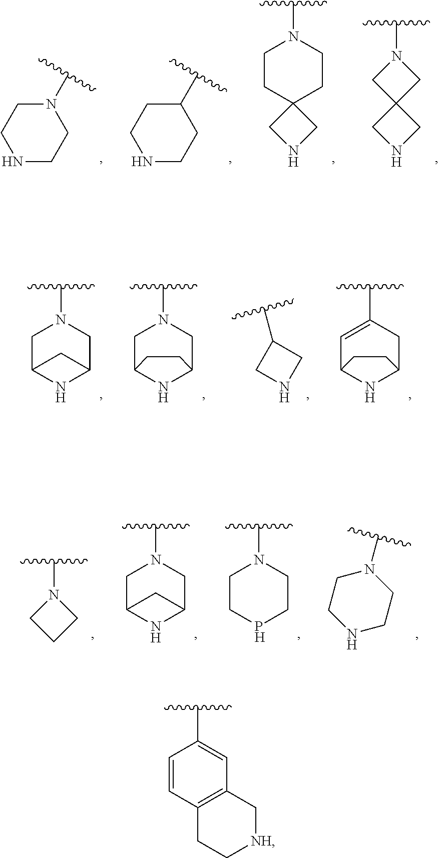

each of which are substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (A*), R¹ is substituted piperazine.

In some cases, for a compound or salt of Formula (A*), when R¹ is piperazine, and Z is phenyl substituted with at least one more substituents, the piperazine is substituted. In some cases, when R¹ is piperazine, and Z is phenyl, the piperazine is substituted.

In some embodiments, for a compound or salt of Formula (A*), R¹ is an optionally substituted 6- to 10-membered heterocycloalkyl. In some cases, the optional substituents of the optionally substituted 6- to 10-membered heterocycloalkyl for R¹ are selected from C₁₋₆ alkyl. In some cases, the 6- to 10-membered heterocycloalkyl is a spiro heterocycloalkyl. In some cases, R¹ is selected from optionally substituted piperazine, optionally substituted diazabicyclo [3.2.1]octane, optionally substituted diazabicyclo[3.1.1]heptane, optionally substituted diazaspiro[3.5]nonane, and optionally substituted diazaspiro[3.3]heptane. In some cases, the optional are selected from C₁₋₆ alkyl.

In another aspect, the present disclosure provides a compound represented by Formula (B*):

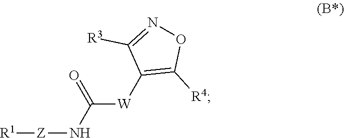

or a pharmaceutically acceptable salt thereof, wherein:
R¹ is selected from:
   optionally substituted 3 to 10-membered heterocycle; wherein the 3- to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
W is selected from optionally substituted 3- to 12-membered heterocycle, wherein the 3- to 12-membered heterocycle is independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

In some embodiments, a compound represented by Formula (B*):

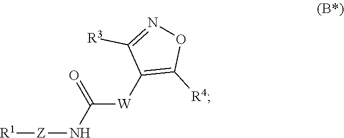

or a pharmaceutically acceptable salt thereof, wherein:
R¹ is selected from:
   optionally substituted 3 to 10-membered heterocycle;
   —N(R⁵)₂, wherein R⁵ is selected from hydrogen, and optionally substituted C₁-C₆ alkyl;
R³ is selected from an optionally substituted C₃₋₁₂ carbocycle;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, optionally substituted C₁₋₁₀ alkyl, C₂₋₁₀ alkenyl, C₂₋₁₀ alkynyl, optionally substituted C₃₋₁₂ carbocycle, and optionally substituted 3- to 12-membered heterocycle;
Z is selected from optionally substituted 3- to 12-membered heterocycle and optionally substituted C₃-C₁₂ carbocycle;
W is selected from optionally substituted 3- to 12-membered heterocycle.

In some embodiments, for a compound or salt of Formula (B*), Z is selected from optionally substituted 3- to 12-membered heterocycle and optionally substituted C₃-C₁₂ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl. In some cases, for Z, the heterocycle includes at least one nitrogen atom. In some cases, Z is selected from optionally substituted phenyl and optionally substituted pyridine. In some cases, the optional substituents of the optionally substituted phenyl of Z is optionally substituted with one or more substituents independently at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl. In some cases, the optional substituents of the optionally substituted phenyl of Z is optionally substituted with one or more substituents independently selected at each occurrence from halogen and C₁₋₁₀ alkyl. In some cases, Z is selected from substituted phenyl and unsubstituted pyridine. In some cases, Z is selected from phenyl substituted with one or more substituents selected from halogen and C₁₋₁₀ alkyl. In some cases, Z is selected from

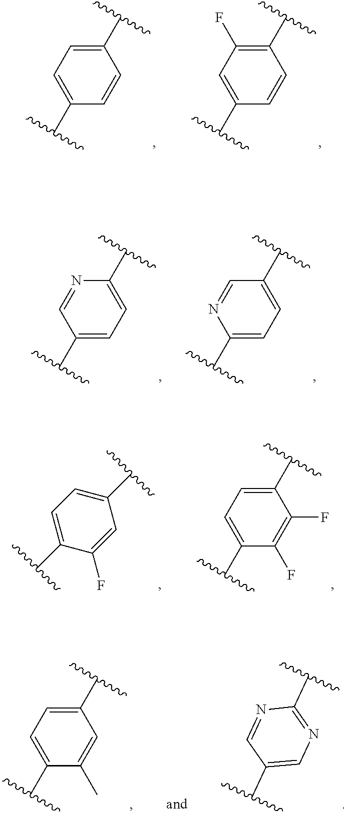

In some cases, Z is selected from

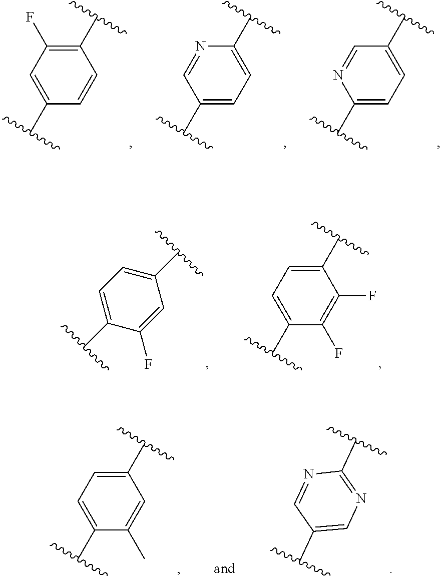

In some cases, the optional substituents of the optionally substituted phenyl of Z is halogen. In some cases, Z is selected from

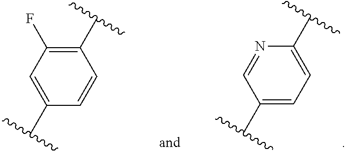 [SMILES: *c1ccc(*)c(F)c1 ; *c1ccc(*)nc1]

In some cases, Z is substituted phenyl. In some cases, Z is phenyl substituted with halogen.

In some embodiments, for a compound or salt of Formula (B*), W is selected from optionally substituted 5- to 8-membered heterocycle. In some cases, the heterocycle of W is a 5- to 8-membered heteroaryl. In some cases, the heterocycle of W is an unsubstituted 5- to 8-membered heteroaryl. In some cases, the heterocycle of W is an unsubstituted 5-membered heteroaryl. In some cases, the heterocycle of W has at least 2 heteroatoms. In some cases, the heterocycle of W has at most 2 heteroatoms. In some cases, the heterocycle of W has only 2 heteroatoms. In some cases, the heterocycle of W is unsubstituted. In some cases, the heterocycle of W has 2 heteroatoms selected from nitrogen, sulfur, and oxygen. In some cases, the heterocycle of W has at least 2 different heteroatoms. In some cases, the heterocycle of W has 2 nitrogen atoms. In some cases, the heterocycle of W has 1 nitrogen atom and 1 sulfur atom. In some cases, the heterocycle of W has 1 nitrogen atom and 1 oxygen atom. In some cases, the heterocycle of W is selected from imidazole, thiazole, and isoxazole. In some cases, the heterocycle of W is selected from thiazole, and isoxazole. In some cases, the heterocycle of W is thiazole. In some cases, the heterocycle of W is isoxazole. In some cases, the heterocycle of W is selected from

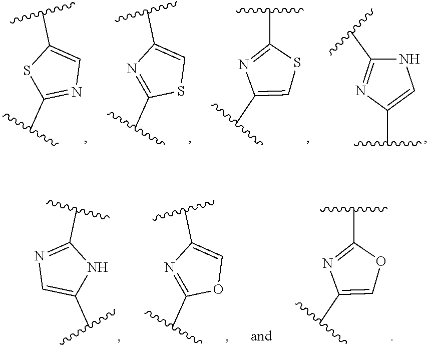

In some cases, the heterocycle of W is selected from

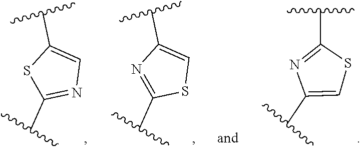 [SMILES: *c1cnc(*)s1 ; *c1csc(*)n1 ; *c1csc(*)n1]

In some cases, the heterocycle of W is selected from

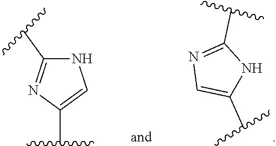 [SMILES: *c1c[nH]c(*)n1 ; *c1cnc(*)[nH]1]

In some cases, the heterocycle of W is selected from

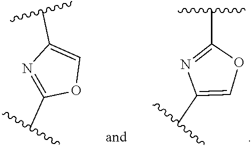 [SMILES: *c1coc(*)n1 ; *c1coc(*)n1]

In some cases, the heterocycle of W is selected from

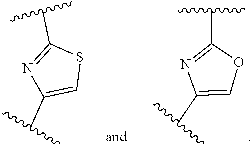 [SMILES: *c1csc(*)n1 ; *c1coc(*)n1]

In some cases, the heterocycle of W is

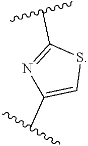 [SMILES: *c1csc(*)n1]

In some cases, the heterocycle of W is

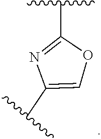 [SMILES: *c1coc(*)n1]

In some embodiments, for a compound or salt of Formula (B*), R⁴ is selected from optionally substituted C₁-C₆ alkyl, optionally substituted C₃₋₆ carbocycle and optionally substituted 3- to 8-membered heterocycle.

In some embodiments, for a compound or salt of Formula (B*), R⁴ is selected from optionally substituted C₁-C₆ alkyl. In some cases, R⁴ is selected from substituted C₁-C₆ alkyl. In some cases, the substituents are independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle. In some cases, the substituents are independently selected at each occurrence from halogen, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle. In some cases, R⁴ is selected from unsubstituted C₁-C₆ alkyl. In some cases, R⁴ is a branched C₁-C₆ alkyl. In some cases, for R⁴, the optional substituents of the optionally substituted C₁-C₆ alkyl are independently selected from halogen, —OH, —CN, —NO₂, —NH₂, oxo, —O—C₁₋₁₀ alkyl, —C₁₋₁₀ haloalkyl, and —O—C₁₋₁₀ alkyl.

In some embodiments, for a compound or salt of Formula (B*), R⁴ is selected from optionally substituted C₃₋₆ carbocycle and optionally substituted 3- to 8-membered heterocycle. In some cases, R⁴ is selected from optionally substituted 3- to 8-membered heterocycle. In some cases, for R⁴, the optional substituents of the optionally substituted C₃₋₆ carbocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, for R⁴, the optional substituents of the optionally substituted C₃₋₆ carbocycle are independently selected from halogen and —C₁₋₁₀ haloalkyl. In some cases, for R⁴, the optional substituents of the optionally substituted 3- to 8-membered heterocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl.

In some embodiments, for a compound or salt of Formula (B*), R⁴ is selected from unsubstituted C₁-C₆ alkyl, unsubstituted 3- to 6-membered heterocycle, and C₃₋₆ carbocycle optionally substituted with one or more halogens. In some cases, R⁴ is selected from

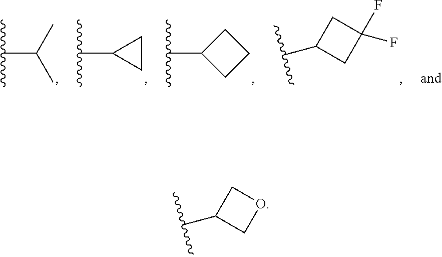

In some cases, R⁴ is selected from

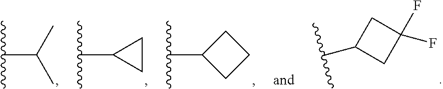

In some cases, R⁴ is

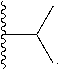 [SMILES: *C(C)C]

In some cases, R⁴ is selected from

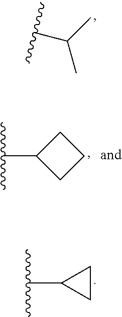

In some embodiments, for a compound or salt of Formula (B*), R¹ is monocyclic. In some embodiments, R¹ is bicyclic. In some embodiments, R¹ is a fused bicyclic group. In some embodiments, R¹ is a bridged bicyclic group. In some embodiments, R¹ is optionally substituted 5 membered heterocycle. In some embodiments, R¹ is optionally substituted heteroaryl. In some embodiments, R¹ is optionally substituted heterocycloalkyl. In some embodiments, R¹ contains 0-3 nitrogen and 0-1 oxygen atoms on the ring. In some embodiments, R¹ contains 1-2 nitrogen and 0-1 oxygen atoms on the ring. In some embodiments, R¹ contains 1-2 ring nitrogen atoms. In some embodiments, R¹ contains 2 ring nitrogen atoms. In some embodiments, R¹ contains 1 ring nitrogen atom.

In some embodiments, for a compound or salt of Formula (B*), R¹ is substituted C₁-C₆alkyl. In some cases, C₁-C₆ alkyl is substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle. In some cases, R¹ is optionally substituted C₁-C₆ alkyl. In some cases, C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle. In some cases, the C₁-C₆ alkyl is optionally substituted with one or more substituents independently selected at each occurrence from halogen, —CN, —NO₂, —NH₂, oxo, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle. In some cases, the C₁-C₆ alkyl is optionally substituted with one or more substituents independently selected at each occurrence from halogen, —CN, —NO₂, —NH₂, oxo, —C₁₋₁₀ haloalkyl, and —O—C₁₋₁₀ alkyl. In some cases, the C₁-C₆alkyl is substituted with one or more substituents independently selected at each occurrence from halogen, —CN, —NO₂, —NH₂, oxo, —C₁₋₁₀ haloalkyl, and —O—C₁₋₁₀ alkyl.

In some embodiments, for a compound or salt of Formula (B*), R¹ is an optionally substituted 3 to 10-membered heterocycle. In some cases, for R¹, the 3- to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, —O—C₁₋₁₀ alkyl, and —NO₂. In some cases, for R¹, the 3- to 10-membered heterocycle is optionally substituted with one or more substituents independently selected at each occurrence from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl. In some cases, for R¹, the heterocycle has at least one nitrogen atom, phosphorous atom, or oxygen atom. In some cases, for R¹, the heterocycle has at least one nitrogen atom. In some cases, for R¹, the heterocycle has at least two nitrogen atoms. In some cases, for R¹, the heterocycle has at most two nitrogen atoms. In some cases, for R¹, the heterocycle has at most one nitrogen atom. In some case, for R¹, the heterocycle has at one oxygen atom. In some cases, for R¹, the heterocycle is a spirocycle. In some cases, for R¹, the heterocycle is a bridged heterocycle. In some cases, for R¹, the heterocycle is unsaturated. In some cases, for R¹, the heterocycle is saturated. In some cases, R¹ is selected from

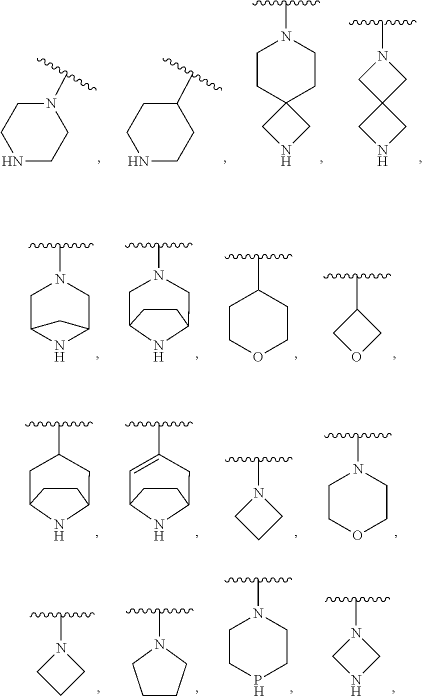

any of which are optionally substituted. In some cases, R¹ is selected from

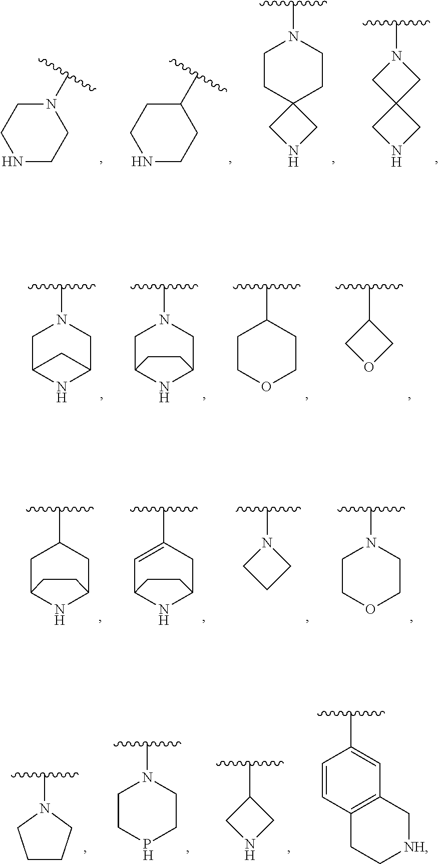

any of which are optionally substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl. In some cases, R¹ is selected from

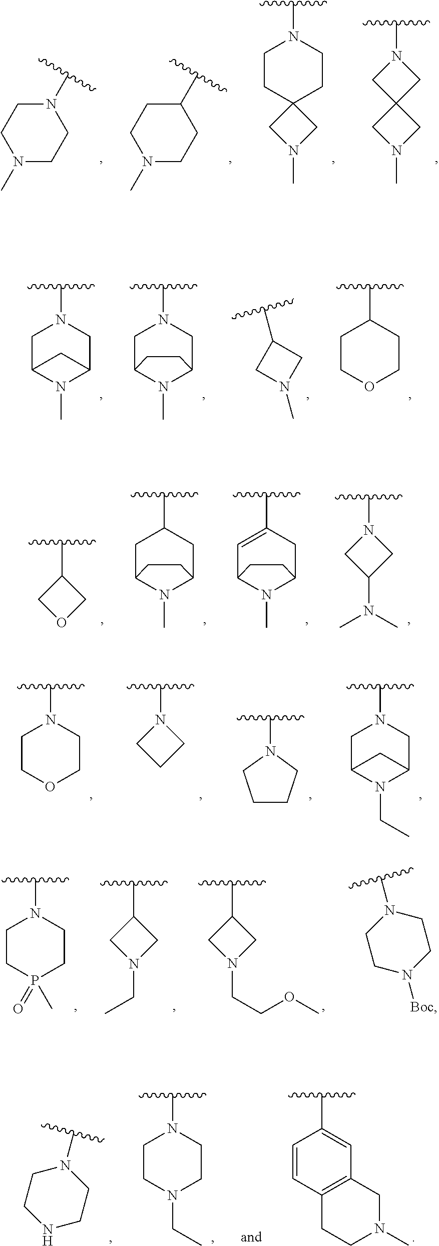

In some cases, R¹ is selected from

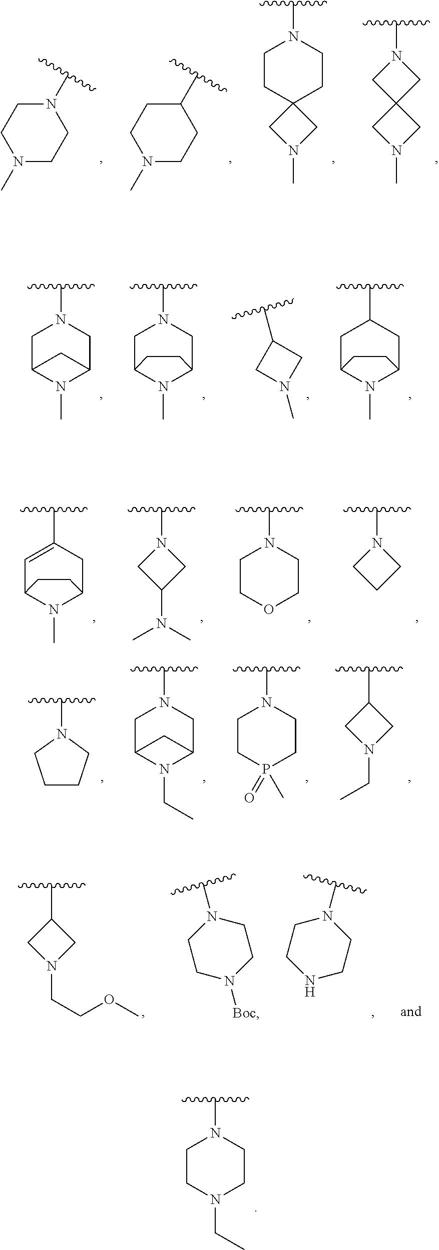

In some cases, R¹ is selected from

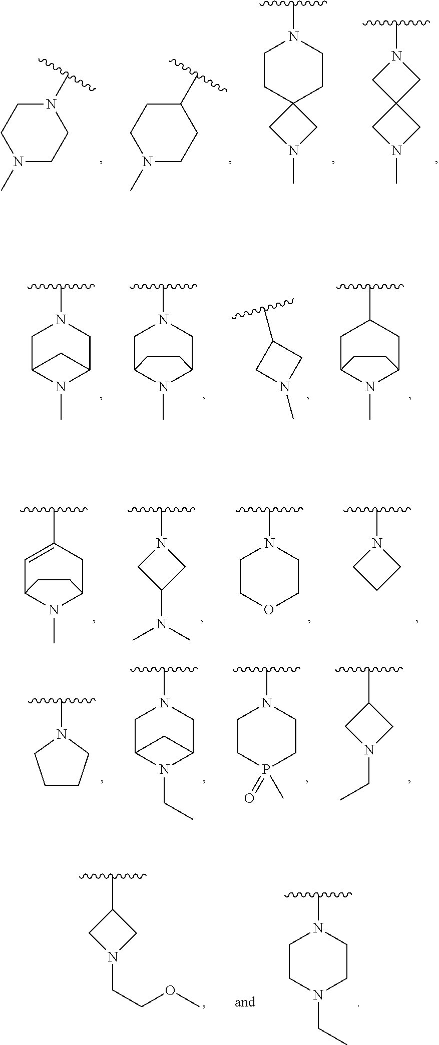

In some cases, R¹ is selected from

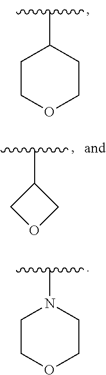

In some cases, R¹ is selected from

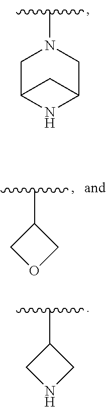

any of which are optionally substituted with one or more substituents selected from unsubstituted C₁₋₁₀ alkyl. In some cases, R¹ is selected from

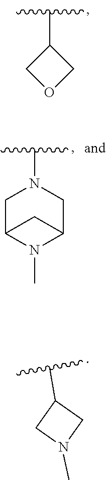

In some embodiments, for a compound or salt of Formula (B*), R¹ is selected from

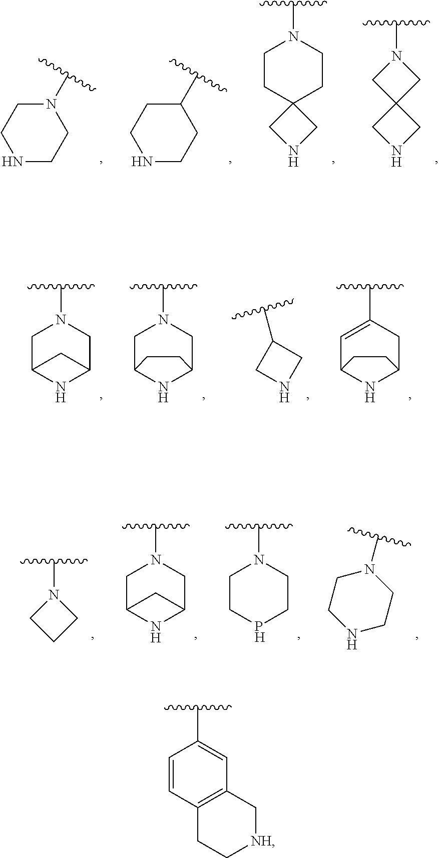

each of which are substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (B*), R¹ is substituted piperazine.

In some cases, for a compound or salt of Formula (B*), when R¹ is piperazine, and Z is phenyl substituted with at least one more substituents, the piperazine is substituted. In some cases, when R¹ is piperazine, and Z is phenyl, the piperazine is substituted.

In some embodiments, for a compound or salt of Formula (B*), R¹ is an optionally substituted 6- to 10-membered heterocycloalkyl. In some cases, the optional substituents of the optionally substituted 6- to 10-membered heterocycloalkyl for R¹ are selected from C₁₋₆ alkyl. In some cases, the 6- to 10-membered heterocycloalkyl is a spiro heterocycloalkyl. In some cases, R¹ is selected from optionally substituted piperazine, optionally substituted diazabicyclo [3.2.1]octane, optionally substituted diazabicyclo[3.1.1]heptane, optionally substituted diazaspiro[3.5]nonane, and optionally substituted diazaspiro[3.3]heptane. In some cases, the optional are selected from C₁₋₆ alkyl.

In another aspect, the present disclosure provides a compound represented by (C*):

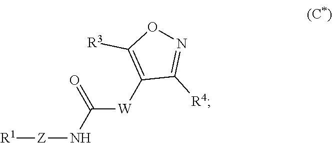

or a pharmaceutically acceptable salt thereof, wherein:
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
R¹ is selected from:
   optionally substituted 3 to 10-membered heterocycle; wherein the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
W is selected from optionally substituted 3- to 12-membered heterocycle, wherein the 3- to 12-membered heterocycle is independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

In some embodiments, for a compound represented by (C*):

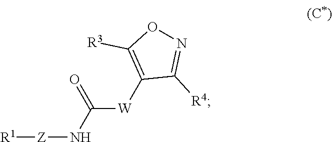

or a pharmaceutically acceptable salt thereof, wherein:
R³ is selected from an optionally substituted C₃₋₁₂ carbocycle;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, optionally substituted C₁₋₁₀ alkyl, C₂₋₁₀ alkenyl, C₂₋₁₀ alkynyl, optionally substituted C₃₋₁₂ carbocycle, optionally substituted 3- to 12-membered heterocycle;
Z is selected from optionally substituted 3- to 12-membered heterocycle and optionally substituted C₃-C₁₂ carbocycle;
R¹ is selected from:
   optionally substituted 3 to 10-membered heterocycle;
   —N(R⁵)₂, wherein R⁵ is selected from hydrogen, and optionally substituted C₁-C₆ alkyl;
   optionally substituted C₁-C₆ alkyl;
W is selected from optionally substituted 3- to 12-membered heterocycle.

In some embodiments, for a compound or salt of Formula (C*), R¹ is monocyclic. In some embodiments, R¹ is bicyclic. In some embodiments, R¹ is a fused bicyclic group. In some embodiments, R¹ is a bridged bicyclic group. In some embodiments, R¹ is optionally substituted 5 membered heterocycle. In some embodiments, R¹ is optionally substituted heteroaryl. In some embodiments, R¹ is optionally substituted heterocycloalkyl. In some embodiments, R¹ contains 0-3 nitrogen and 0-1 oxygen atoms on the ring. In some embodiments, R¹ contains 1-2 nitrogen and 0-1 oxygen atoms on the ring. In some embodiments, R¹ contains 1-2 ring nitrogen atoms. In some embodiments, R¹ contains 2 ring nitrogen atoms. In some embodiments, R¹ contains 1 ring nitrogen atom.

In some embodiments, for a compound or salt of Formula (C*), Z is selected from optionally substituted 3- to 12-membered heterocycle and optionally substituted C₃-C₁₂ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl. In some cases, for Z, the heterocycle includes at least one nitrogen atom. In some cases, Z is selected from optionally substituted phenyl and optionally substituted pyridine. In some cases, phenyl of Z is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl. In some cases, phenyl of Z is optionally substituted with one or more substituents independently selected at each occurrence from halogen and C₁₋₁₀ alkyl. In some cases, the heterocycle is unsubstituted. In some cases, Z is selected from substituted phenyl and unsubstituted pyridine. In some cases, the heterocycle has 1 or 2 nitrogen atoms. In some cases, the heterocycle has only 1 nitrogen atom. In some cases, the heterocycle has only 2 nitrogen atoms. In some cases, the heterocycle is a 6-membered heterocycle. In some cases, Z is selected from

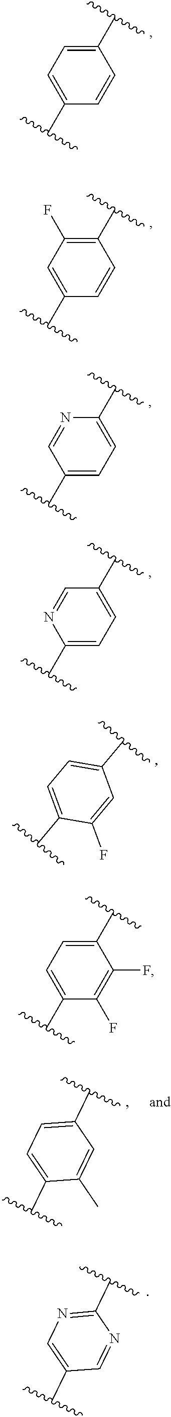

In some cases, the optional substituents of the optionally substituted phenyl of Z is halogen. In some cases, Z is selected from

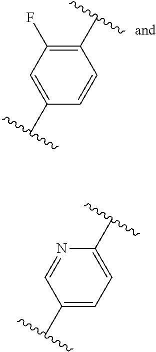 [SMILES: *c1ccc(*)c(F)c1 ; *c1ccc(*)nc1]

In some cases, Z is substituted phenyl. In some cases, Z is phenyl substituted with halogen.

In some embodiments, for a compound or salt of Formula (C*), W is selected from optionally substituted 5- to 8-membered heterocycle. In some cases, the heterocycle of W is a 5- to 8-membered heteroaryl. In some cases, the heterocycle of W is an unsubstituted 5- to 8-membered heteroaryl. In some cases, the heterocycle of W is an unsubstituted 5-membered heteroaryl. In some cases, the heterocycle of W has at least 2 heteroatoms. In some cases, the heterocycle of W has at most 2 heteroatoms. In some cases, the heterocycle of W has only 2 heteroatoms. In some cases, the heterocycle of W is unsubstituted. In some cases, the heterocycle of W has 2 heteroatoms selected from nitrogen, sulfur, and oxygen. In some cases, the heterocycle of W has at least 2 different heteroatoms. In some cases, the heterocycle of W has 2 nitrogen atoms. In some cases, the heterocycle of W has 1 nitrogen atom and 1 sulfur atom. In some cases, the heterocycle of W has 1 nitrogen atom and 1 oxygen atom. In some cases, the heterocycle of W is selected from imidazole, thiazole, and isoxazole. In some cases, the heterocycle of W is selected from thiazole, and isoxazole. In some cases, the heterocycle of W is thiazole. In some cases, the heterocycle of W is isoxazole. In some cases, the heterocycle of W is selected from

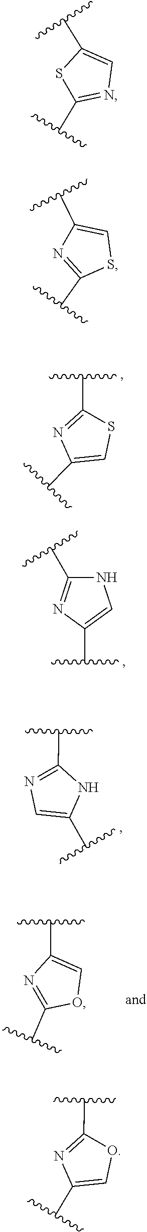

In some cases, the heterocycle of W is selected from

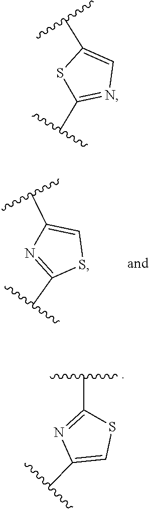 [SMILES: *c1cnc(*)s1 ; *c1csc(*)n1 ; *c1csc(*)n1]

In some cases, the heterocycle of W is selected from

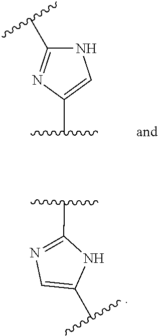 [SMILES: *c1c[nH]c(*)n1 ; *c1cnc(*)[nH]1]

In some cases, the heterocycle of W is selected from

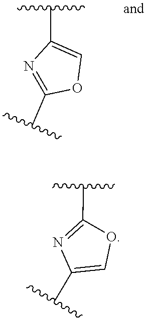 [SMILES: *c1coc(*)n1 ; *c1coc(*)n1]

In some cases, the heterocycle of W is selected from

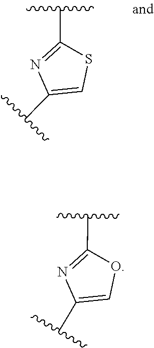 [SMILES: *c1csc(*)n1 ; *c1coc(*)n1]

In some cases, the heterocycle of W is

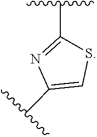 [SMILES: *c1csc(*)n1]

In some cases, the heterocycle of W is

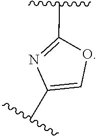 [SMILES: *c1coc(*)n1]

In some embodiments, for a compound or salt of Formula (C*), R⁴ is selected from optionally substituted C₁-C₆ alkyl, optionally substituted C₃₋₆ carbocycle and optionally substituted 3- to 8-membered heterocycle. In some cases, R⁴ is selected from optionally substituted C₁-C₆ alkyl. In some cases, R⁴ is selected from optionally substituted C₃₋₆ carbocycle and optionally substituted 3- to 8-membered heterocycle. In some cases, R⁴ is selected from optionally substituted 3- to 8-membered heterocycle. In some cases, for R⁴, the optional substituents of the optionally substituted C₁-C₆ alkyl are independently selected from halogen, —OH, —CN, —NO₂, —NH₂, oxo, —O—C₁₋₁₀ alkyl, —C₁₋₁₀ haloalkyl, and —O—C₁₋₁₀ alkyl.

In some embodiments, for a compound or salt of Formula (C*), for R⁴, the optional substituents of the optionally substituted C₃₋₆ carbocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, for R⁴, the optional substituents of the optionally substituted C₃₋₆ carbocycle are independently selected from halogen and —C₁₋₁₀ haloalkyl. In some cases, for R⁴, the optional substituents of the optionally substituted 3- to 8-membered heterocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, R⁴ is selected from unsubstituted C₁-C₆ alkyl, unsubstituted 3- to 6-membered heterocycle, and C₃₋₆ carbocycle optionally substituted with one or more halogens. In some cases, R⁴ is selected from

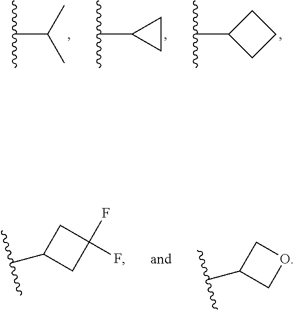

In some cases, R⁴ is selected from

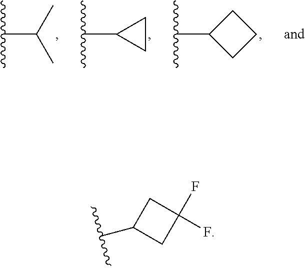

In some cases, R⁴ is

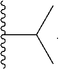 [SMILES: *C(C)C]

In some cases, R⁴ is selected from

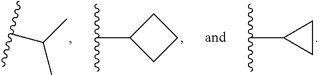

In some embodiments, for a compound or salt of Formula (C*), R¹ is substituted C₁-C₆alkyl.

In some embodiments, for a compound or salt of Formula (C*), R¹ is an optionally substituted 3 to 10-membered heterocycle. In some cases, for R¹, the 3- to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, —O—C₁₋₁₀ alkyl, and —NO₂. In some cases, for R¹, 3- to 10-membered heterocycle is optionally substituted with one or more substituents independently selected at each occurrence from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (C*), for R¹, the heterocycle has at least one nitrogen atom, phosphorous atom, or oxygen atom. In some cases, for R¹, the heterocycle has at least one nitrogen atom. In some cases, for R¹, the heterocycle has at least two nitrogen atoms. In some cases, for R¹, the heterocycle has at most two nitrogen atoms. In some cases, for R¹, the heterocycle has at most one nitrogen atom. In some cases, for R¹, the heterocycle has at one oxygen atom. In some cases, for R¹, the heterocycle is a spirocycle. In some cases, for R¹, the heterocycle is a bridged heterocycle. In some cases, for R¹, the heterocycle is unsaturated. In some cases, for R¹, the heterocycle is saturated.

In some embodiments, for a compound or salt of Formula (C*), R¹ is selected from

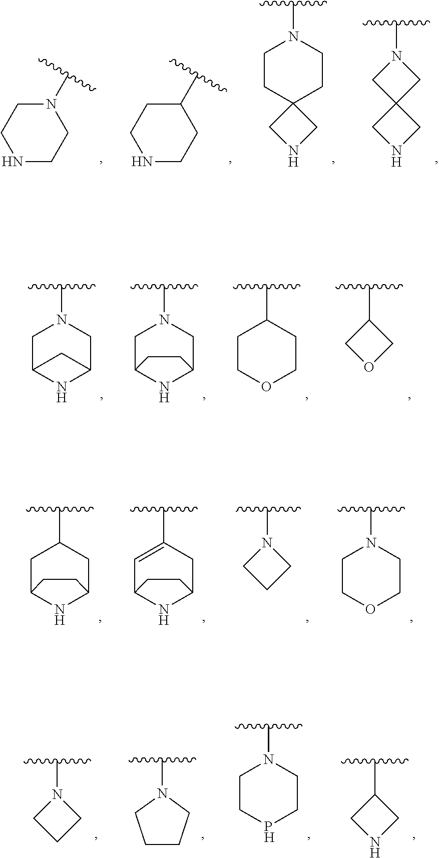

any of which are optionally substituted. In some cases, R¹ is selected from

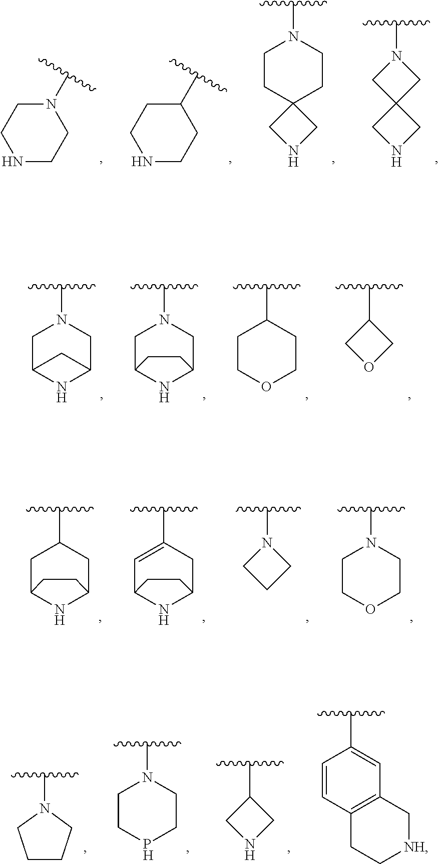

any of which are optionally substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl. In some cases, R¹ is selected from

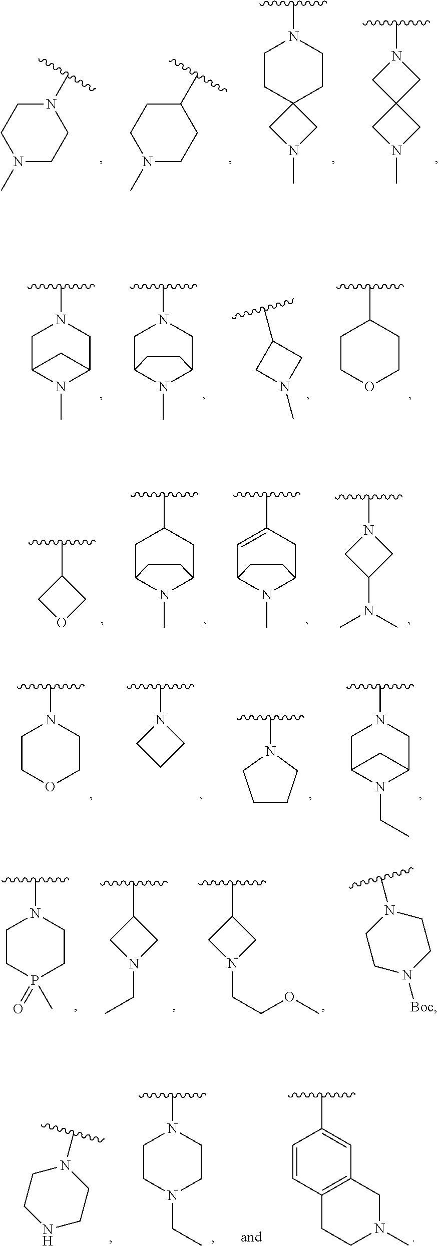

In some cases R¹ is selected from

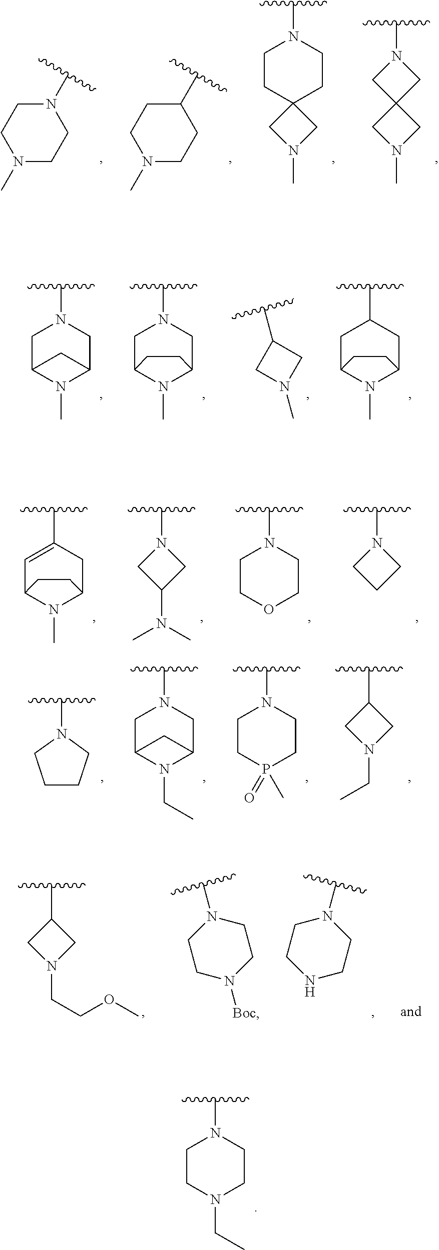

In some cases, R¹ is selected from

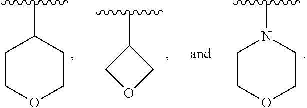

In some cases, R¹ is selected from

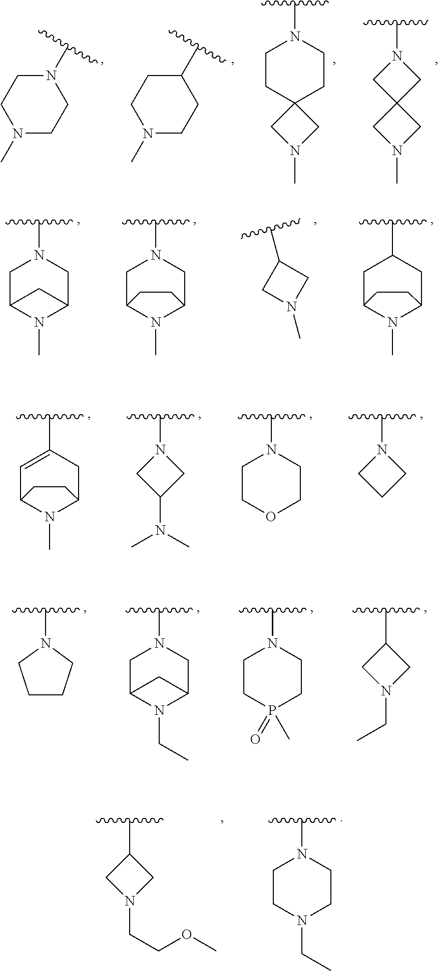

In some cases, R¹ is selected from

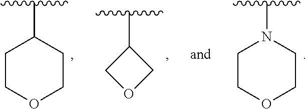

In some cases, R¹ is selected from

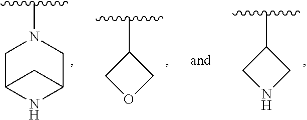

any of which are optionally substituted with one or more substituents selected from unsubstituted C₁₋₁₀ alkyl. In some cases, R¹ is selected from

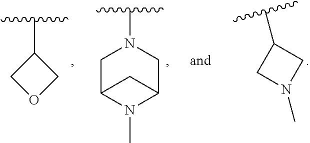

In some embodiments, for a compound or salt of Formula (C*), R¹ is selected from

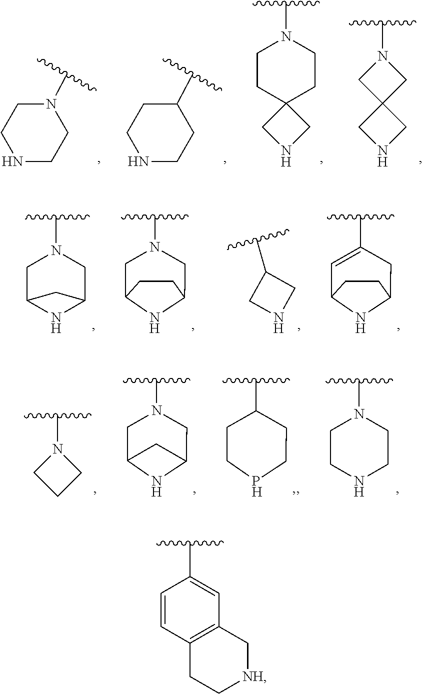

each of which are substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (C*), R¹ is substituted piperazine.

In some cases, for a compound or salt of Formula (C*), when R¹ is piperazine, and Z is phenyl substituted with at least one more substituents, the piperazine is substituted. In some cases, when R¹ is piperazine, and Z is phenyl, the piperazine is substituted.

In some embodiments, for a compound or salt of Formula (C*), R¹ is an optionally substituted 6- to 10-membered heterocycloalkyl. In some cases, the optional substituents of the optionally substituted 6- to 10-membered heterocycloalkyl for R¹ are selected from C₁₋₆ alkyl. In some cases, the 6- to 10-membered heterocycloalkyl is a spiro heterocycloalkyl. In some cases, R¹ is selected from optionally substituted piperazine, optionally substituted diazabicyclo [3.2.1]octane, optionally substituted diazabicyclo[3.1.1]heptane, optionally substituted diazaspiro[3.5]nonane, and optionally substituted diazaspiro[3.3]heptane. In some cases, the optional are selected from C₁₋₆ alkyl.

In another aspect, the present disclosure provides a compound represented by Formula (D*):

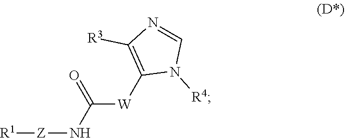

or a pharmaceutically acceptable salt thereof, wherein:
R¹ is selected from:
   optionally substituted 3 to 10-membered heterocycle; wherein the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from halogen, oxo, —C₁₋₁₀ haloalkyl, —NH₂, —CN, —O—C₁₋₁₀ alkyl, and —NO₂;
   —N(R⁵)₂, wherein R⁵ is selected from hydrogen, and optionally substituted C₁-C₆ alkyl, wherein the C₁-C₆ alkyl is optionally substituted with one or more substituents independently selected at each occurrence from selected from halogen, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
   optionally substituted C₁-C₆ alkyl, wherein the C₁-C₆ alkyl is optionally substituted with one or more substituents independently selected at each occurrence from halogen, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
W is selected from optionally substituted 3- to 12-membered heterocycle having at least two heteroatoms, wherein the 3- to 12-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle; and
wherein when W is imidazole, Y is imidazole, Z is phenyl, and R¹ is piperazine, the piperazine is substituted.

In some embodiments, for a compound represented by Formula (D*):

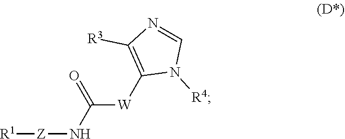

or a pharmaceutically acceptable salt thereof, wherein:
R¹ is selected from:
   optionally substituted 3 to 10-membered heterocycle;
   —N(R⁵)₂, wherein R⁵ is selected from hydrogen, and optionally substituted C₁-C₆ alkyl;
   optionally substituted C₁-C₆ alkyl;
R³ is selected from a optionally substituted C₃₋₁₂ carbocycle;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, optionally substituted C₁₋₁₀ alkyl, C₂₋₁₀ alkenyl, C₂₋₁₀ alkynyl, optionally substituted C₃₋₁₂ carbocycle, and optionally substituted 3- to 12-membered heterocycle;
Z is selected from optionally substituted 3- to 12-membered heterocycle and optionally substituted C₃-C₁₂ carbocycle;
W is selected from optionally substituted 3- to 12-membered heterocycle having at least two heteroatoms, and
wherein when W is imidazole, Y is imidazole, Z is phenyl, and R¹ is piperazine, the piperazine is substituted.

In some embodiments, for a compound or salt of Formula (D*), Z is selected from optionally substituted 3- to 12-membered heterocycle and optionally substituted C₃-C₁₂ carbocycle, wherein the substituents on each are independently selected at each occurrence from one or more halogen, —OH, —CN, —NO₂, —NH₂, oxo, ═S, —C₁₋₁₀ haloalkyl, —O—C₁₋₁₀ alkyl.

In some embodiments, for a compound or salt of Formula (D*), for Z, the heterocycle includes at least one nitrogen atom. In some cases, Z is selected from optionally substituted phenyl and optionally substituted pyridine. In some cases, the phenyl of Z is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl. In some cases, phenyl of Z is optionally substituted with one or more substituents independently selected at each occurrence from halogen and C₁₋₁₀ alkyl. In some cases, Z is selected from

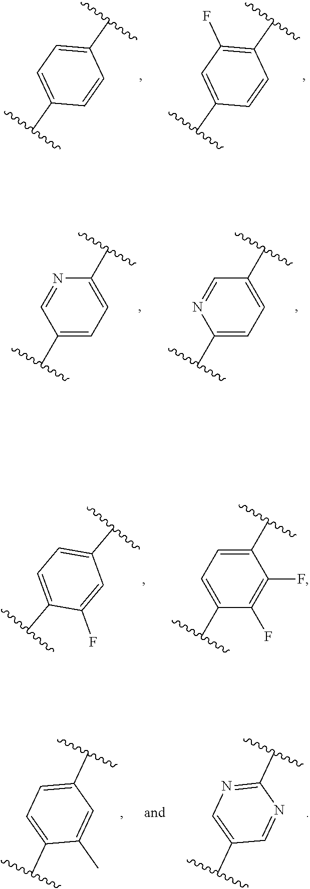

In some cases, the optional substituents of the optionally substituted phenyl of Z is halogen. In some cases, Z is selected from

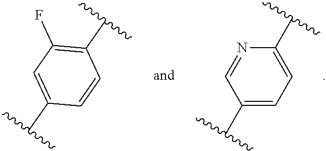 [SMILES: *c1ccc(*)c(F)c1 ; *c1ccc(*)nc1]

In some cases, Z is substituted phenyl. In some cases, Z is phenyl substituted with halogen.

In some embodiments, for a compound or salt of Formula (D*), R¹ is monocyclic. In some embodiments, R¹ is bicyclic. In some embodiments, R¹ is a fused bicyclic group. In some embodiments, R¹ is a bridged bicyclic group. In some embodiments, R¹ is optionally substituted 5 membered heterocycle. In some embodiments, R¹ is optionally substituted heteroaryl. In some embodiments, R¹ is optionally substituted heterocycloalkyl. In some embodiments, R¹ contains 0-3 nitrogen and 0-1 oxygen atoms on the ring. In some embodiments, R¹ contains 1-2 nitrogen and 0-1 oxygen atoms on the ring. In some embodiments, R¹ contains 1-2 ring nitrogen atoms. In some embodiments, R¹ contains 2 ring nitrogen atoms. In some embodiments, R¹ contains 1 ring nitrogen atom.

In some cases, for a compound or salt of Formula (D*), when R¹ is piperazine, and Z is phenyl substituted with at least one more substituents, the piperazine is substituted. In some cases, when R¹ is piperazine, and Z is phenyl, the piperazine is substituted.

In some cases, W is selected from optionally substituted 5- to 8-membered heterocycle.

In some embodiments, for a compound or salt of Formula (D*), the heterocycle of W is a 5- to 8-membered heteroaryl. In some cases, the heterocycle of W is an unsubstituted 5- to 8-membered heteroaryl. In some cases, the heterocycle of W is an unsubstituted 5-membered heteroaryl. In some cases, the heterocycle of W has at least 2 heteroatoms. In some cases, the heterocycle of W has at most 2 heteroatoms. In some cases, the heterocycle of W has only 2 heteroatoms. In some cases, the heterocycle of W is unsubstituted. In some cases, the heterocycle of W has 2 heteroatoms selected from nitrogen, sulfur, and oxygen. In some cases, the heterocycle of W has at least 2 different heteroatoms. In some cases, the heterocycle of W has 2 nitrogen atoms. In some cases, the heterocycle of W has 1 nitrogen atom and 1 sulfur atom. In some cases, the heterocycle of W has 1 nitrogen atom and 1 oxygen atom. In some cases, the heterocycle of W is selected from imidazole, thiazole, and isoxazole. In some cases, the heterocycle of W is selected from thiazole, and isoxazole. In some cases, the heterocycle of W is thiazole. In some cases, the heterocycle of W is isoxazole. In some cases, the heterocycle of W is selected from

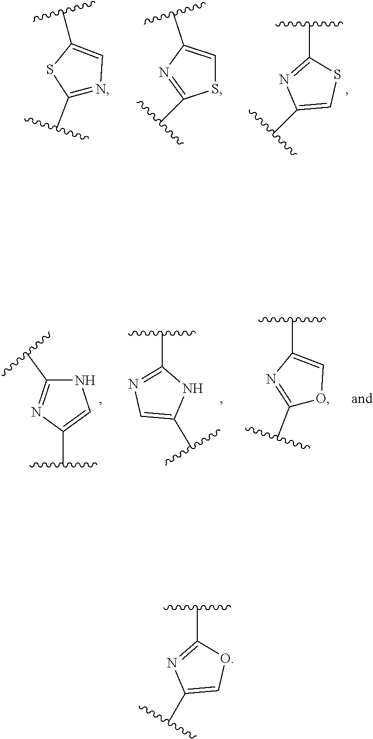

In some cases, the heterocycle of W is selected from

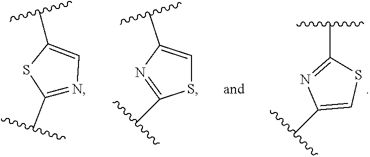 [SMILES: *c1cnc(*)s1 ; *c1csc(*)n1 ; "and" ; *c1csc(*)n1]

In some cases, the heterocycle of W is selected from

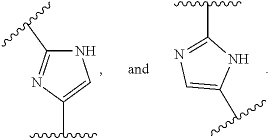 [SMILES: *c1c[nH]c(*)n1 ; *c1cnc(*)[nH]1]

In some cases, the heterocycle of W is selected from

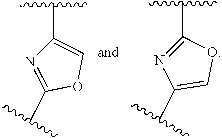 [SMILES: *c1coc(*)n1 ; *c1coc(*)n1]

In some cases, the heterocycle of W is selected from

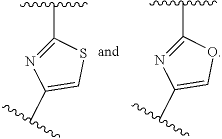 [SMILES: *c1csc(*)n1 ; *c1coc(*)n1]

In some cases, the heterocycle of W is

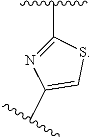 [SMILES: *c1csc(*)n1]

In some cases, the heterocycle of W is

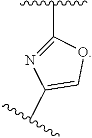 [SMILES: *c1coc(*)n1]

In some embodiments, for a compound or salt of Formula (D*), R⁴ is selected from optionally substituted C₁-C₆ alkyl, optionally substituted C₃₋₆ carbocycle and optionally substituted 3- to 8-membered heterocycle. In some cases, R⁴ is selected from optionally substituted C₁-C₆ alkyl. In some cases, R⁴ is selected from optionally substituted C₃₋₆ carbocycle and optionally substituted 3- to 8-membered heterocycle. In some cases, R⁴ is selected from optionally substituted 3- to 8-membered heterocycle. In some cases, for R⁴, the optional substituents of the optionally substituted C₁-C₆ alkyl are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, for R⁴, the optional substituents of the optionally substituted C₃₋₆ carbocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, for R⁴, the optional substituents of the optionally substituted C₃₋₆ carbocycle are independently selected from halogen and —C₁₋₁₀ haloalkyl. In some cases, for R⁴, the optional substituents of the optionally substituted 3- to 8-membered heterocycle are independently selected from halogen, —OH, —CN, —NO₂, —NH₂, oxo, —O—C₁₋₁₀ alkyl, —C₁₋₁₀ haloalkyl, and —O—C₁₋₁₀ alkyl. In some cases, R⁴ is selected from unsubstituted C₁-C₆ alkyl, unsubstituted 3- to 6-membered heterocycle, and C₃₋₆ carbocycle optionally substituted with one or more halogens. In some cases, R⁴ is selected from

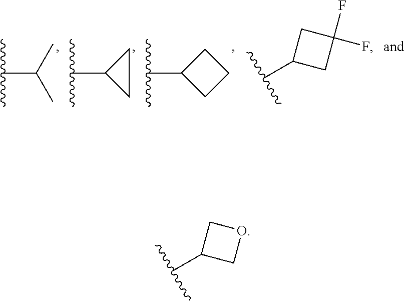

In some cases, R⁴ is selected from

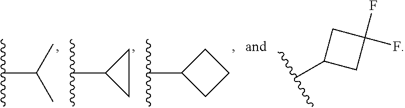

In some cases, R⁴ is

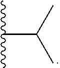 [SMILES: *C(C)C]

In some cases, R⁴ is selected from

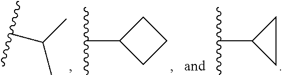

In some embodiments, for a compound or salt of Formula (D*), R¹ is substituted C₁-C₆alkyl.

In some embodiments, for a compound or salt of Formula (D*), R¹ is an optionally substituted 3 to 10-membered heterocycle. In some cases, for R¹, the 3- to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, —O—C₁₋₁₀ alkyl, and —NO₂. In some cases, for R¹, the 3- to 10-membered heterocycle is optionally substituted with one or more substituents independently selected at each occurrence from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (D*), for R¹, the heterocycle has at least one nitrogen atom, phosphorous atom, or oxygen atom. In some cases, for R¹, the heterocycle has at least one nitrogen atom. In some cases, for R¹, the heterocycle has at least two nitrogen atoms. In some cases, for R¹, the heterocycle has at most two nitrogen atoms. In some cases, for R¹, the heterocycle has at most one nitrogen atom. In some cases, for R¹, the heterocycle has at one oxygen atom. In some cases, for R¹, the heterocycle is a spirocycle. In some cases, for R¹, the heterocycle is a bridged heterocycle. In some cases, for R¹, the heterocycle is unsaturated. In some cases, for R¹, the heterocycle is saturated.

In some embodiments, for a compound or salt of Formula (D*), R¹ is selected from

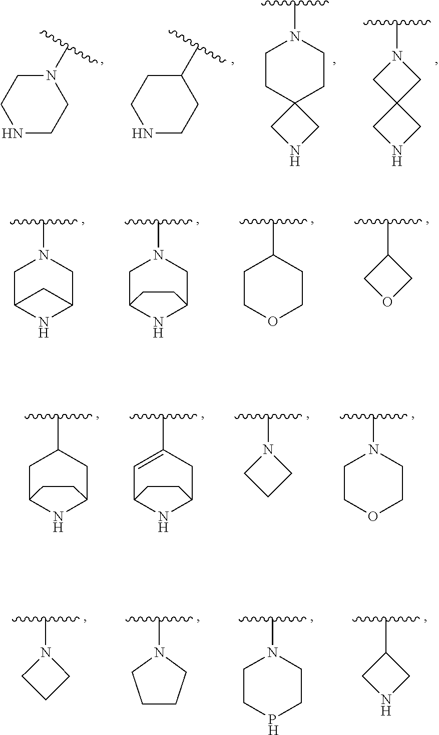

any of which are optionally substituted. In some cases, R¹ is selected from

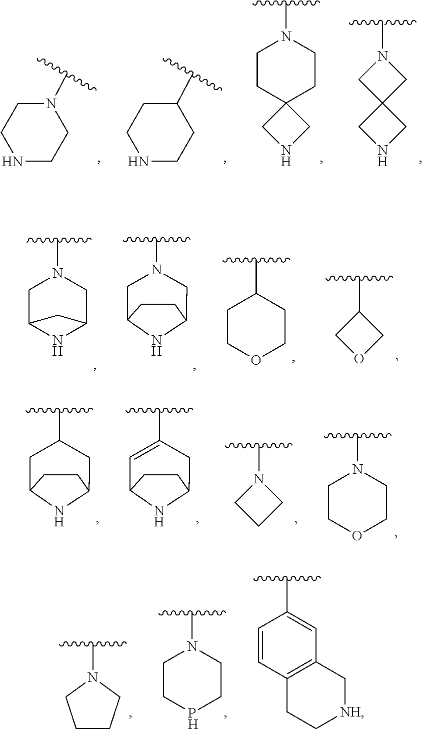

any of which are optionally substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl. In some cases, R¹ is selected from

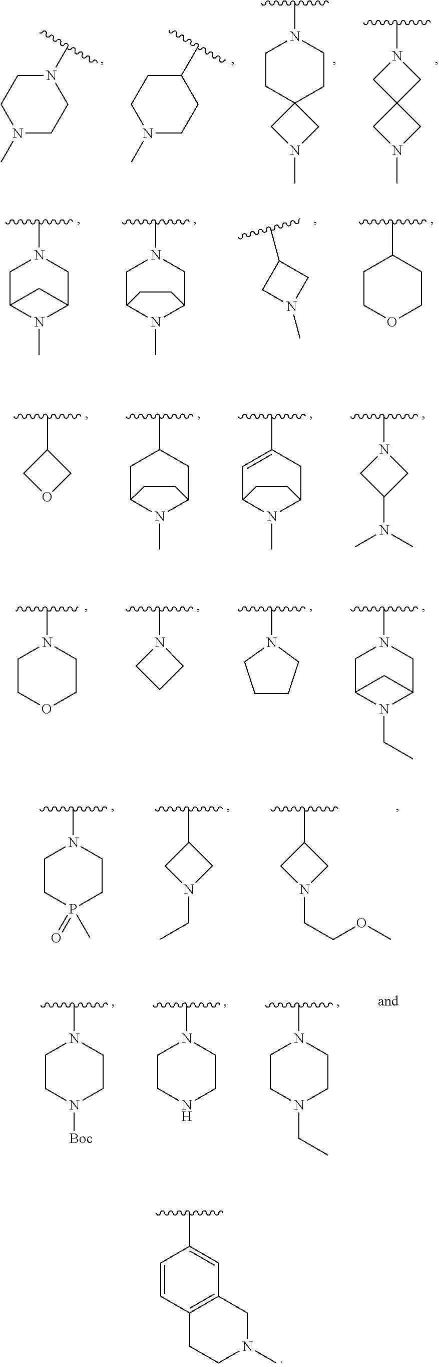

In some cases, R¹ is selected from

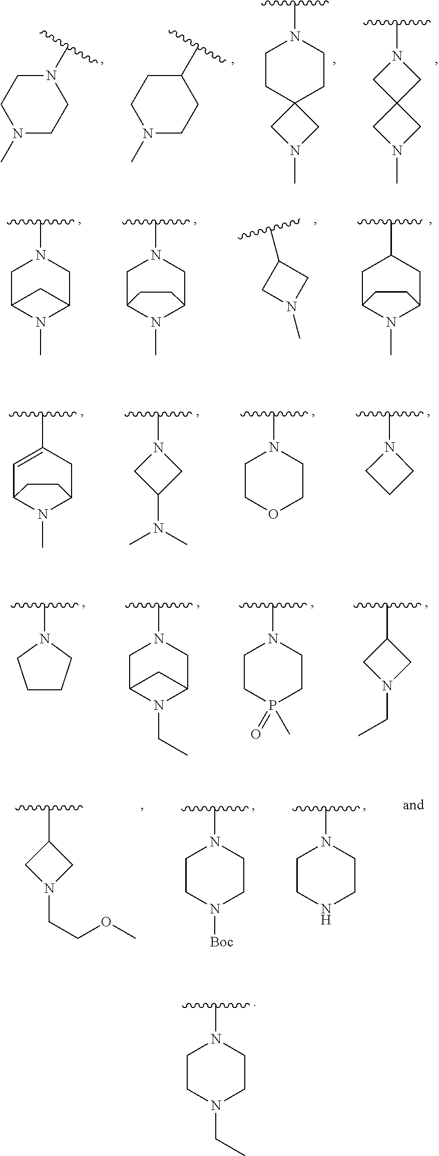

In some cases, R¹ is selected from

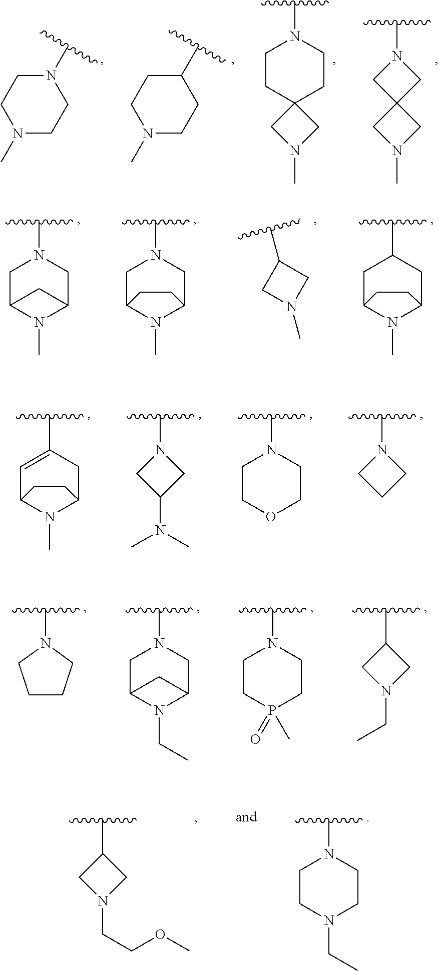

In some cases, R¹ is selected from

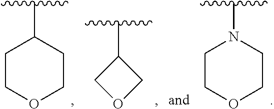

in some cases, R¹ is selected from

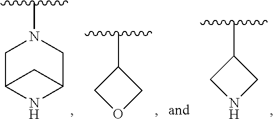

any of which are optionally substituted with one or more substituents selected from unsubstituted C₁₋₁₀ alkyl. In some cases, R¹ is selected from

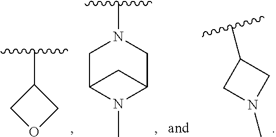

In some embodiments, for a compound or salt of Formula (D*), R¹ is selected from

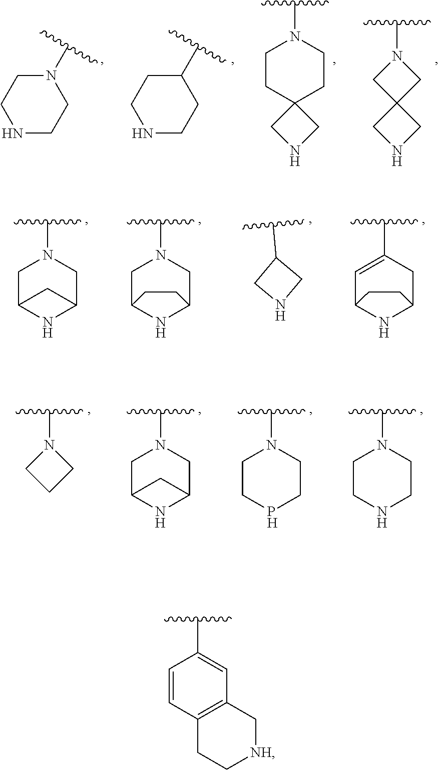

each of which are substituted with one or more substituents selected from —NH₂, —N(H)C₁-C₆ alkyl, —N(C₁-C₆ alkyl)₂, oxo, and optionally substituted C₁₋₁₀ alkyl, wherein the optional substituents on the C₁₋₁₀ alkyl are independently selected at each occurrence from one or more oxo and —O—C₁₋₁₀ alkyl.

In some embodiments, for a compound or salt of Formula (D*), R¹ is substituted piperazine.

In some embodiments, for a compound or salt of Formula (D*), R¹ is an optionally substituted 6- to 10-membered heterocycloalkyl. In some cases, the optional substituents of the optionally substituted 6- to 10-membered heterocycloalkyl for R¹ are selected from C₁₋₆ alkyl. In some cases, the 6- to 10-membered heterocycloalkyl is a spiro heterocycloalkyl. In some cases, R¹ is selected from optionally substituted piperazine, optionally substituted diazabicyclo [3.2.1]octane, optionally substituted diazabicyclo[3.1.1]heptane, optionally substituted diazaspiro[3.5]nonane, and optionally substituted diazaspiro[3.3]heptane. In some cases, the optional are selected from C₁₋₆ alkyl.

Included in the present disclosure are salts, particularly pharmaceutically acceptable salts, of the compounds described herein. The compounds of the present invention that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

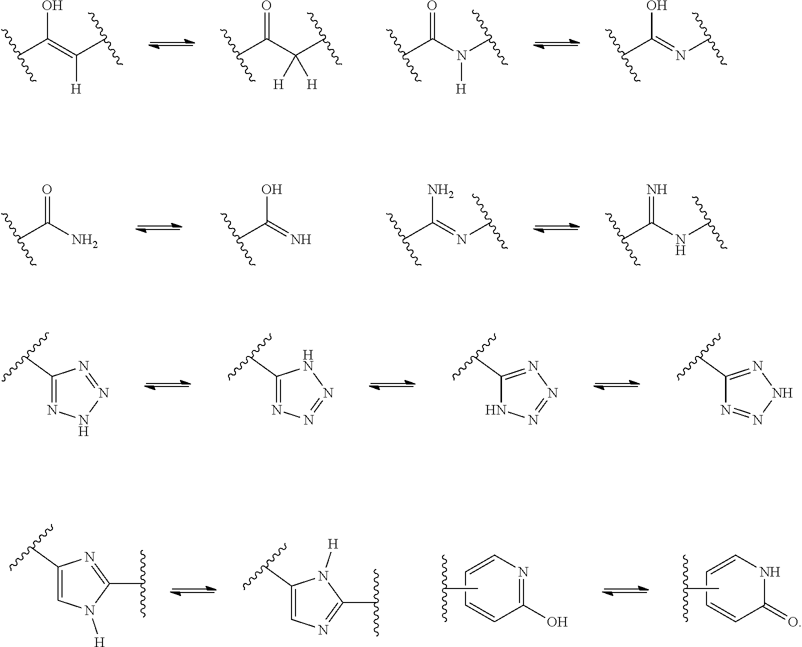

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (3H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention. In some embodiments, where isotopic variations are illustrated, the remaining atoms of the compound may optionally contain unnatural proportions of atomic isotopes.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

In some embodiments of a compound disclosed herein, one or more of R¹, R³, R⁴, R⁵, W, Z, Y, and R¹⁰ groups comprise deuterium at a percentage higher than the natural abundance of deuterium.

In some embodiments of a compound disclosed herein, one or more hydrogens are replaced with one or more deuteriums in one or more of the following groups R¹, R³, R⁴, R⁵, W, Z, Y, and R¹⁰.

In some embodiments of a compound disclosed herein, the abundance of deuterium in each of R¹, R³, R⁴, R⁵, W, Z, Y, and R¹⁰ is independently at least 1%, at least 10%, at least 20%, at least 30%, at least 40%, at least 50%, at least 60%, at least 70%, at least 80%, at least 90%, or 100% of a total number of hydrogen and deuterium.

In some embodiments of a compound disclosed herein, one or more hydrogens of ring W are replaced with one or more deuteriums. In some embodiments of a compound disclosed herein, one or more hydrogens of the optional substituents of the heterocycle for R¹ is replaced with one or more deuteriums (for example,

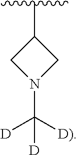

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

The compounds described herein may in some cases exist as diastereomers, enantiomers, or other stereoisomeric forms. Where absolute stereochemistry is not specified, the compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### C. Pharmaceutical Compositions

Provided herein, in certain embodiments, are compositions comprising a therapeutically effective amount of any compound or salt of any one of Formulas (I), Formula (II), Formula (IIA), Formula (AA), Formula (B), Formula (C), Formula (D), Formula (A*), Formula (B*), Formula (C*), or Formula (D*) (also referred to herein as “a pharmaceutical agent”).

Pharmaceutical compositions may be formulated using one or more physiologically acceptable carriers including excipients and auxiliaries which facilitate processing of the pharmaceutical agent into preparations which are used pharmaceutically. Proper formulation is dependent upon the route of administration chosen. A summary of pharmaceutical compositions is found, for example, in Remington: The Science and Practice of Pharmacy, Nineteenth Ed (Easton, Pa., Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

The compositions and methods of the present disclosure may be utilized to treat an individual in need thereof. In certain embodiments, the individual is a mammal such as a human, or a non-human mammal. When administered to an animal, such as a human, the composition or the pharmaceutical agent, is preferably administered as a pharmaceutical composition comprising, for example, a pharmaceutical agent and a pharmaceutically acceptable carrier or excipient. Pharmaceutically acceptable carriers are well known in the art and include, for example, aqueous solutions such as water or physiologically buffered saline or other solvents or vehicles such as glycols, glycerol, oils such as olive oil, or injectable organic esters. In a preferred embodiment, when such pharmaceutical compositions are for human administration, particularly for invasive routes of administration, e.g., routes, such as injection or implantation, that circumvent transport or diffusion through an epithelial barrier, the aqueous solution is pyrogen-free, or substantially pyrogen-free. The excipients can be chosen, for example, to effect delayed release of an agent or to selectively target one or more cells, tissues or organs. The pharmaceutical composition can be in dosage unit form such as tablet, capsule, granule, lyophile for reconstitution, powder, solution, syrup, suppository, injection or the like. The composition can also be present in a transdermal delivery system, e.g., a skin patch. The composition can also be present in a solution suitable for topical administration, such as an eye drop.

The pharmaceutical compositions can be used as an inhibitor of tumor immunosuppression in combination with chemotherapy or immune checkpoint inhibitor therapy for cancer. In some cases, the pharmaceutical composition can be used to treat a fibrotic disease or condition including but not limited to chronic kidney fibrosis (“CKD”), liver cirrhosis, pulmonary fibrosis, renal interstitial fibrosis, myocardial infarction, skin fibrosis, systemic sclerosis (“SSc”), and graft-versus-host disease (“GVHD”). In some cases, the pharmaceutical composition can be used to treat kidney fibrosis. In some cases, the pharmaceutical composition can be used to treat skin fibrosis. In some cases, the pharmaceutical composition can be used to treat idiopathic pulmonary fibrosis (IPF). In some cases, the pharmaceutical composition can be used to treat a disease is associated with TNIK kinase.

A pharmaceutically acceptable excipient can contain physiologically acceptable agents that act, for example, to stabilize, increase solubility or to increase the absorption of a compound such as a pharmaceutical agent. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable excipient, including a physiologically acceptable agent, depends, for example, on the route of administration of the composition. The preparation or pharmaceutical composition can be a self emulsifying drug delivery system or a self microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable and metabolizable carriers that are relatively simple to make and administer.

A pharmaceutical composition (preparation) can be administered to a subject by any of a number of routes of administration including, for example, orally, for example, drenches as in aqueous or non-aqueous solutions or suspensions, tablets, capsules, including sprinkle capsules and gelatin capsules, boluses, powders, granules, pastes for application to the tongue; absorption through the oral mucosa. e.g., sublingually; anally, rectally or vaginally, for example, as a pessary, cream or foam; parenterally, including intramuscularly, intravenously, subcutaneously or intrathecally as, for example, a sterile solution or suspension; nasally; intraperitoneally; subcutaneously; transdermally, for example, as a patch applied to the skin; and topically, for example, as a cream, ointment or spray applied to the skin, or as an eye drop. The compound may also be formulated for inhalation. In certain embodiments, a compound may be simply dissolved or suspended in sterile water.

A pharmaceutical composition may be a sterile aqueous or non-aqueous solution, suspension or emulsion, e.g., a microemulsion. The excipients described herein are examples and are in no way limiting. An effective amount or therapeutically effective amount refers to an amount of the one or more pharmaceutical agents administered to a subject, either as a single dose or as part of a series of doses, which is effective to produce a desired therapeutic effect.

Subjects may generally be monitored for therapeutic effectiveness using assays and methods suitable for the condition being treated, which assays will be familiar to those having ordinary skill in the art and are described herein. Pharmacokinetics of a pharmaceutical agent, or one or more metabolites thereof, that is administered to a subject may be monitored by determining the level of the pharmaceutical agent or metabolite in a biological fluid, for example, in the blood, blood fraction, e.g., serum, and/or in the urine, and/or other biological sample or biological tissue from the subject. Any method practiced in the art and described herein to detect the agent may be used to measure the level of the pharmaceutical agent or metabolite during a treatment course.

The dose of a pharmaceutical agent described herein for treating a disease or disorder may depend upon the subject's condition, that is, stage of the disease, severity of symptoms caused by the disease, general health status, as well as age, gender, and weight, and other factors apparent to a person skilled in the medical art. Pharmaceutical compositions may be administered in a manner appropriate to the disease to be treated as determined by persons skilled in the medical arts. In addition to the factors described herein and above related to use of pharmaceutical agent for treating a disease or disorder, suitable duration and frequency of administration of the pharmaceutical agent may also be determined or adjusted by such factors as the condition of the patient, the type and severity of the patient's disease, the particular form of the active ingredient, and the method of administration. Optimal doses of an agent may generally be determined using experimental models and/or clinical trials. The optimal dose may depend upon the body mass, weight, or blood volume of the subject. The use of the minimum dose that is sufficient to provide effective therapy is usually preferred. Design and execution of pre-clinical and clinical studies for a pharmaceutical agent, including when administered for prophylactic benefit, described herein are well within the skill of a person skilled in the relevant art. When two or more pharmaceutical agents are administered to treat a disease or disorder, the optimal dose of each pharmaceutical agent may be different, such as less than when either agent is administered alone as a single agent therapy. In certain particular embodiments, two pharmaceutical agents in combination may act synergistically or additively, and either agent may be used in a lesser amount than if administered alone. An amount of a pharmaceutical agent that may be administered per day may be, for example, between about 0.01 mg/kg and 100 mg/kg, e.g., between about 0.1 to 1 mg/kg, between about 1 to 10 mg/kg, between about 10-50 mg/kg, between about 50-100 mg/kg body weight. In other embodiments, the amount of a pharmaceutical agent that may be administered per day is between about 0.01 mg/kg and 1000 mg/kg, between about 100-500 mg/kg, or between about 500-1000 mg/kg body weight. The optimal dose, per day or per course of treatment, may be different for the disease or disorder to be treated and may also vary with the administrative route and therapeutic regimen.

Pharmaceutical compositions comprising a pharmaceutical agent can be formulated in a manner appropriate for the delivery method by using techniques routinely practiced in the art. The composition may be in the form of a solid, e.g., tablet, capsule, semi-solid, e.g., gel, liquid, or gas, e.g., aerosol. In other embodiments, the pharmaceutical composition is administered as a bolus infusion.

Pharmaceutical acceptable excipients are well known in the pharmaceutical art and described, for example, in Rowe et al., Handbook of Pharmaceutical Excipients: A Comprehensive Guide to Uses, Properties, and Safety, 5^{th} Ed., 2006, and in Remington: The Science and Practice of Pharmacy (Gennaro, 21^{st} Ed. Mack Pub. Co., Easton, PA (2005)). Exemplary pharmaceutically acceptable excipients include sterile saline and phosphate buffered saline at physiological pH. Preservatives, stabilizers, dyes, buffers, and the like may be provided in the pharmaceutical composition. In addition, antioxidants and suspending agents may also be used. In general, the type of excipient is selected based on the mode of administration, as well as the chemical composition of the active ingredient(s). Alternatively, compositions described herein may be formulated as a lyophilizate. A composition described herein may be lyophilized or otherwise formulated as a lyophilized product using one or more appropriate excipient solutions for solubilizing and/or diluting the pharmaceutical agent(s) of the composition upon administration. In other embodiments, the pharmaceutical agent may be encapsulated within liposomes using technology known and practiced in the art. In certain particular embodiments, a pharmaceutical agent is not formulated within liposomes for application to a stent that is used for treating highly, though not totally, occluded arteries. Pharmaceutical compositions may be formulated for any appropriate manner of administration described herein and in the art.

A pharmaceutical composition, e.g., for oral administration or for injection, infusion, subcutaneous delivery, intramuscular delivery, intraperitoneal delivery or other method, may be in the form of a liquid. A liquid pharmaceutical composition may include, for example, one or more of the following: a sterile diluent such as water, saline solution, preferably physiological saline, Ringer's solution, isotonic sodium chloride, fixed oils that may serve as the solvent or suspending medium, polyethylene glycols, glycerin, propylene glycol or other solvents; antibacterial agents; antioxidants; chelating agents; buffers and agents for the adjustment of tonicity such as sodium chloride or dextrose. A parenteral composition can be enclosed in ampoules, disposable syringes or multiple dose vials made of glass or plastic. The use of physiological saline is preferred, and an injectable pharmaceutical composition is preferably sterile. In another embodiment, for treatment of an ophthalmological condition or disease, a liquid pharmaceutical composition may be applied to the eye in the form of eye drops. A liquid pharmaceutical composition may be delivered orally.

For oral formulations, at least one of the pharmaceutical agents described herein can be used alone or in combination with appropriate additives to make tablets, powders, granules or capsules, and if desired, with diluents, buffering agents, moistening agents, preservatives, coloring agents, and flavoring agents. The pharmaceutical agents may be formulated with a buffering agent to provide for protection of the compound from low pH of the gastric environment and/or an enteric coating. A pharmaceutical agent included in a pharmaceutical composition may be formulated for oral delivery with a flavoring agent, e.g., in a liquid, solid or semi-solid formulation and/or with an enteric coating.

A pharmaceutical composition comprising any one of the pharmaceutical agents described herein may be formulated for sustained or slow release, also called timed release or controlled release. Such compositions may generally be prepared using well known technology and administered by, for example, oral, rectal, intradermal, or subcutaneous implantation, or by implantation at the desired target site. Sustained-release formulations may contain the compound dispersed in a carrier matrix and/or contained within a reservoir surrounded by a rate controlling membrane. Excipients for use within such formulations are biocompatible, and may also be biodegradable; preferably the formulation provides a relatively constant level of active component release. The amount of pharmaceutical agent contained within a sustained release formulation depends upon the site of implantation, the rate and expected duration of release, and the nature of the condition, disease or disorder to be treated or prevented.

In certain embodiments, the pharmaceutical compositions comprising a pharmaceutical agent are formulated for transdermal, intradermal, or topical administration. The compositions can be administered using a syringe, bandage, transdermal patch, insert, or syringe-like applicator, as a powder/talc or other solid, liquid, spray, aerosol, ointment, foam, cream, gel, paste. This preferably is in the form of a controlled release formulation or sustained release formulation administered topically or injected directly into the skin adjacent to or within the area to be treated, e.g., intradermally or subcutaneously. The active compositions can also be delivered via iontophoresis. Preservatives can be used to prevent the growth of fungi and other microorganisms. Suitable preservatives include, but are not limited to, benzoic acid, butylparaben, ethyl paraben, methyl paraben, propylparaben, sodium benzoate, sodium propionate, benzalkonium chloride, benzethonium chloride, benzyl alcohol, cetylpyridinium chloride, chlorobutanol, phenol, phenylethyl alcohol, thimerosal, and combinations thereof.

Pharmaceutical compositions comprising a pharmaceutical agent can be formulated as emulsions for topical application. An emulsion contains one liquid distributed in the body of a second liquid. The emulsion may be an oil-in-water emulsion or a water-in-oil emulsion. Either or both of the oil phase and the aqueous phase may contain one or more surfactants, emulsifiers, emulsion stabilizers, buffers, and other excipients. The oil phase may contain other oily pharmaceutically approved excipients. Suitable surfactants include, but are not limited to, anionic surfactants, non-ionic surfactants, cationic surfactants, and amphoteric surfactants. Compositions for topical application may also include at least one suitable suspending agent, antioxidant, chelating agent, emollient, or humectant.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. Liquid sprays may be delivered from pressurized packs, for example, via a specially shaped closure. Oil-in-water emulsions can also be used in the compositions, patches, bandages and articles. These systems are semisolid emulsions, micro-emulsions, or foam emulsion systems.

In some embodiments, the pharmaceutical agent described herein can be formulated as in inhalant. Inhaled methods can deliver medication directly to the airway. The pharmaceutical agent can be formulated as aerosols, microspheres, liposomes, or nanoparticles. The pharmaceutical agent can be formulated with solvents, gases, nitrates, or any combinations thereof. Compositions described herein are optionally formulated for delivery as a liquid aerosol or inhalable dry powder. Liquid aerosol formulations are optionally nebulized predominantly into particle sizes that can be delivered to the terminal and respiratory bronchioles. Liquid aerosol and inhalable dry powder formulations are preferably delivered throughout the endobronchial tree to the terminal bronchioles and eventually to the parenchymal tissue.

Aerosolized formulations described herein are optionally delivered using an aerosol forming device, such as a jet, vibrating porous plate or ultrasonic nebulizer, preferably selected to allow the formation of aerosol particles having with a mass medium average diameter predominantly between 1 to 5μ. Further, the formulation preferably has balanced osmolarity ionic strength and chloride concentration, and the smallest aerosolizable volume able to deliver effective dose of the pharmaceutical agent. Additionally, the aerosolized formulation preferably does not impair negatively the functionality of the airways and does not cause undesirable side effects.

Aerosolization devices suitable for administration of aerosol formulations described herein include, for example, jet, vibrating porous plate, ultrasonic nebulizers and energized dry powder inhalers, that are able to nebulize the formulation into aerosol particle size predominantly in the size range from 1-5μ. Predominantly in this application means that at least 70% but preferably more than 90% of all generated aerosol particles are within 1-5μ range. A jet nebulizer works by air pressure to break a liquid solution into aerosol droplets. Vibrating porous plate nebulizers work by using a sonic vacuum produced by a rapidly vibrating porous plate to extrude a solvent droplet through a porous plate. An ultrasonic nebulizer works by a piezoelectric crystal that shears a liquid into small aerosol droplets. A variety of suitable devices are available, including, for example, AeroNeb™ and AeroDose™ vibrating porous plate nebulizers (AeroGen, Inc., Sunnyvale, California), Sidestream® nebulizers (Medic-Aid Ltd., West Sussex, England), Pari LC® and Pari LC Star® jet nebulizers (Pari Respiratory Equipment, Inc., Richmond, Virginia), and Aerosonic™ (DeVilbiss Medizinische Produkte (Deutschland) GmbH, Heiden, Germany) and UltraAire® (Omron Healthcare, Inc., Vernon Hills, Illinois) ultrasonic nebulizers.

In some embodiments, the pharmaceutical agent(s) can be formulated with oleaginous bases or ointments to form a semisolid composition with a desired shape. In addition to the pharmaceutical agent, these semisolid compositions can contain dissolved and/or suspended bactericidal agents, preservatives and/or a buffer system. A petrolatum component that may be included may be any paraffin ranging in viscosity from mineral oil that incorporates isobutylene, colloidal silica, or stearate salts to paraffin waxes. Absorption bases can be used with an oleaginous system. Additives may include cholesterol, lanolin (lanolin derivatives, beeswax, fatty alcohols, wool wax alcohols, low HLB (hydrophobellipophobe balance) emulsifiers, and assorted ionic and nonionic surfactants, singularly or in combination.

Controlled or sustained release transdermal or topical formulations can be achieved by the addition of time-release additives, such as polymeric structures, matrices, that are available in the art. For example, the compositions may be administered through use of hot-melt extrusion articles, such as bioadhesive hot-melt extruded film. The formulation can comprise a cross-linked polycarboxylic acid polymer formulation. A cross-linking agent may be present in an amount that provides adequate adhesion to allow the system to remain attached to target epithelial or endothelial cell surfaces for a sufficient time to allow the desired release of the compound.

An insert, transdermal patch, bandage or article can comprise a mixture or coating of polymers that provide release of the pharmaceutical agents at a constant rate over a prolonged period of time. In some embodiments, the article, transdermal patch or insert comprises water-soluble pore forming agents, such as polyethylene glycol (PEG) that can be mixed with water insoluble polymers to increase the durability of the insert and to prolong the release of the active ingredients.

Transdermal devices (inserts, patches, bandages) may also comprise a water insoluble polymer. Rate controlling polymers may be useful for administration to sites where pH change can be used to effect release. These rate controlling polymers can be applied using a continuous coating film during the process of spraying and drying with the active compound. In one embodiment, the coating formulation is used to coat pellets comprising the active ingredients that are compressed to form a solid, biodegradable insert.

A polymer formulation can also be utilized to provide controlled or sustained release. Bioadhesive polymers described in the art may be used. By way of example, a sustained-release gel and the compound may be incorporated in a polymeric matrix, such as a hydrophobic polymer matrix. Examples of a polymeric matrix include a microparticle. The microparticles can be microspheres, and the core may be of a different material than the polymeric shell. Alternatively, the polymer may be cast as a thin slab or film, a powder produced by grinding or other standard techniques, or a gel such as a hydrogel. The polymer can also be in the form of a coating or part of a bandage, stent, catheter, vascular graft, or other device to facilitate delivery of the pharmaceutical agent. The matrices can be formed by solvent evaporation, spray drying, solvent extraction and other methods known to those skilled in the art.

Kits with unit doses of one or more of the agents described herein, usually in oral or injectable doses, are provided. Such kits may include a container containing the unit dose, an informational package insert describing the use and attendant benefits of the drugs in treating disease, and optionally an appliance or device for delivery of the composition.

### D. Methods of Treatment

The compounds described herein can be used in the preparation of medicaments for the prevention or treatment of diseases or conditions. In addition, a method for treating any of the diseases or conditions described herein in a subject in need of such treatment, involves administration of pharmaceutical compositions containing at least one compound described herein, or a pharmaceutically acceptable salt, pharmaceutically acceptable prodrug, or pharmaceutically acceptable solvate thereof, in therapeutically effective amounts to said subject.

The compositions containing the compound(s) described herein can be administered for prophylactic and/or therapeutic treatments. In therapeutic applications, the compositions are administered to a patient already suffering from a disease or condition, in an amount sufficient to cure or at least partially arrest the symptoms of the disease or condition. Amounts effective for this use will depend on the severity and course of the disease or condition, previous therapy, the patient's health status, weight, and response to the drugs, and the judgment of the treating physician.

In prophylactic applications, compositions containing the compounds described herein are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in a patient, effective amounts for this use will depend on the severity and course of the disease, disorder or condition, previous therapy, the patient's health status and response to the drugs, and the judgment of the treating physician.

In the case wherein the patient's condition does not improve, upon the doctor's discretion the administration of the compounds may be administered chronically, that is, for an extended period of time, including throughout the duration of the patient's life in order to ameliorate or otherwise control or limit the symptoms of the patient's disease or condition.

Once improvement of the patient's conditions has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

The amount of a given agent that will correspond to such an amount will vary depending upon factors such as the particular compound, disease or condition and its severity, the identity (e.g., weight) of the subject or host in need of treatment, but can nevertheless be determined in a manner recognized in the field according to the particular circumstances surrounding the case, including, e.g., the specific agent being administered, the route of administration, the condition being treated, and the subject or host being treated. In general, however, doses employed for adult human treatment will typically be in the range of about 0.02-about 5000 mg per day, in some embodiments, about 1-about 1500 mg per day. The desired dose may conveniently be presented in a single dose or as divided doses administered simultaneously (or over a short period of time) or at appropriate intervals, for example as two, three, four or more sub-doses per day. In some embodiments, compounds and pharmaceutical compositions described herein are administered once daily. In some embodiments, compounds and pharmaceutical compositions described herein are administered twice daily. In some embodiments, compounds and pharmaceutical compositions described herein are administered 3 times a day. In some embodiments, compounds and pharmaceutical compositions described herein are administered once weekly. In some embodiments, compounds and pharmaceutical compositions described herein are administered twice weekly. In some embodiments, compounds and pharmaceutical compositions described herein are administered 3 to 7 times a week. In some embodiments, compounds and pharmaceutical compositions described herein are administered orally. In some embodiments, compounds and pharmaceutical compositions described herein are administered intravenously. In some embodiments, compounds and pharmaceutical compositions described herein are administered topically. For example, a compound described herein can be administered topically at doses of 0.001% to 10%. In some embodiments, compounds and pharmaceutical compositions described herein are administered to treat chronic diseases.

The pharmaceutical composition described herein may be in unit dosage forms suitable for single administration of precise dosages. In unit dosage form, the formulation is divided into unit doses containing appropriate quantities of one or more compound. The unit dosage may be in the form of a package containing discrete quantities of the formulation. Non-limiting examples are packaged tablets or capsules, and powders in vials or ampoules. Aqueous suspension compositions can be packaged in single-dose non-reclosable containers. Alternatively, multiple-dose reclosable containers can be used, in which case it is typical to include a preservative in the composition. By way of example only, formulations for parenteral injection may be presented in unit dosage form, which include, but are not limited to ampoules, or in multi-dose containers, with an added preservative.

Toxicity and therapeutic efficacy of such therapeutic regimens can be determined by standard pharmaceutical procedures in cell cultures or experimental animals, including, but not limited to, the determination of the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

In an aspect provided herein, the invention provides for inhibitors of TNIK kinase. Accordingly, the TNIK kinase inhibitors can be used to inhibit a biological pathway downstream from inhibiting TNIK. In some aspects, the TNIK inhibitor can inhibit fibrillar collagen, and thereby can inhibit biological activity related to regulation of the extracellular matrix, and regulation of remodeling the extracellular matrix. The TNIK inhibitor can inhibit regulation of cell growth, differentiation, cell migration, proliferation, and metabolism.

In certain embodiments, inhibiting the TNIK can inhibit certain TNIK related biological pathways. In certain aspects, the inhibiting of TNIK inhibits the Wnt pathway.

In certain embodiments, the inhibiting of TNIK inhibits cytoskeletal rearrangements. The inhibition of TNIK can inhibit the c-Jun N-terminal kinase pathway. The inhibition of TNIK can inhibit the phosphorylation of Gelsolin. The inhibition of TNIK can inhibit the regulation of the cytoskeleton, such as cytoskeletal rearrangements.

In certain embodiments, the inhibiting of TNIK inhibits carcinogenesis. In certain aspect, the administering of the TNIK inhibitor includes a therapeutically effective amount of the compound sufficient to treat cancer by: inhibiting cancer cell growth; inhibiting cancer cell migration; inhibiting cancer cell proliferation; or inhibiting cancer cell migration. In certain embodiments, the invention provides a method of treating or preventing a disease, state or condition in a patient in need thereof comprising administering to the patient an effective amount of a compound of any one of embodiments of the invention or a pharmaceutically acceptable salt thereof. The disease, state or condition may be selected from the group consisting of colorectal cancer, gastric cancer, breast cancer, lung cancer, pancreatic cancer, prostate cancer, multiple myeloma, chronic myelogenous leukemia, cancer metastasis, fibrosis and psychiatric disorders. In some embodiments, the cancer is colorectal cancer. In some embodiments, the cancer is gastric cancer. In some embodiments, the cancer is breast cancer. In some embodiments, the cancer is lung cancer. In some embodiments, the cancer is pancreatic cancer. In some embodiments, the cancer is prostate cancer. In some embodiments, the cancer is multiple myeloma. In some embodiments, the cancer is chronic myelogenous leukemia. In some embodiments, the cancer is cancer metastasis.

In certain embodiments, the cancer is a solid tumor. In certain embodiments, the cancer is not a solid tumor.

In certain embodiments, the inhibiting of TNIK inhibits embryonic development. As such, the TNIK inhibitor can inhibit pregnancy progression and thereby be used for terminating a pregnancy.

In some embodiments, the inhibiting of TNIK inhibits TGF beta signaling. The TGF beta signaling pathway is involved in a various processes, and thereby inhibiting the TGF beta signaling pathway can inhibit these processes, some of which are described herein. This can include inhibiting development of an embryo as described herein for inhibiting progression of pregnancy. This can include inhibiting cell growth, cell differentiation, which may be used to inhibit pregnancy progression as well as inhibiting cancer.

In certain embodiments, inhibiting the TGF beta signaling can be used for inhibiting formation of extracellular matrix or over formation of extracellular matrix and the problems associated therewith (e.g., fibrosis). In some aspects, the inhibiting of TGF beta signaling by inhibiting TNIK inhibits glycosaminoglycan formation. In some aspects, the inhibiting of TGF beta by inhibiting TNIK inhibits collagen formation. In some aspects, the inhibiting of TNIK inhibits fibrosis. In some aspects, the inhibited fibrosis is selected from pulmonary fibrosis (e.g., idiopathic or radiation induced), cystic fibrosis, liver fibrosis (e.g., cirrhosis), myocardial fibrosis (e.g., atrial fibrosis, endomyocardial fibrosis, old myocardial infarction), kidney fibrosis, brain fibrosis (e.g., glial scar), arterial fibrosis, arthrofibrosis (e.g., knee, shoulder, other joints), intestinal fibrosis (e.g., Crohn's disease), Dupytren's contracture fibrosis (e.g., hands, fingers), keloid fibrosis (e.g., skin), mediastinal fibrosis (e.g., soft tissue of the mediastinum), myelofibrosis (e.g., bone marrow), peyronie's disease fibrosis (e.g., penis), progressive massive fibrosis (e.g., lungs, complication of coal worker's pneumoconiosis), retroperitoneal fibrosis (e.g., soft tissue of the retroperitoneum), scleroderma sclerosis fibrosis (e.g., skin, lungs), adhesive capsulitis fibrosis (e.g., shoulder), or combinations thereof. In some aspects, the fibrosis is skin fibrosis.

In certain embodiments, the TNIK inhibitor can be used to inhibit the epithelial to mesenchymal transition of cancer cells and/or development of fibrosis. In some aspects, this can include inhibiting the Smad signaling pathways. In some aspects, this can include inhibiting the non-Smad signaling pathways. In some aspects, this can include inhibiting Wnt, NF-KB, FAC-Src-paxillin-related focal adhesion, and MAP kinases (e.g., ERK and JNK) signaling pathways.

In certain embodiments, the disclosure provides for methods of treating or preventing a fibrotic disease or condition. In some embodiments, the fibrotic disease or condition is selected from pulmonary fibrosis, cystic fibrosis, liver fibrosis, myocardial fibrosis, kidney fibrosis, brain fibrosis, arterial fibrosis, arthrofibrosis, intestinal fibrosis, Dupytren's contracture fibrosis, keloid fibrosis, mediastinal fibrosis, myelofibrosis, peyronie's disease fibrosis, progressive massive fibrosis, retroperitoneal fibrosis, scleroderma sclerosis fibrosis, adhesive capsulitis fibrosis, or combinations thereof. In some embodiments, the fibrotic disease is selected from liver cirrhosis, pulmonary fibrosis, renal interstitial fibrosis, myocardial infarction, systemic sclerosis (SSc), and graft-versus-host disease (GVHD). In some embodiments, the fibrotic disease is kidney fibrosis.

In certain embodiments, the disclosure provides for methods of treating a kidney disease. In some embodiments, the kidney disease is chronic kidney fibrosis (CKD). In some embodiments, the kidney disease is a kidney fibrosis.

In some embodiments, the fibrotic disease is liver cirrhosis. In some embodiments, the fibrotic disease is pulmonary fibrosis. In some embodiments, the fibrotic disease is idiopathic pulmonary fibrosis (IPF).

In some embodiments, the fibrotic disease is kidney fibrosis wherein the disease is chronic or acute. In some embodiments, the kidney fibrosis causes glomerulosclerosis or tubulointerstitial fibrosis. In some embodiments, the fibrotic disease is renal interstitial fibrosis. In some embodiments, the fibrotic disease is acute interstitial nephritis (AlN).

In some embodiments, the fibrotic disease is systemic sclerosis (SSc). In some embodiments, the fibrotic disease is graft-versus-host disease (GVHD). In some embodiments, the fibrotic disease is hypertrophic scarring (HTS).

In some embodiments, provided herein are methods of suppressing fibrosis markers in a subject such as alpha-smooth muscle actin, or α-SMA, and collagen by administering compounds and pharmaceutical compositions of the present disclosure.

In some embodiments, provided herein are methods of antagonizing fibroblast-to-myofibroblast transition (FMT) in primary human lung fibroblasts. In some embodiments, provided herein are methods of antagonizing epithelial-mesenchymal transition (EMT) in primary human epithelial cells.

In some embodiments, provided herein are methods of reducing collagen and hydroxyproline in the skin by administering compounds and pharmaceutical compositions of the present disclosure, e.g., via oral or topical administration.

In some embodiments, compounds and pharmaceutical compositions described herein are administered with a second therapeutic agent. In some embodiments, the second therapeutic agent is Pirfenidone. In some embodiments, compounds and pharmaceutical compositions described herein are administered with sub-therapeutic doses of Pirfenidone.

Compounds and pharmaceutical compositions described herein can be administered to a subject for a period of about 1 day to about 30 years or more. In some embodiments, compounds and pharmaceutical compositions described herein are administered to a subject for a period of over a year. In some embodiments, compounds and pharmaceutical compositions described herein are administered to a subject for a period of 3 months to 5 years. In some embodiments, compounds and pharmaceutical compositions described herein are administered to a subject for a period of 1 month to 1 year or any numbers or ranges therebetween (e.g., 2-3 months, 1-6 months, 6-12 months, 1-3 months, etc.)

### EXAMPLES

The following examples are offered to illustrate, but not to limit the claimed invention. The following examples further illustrate the invention but, of course, should not be construed as in any way limiting its scope.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### Illustrative Synthesis Schemes

The compounds and salts of Formulas (I), Formula (II), Formula (IIA), Formula (AA), Formula (B), Formula (C), Formula (D), Formula (A*), Formula (B*), Formula (C*), or Formula (D*), can be synthesized according to one or more illustrative schemes herein and/or techniques known in the art. Materials used herein are either commercially available or prepared by synthetic methods generally known in the art. These schemes are not limited to the compounds listed in the examples or by any particular substituents, which are employed for illustrative purposes. Although various steps are described and depicted in the synthesis schemes below, the steps in some cases may be performed in a different order than the order shown below. Numberings or R groups in each scheme do not necessarily correspond to that of the claims or other schemes or tables herein.

In some embodiments, compounds of Table 1 may be prepared according to the synthesis schemes below.

### Example 1: Synthesis of Compound 2

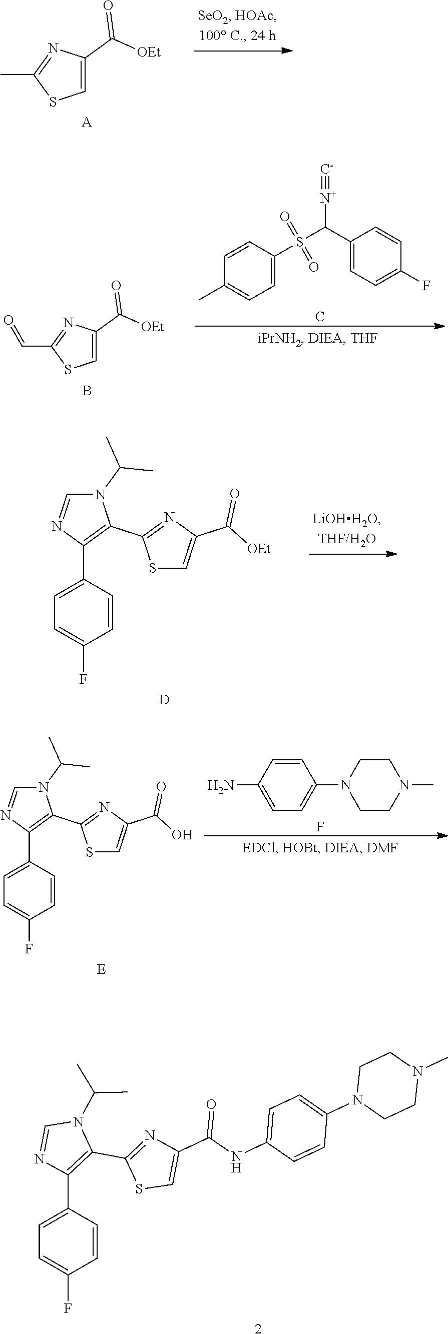

### Example 2: General Procedure for Preparation of ethyl 2-formylthiazole-4-carboxylate (Compound B)

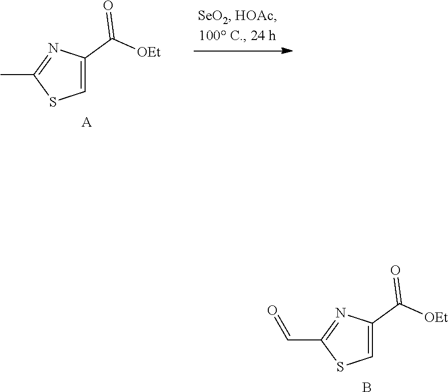

To a solution of ethyl 2-methylthiazole-4-carboxylate (Compound A) (3 g, 17.52 mmol, 1 eq) in HOAc (50 mL) was added SeO₂ (9.72 g, 87.61 mmol, 9.53 mL, 5 eq), the mixture was stirred at 100° C. for 48 hours. The mixture was filtered and concentrated under reduced pressure to give a residue. The residue was purified by column chromatography (SiO₂, Petroleum ether/Ethyl acetate=1/0 to 10/1) to afford ethyl 2-formylthiazole-4-carboxylate (Compound B) (1.5 g, 8.10 mmol, 46.22% yield, 100% purity) as a white solid, which was determined by ¹HNMR and LCMS. LCMS: Retention time: 0.610 min, (M+H)=186.1.

HNMR: (400 MHz, CDCl₃), δ=10.07 (s, 1H), 8.52 (s, 1H), 4.49 (q, J=7.2 Hz, 2H), 1.45 (t, J=7.2 Hz, 3H).

### Example 3: General Procedure for Preparation of ethyl 2-(4-(4-fluorophenyl)-1-isopropyl-1H-imidazol-5-yl)thiazole-4-carboxylate (Compound D)

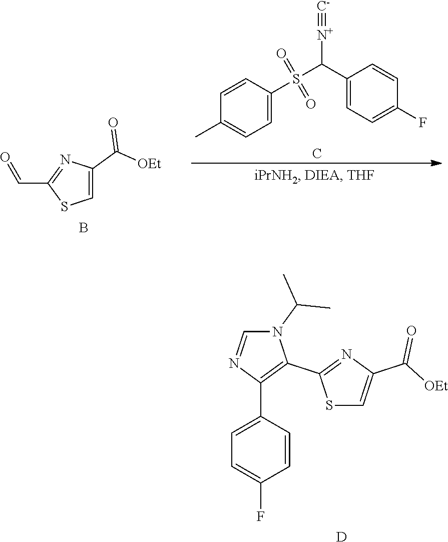

To a solution of ethyl 2-formylthiazole-4-carboxylate (Compound B) (200 mg, 539.96 umol, 1 eq) in THF (10 mL) was added propan-2-amine (127.67 mg, 2.16 mmol, 185.56 uL, 4 eq), the mixture was stirred at 40° C. for 16 hours. 1-fluoro-4-(isocyano(tosyl)methyl)benzene (Compound C) (187.47 mg, 647.95 umol, 1.2 eq) and DIEA (209.35 mg, 1.62 mmol, 282.15 uL, 3 eq) was added to the mixture, the mixture was stirred at 40° C. for 16 hours, and then stirred at 60° C. for 16 hours. The mixture was concentrated in vacuum to give a residue. The residue was purified by Prep-HPLC (neutral condition) to give the compound (180 mg, 24% purity) as a yellow solid, which was further purified by column chromatography (SiO₂, Petroleum ether/Ethyl acetate=3/1 to 3/2) to give ethyl 2-(4-(4-fluorophenyl)-1-isopropyl-1H-imidazol-5-yl)thiazole-4-carboxylate (Compound D) (46 mg, 119.15 umol, 22.07% yield, 93.1% purity) as a colorless oil, which was determined by ¹HNMR and LCMS. LCMS: Retention time: 0.728 min, (M+H)=360.1. HNMR: (400 MHz, CDCl₃), δ=8.18 (s, 1H), 7.77 (s, 1H), 7.46-7.40 (m, 2H), 7.05-6.98 (m, 2H), 4.93 (m, 1H), 4.45 (q, J=7.2 Hz, 2H), 1.50 (d, J=6.8 Hz, 6H), 1.46-1.42 (m, 3H).

### Example 3: General Procedure for Preparation of 2-(4-(4-fluorophenyl)-1-isopropyl-1H-imidazol-5-yl)thiazole-4-carboxylic acid (Compound E)

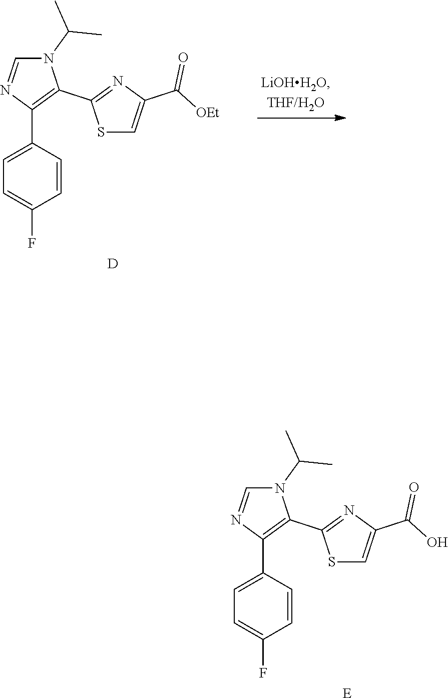

To a solution of ethyl 2-(4-(4-fluorophenyl)-1-isopropyl-1H-imidazol-5-yl)thiazole-4-carboxylate (Compound D)(45 mg, 125.20 umol, 1 eq) in THF (1 mL) and H₂O (1 mL) was added LiOH·H₂O (7.88 mg, 187.80 umol, 1.5 eq) at 20° C., the mixture was stirred at 20° C. for 3 hour. 1M HCl was added to the mixture to adjust pH=7, water (10 mL) was added to the mixture, and the mixture was extracted with EA (10 mL*2). The combined organic layers were washed with brine (5 mL*2), dried over Na₂SO₄, filtered and concentrated under reduced pressure to afford 2-(4-(4-fluorophenyl)-1-isopropyl-1H-imidazol-5-yl)thiazole-4-carboxylic acid (Compound E)(40 mg, 114.44 umol, 91.40% yield, 94.8% purity) as a white solid, which was determined by LCMS. LCMS: Retention time: 0.703 min, (M+H)=332.1.

### Example 4: General Procedure for Preparation of 2-(4-(4-fluorophenyl)-1-isopropyl-1H-imidazol-5-yl)-N-(4-(4-methylpiperazin-1-yl)phenyl)thiazole-4-carboxamide (Compound 2)

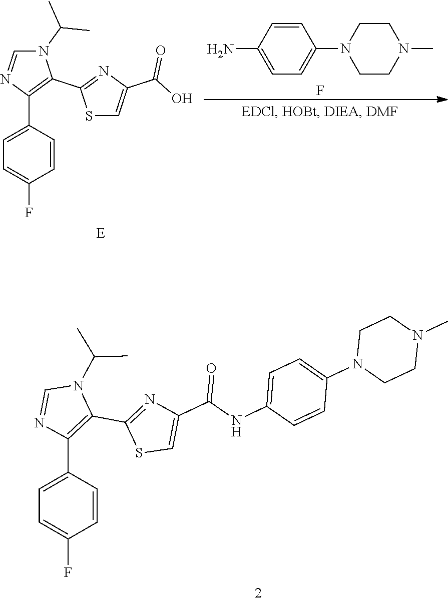

To a solution of 2-(4-(4-fluorophenyl)-1-isopropyl-1H-imidazol-5-yl)thiazole-4-carboxylic acid (Compound E) (40 mg, 120.71 umol, 1 eq) in DMF (2 mL) was added 4-(4-methylpiperazin-1-yl)aniline (F)(34.63 mg, 181.07 umol, 1.5 eq), HOBt (24.47 mg, 181.07 umol, 1.5 eq), DIEA (46.80 mg, 362.14 umol, 63.08 uL, 3 eq) and EDCI (34.71 mg, 181.07 umol, 1.5 eq), the mixture was stirred at 20° C. for 4 hours. The residue was purified by prep-HPLC (FA condition: column: Phenomenex Gemini-NX C18 75*30 mm*3 um; mobile phase: [water (0.225% FA)-ACN]; B %: 5%-35%, 7 min) to afford 2-(4-(4-fluorophenyl)-1-isopropyl-1H-imidazol-5-yl)-N-(4-(4-methylpiperazin-1-yl)phenyl)thiazole-4-carboxamide (Compound 2)(30 mg, 59.45 umol, 49.25% yield, 100% purity) as a white solid, which was determined by ¹HNMR, LCMS and HPLC. LCMS: Retention time: 0.739 min, (M+H)=505.2. HPLC: Retention time: 2.084 min.

HNMR: (400 MHz, DMSO-d₆), δ=10.08 (s, 1H), 8.56 (s, 1H), 8.17 (s, 1H), 7.66 (d, J=9.2 Hz, 2H), 7.50-7.39 (m, 2H), 7.22-7.12 (m, 2H), 6.93 (d, J=9.2 Hz, 2H), 4.70-4.54 (m, 1H), 3.15-3.05 (m, 4H), 2.46-2.43 (m, 4H), 2.22 (s, 3H), 1.45 (d, J=6.8 Hz, 6H).

### Example 5: Synthesis of Compound 1

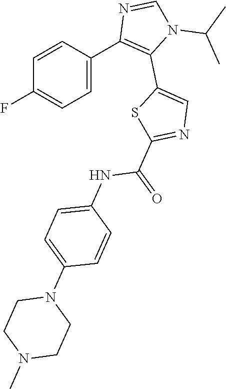 [SMILES: CC(C)n1cnc(-c2ccc(F)cc2)c1-c1cnc(C(=O)Nc2ccc(N3CCN(C)CC3)cc2)s1]

5-(4-(4-fluorophenyl)-1-isopropyl-1H-imidazol-5-yl)-N-(4-(4-methylpiperazin-1-yl)phenyl)thiazole-2-carboxamide (Compound 1) was synthesized under the same synthetic route as for Compound 2 as white solid, which was determined by ¹HNMR, LCMS and HPLC. LCMS: Retention time: 1.982 min, (M+H)=505.1. HPLC: Retention time: 5.20 min. ¹H NMR (400 MHz, CD₃OD) δ=8.11 (s, 1H), 7.96 (s, 1H), 7.67-7.61 (m, 2H), 7.46-7.39 (m, 2H), 7.07-6.97 (m, 4H), 4.38-4.27 (m, 1H), 3.26-3.16 (m, 4H), 2.67-2.58 (m, 4H), 2.35 (s, 3H), 1.52 (d, J=6.8 Hz, 6H).

### Example 6: Synthesis of Compound 3

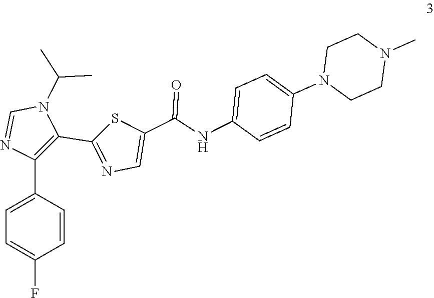 [SMILES: CC(C)n1cnc(-c2ccc(F)cc2)c1-c1ncc(C(=O)Nc2ccc(N3CCN(C)CC3)cc2)s1]

2-(4-(4-fluorophenyl)-1-isopropyl-1H-imidazol-5-yl)-N-(4-(4-methylpiperazin-1-yl)phenyl)thiazole-5-carboxamide (Compound 3) was synthesized under the same synthetic route as for Compound 14 as white solid, which was determined by ¹HNMR, LCMS and HPLC. LCMS: Retention time: 0.635 min, (M+H)=505.1. HPLC: Retention time: 1.938 min. HNMR: (400 MHz, DMSO-d₆), δ=10.31 (s, 1H), 8.69 (s, 1H), 8.18 (s, 1H), 7.57-7.37 (m, 4H), 7.20 (t, J=8.8 Hz, 2H), 6.93 (d, J=9.2 Hz, 2H), 4.76 (m, 1H), 3.17-3.00 (m, 4H), 2.46-2.44 (m, 4H), 2.22 (s, 3H), 1.45 (d, J=6.8 Hz, 6H).

### Example 7: Synthesis of Compound 4

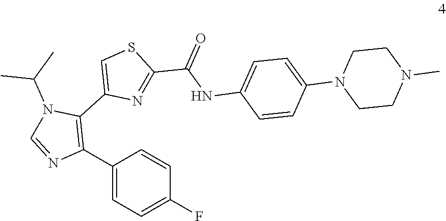 [SMILES: CC(C)n1cnc(-c2ccc(F)cc2)c1-c1csc(C(=O)Nc2ccc(N3CCN(C)CC3)cc2)n1]

4-(4-(4-fluorophenyl)-1-isopropyl-1H-imidazol-5-yl)-N-(4-(4-methylpiperazin-1-yl)phenyl)thiazole-2-carboxamide (Compound 4) was synthesized under the same synthetic route as for Compound 2 as white solid, which was determined by ¹HNMR, LCMS and HPLC. LCMS: Retention time: 1.748 min, (M+H)=505.3. HPLC: Retention time: 4.00 min. HNMR: (400 MHz, DMSO-d₆), δ=10.62 (s, 1H), 8.25 (s, 1H), 8.04 (s, 1H), 7.68 (d, J=9.2 Hz, 2H), 7.47-7.37 (m, 2H), 7.14-7.01 (m, 2H), 6.92 (d, J=9.2 Hz, 2H), 4.31-4.07 (m, 1H), 3.17-3.06 (m, 4H), 2.45 (br s, 3H), 2.22 (s, 4H), 1.40 (d, J=6.8 Hz, 6H).

### Example 8: Synthesis of Compound 5

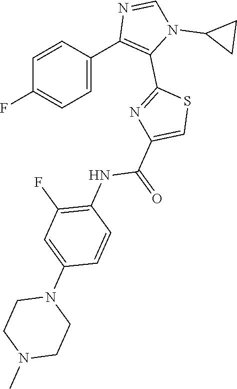 [SMILES: CN1CCN(c2ccc(NC(=O)c3csc(-c4c(-c5ccc(F)cc5)ncn4C4CC4)n3)c(F)c2)CC1]

2-(1-cyclopropyl-4-(4-fluorophenyl)-1H-imidazol-5-yl)-N-(2-fluoro-4-(4-methylpiperazin-1-yl)phenyl)thiazole-4-carboxamide (Compound 5) was synthesized under the same synthetic route as for Compound 2 as white solid, which was determined by ¹HNMR, LCMS and HPLC.

### Example 9: Synthesis of Compound 6

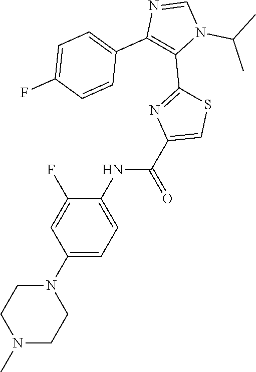 [SMILES: CC(C)n1cnc(-c2ccc(F)cc2)c1-c1nc(C(=O)Nc2ccc(N3CCN(C)CC3)cc2F)cs1]

N-(2-fluoro-4-(4-methylpiperazin-1-yl)phenyl)-2-(4-(4-fluorophenyl)-1-isopropyl-1H-imidazol-5-yl)thiazole-4-carboxamide (Compound 6) was synthesized under the same synthetic route as for Compound 2 as white solid, which was determined by ¹HNMR, LCMS and HPLC. LCMS: Retention time: 1.536 min, (M+H)=523.2, 0-60AB_3 min_220&254_Shimadzu.lcm

HPLC: Retention time: 4.00 min, 0-60AB_8 min.met

HNMR: (400 MHz, CDCl₃), δ=9.34-9.26 (m, 1H), 8.40-8.32 (m, 1H), 8.17 (s, 1H), 7.83 (s, 1H), 7.52-7.45 (m, 2H), 7.11-7.02 (m, 2H), 6.79-6.69 (m, 2H), 5.00-4.87 (m, 1H), 3.32-3.21 (m, 4H), 2.79-2.65 (m, 4H), 2.45 (s, 3H), 1.63 (d, J=6.8 Hz, 6H)

### Example 10: Synthesis of Compound 7

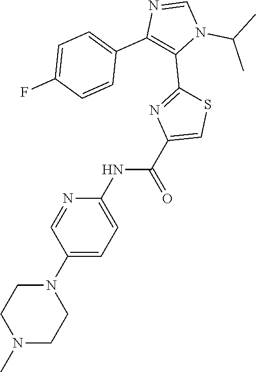 [SMILES: CC(C)n1cnc(-c2ccc(F)cc2)c1-c1nc(C(=O)Nc2ccc(N3CCN(C)CC3)cn2)cs1]

2-(4-(4-fluorophenyl)-1-isopropyl-1H-imidazol-5-yl)-N-(5-(4-methylpiperazin-1-yl)pyridin-2-yl)thiazole-4-carboxamide (Compound 7) was synthesized under the same synthetic route as for Compound 2 as white solid, which was determined by ¹HNMR, LCMS and HPLC.

LCMS: Retention time: 1.791 min, (M+H)=506.2, 10-80AB_7 min_220&254_Shimadzu.lcm

HPLC: Retention time: 5.10 min, 10-80AB_15 min.met

HNMR: (400 MHz, DMSO-d₆), δ=9.79 (s, 1H), 8.61 (s, 1H), 8.20 (s, 1H), 8.12-8.01 (m, 2H), 7.55-7.40 (m, 3H), 7.24-7.09 (m, 2H), 4.80-4.63 (m, 1H), 3.21-3.11 (m, 4H), 2.48-2.43 (m, 4H), 2.22 (s, 3H), 1.49 (s, 3H), 1.48 (s, 3H).

### Example 11: Synthesis of Compound 8

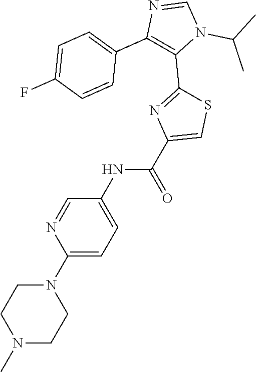 [SMILES: CC(C)n1cnc(-c2ccc(F)cc2)c1-c1nc(C(=O)Nc2ccc(N3CCN(C)CC3)nc2)cs1]

2-(4-(4-fluorophenyl)-1-isopropyl-1H-imidazol-5-yl)-N-(6-(4-methylpiperazin-1-yl)pyridin-3-yl)thiazole-4-carboxamide (Compound 8) was synthesized under the same synthetic route as for Compound 2 as white solid, which was determined by ¹HNMR, LCMS and HPLC.

LCMS: Retention time: 3.746 min, (M+H)=506.1, 10-80CD_7 min_220&254_Shimadzu.lcm

HPLC: Retention time: 4.87 min, 10-80AB_15 min.met

HNMR: (400 MHz, DMSO-d₆), δ=10.24 (s, 1H), 8.60 (s, 1H), 8.51 (d, J=2.4 Hz, 1H), 8.17 (s, 1H), 7.97-7.90 (m, 1H), 7.49-7.39 (m, 2H), 7.23-7.12 (m, 2H), 6.85 (d, J=9.2 Hz, 1H), 4.65-4.55 (m, 1H), 3.46-3.42 (m, 4H), 2.42-2.37 (m, 4H), 2.21 (s, 3H), 1.48-1.42 (m, 6H).

### Example 12: Synthesis of Compound 9

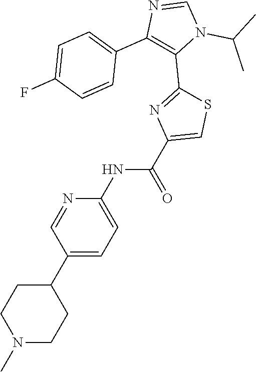 [SMILES: CC(C)n1cnc(-c2ccc(F)cc2)c1-c1nc(C(=O)Nc2ccc(C3CCN(C)CC3)cn2)cs1]

2-(4-(4-fluorophenyl)-1-isopropyl-1H-imidazol-5-yl)-N-(5-(1-methylpiperidin-4-yl)pyridin-2-yl)thiazole-4-carboxamide (Compound 9) was synthesized under the same synthetic route as for Compound 2 as white solid, which was determined by ¹HNMR, LCMS and HPLC.

LCMS: Retention time: 0.682 min, (M+H)=505.3, 5-95AB_220&254_Agilent.

HPLC: Retention time: 4.810 min, 10-80CD_8 min.lcm

HNMR: (400 MHz, CDCl₃), δ=9.62 (s, 1H), 8.32 (d, J=8.4 Hz, 1H), 8.27 (s, 1H), 8.23 (d, J=1.6 Hz, 1H), 7.82 (s, 1H), 7.65 (dd, J=2.0, 8.4 Hz, 1H), 7.46 (dd, J=5.6, 8.4 Hz, 2H), 7.04 (t, J=8.8 Hz, 2H), 4.93-4.82 (m, 1H), 3.01 (br d, J=11.6 Hz, 2H), 2.58-2.47 (m, 1H), 2.35 (s, 3H), 2.15-2.03 (m, 2H), 1.89-1.86 (m, 2H), 1.83-1.71 (m, 2H), 1.59 (d, J=6.8 Hz, 6H)

### Example 13: Synthesis of Compound 10

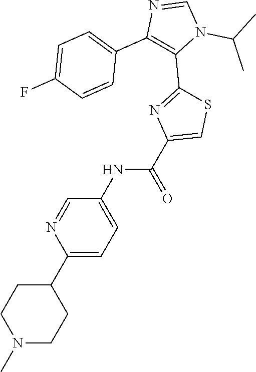 [SMILES: CC(C)n1cnc(-c2ccc(F)cc2)c1-c1nc(C(=O)Nc2ccc(C3CCN(C)CC3)nc2)cs1]

2-(4-(4-fluorophenyl)-1-isopropyl-1H-imidazol-5-yl)-N-(6-(1-methylpiperidin-4-yl)pyridin-3-yl)thiazole-4-carboxamide (Compound 10) was synthesized under the same synthetic route as for Compound 2 as white solid, which was determined by ¹HNMR, LCMS and HPLC.

### Example 14: Synthesis of Compound 11

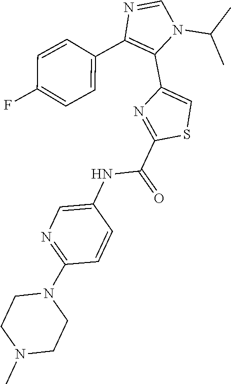 [SMILES: CC(C)n1cnc(-c2ccc(F)cc2)c1-c1csc(C(=O)Nc2ccc(N3CCN(C)CC3)nc2)n1]

4-(4-(4-fluorophenyl)-1-isopropyl-1H-imidazol-5-yl)-N-(6-(4-methylpiperazin-1-yl)pyridin-3-yl)thiazole-2-carboxamide (Compound 11) was synthesized under the same synthetic route as for Compound 2 as white solid, which was determined by ¹HNMR, LCMS and HPLC.

### Example 15: Synthesis of Compound 12

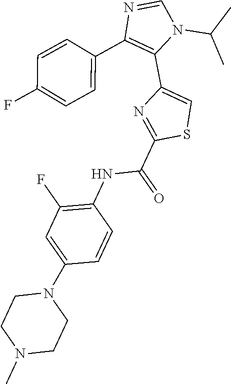 [SMILES: CC(C)n1cnc(-c2ccc(F)cc2)c1-c1csc(C(=O)Nc2ccc(N3CCN(C)CC3)cc2F)n1]

N-(2-fluoro-4-(4-methylpiperazin-1-yl)phenyl)-4-(4-(4-fluorophenyl)-1-isopropyl-1H-imidazol-5-yl)thiazole-2-carboxamide (Compound 12) was synthesized under the same synthetic route as for Compound 2 as white solid, which was determined by ¹HNMR, LCMS and HPLC.

LCMS: Retention time: 1.849 min, (M+H)=523.3, 10-80CD_3 min_220&254_Shimadzu.lcm

HPLC: Retention time: 4.280 min, 10-80AB_8 min.lcm

HNMR: (400 MHz, DMSO-d₆), δ=10.28 (s, 1H), 8.24 (s, 1H), 8.05 (s, 1H), 7.49-7.36 (m, 3H), 7.18-7.06 (m, 2H), 6.93-6.83 (m, 1H), 6.80-6.69 (m, 1H), 4.29-4.15 (m, 1H), 3.29-3.18 (m, 4H), 2.57-2.51 (m, 4H), 2.29 (s, 3H), 1.41 (d, J=6.8 Hz, 6H)

### Example 16: Synthesis of Compound 13

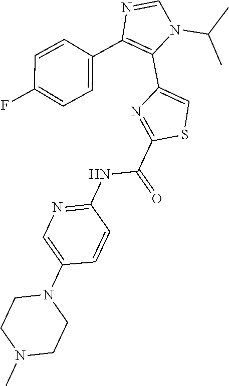 [SMILES: CC(C)n1cnc(-c2ccc(F)cc2)c1-c1csc(C(=O)Nc2ccc(N3CCN(C)CC3)cn2)n1]

4-(4-(4-fluorophenyl)-1-isopropyl-1H-imidazol-5-yl)-N-(5-(4-methylpiperazin-1-yl)pyridin-2-yl)thiazole-2-carboxamide (Compound 13) was synthesized under the same synthetic route as for Compound 2 as white solid, which was determined by ¹HNMR, LCMS and HPLC.

LCMS: Retention time: 1.972 min, (M+H)=506.2, 10-80CD_3 min_220&254_Shimadzu.lcm.

HPLC: Retention time: 3.956 min, 10-80CD_8 min.lcm

HNMR: (400 MHz, DMSO-d₆), 10.14 (s, 1H), 8.20 (s, 1H), 8.09 (d, J=3.0 Hz, 1H), 8.06 (s, 1H), 7.94 (d, J=9.0 Hz, 1H), 7.49 (dd, J=2.8, 9.0 Hz, 1H), 7.42 (dd, J=5.8, 8.8 Hz, 2H), 7.10 (t, J=8.9 Hz, 2H), 4.35-4.20 (m, 1H), 3.21-3.14 (m, 4H), 2.48-2.43 (m, 4H), 2.22 (s, 3H), 1.41 (d, J=6.8 Hz, 6H)

### Example 16: Synthesis of Compound 14

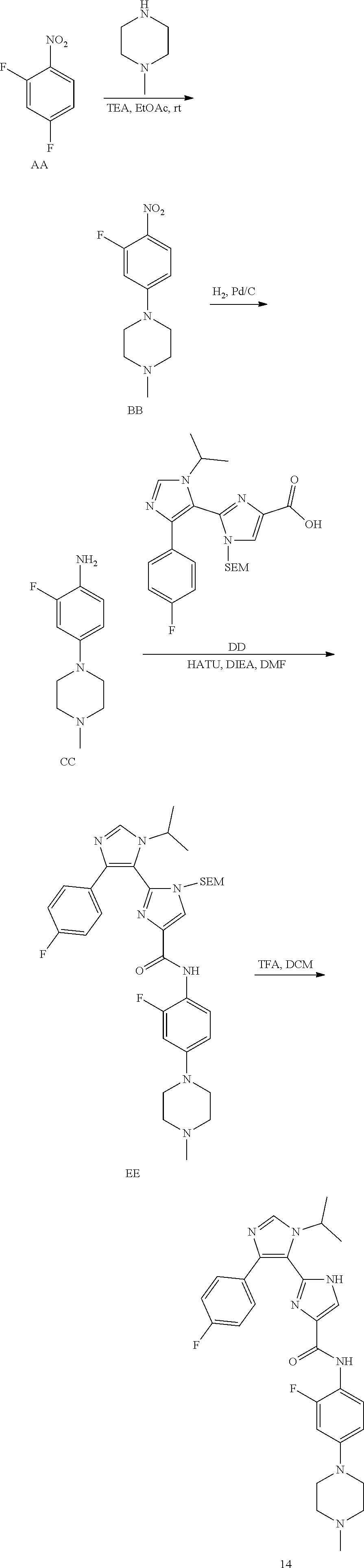

### Example 17: General Procedure for Preparation of 1-(3-fluoro-4-nitrophenyl)-4-methylpiperazine (Compound BB)

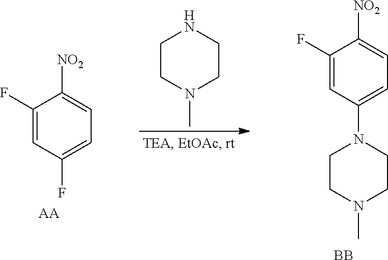

To a solution of 2,4-difluoro-1-nitrobenzene (Compound AA) (2.10 g, 20.95 mmol, 2.32 mL, 1 eq) and TEA (2.33 g, 23.05 mmol, 3.21 mL, 1.1 eq) in EA (30 mL) was added 2,4-difluoro-1-nitro-benzene (5 g, 31.43 mmol, 3.45 mL, 1.5 eq) dropwise with stirring and the mixture was stirred at 50° C. for 3 hours. The mixture was cooled to room temperature and concentrated under reduced pressure. The residue was purified by column chromatography (SiO₂, Petroleum ether/Ethyl acetate=1:0 to 0:1) to afford 1-(3-fluoro-4-nitrophenyl)-4-methylpiperazine (Compound BB) (720 mg, 3.01 mmol, 14.35% yield, 99.898% purity) as yellow solid, which was determined by ¹HNMR and LCMS. LCMS: Retention time: 1.213 min, (M+H)=239.6. HNMR: (400 MHz, DMSO-d₆), δ=7.98 (t, J=9.6 Hz, 1H), 6.97-6.81 (m, 2H), 3.52-3.43 (m, 4H), 2.44-2.36 (m, 4H), 2.21 (s, 3H).

### Example 18: General Procedure for Preparation of 2-fluoro-4-(4-methylpiperazin-1-yl)aniline (Compound CC)

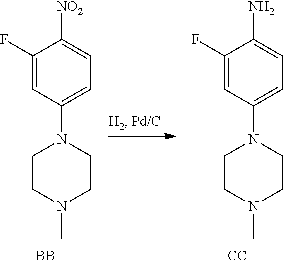

To a solution of 1-(3-fluoro-4-nitrophenyl)-4-methylpiperazine (Compound BB) (400 mg, 1.67 mmol, 1 eq) in THF (20 mL) was added Pd/C (100 mg, 93.97 μmol, 10% w). The mixture was stirred under H₂ (15 psi) at 25° C. for 4 hrs. The mixture was filtered. The filtrate was poured into water (10 mL) and extracted with ethyl acetate (10 mL×2). The combined organic extracts were washed with brine (10 mL), dried over anhydrous Na₂SO₄, filtered and concentrated under reduced pressure to afford 2-fluoro-4-(4-methylpiperazin-1-yl)aniline (Compound CC) (298 mg, 1.39 mmol, 83.37% yield, 97.877% purity) as purple solid, which was determined by ¹HNMR and LCMS. LCMS: Retention time: 0.207 min, (M+H)=209.6. HNMR: (400 MHz, DMSO-d₆), δ=6.69-6.61 (m, 2H), 6.54-6.49 (m, 1H), 4.55 (s, 2H), 2.99-2.87 (m, 4H), 2.45-2.35 (m, 4H), 2.19 (s, 3H)

### Example 19: General Procedure for Preparation of N-(2-fluoro-4-(4-methylpiperazin-1-yl)phenyl)-5′-(4-fluorophenyl)-3′-isopropyl-1-((2-(trimethylsilyl)ethoxy)methyl)-1H,3′H-[2,4′-biimidazole]-4-carboxamide (Compound EE)

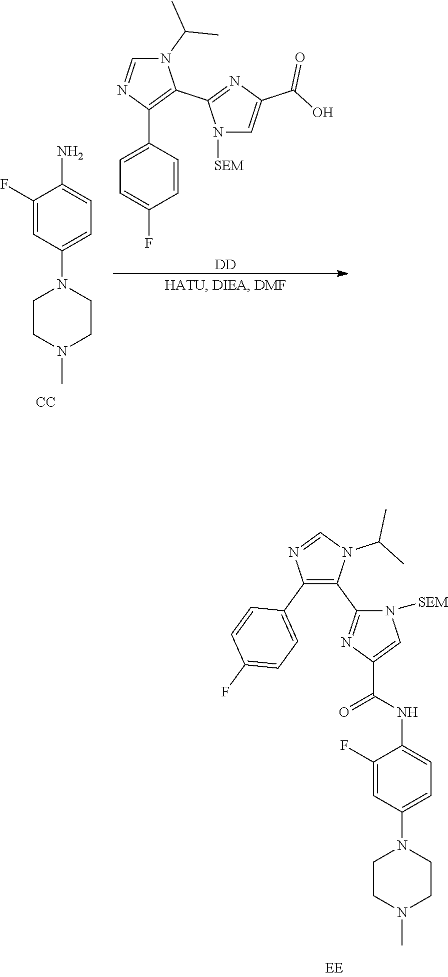

To a mixture of 2-[5-(4-fluorophenyl)-3-isopropyl-imidazol-4-yl]-1-(2-trimethylsilylethoxymethyl)imidazole-4-carboxylic acid (Compound DD) (141.63 mg, 318.58 μmol, 1 eq), DIPEA (123.52 mg, 955.74 μmol, 166.47 μL, 3 eq) and HATU (242.27 mg, 637.16 μmol, 2 eq) in DMF (5 mL) was added 2-fluoro-4-(4-methylpiperazin-1-yl)aniline (Compound CC) (100 mg, 477.87 μmol, 1.5 eq) and the mixture was stirred at 25° C. for 2 hrs. The mixture was poured into water (10 mL) and extracted with ethyl acetate (10 mL×2). The combined organic extracts were washed with brine (10 mL), dried over anhydrous Na₂SO₄, filtered and concentrated under reduced pressure. The residue was purified by flash silica gel chromatography (ISCO®; 4 g SepaFlash® Silica Flash Column, Eluent of 0˜100% EA/PE to 0-10% MeOH/DCM @ 15 mL/min) to afford N-(2-fluoro-4-(4-methylpiperazin-1-yl)phenyl)-5′-(4-fluorophenyl)-3′-isopropyl-1-((2-(trimethylsilyl)ethoxy)methyl)-1H,3′H-[2,4′-biimidazole]-4-carboxamide (Compound EE) (200 mg, 308.72 umol, 96.90% yield, 98.144% purity) as yellow oil, which was determined by ¹HNMR and LCMS. LCMS: Retention time: 1.162 min, (M+H)=636.4. HNMR: (400 MHz, DMSO-d₆), δ=9.50-9.47 (m, 1H), 9.48 (s, 1H), 8.23 (s, 1H), 8.17-8.10 (m, 1H), 7.62 (t, J=8.8 Hz, 1H), 7.34-7.26 (m, 2H), 7.14 (t, J=8.8 Hz, 2H), 6.91-6.73 (m, 2H), 5.05-4.88 (m, 2H), 4.16-4.06 (m, 1H), 3.29-3.24 (m, 2H), 3.17-3.12 (m, 4H), 2.46-2.41 (m, 4H), 2.22 (s, 3H), 1.52-1.34 (m, 6H), 0.66-0.51 (m, 2H), −0.11 (s, 8H).

### Example 20: General Procedure for Preparation of N-(2-fluoro-4-(4-methylpiperazin-1-yl)phenyl)-5′-(4-fluorophenyl)-3′-isopropyl-1H,3′H-[2,4′-biimidazole]-4-carboxamide (Compound 14)

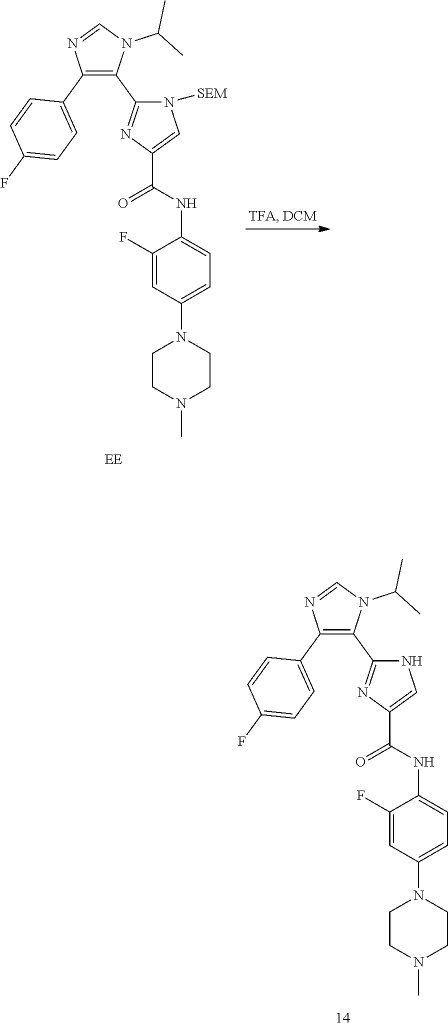 [SMILES: CC(C)n1cnc(-c2ccc(F)cc2)c1-c1nc(C(=O)Nc2ccc(N3CCN(C)CC3)cc2F)cn1COCC[Si](C)(C)C ; CC(C)n1cnc(-c2ccc(F)cc2)c1-c1nc(C(=O)Nc2ccc(N3CCN(C)CC3)cc2F)c[nH]1]

A mixture of N-(2-fluoro-4-(4-methylpiperazin-1-yl)phenyl)-5′-(4-fluorophenyl)-3′-isopropyl-1-((2-(trimethylsilyl)ethoxy)methyl)-1H,3′H-[2,4′-biimidazole]-4-carboxamide (EE) (160 mg, 251.64 μmol, 1 eq) in TFA (9 mL) and DCM (1 mL) was stirred at 25° C. for 3 hrs. The mixture was diluted with DMSO (2 mL) and basified with TEA to pH 7-8. The mixture was concentrated under reduced pressure and purified by prep-HPLC (column: Welch Xtimate C18 150*30 mm*5 um; mobile phase: [water (0.05% NH₃H₂O+10 mM NH₄HCO₃)-ACN]; B %: 33%-63%, 9 min) and lyophilized to afford N-(2-fluoro-4-(4-methylpiperazin-1-yl)phenyl)-5′-(4-fluorophenyl)-3′-isopropyl-1H,3′H-[2,4′-biimidazole]-4-carboxamide (Compound 14) (55.93 mg, 107.43 umol, 42.69% yield, 97.106% purity) as white solid, which was determined by ¹HNMR, LCMS and HPLC. LCMS: (M+H)=506.3. HPLC: Retention time: 3.397 min. HNMR: (400 MHz, CDCl₃), δ=9.97 (s, 1H), 9.12 (s, 1H), 8.28-8.40 (m, 1H), 7.64-7.74 (m, 2H), 7.35-7.46 (m, 2H), 6.99-7.12 (m, 2H), 6.66-6.79 (m, 2H), 4.88-4.98 (m, 1H), 3.31-3.41 (m, 4H), 2.78-2.95 (m, 4H), 2.55 (s, 3H), 1.57 (s, 3H), 1.56 (s, 3H).

### Example 21: Synthesis of Compound 15

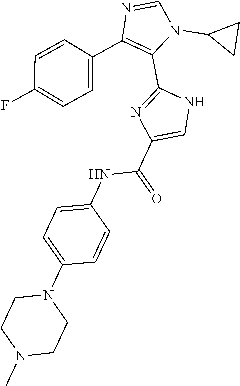 [SMILES: CN1CCN(c2ccc(NC(=O)c3c[nH]c(-c4c(-c5ccc(F)cc5)ncn4C4CC4)n3)cc2)CC1]

3′-cyclopropyl-5′-(4-fluorophenyl)-N-(4-(4-methylpiperazin-1-yl)phenyl)-1H,3′H-[2,4′-biimidazole]-4-carboxamide (Compound 15) was synthesized under the same synthetic route as for Compound 14 as white solid, which was determined by ¹HNMR, LCMS and HPLC. LCMS: (M+H)=486.1. HPLC: Retention time: 4.32 min. HNMR: (400 MHz, CD₃OD), δ=7.95-7.90 (m, 2H), 7.61-7.55 (m, 2H), 7.47-7.41 (m, 2H), 7.08-6.97 (m, 4H), 3.48 (m, 1H), 3.23-3.17 (m, 4H), 2.69-2.62 (m, 4H), 2.37 (s, 3H), 0.97-0.89 (m, 4H).

### Example 22: Synthesis of Compound 16

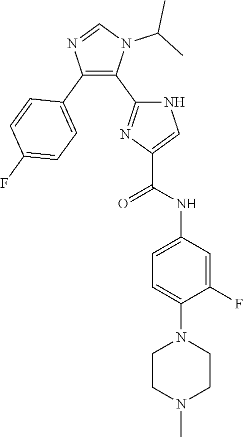 [SMILES: CC(C)n1cnc(-c2ccc(F)cc2)c1-c1nc(C(=O)Nc2ccc(N3CCN(C)CC3)c(F)c2)c[nH]1]

N-(3-fluoro-4-(4-methylpiperazin-1-yl)phenyl)-5′-(4-fluorophenyl)-3′-isopropyl-1H,3′H-[2,4′-biimidazole]-4-carboxamide (Compound 16) was synthesized under the same synthetic route as for Compound 14 as white solid, which was determined by ¹HNMR, F NMR, LCMS and HPLC. LCMS: Retention time: 0.695 min, (M+H)=506.2. HPLC: Retention time: 2.32 min. HNMR: (400 MHz, DMSO-d₆), δ=9.99 (s, 1H), 8.04 (s, 1H), 8.00 (s, 1H), 7.75 (dd, J=2.4, 15.2 Hz, 1H), 7.59-7.52 (m, 1H), 7.43-7.36 (m, 2H), 7.17-7.09 (m, 2H), 7.03-6.94 (m, 1H), 6.16-5.97 (m, 1H), 4.29-4.17 (m, 1H), 3.01-2.92 (m, 4H), 2.48-2.42 (m, 4H), 2.22 (s, 3H), 1.38 (d, J=6.8 Hz, 6H).

### Example 23: Synthesis of Compound 17

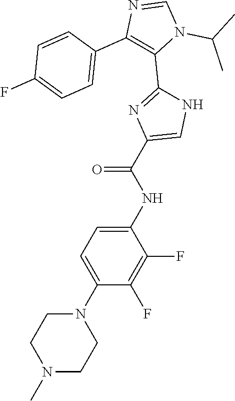 [SMILES: CC(C)n1cnc(-c2ccc(F)cc2)c1-c1nc(C(=O)Nc2ccc(N3CCN(C)CC3)c(F)c2F)c[nH]1]

N-(2,3-difluoro-4-(4-methylpiperazin-1-yl)phenyl)-5′-(4-fluorophenyl)-3′-isopropyl-1H,3′H-[2,4′-biimidazole]-4-carboxamide (Compound 17) was synthesized under the same synthetic route as for Compound 14 as white solid, which was determined by ¹HNMR, LCMS and HPLC. LCMS: Retention time: 0.939 min, (M+H)=524.3. HPLC: Retention time: 1.871 min.

¹H NMR (400 MHz, DMSO-d₆) δ=13.12 (br. s., 1H), 9.69 (br. s., 1H), 8.05 (s, 1H), 8.08 (s, 1H), 7.47 (s, 1H), 7.42-7.35 (m, 2H), 7.19-7.11 (m, 2H), 6.91-6.82 (m, 1H), 4.28-4.23 (m, 1H), 3.12-3.10 (m, 4H), 2.61-2.57 (m, 4H), 2.29 (s, 3H), 1.40 (d, J=5.2 Hz, 6H).

### Example 24: Synthesis of Compound 18

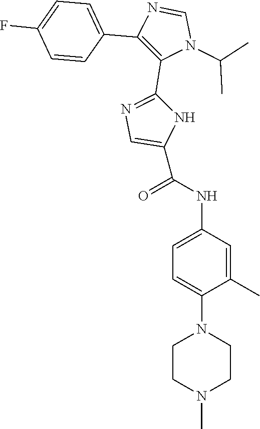 [SMILES: Cc1cc(NC(=O)c2cnc(-c3c(-c4ccc(F)cc4)ncn3C(C)C)[nH]2)ccc1N1CCN(C)CC1]

5′-(4-fluorophenyl)-3′-isopropyl-N-(3-methyl-4-(4-methylpiperazin-1-yl)phenyl)-1H,3′H-[2,4′-biimidazole]-5-carboxamide (Compound 18) was synthesized under the same synthetic route as for Compound 14 as white solid, which was determined by ¹HNMR, LCMS and HPLC. LCMS: Retention time: 0.609 min, (M+H)=502.1. HPLC: Retention time: 1.729 min. HNMR: (400 MHz, DMSO-d₆), δ=13.20-12.83 (s, 1H), 9.71 (s, 1H), 8.15-7.92 (m, 2H), 7.62-7.56 (m, 2H), 7.38 (m, 2H), 7.13 (t, J=8.8 Hz, 2H), 6.97 (d, J=8.0 Hz, 1H), 4.30-4.16 (m, 1H), 2.80 (m, 4H), 2.48-2.43 (m, 4H), 2.22 (d, J=3.6 Hz, 6H), 1.39 (d, J=6.4 Hz, 6H).

### Example 25: Synthesis of Compound 19

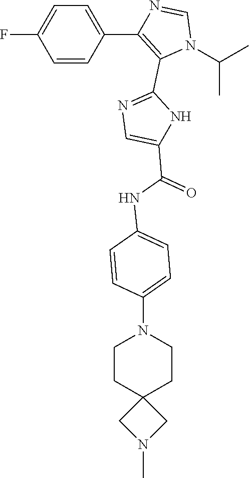 [SMILES: CC(C)n1cnc(-c2ccc(F)cc2)c1-c1ncc(C(=O)Nc2ccc(N3CCC4(CC3)CN(C)C4)cc2)[nH]1]

5′-(4-fluorophenyl)-3′-isopropyl-N-(4-(2-methyl-2,7-diazaspiro[3.5]nonan-7-yl)phenyl)-1H,3′H-[2,4′-biimidazole]-5-carboxamide (Compound 19) was synthesized under the same synthetic route as for Compound 14 as white solid, which was determined by ¹HNMR, LCMS and HPLC. LCMS: Retention time: 2.579 min, (M+H)=528.2. HPLC: Retention time: 5.282 min. HNMR: (400 MHz, DMSO-d₆), δ=13.26-12.54 (m, 1H), 9.71 (br s, 1H), 8.09-7.93 (m, 1H), 8.09-7.93 (m, 1H), 7.63 (d, J=8.4 Hz, 2H), 7.42-7.32 (m, 2H), 7.13 (t, J=8.8 Hz, 2H), 6.88 (d, J=9.2 Hz, 2H), 4.30-4.16 (m, 1H), 3.07-2.97 (m, 4H), 2.92 (s, 4H), 2.22 (s, 3H), 1.83-1.66 (m, 4H), 1.38 (d, J=6.4 Hz, 6H).

### Example 26: Synthesis of Compound 20

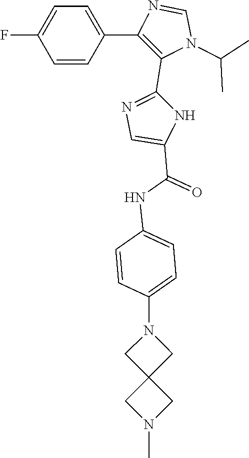 [SMILES: CC(C)n1cnc(-c2ccc(F)cc2)c1-c1ncc(C(=O)Nc2ccc(N3CC4(CN(C)C4)C3)cc2)[nH]1]

5′-(4-fluorophenyl)-3′-isopropyl-N-(4-(6-methyl-2,6-diazaspiro[3.3]heptan-2-yl)phenyl)-1H,3′H-[2,4′-biimidazole]-5-carboxamide (Compound 20) was synthesized under the same synthetic route as for Compound 14 as white solid, which was determined by ¹HNMR, LCMS and HPLC. LCMS: Retention time: 0.659 min, (M+H)=500.3. HPLC: Retention time: 3.941 min. HNMR: (400 MHz, DMSO-d₆), δ=13.04 (br s, 1H), 9.67 (br s, 1H), 8.05 (s, 1H), 7.96 (s, 1H), 7.59 (br d, J=7.6 Hz, 2H), 7.41-7.34 (m, 2H), 7.13 (t, J=8.8 Hz, 2H), 6.38 (br d, J=8.8 Hz, 2H), 4.29-4.16 (m, 1H), 3.80 (s, 4H), 3.24 (s, 4H), 2.17 (s, 3H), 1.38 (d, J=6.4 Hz, 6H).

### Example 27: Synthesis of Compound 21

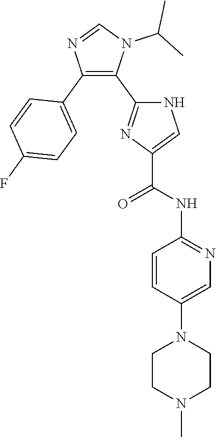 [SMILES: CC(C)n1cnc(-c2ccc(F)cc2)c1-c1nc(C(=O)Nc2ccc(N3CCN(C)CC3)cn2)c[nH]1]

5′-(4-fluorophenyl)-3′-isopropyl-N-(5-(4-methylpiperazin-1-yl)pyridin-2-yl)-1H,3′H-[2,4′-biimidazole]-4-carboxamide (Compound 21) was synthesized under the same synthetic route as for Compound 14 as white solid, which was determined by ¹HNMR, LCMS and HPLC. LCMS: Retention time: 1.342 min, (M+H)=489.3. HPLC: Retention time: 1.150 min. HNMR: (400 MHz, CDCl₃), δ=10.39 (s, 1H), 9.38 (s, 1H), 8.26 (d, J=8.8 Hz, 1H), 8.01 (d, J=2.8 Hz, 1H), 7.75-7.69 (m, 1H), 7.62 (s, 1H), 7.40-7.30 (m, 3H), 7.05-6.96 (m, 2H), 5.03-4.91 (m, 1H), 3.28-3.17 (m, 4H), 2.72-2.60 (m, 4H), 2.39 (s, 3H), 1.51 (d, J=6.4 Hz, 6H).

### Example 28: Synthesis of Compound 22

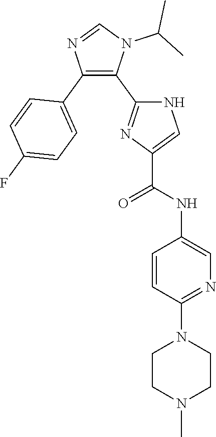 [SMILES: CC(C)n1cnc(-c2ccc(F)cc2)c1-c1nc(C(=O)Nc2ccc(N3CCN(C)CC3)nc2)c[nH]1]

5′-(4-fluorophenyl)-3′-isopropyl-N-(6-(4-methylpiperazin-1-yl)pyridin-3-yl)-1H,3′H-[2,4′-biimidazole]-4-carboxamide (Compound 22) was synthesized under the same synthetic route as for Compound 14 as white solid, which was determined by ¹HNMR, LCMS and HPLC. LCMS: Retention time: 1.307 min, (M+H)=489.3. HPLC: Retention time: 1.609 min. HNMR: (400 MHz, CDCl₃), δ=10.98 (s, 1H), 8.78 (s, 1H), 8.33 (d, J=2.4 Hz, 1H), 8.03 (dd, J=2.4, 9.2 Hz, 1H), 7.69 (s, 1H), 7.58 (s, 1H), 7.37-7.29 (m, 2H), 7.03-6.93 (m, 2H), 6.69 (d, J=8.8 Hz, 1H), 4.83-4.70 (m, 1H), 3.59-3.44 (m, 4H), 2.57-2.50 (m, 4H), 2.33 (s, 3H), 1.49 (d, J=6.8 Hz, 6H).

### Example 29: Synthesis of Compound 23

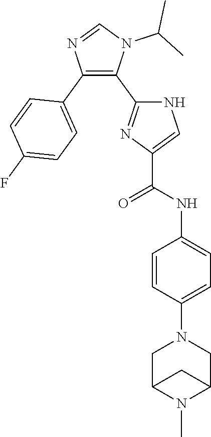 [SMILES: CC(C)n1cnc(-c2ccc(F)cc2)c1-c1nc(C(=O)Nc2ccc(N3CC4CC(C3)N4C)cc2)c[nH]1]

5′-(4-fluorophenyl)-3′-isopropyl-N-(4-(6-methyl-3,6-diazabicyclo[3.1.1]heptan-3-yl)phenyl)-1H,3′H-[2,4′-biimidazole]-4-carboxamide (Compound 23) was synthesized under the same synthetic route as for Compound 14 as white solid, which was determined by ¹HNMR, LCMS and HPLC. LCMS: Retention time: 1.008 min, (M+H)=499.9. HPLC: Retention time: 1.760 min. HNMR: (400 MHz, CDCl₃), δ=10.40 (s, 1H), 8.84 (s, 1H), 7.72 (d, J=2.4 Hz, 2H), 7.66 (d, J=8.8 Hz, 2H), 7.44-7.36 (m, 2H), 7.02 (t, J=9.2 Hz, 2H), 6.77 (d, J=8.8 Hz, 2H), 4.87-4.76 (m, 1H), 4.20 (d, J=4.8 Hz, 2H), 3.73-3.61 (m, 4H), 3.02 (s, 1H), 2.36 (s, 3H), 1.85 (d, J=8.8 Hz, 1H), 1.54 (d, J=6.8 Hz, 6H).

### Example 30: Synthesis of Compound 24

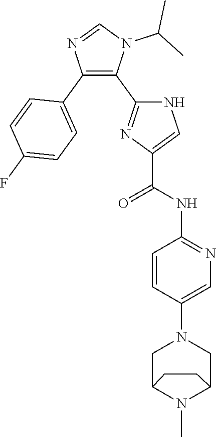 [SMILES: CC(C)n1cnc(-c2ccc(F)cc2)c1-c1nc(C(=O)Nc2ccc(N3CC4CCC(C3)N4C)cn2)c[nH]1]

5′-(4-fluorophenyl)-3′-isopropyl-N-(5-((1R,5S)-8-methyl-3,8-diazabicyclo[3.2.1]octan-3-yl)pyridin-2-yl)-1H,3′H-[2,4′-biimidazole]-4-carboxamide (Compound 24) was synthesized under the same synthetic route as for Compound 14 as white solid, which was determined by ¹HNMR, LCMS and HPLC. LCMS: Retention time: 3.409 min, (M+H)=515.2. HPLC: Retention time: 2.91 min. HNMR: (400 MHz, DMSO-d₆), δ=13.09 (br. s., 1H), 9.37 (s, 1H), 8.13-8.05 (m, 2H), 8.05-7.97 (m, 1H), 7.89 (s, 1H), 7.47-7.25 (m, 3H), 7.23-6.98 (m, 2H), 4.36-4.17 (m, 1H), 3.39-3.35 (m, 2H), 3.26-3.14 (m, 2H), 2.94-2.77 (m, 2H), 2.22 (s, 3H), 2.02-1.87 (m, 2H), 1.73-1.56 (m, 2H), 1.41 (s, 3H), 1.39 (s, 3H).

### Example 31: Synthesis of Compound 25

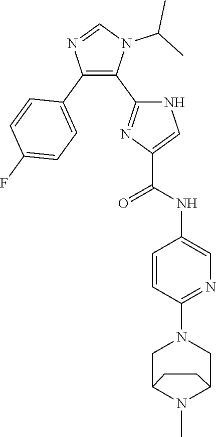 [SMILES: CC(C)n1cnc(-c2ccc(F)cc2)c1-c1nc(C(=O)Nc2ccc(N3CC4CCC(C3)N4C)nc2)c[nH]1]

5′-(4-fluorophenyl)-3′-isopropyl-N-(6-((1R,5S)-8-methyl-3,8-diazabicyclo[3.2.1]octan-3-yl)pyridin-3-yl)-1H,3′H-[2,4′-biimidazole]-4-carboxamide (Compound 25) was synthesized under the same synthetic route as for Compound 14 as white solid, which was determined by ¹HNMR, LCMS and HPLC. LCMS: Retention time: 3.504 min, (M+H)=515.2. HPLC: Retention time: 4.10 min. HNMR: (400 MHz, DMSO-d₆), δ=13.28-12.90 (m, 1H), 9.87 (s, 1H), 8.46 (s, 1H), 8.12-7.97 (m, 2H), 7.94-7.85 (m, 1H), 7.43-7.32 (m, 2H), 7.20-7.08 (m, 2H), 6.66 (d, J=10.0 Hz, 1H), 4.26-4.16 (m, 1H), 3.71 (d, J=10.0 Hz, 2H), 3.19 (s, 2H), 2.88 (d, J=10.4 Hz, 2H), 2.22 (s, 3H), 1.95-1.89 (m, 2H), 1.60-1.49 (m, 2H), 1.40-1.36 (m, 6H).

### Example 32: Synthesis of Compound 26

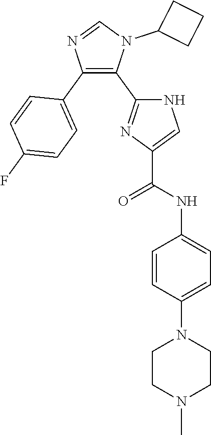 [SMILES: CN1CCN(c2ccc(NC(=O)c3c[nH]c(-c4c(-c5ccc(F)cc5)ncn4C4CCC4)n3)cc2)CC1]

3′-cyclobutyl-5′-(4-fluorophenyl)-N-(4-(4-methylpiperazin-1-yl)phenyl)-1H,3′H-[2,4′-biimidazole]-4-carboxamide (Compound 26) was synthesized under the same synthetic route as for Compound 14 as white solid, which was determined by ¹HNMR, LCMS and HPLC. LCMS: Retention time: 3.390 min, (M+H)=500.2. HPLC: Retention time: 4.52 min. HNMR: (400 MHz, DMSO-d₆), δ=13.22-12.87 (m, 1H), 9.72 (s, 1H), 8.14 (s, 1H), 7.97 (s, 1H), 7.70-7.63 (m, 2H), 7.47-7.37 (m, 2H), 7.18-7.10 (m, 2H), 6.93-6.86 (m, 2H), 4.57-4.45 (m, 1H), 3.08 (s, 4H), 2.46-2.43 (m, 4H), 2.39-2.29 (m, 2H), 2.23-2.16 (m, 5H), 1.77-1.66 (m, 2H).

### Example 33: Synthesis of Compound 27

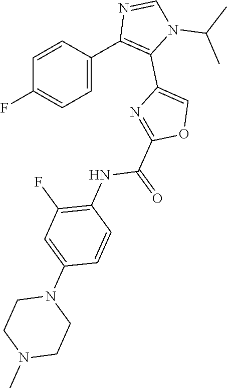 [SMILES: CC(C)n1cnc(-c2ccc(F)cc2)c1-c1coc(C(=O)Nc2ccc(N3CCN(C)CC3)cc2F)n1]

N-(2-fluoro-4-(4-methylpiperazin-1-yl)phenyl)-4-(4-(4-fluorophenyl)-1-isopropyl-1H-imidazol-5-yl)oxazole-2-carboxamide (Compound 27) was synthesized under the same synthetic route as for Compound 2 as white solid, which was determined by ¹HNMR, LCMS and HPLC. LCMS: Retention time: 1.486 min, (M+H)=507.1. HPLC: Retention time: 3.777 min. HNMR: (400 MHz, DMSO-d₆), δ=10.42 (s, 1H), 8.64 (s, 1H), 8.08 (s, 1H), 7.62-7.50 (m, 2H), 7.40-7.28 (m, 1H), 7.19-7.08 (m, 2H), 6.90-6.81 (m, 1H), 6.80-6.73 (m, 1H), 4.33-4.19 (m, 1H), 3.21-3.11 (m, 4H), 2.46-2.38 (m, 4H), 2.21 (s, 3H), 1.43 (s, 3H), 1.41 (s, 3H).

### Example 34: Synthesis of Compound 28

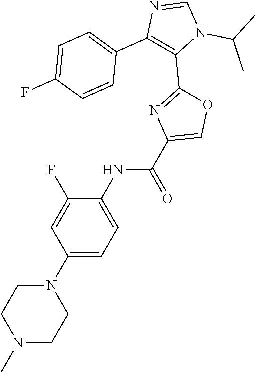 [SMILES: CC(C)n1cnc(-c2ccc(F)cc2)c1-c1nc(C(=O)Nc2ccc(N3CCN(C)CC3)cc2F)co1]

N-(2-fluoro-4-(4-methylpiperazin-1-yl)phenyl)-2-(4-(4-fluorophenyl)-1-isopropyl-1H-imidazol-5-yl)oxazole-4-carboxamide (Compound 28) was synthesized under the same synthetic route as for Compound 2 as white solid, which was determined by ¹HNMR, LCMS and HPLC.

### Example 35: Synthesis of Compound 29

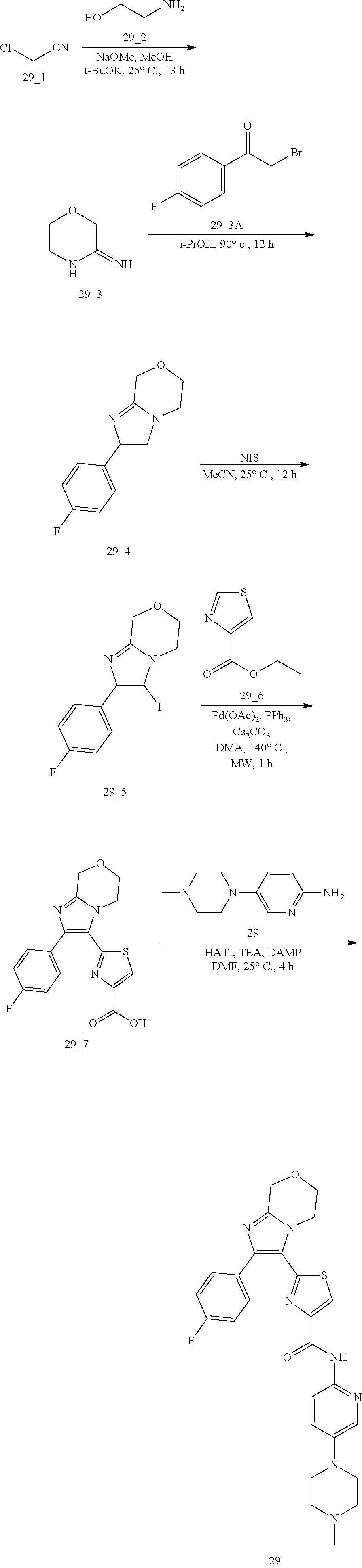

### General Procedure for Preparation of morpholin-3-imine (Compound 29_3)

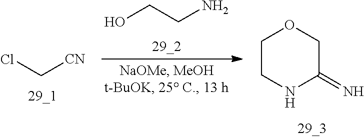

MeONa (5.4 M, 1.23 mL, 30% purity, 0.05 eq) was added dropwise over about 10 minutes to the solution of 2-chloroacetonitrile Compound 29_1 (10 g, 132.46 mmol, 8.40 mL, 1 eq) in MeOH (50 mL) under N₂ atmosphere. Then 2-aminoethanol Compound 29_2 (8.09 g, 132.46 mmol, 8.01 mL, 1 eq) was dissolved in MeOH (10 mL) slowly to the mixture. The mixture was stirred at 25° C. for 12 hours. Then t-BuOK (17.09 g, 152.32 mmol, 1.15 eq) was added to the mixture and stirred at 25° C. for 1 hour. The resulting product was dissolved in MeOH (50 mL) and filtered. The filtrate was re-filtered 3 times with (ACN 20 mL) until the salts were mostly removed and then concentrated to give morpholin-3-imine Compound 29_3 (16 g, crude) as black oil.

### General Procedure for Preparation of 2-(4-fluorophenyl)-6,8-dihydro-5H-imidazo[2,1-c][1,4]oxazine (Compound 29_4)

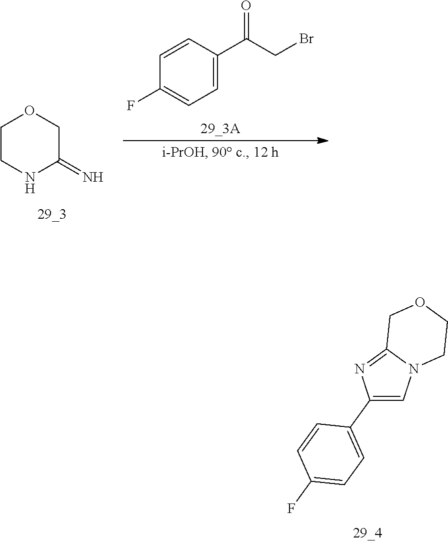 [SMILES: O=C(CBr)c1ccc(F)cc1 ; N=C1COCCN1 ; Fc1ccc(-c2cn3c(n2)COCC3)cc1]

To a solution of 2-bromo-1-(4-fluorophenyl)ethanone Compound 29_3A (5 g, 23.04 mmol, 1 eq) in isopropyl alcohol (50 mL) was added morpholin-3-imine Compound 29_3 (6.92 g, 69.11 mmol, 3 eq). The mixture was stirred at 90° C. for 12 hours. The mixture was partitioned between EtOAc and the saturated solution of NaHCO₃ (100 mL). The aqueous layer was extracted with EtOAc (50 mL×3). The combined organic phases were washed with brine and dried over anhydrous Na₂SO₄, filtered and concentrated to give the crude. The crude was purified by column chromatography (SiO₂, Petroleum ether/Ethyl acetate=1/0 to 1/1) to give 2-(4-fluorophenyl)-6,8-dihydro-5H-imidazo[2,1-c][1,4]oxazine Compound 294 (2.6 g, 11.11 mmol, 48.24% yield, 93.282% purity) as a yellow solid, which was determined by LCMS. LCMS: Retention time: 2.165 min, (M+H)=219.1.

### General Procedure for Preparation of 2-(4-fluorophenyl)-3-iodo-6,8-dihydro-5H-imidazo[2,1-c][1,4]oxazine (Compound 29_5)

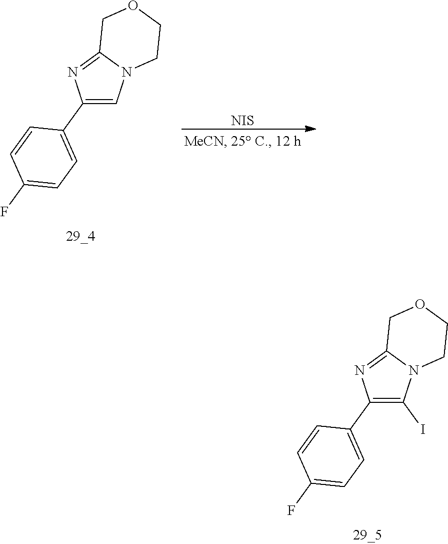 [SMILES: Fc1ccc(-c2cn3c(n2)COCC3)cc1 ; "NIS" ; Fc1ccc(-c2nc3n(c2I)CCOC3)cc1]

To a solution of 2-(4-fluorophenyl)-6,8-dihydro-5H-imidazo[2,1-c][1,4]oxazine Compound 29_4 (1 g, 4.58 mmol, 1 eq) in ACN (20 mL) was added NIS (2.06 g, 9.16 mmol, 2 eq). The mixture was stirred at 25° C. for 12 hours. Saturated Na₂CO₃ aqueous (30 mL) was added and extracted with EA (30 mL×3). The combined organic layers were washed with brine (30 mL), dried over Na₂SO₄, filtered and concentrated in vacuum. The residue was purified by column chromatography (SiO2, Petroleum ether/Ethyl acetate=1/0 to 1/1) to give 2-(4-fluorophenyl)-3-iodo-6,8-dihydro-5H-imidazo[2,1-c][1,4]oxazine Compound 295 (1.3 g, 3.48 mmol, 75.99% yield, 92.172% purity) a yellow solid, which was determined by LCMS and ¹HNMR. LCMS: Retention time: 0.720 min, (M+H)=344.9. HNMR: (400 MHz, DMSO-d₆), δ=7.94-7.86 (m, 2H), 7.29-7.21 (m, 2H), 4.76 (s, 2H), 4.09-4.02 (m, 2H), 3.90-3.82 (m, 2H).

### General Procedure for Preparation of 2-(2-(4-fluorophenyl)-6,8-dihydro-5H-imidazo[2,1-c][1,4]oxazin-3-yl)thiazole-4-carboxylic acid (Compound 29_7)

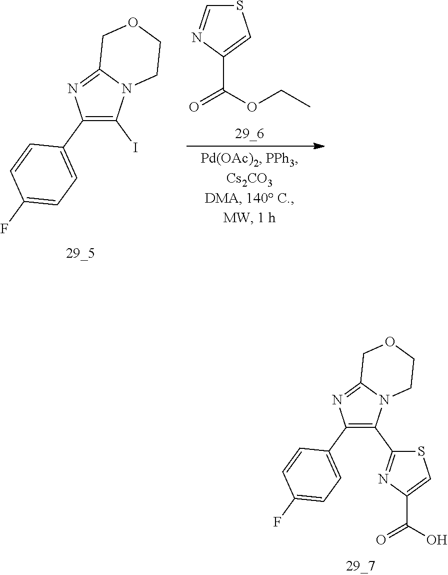

A mixture of 2-(4-fluorophenyl)-3-iodo-6,8-dihydro-5H-imidazo[2,1-c][1,4]oxazine Compound 29_5 (50 mg, 145.30 umol, 1 eq), ethyl thiazole-4-carboxylate Compound 296 (36.54 mg, 232.48 umol, 1.6 eq), diacetoxypalladium (1.63 mg, 7.26 umol, 0.05 eq), triphenylphosphane (3.81 mg, 14.53 umol, 0.1 eq) and dicesium carbonate (142.02 mg, 435.89 umol, 3 eq) in DMA (1 mL) was degassed and purged with N₂ for 3 times, and then the mixture was stirred at 140° C. for 1 hour under N₂ atmosphere under microwave. The reaction mixture was cooled to room temperature. The mixture was concentrated and extracted with EA (10 mL×2). Then the aqueous phase was adjusted to pH=3 by the solution of hydrochloric acid (1M). The aqueous layer was extracted with EA (10 mL×2). The combined organic layers were dried over anhydrous Na₂SO₄, filtered and concentrated under reduced pressure to give 2-(2-(4-fluorophenyl)-6,8-dihydro-5H-imidazo[2,1-c][1,4]oxazin-3-yl)thiazole-4-carboxylic acid Compound 297 (180 mg, crude) as a yellow solid, which was used to the next step directly without further purification.

### General Procedure for Preparation of 2-(2-(4-fluorophenyl)-6,8-dihydro-5H-imidazo[2,1-c][1,4]oxazin-3-yl)-N-(5-(4-methylpiperazin-1-yl)pyridin-2-yl)thiazole-4-carboxamide (Compound 29)

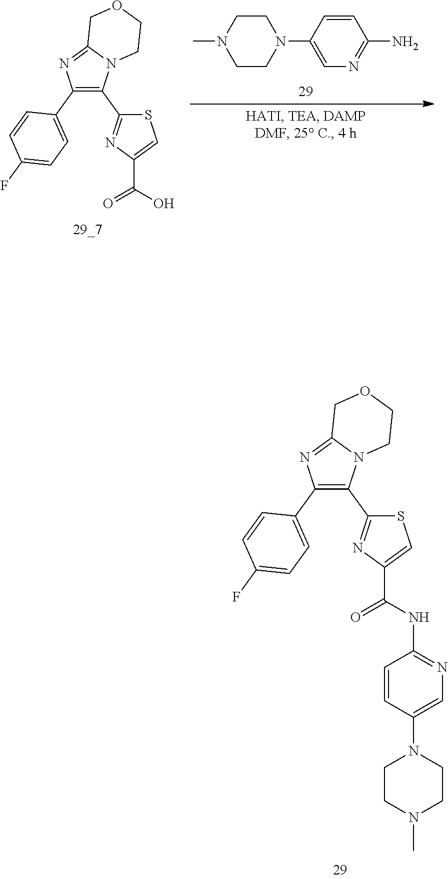 [SMILES: O=C(O)c1csc(-c2c(-c3ccc(F)cc3)nc3n2CCOC3)n1 ; CN1CCN(c2ccc(N)nc2)CC1 ; CN1CCN(c2ccc(NC(=O)c3csc(-c4c(-c5ccc(F)cc5)nc5n4CCOC5)n3)nc2)CC1]

To a solution of 2-(2-(4-fluorophenyl)-6,8-dihydro-5H-imidazo[2,1-c][1,4]oxazin-3-yl)thiazole-4-carboxylic acid Compound 29_7 (110 mg, 318.52 umol, 1 eq) and 5-(4-methylpiperazin-1-yl)pyridin-2-amine Compound 29_8 (122.48 mg, 637.04 umol, 2 eq) in DMF (3 mL) was added HATU (242.22 mg, 637.04 umol, 2 eq), DMAP (5.50 mg, 45.02 umol, 1.41e-1 eq) and DIEA (123.50 mg, 955.56 umol, 166.44 uL, 3 eq). The mixture was stirred at 25° C. for 4 hours. The mixture was concentrated under reduced pressure and purified by Prep-HPLC (Column: Phenomenex C18 75*30 mm*3 um, Mobile Phase A: water (NH₃H₂O+NH₄HCO₃), Mobile Phase B: acetonitrile, Flow rate: 25 mL/min, gradient condition from 38% B to 68%). The pure fractions were collected and the solvent was evaporated under vacuum to give a residue. The residue was partitioned between acetonitrile (2 mL) and water (10 mL). The solution was lyophilized to give 2-(2-(4-fluorophenyl)-6,8-dihydro-5H-imidazo[2,1-c][1,4]oxazin-3-yl)-N-(5-(4-methylpiperazin-1-yl)pyridin-2-yl)thiazole-4-carboxamide Compound 29 (20 mg, 33.84 umol, 10.62% yield, 87.92% purity) as off-white solids, which was determined by ¹HNMR, LCMS and HPLC. LCMS: Retention time: 3.331 min, (M+H)=520.4. HPLC: Retention time: 2.66 min. HNMR: (400 MHz, CD₃OD), δ=8.21 (s, 1H), 8.17 (d, J=9.0 Hz, 1H), 8.03 (d, J=2.8 Hz, 1H), 7.49 (br. dd., J=2.8, 9.2 Hz, 4H), 7.19 (t, J=8.8 Hz, 2H), 4.90 (br. s., 2H), 4.53 (t, J=5.2 Hz, 2H), 4.18 (t, J=5.2 Hz, 2H), 3.27-3.22 (m, 4H), 2.67-2.61 (m, 4H), 2.36 (s, 3H).

### Example 36: Synthesis of Compound 30

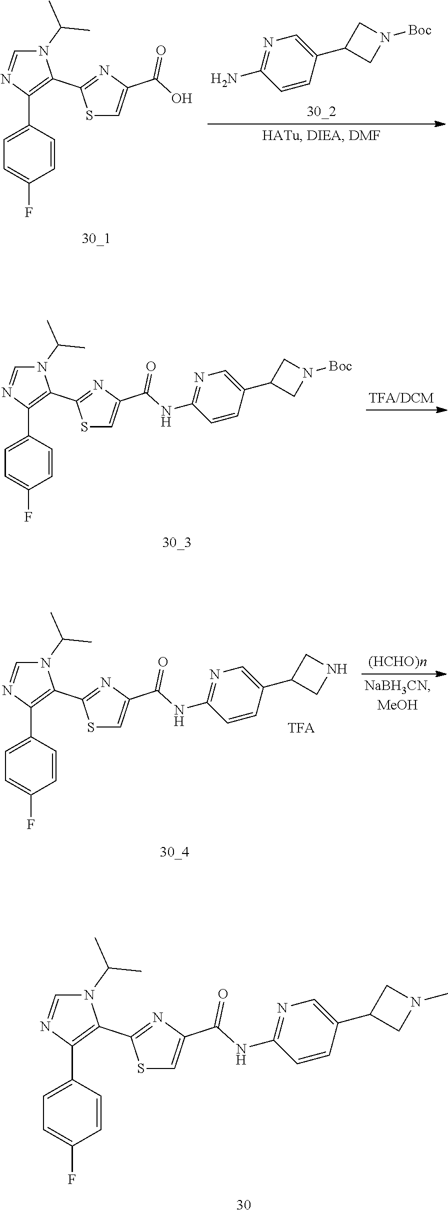 [SMILES: CC(C)n1cnc(-c2ccc(F)cc2)c1-c1nc(C(=O)O)cs1 ; CC(C)(C)OC(=O)N1CC(c2ccc(N)nc2)C1 ; CC(C)n1cnc(-c2ccc(F)cc2)c1-c1nc(C(=O)Nc2ccc(C3CN(C(=O)OC(C)(C)C)C3)cn2)cs1 ; CC(C)n1cnc(-c2ccc(F)cc2)c1-c1nc(C(=O)Nc2ccc(C3CNC3)cn2)cs1 ; CC(C)n1cnc(-c2ccc(F)cc2)c1-c1nc(C(=O)Nc2ccc(C3CN(C)C3)cn2)cs1]

### General Procedure for Preparation of tert-butyl 3-(6-(2-(4-(4-fluorophenyl)-1-isopropyl-1H-imidazol-5-yl)thiazole-4-carboxamido)pyridin-3-yl)azetidine-1-carboxylate (Compound 30_3)

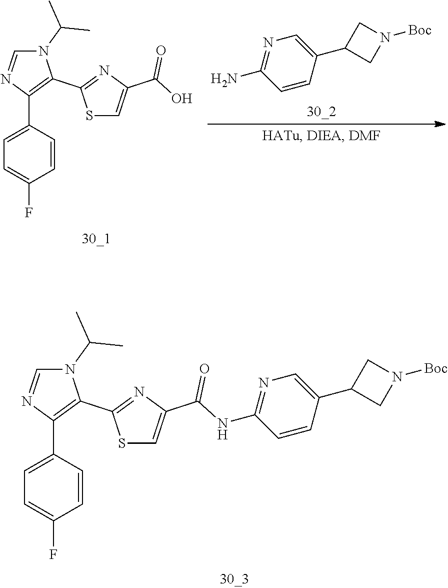 [SMILES: CC(C)n1cnc(-c2ccc(F)cc2)c1-c1nc(C(=O)O)cs1 ; CC(C)(C)OC(=O)N1CC(c2ccc(N)nc2)C1 ; CC(C)n1cnc(-c2ccc(F)cc2)c1-c1nc(C(=O)Nc2ccc(C3CN(C(=O)OC(C)(C)C)C3)cn2)cs1]

To a mixture of tert-butyl 3-(6-amino-3-pyridyl)azetidine-1-carboxylate (Compound 30_2) (574 mg, 2.30 mmol, 2.54 eq) and 2-[5-(4-fluorophenyl)-3-isopropyl-imidazol-4-yl]thiazole-4-carboxylic acid (Compound 30_1) (300 mg, 905.35 umol, 1 eq) in DMF (2 mL) was added DIEA (351.03 mg, 2.72 mmol, 473.09 uL, 3 eq) and HATU (378.66 mg, 995.88 umol, 1.1 eq) in one portion. The mixture was stirred at 25° C. and stirred for 0.5 hours. The mixture was poured into water (20 mL) and extracted with ethyl acetate (30 mL*3). The combined organic phase was washed with brine (10 mL*2), dried with anhydrous Na₂SO₄, filtered and concentrated under reduced pressure. The residue was purified by flash silica gel chromatography (ISCO®; 4 g SepaFlash® Silica Flash Column, Eluent of 0˜100% Ethyl acetate/Petroleum ether gradient @ 30 mL/min) to give tert-butyl 3-[6-[[2-[5-(4-fluorophenyl)-3-isopropyl-imidazol-4-yl]thiazole-4-carbonyl]amino]-3-pyridyl]azetidine-1-carboxylate (Compound 30_3) (0.4542 g, 753.43 umol, 83.22% yield, 93.334% purity) as yellow oil, which was determined by ¹HNMR and LCMS. LCMS: Retention time: 1.072 min, (M+H)=563.3. HNMR: (400 MHz, DMSO-d₆), δ=10.00 (s, 1H), 8.68 (s, 1H), 8.33 (d, J=2.4 Hz, 1H), 8.25-8.21 (m, 1H), 8.20 (s, 1H), 7.59-7.52 (m, 3H), 7.22-7.15 (m, 2H), 4.79-4.67 (m, 1H), 4.31-4.19 (m, 2H), 3.93-3.80 (m, 3H), 1.49 (d, J=6.8 Hz, 6H), 1.42-1.40 (m, 9H).

### General Procedure for Preparation of N-(5-(azetidin-3-yl)pyridin-2-yl)-2-(4-(4-fluorophenyl)-1-isopropyl-1H-imidazol-5-yl)thiazole-4-carboxamide (Compound 30_4)

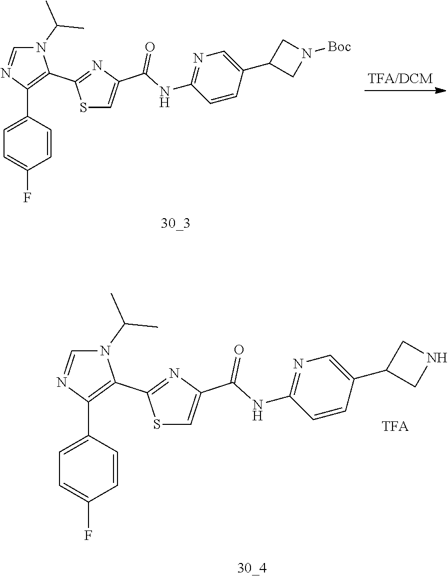 [SMILES: CC(C)n1cnc(-c2ccc(F)cc2)c1-c1nc(C(=O)Nc2ccc(C3CN(C(=O)OC(C)(C)C)C3)cn2)cs1 ; CC(C)n1cnc(-c2ccc(F)cc2)c1-c1nc(C(=O)Nc2ccc(C3CNC3)cn2)cs1]

To a mixture of tert-butyl 3-[6-[[2-[5-(4-fluorophenyl)-3-isopropyl-imidazol-4-yl]thiazole-4-carbonyl]amino]-3-pyridyl]azetidine-1-carboxylate (Compound 30_3) (0.4542 g, 807.24 umol, 1 eq) in DCM (4 mL) was added TFA (1 mL) in one portion. The mixture was stirred at 25° C. and stirred for 0.5 hours. The mixture was concentrated under reduced pressure to give N-[5-(azetidin-3-yl)-2-pyridyl]-2-[5-(4-fluorophenyl)-3-isopropyl-imidazol-4-yl]thiazole-4-carboxamide (Compound 30_4) (0.5 g, crude, TFA) as yellow oil, which was determined by LCMS. LCMS: Retention time: 0.584 min, (M+H)=463.1.

### General Procedure for Preparation of 2-(4-(4-fluorophenyl)-1-isopropyl-1H-imidazol-5-yl)-N-(5-(1-methylazetidin-3-yl)pyridin-2-yl)thiazole-4-carboxamide (Compound 30)

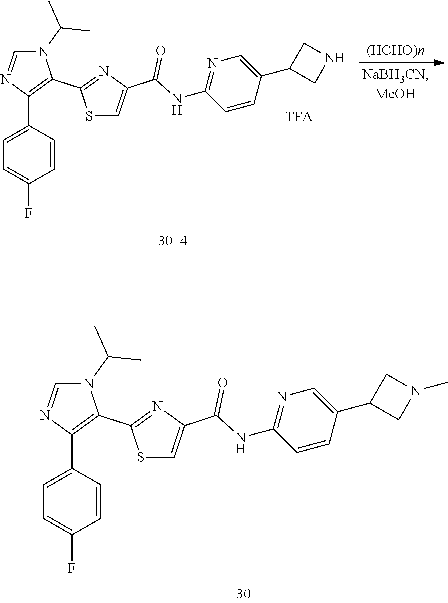 [SMILES: CC(C)n1cnc(-c2ccc(F)cc2)c1-c1nc(C(=O)Nc2ccc(C3CNC3)cn2)cs1 ; CC(C)n1cnc(-c2ccc(F)cc2)c1-c1nc(C(=O)Nc2ccc(C3CN(C)C3)cn2)cs1]

A solution of N-[5-(azetidin-3-yl)-2-pyridyl]-2-[5-(4-fluorophenyl)-3-isopropyl-imidazol-4-yl]thiazole-4-carboxamide (Compound 30_4) (0.5 g, 867.21 umol, 1 eq, TFA) and PARAFORMALDEHYDE (195.23 mg, 2.17 mmol, 2.5 eq) in MeOH (10 mL) was stirred at 25° C. for 0.5 hours. Then the NaBH₃CN (81.75 mg, 1.30 mmol, 1.5 eq) was added and the mixture was stirred at 25° C. for 0.5 hours. The mixture was purified by prep. HPLC (column: Welch Xtimate C18 150*30 mm*5 um; mobile phase: [water(FA)-ACN]; B %: 8%-38%, 7.7 min) and lyophilized to afford 2-[5-(4-fluorophenyl)-3-isopropyl-imidazol-4-yl]-N-[5-(1-methylazetidin-3-yl)-2-pyridyl]thiazole-4-carboxamide (Compound 30) (139.93 mg, 236.93 umol, 27.32% yield, 100% purity, TFA) as white solid, which was determined by ¹HNMR, LCMS and HPLC. LCMS: Retention time: 0.591 min, (M+H)=477.1. HPLC: Retention time: 1.450 min. HNMR: (400 MHz, DMSO-d₆), δ=9.98 (s, 1H), 8.67 (s, 1H), 8.36 (s, 1H), 8.26-8.13 (m, 2H), 8.02-7.89 (m, 1H), 7.51-7.41 (m, 2H), 7.25-7.13 (m, 2H), 4.79-4.65 (m, 1H), 3.85-3.77 (m, 2H), 3.76-3.71 (m, 1H), 3.45-3.41 (m, 2H), 2.45 (s, 3H), 1.49 (d, J=6.8 Hz, 6H).

### Example 37: Synthesis of Compound 31

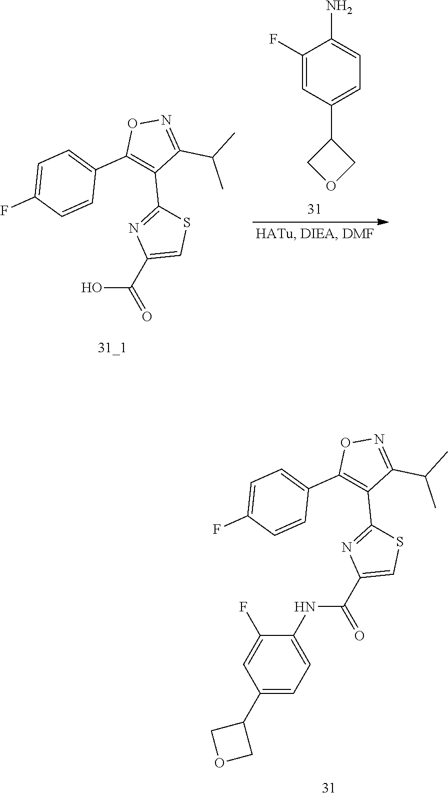 [SMILES: CC(C)c1noc(-c2ccc(F)cc2)c1-c1nc(C(=O)O)cs1 ; Nc1ccc(C2COC2)cc1F ; CC(C)c1noc(-c2ccc(F)cc2)c1-c1nc(C(=O)Nc2ccc(C3COC3)cc2F)cs1]

### General Procedure for Preparation of N-(2-fluoro-4-(oxetan-3-yl)phenyl)-2-(5-(4-fluorophenyl)-3-isopropylisoxazol-4-yl)thiazole-4-carboxamide (Compound 31)

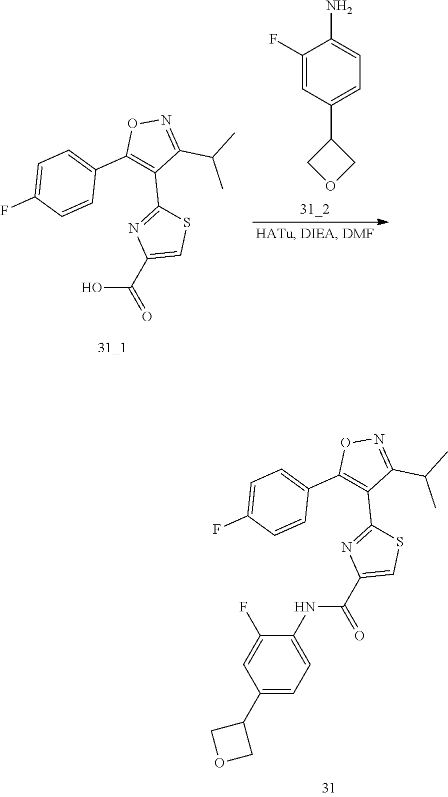 [SMILES: CC(C)c1noc(-c2ccc(F)cc2)c1-c1nc(C(=O)O)cs1 ; Nc1ccc(C2COC2)cc1F ; CC(C)c1noc(-c2ccc(F)cc2)c1-c1nc(C(=O)Nc2ccc(C3COC3)cc2F)cs1]

To a mixture of 2-fluoro-4-(oxetan-3-yl)aniline (Compound 31_2) (50 mg, 299.08 umol, 1.33 eq) and 2-[5-(4-fluorophenyl)-3-isopropyl-isoxazol-4-yl]thiazole-4-carboxylic acid (Compound 31_1) (75 mg, 225.67 umol, 1 eq) in DMF (1 mL) was added DIEA (87.50 mg, 677.00 umol, 117.92 uL, 3 eq) and HATU (85.81 mg, 225.67 umol, 1 eq) in one portion. The mixture was stirred at 25° C. and stirred for 8 hours. The mixture was filtered. The filtrate was pre-purified by Prep-HPLC (column: Welch Xtimate C18 150*25 mm*5 um; mobile phase: [water (FA)-ACN]; B %: 64%-94%, 6 min) and lyophilized to afford Compound N-[2-fluoro-4-(oxetan-3-yl)phenyl]-2-[5-(4-fluorophenyl)-3-isopropyl-isoxazol-4-yl]thiazole-4-carboxamide.

Compound 31 (58.23 mg, 120.93 umol, 53.59% yield, 100% purity) as a white solid, which was determined by ¹HNMR, LCMS and HPLC. LCMS: Retention time: 0.838 min, (M+H)=521.3. HPLC: Retention time: 2.77 min. HNMR: (400 MHz, DMSO-d₆), δ=9.65 (s, 1H), 8.55 (s, 1H), 8.01 (s, 1H), 7.70-7.62 (m, 1H), 7.59-7.52 (m, 2H), 7.24-7.16 (m, 2H), 6.97-6.89 (m, 1H), 6.85-6.77 (m, 1H), 3.74-3.65 (m, 1H), 3.33-3.27 (m, 4H), 2.87-2.72 (m, 4H), 2.49-2.45 (m, 3H), 1.08-0.99 (m, 2H), 0.99-0.91 (m, 2H).

### Example 38: Synthesis of Compound 38

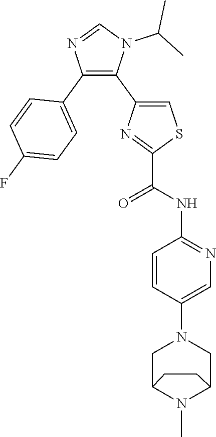 [SMILES: CC(C)n1cnc(-c2ccc(F)cc2)c1-c1csc(C(=O)Nc2ccc(N3CC4CCC(C3)N4C)cn2)n1]

4-(4-(4-fluorophenyl)-1-isopropyl-1H-imidazol-5-yl)-N-(5-((1R,5S)-8-methyl-3,8-diazabicyclo[3.2.1]octan-3-yl)pyridin-2-yl)thiazole-2-carboxamide (Compound 38) was synthesized under the same synthetic route as for Compound 2 as white solid, which was determined by ¹HNMR, LCMS and HPLC. LCMS: Retention time: 0.819 min, (M+H)=532.3. HPLC: Retention time: 2.086 min. HNMR: (400 MHz, DMSO-d₆), 10.12 (s, 1H), 8.19 (s, 1H), 8.04 (s, 1H), 7.99-7.94 (m, 1H), 7.90 (d, J=9.2 Hz, 1H), 7.44-7.29 (m, 3H), 7.13-7.02 (m, 2H), 4.32-4.18 (m, 1H), 3.48-3.37 (m, 2H), 3.28 (s, 3H), 2.98-2.84 (m, 2H), 2.37-2.33 (m, 2H), 2.05-1.96 (m, 2H), 1.77-1.65 (m, 2H), 1.39 (d, J=6.8 Hz, 6H).

### Example 39: Synthesis of Compound 39

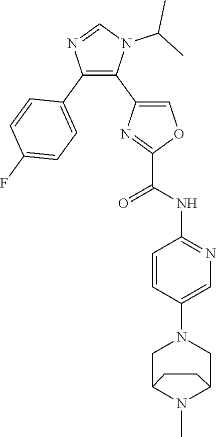 [SMILES: CC(C)n1cnc(-c2ccc(F)cc2)c1-c1coc(C(=O)Nc2ccc(N3CC4CCC(C3)N4C)cn2)n1]

4-(4-(4-fluorophenyl)-1-isopropyl-1H-imidazol-5-yl)-N-(5-(8-methyl-3,8-diazabicyclo[3.2.1]octan-3-yl)pyridin-2-yl)oxazole-2-carboxamide (Compound 39) was synthesized under the same synthetic route as for Compound 2 as light yellow solid, which was determined by ¹HNMR, LCMS and HPLC. LCMS: Retention time: 1.605 min, (M+H)=516.1. HPLC: Retention time: 2.44 min. HNMR: (400 MHz, DMSO-d₆), δ=10.47 (s, 1H), 8.60 (s, 1H), 8.08 (s, 1H), 7.96 (d, J=2.8 Hz, 1H), 7.86 (d, J=8.8 Hz, 1H), 7.59-7.48 (m, 2H), 7.38-7.30 (m, 1H), 7.19-7.06 (m, 2H), 4.40-4.22 (m, 1H), 3.42-3.37 (m, 2H), 3.25-3.13 (m, 2H), 2.94-2.80 (m, 2H), 2.23 (s, 3H), 2.02-1.88 (m, 2H), 1.67-1.58 (m, 2H), 1.42 (s, 3H), 1.41 (s, 3H).

### Example 40: Synthesis of Compound 40

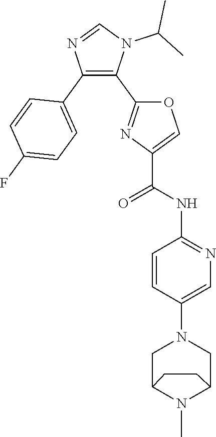 [SMILES: CC(C)n1cnc(-c2ccc(F)cc2)c1-c1nc(C(=O)Nc2ccc(N3CC4CCC(C3)N4C)cn2)co1]

2-(4-(4-fluorophenyl)-1-isopropyl-1H-imidazol-5-yl)-N-(5-((1R,5S)-8-methyl-3,8-diazabicyclo[3.2.1]octan-3-yl)pyridin-2-yl)oxazole-4-carboxamide (Compound 40) was synthesized under the same synthetic route as for Compound 2 as light yellow solid, which was determined by ¹HNMR, LCMS and HPLC. LCMS: Retention time: 0.822 min, (M+H)=516.3. HPLC: Retention time: 2.60 min. HNMR: (400 MHz, DMSO-d₆), 9.76 (s, 1H), 8.89 (s, 1H), 8.22 (s, 1H), 8.00-7.91 (m, 2H), 7.60-7.48 (m, 2H), 7.39-7.29 (m, 1H), 7.25-7.15 (m, 2H), 5.02-4.88 (m, 1H), 3.41-3.37 (m, 4H), 2.92-2.82 (m, 2H), 2.28 (s, 3H), 2.04-1.93 (m, 2H), 1.73-1.62 (m, 2H), 1.50 (d, J=6.8 Hz, 6H).

### Example 41: Synthesis of Compound 41

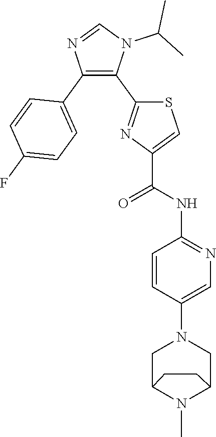 [SMILES: CC(C)n1cnc(-c2ccc(F)cc2)c1-c1nc(C(=O)Nc2ccc(N3CC4CCC(C3)N4C)cn2)cs1]

2-(4-(4-fluorophenyl)-1-isopropyl-1H-imidazol-5-yl)-N-(5-((1R,5S)-8-methyl-3,8-diazabicyclo[3.2.1]octan-3-yl)pyridin-2-yl)thiazole-4-carboxamide (Compound 41) was synthesized under the same synthetic route as for Compound 2 as white solid, which was determined by ¹HNMR, LCMS and HPLC. LCMS: Retention time: 1.523 min, (M+H)=532.1. HPLC: Retention time: 5.21 min. HNMR: (400 MHz, DMSO-d₆), δ=9.73 (s, 1H), 8.60 (s, 1H), 8.18 (s, 1H), 8.02 (d, J=9.2 Hz, 1H), 7.94 (d, J=3.2 Hz, 1H), 7.49-7.43 (m, 2H), 7.37-7.31 (m, 1H), 7.23-7.14 (m, 2H), 4.75-4.67 (m, 1H), 3.38 (d, J=10.0 Hz, 2H), 3.24-3.19 (m, 2H), 2.85 (d, J=10.0 Hz, 2H), 2.23 (s, 3H), 2.01-1.92 (m, 2H), 1.69-1.61 (m, 2H), 1.49 (d, J=6.8 Hz, 6H).

### Example 42: Synthesis of Compound 42

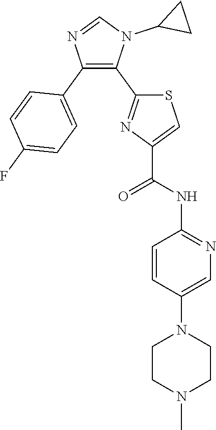 [SMILES: CN1CCN(c2ccc(NC(=O)c3csc(-c4c(-c5ccc(F)cc5)ncn4C4CC4)n3)nc2)CC1]

2-(1-cyclopropyl-4-(4-fluorophenyl)-1H-imidazol-5-yl)-N-(5-(4-methylpiperazin-1-yl)pyridin-2-yl)thiazole-4-carboxamide (Compound 42) was synthesized under the same synthetic route as for Compound 2 as white solid, which was determined by ¹HNMR, LCMS and HPLC. LCMS: Retention time: 0.987 min, (M+H)=504.3. HPLC: Retention time: 2.004 min. HNMR: (400 MHz, DMSO-d₆), δ=9.71 (s, 1H), 8.61 (s, 1H), 8.12-8.05 (m, 2H), 8.02 (s, 1H), 7.60-7.50 (m, 3H), 7.24-7.17 (m, 2H), 3.70-3.62 (m, 1H), 3.33-3.32 (m, 4H), 2.98-2.85 (m, 4H), 2.57-2.53 (m, 3H), 1.09-1.03 (m, 2H), 1.03-0.95 (m, 2H).

### Example 43: Synthesis of Compound 43

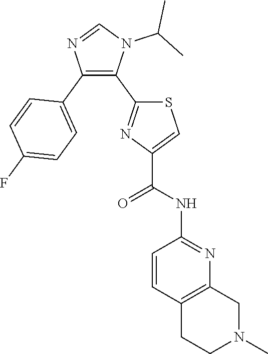 [SMILES: CC(C)n1cnc(-c2ccc(F)cc2)c1-c1nc(C(=O)Nc2ccc3c(n2)CN(C)CC3)cs1]

2-(4-(4-fluorophenyl)-1-isopropyl-1H-imidazol-5-yl)-N-(7-methyl-5,6,7,8-tetrahydro-1,7-naphthyridin-2-yl)thiazole-4-carboxamide (Compound 43) was synthesized under the same synthetic route as for Compound 2 as white powder, which was determined by ¹HNMR, LCMS and HPLC. LCMS: Retention time: 1.931 min, (M+H)=476.9.

HPLC: Retention time: 4.409 min. HNMR: (400 MHz, CDCl₃), δ=9.58 (s, 1H), 8.28 (s, 1H), 8.16 (d, J=8.0 Hz, 1H), 7.82 (s, 1H), 7.52 (d, J=8.4 Hz, 1H), 7.48-7.39 (m, 2H), 7.11-6.96 (m, 2H), 4.92-4.71 (m, 1H), 3.59 (s, 2H), 2.97-2.85 (m, 2H), 2.77-2.67 (m, 2H), 2.50 (s, 3H), 1.58 (d, J=6.8 Hz, 1H).

### Example 44: Synthesis of Compound 44

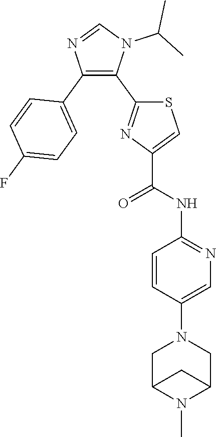 [SMILES: CC(C)n1cnc(-c2ccc(F)cc2)c1-c1nc(C(=O)Nc2ccc(N3CC4CC(C3)N4C)cn2)cs1]

2-(4-(4-fluorophenyl)-1-isopropyl-1H-imidazol-5-yl)-N-(5-(6-methyl-3,6-diazabicyclo[3.1.1]heptan-3-yl)pyridin-2-yl)thiazole-4-carboxamide (Compound 44) was synthesized under the same synthetic route as for Compound 2 as white solid, which was determined by ¹HNMR, LCMS and HPLC. LCMS: Retention time: 4.488 min, (M+H)=518.1. HPLC: Retention time: 10.84 min. HNMR: (400 MHz, DMSO-d₆), δ=9.73 (s, 1H), 8.60 (s, 1H), 8.20 (s, 1H), 8.07 (d, J=8.8 Hz, 1H), 7.91 (d, J=2.8 Hz, 1H), 7.51-7.42 (m, 2H), 7.28-7.23 (m, 1H), 7.22-7.15 (m, 2H), 4.78-4.65 (m, 1H), 3.64-3.56 (m, 2H), 3.49-3.43 (m, 2H), 3.31-3.26 (m, 2H), 2.47-2.40 (m, 1H), 2.01 (s, 3H), 1.58-1.53 (m, 1H), 1.50 (s, 3H), 1.48 (s, 3H).

### Example 45: Synthesis of Compound 45

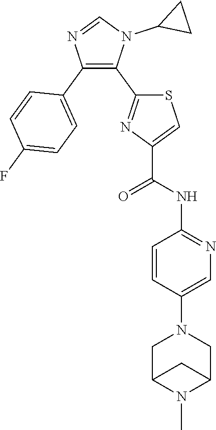 [SMILES: CN1C2CC1CN(c1ccc(NC(=O)c3csc(-c4c(-c5ccc(F)cc5)ncn4C4CC4)n3)nc1)C2]

2-(1-cyclopropyl-4-(4-fluorophenyl)-1H-imidazol-5-yl)-N-(5-(6-methyl-3,6-diazabicyclo[3.1.1]heptan-3-yl)pyridin-2-yl)thiazole-4-carboxamide (Compound 45) was synthesized under the same synthetic route as for Compound 2 as white solid, which was determined by ¹HNMR, LCMS and HPLC. LCMS: Retention time: 2.120 min, (M+H)=516.1. HPLC: Retention time: 1.509 min. HNMR: (400 MHz, DMSO-d₆), δ=9.65 (s, 1H), 8.59 (s, 1H), 8.07 (d, J=9.2 Hz, 1H), 8.02 (s, 1H), 7.89 (d, J=2.8 Hz, 1H), 7.60-7.53 (m, 2H), 7.28-7.16 (m, 3H), 3.72-3.64 (m, 1H), 3.58 (d, J=5.6 Hz, 2H), 3.46 (d, J=10.8 Hz, 2H), 3.32-3.29 (m, 2H), 2.46-2.40 (m, 1H), 2.00 (s, 3H), 1.55 (d, J=8.4 Hz, 1H), 1.10-1.04 (m, 2H), 1.02-0.95 (m, 2H).

### Example 46: Synthesis of Compound 46

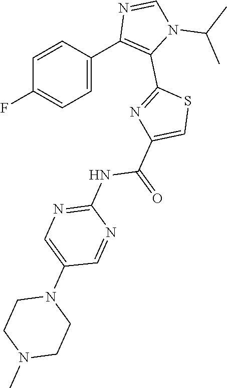 [SMILES: CC(C)n1cnc(-c2ccc(F)cc2)c1-c1nc(C(=O)Nc2ncc(N3CCN(C)CC3)cn2)cs1]

2-(4-(4-fluorophenyl)-1-isopropyl-1H-imidazol-5-yl)-N-(5-(4-methylpiperazin-1-yl)pyrimidin-2-yl)thiazole-4-carboxamide (Compound 46) was synthesized under the same synthetic route as for Compound 2 as white solid, which was determined by ¹HNMR, LCMS and HPLC. LCMS: Retention time: 1.723 min, (M+H)=507.1. HPLC: Retention time: 3.249 min. HNMR: (400 MHz, DMSO-d₆), δ=10.19 (s, 1H), 8.59 (s, 1H), 8.46 (s, 2H), 8.17 (s, 1H), 7.49-7.42 (m, 2H), 7.23-7.15 (m, 2H), 4.75-4.66 (m, 1H), 3.25-3.21 (m, 4H), 2.48-2.46 (m, 4H), 2.23 (s, 3H), 1.46 (d, J=6.8 Hz, 6H).

### Example 47: Synthesis of Compound 47

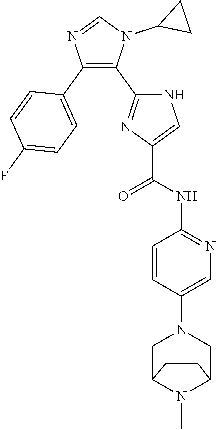 [SMILES: CN1C2CCC1CN(c1ccc(NC(=O)c3c[nH]c(-c4c(-c5ccc(F)cc5)ncn4C4CC4)n3)nc1)C2]

3′-cyclopropyl-5′-(4-fluorophenyl)-N-(5-(8-methyl-3,8-diazabicyclo[3.2.1]octan-3-yl)pyridin-2-yl)-1H,3′H-[2,4′-biimidazole]-4-carboxamide (Compound 47) was synthesized under the same synthetic route as for Compound 14 as white solid, which was determined by ¹HNMR, LCMS and HPLC. LCMS: Retention time: 1.207 min, (M+H)=513.1. HPLC: Retention time: 3.418 min. HNMR: (400 MHz, DMSO-d₆), δ=13.06 (br. s., 1H), 9.36 (s, 1H), 8.08 (s, 1H), 8.03 (d, J=8.8 Hz, 1H), 7.92 (s, 1H), 7.90-7.85 (m, 1H), 7.55-7.42 (m, 2H), 7.39-7.27 (m, 1H), 7.19-7.08 (m, 2H), 3.41-3.36 (m, 3H), 3.24-3.15 (m, 2H), 2.89-2.78 (m, 2H), 2.22 (s, 3H), 2.02-1.88 (m, 2H), 1.71-1.58 (m, 2H), 0.91-0.84 (m, 2H), 0.83-0.76 (m, 2H).

### Example 48: Synthesis of Compound 48

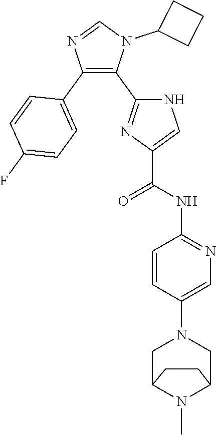 [SMILES: CN1C2CCC1CN(c1ccc(NC(=O)c3c[nH]c(-c4c(-c5ccc(F)cc5)ncn4C4CCC4)n3)nc1)C2]

3′-cyclobutyl-5′-(4-fluorophenyl)-N-(5-((1R,5S)-8-methyl-3,8-diazabicyclo[3.2.1]octan-3-yl)pyridin-2-yl)-1H,3′H-[2,4′-biimidazole]-4-carboxamide (Compound 48) was synthesized under the same synthetic route as for Compound 14 as white solid, which was determined by ¹HNMR, LCMS and HPLC. LCMS: Retention time: 0.706 min, (M+H)=527.2. HPLC: Retention time: 2.03 min. HNMR: (400 MHz, DMSO-d₆), δ=13.07 (s, 1H), 9.39 (s, 1H), 8.13 (s, 1H), 8.10-8.00 (m, 2H), 7.91 (d, J=2.8 Hz, 1H), 7.45-7.37 (m, 2H), 7.34 (dd, J=2.8, 9.2 Hz, 1H), 7.18-7.08 (m, 2H), 4.60-4.47 (m, 1H), 3.34-3.23 (m, 4H), 2.93-2.83 (m, 2H), 2.41-2.31 (m, 2H), 2.29 (s, 3H), 2.25-2.16 (m, 2H), 2.04-1.93 (m, 2H), 1.78-1.65 (m, 4H).

### Example 49: Synthesis of Compound 49

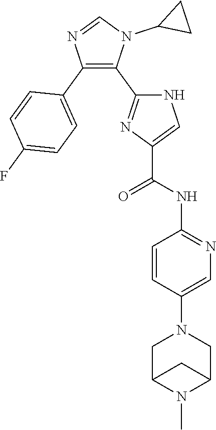 [SMILES: CN1C2CC1CN(c1ccc(NC(=O)c3c[nH]c(-c4c(-c5ccc(F)cc5)ncn4C4CC4)n3)nc1)C2]

3′-cyclopropyl-5′-(4-fluorophenyl)-N-(5-(6-methyl-3,6-diazabicyclo[3.1.1]heptan-3-yl)pyridin-2-yl)-1H,3′H-[2,4′-biimidazole]-4-carboxamide (Compound 49) was synthesized under the same synthetic route as for Compound 14 as white solid, which was determined by ¹HNMR, LCMS and HPLC. LCMS: Retention time: 3.279 min, (M+H)=499.2. HPLC: Retention time: 1.52 min. HNMR: (400 MHz, CD₃OD), δ=8.13 (d, J=8.8 Hz, 1H), 7.96-7.84 (m, 3H), 7.54-7.42 (m, 2H), 7.36-7.27 (m, 1H), 7.14-6.99 (m, 2H), 3.73 (d, J=5.6 Hz, 2H), 3.65-3.39 (m, 5H), 2.65 (s, 1H), 2.17 (s, 3H), 1.72 (br. d., J=8.8 Hz, 1H), 0.97-0.91 (m, 4H).

### Example 50: Synthesis of Compound 50

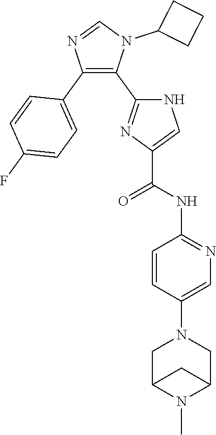 [SMILES: CN1C2CC1CN(c1ccc(NC(=O)c3c[nH]c(-c4c(-c5ccc(F)cc5)ncn4C4CCC4)n3)nc1)C2]

3′-cyclobutyl-5′-(4-fluorophenyl)-N-(5-(6-methyl-3,6-diazabicyclo[3.1.1]heptan-3-yl)pyridin-2-yl)-1H,3′H-[2,4′-biimidazole]-4-carboxamide (Compound 50) was synthesized under the same synthetic route as for Compound 14 as white solid, which was determined by ¹HNMR, LCMS and HPLC. LCMS: Retention time: 1.260 min, (M+H)=513.3. HPLC: Retention time: 1.635 min. HNMR: (400 MHz, DMSO-d₆), δ=13.06 (s, 1H), 9.36 (s, 1H), 8.22-8.12 (m, 1H), 8.11-8.01 (m, 2H), 7.86 (d, J=2.4 Hz, 1H), 7.46-7.36 (m, 2H), 7.28-7.21 (m, 1H), 7.18-7.10 (m, 2H), 4.62-4.48 (m, 1H), 3.60 (d, J=5.2 Hz, 2H), 3.53-3.47 (m, 2H), 3.30-3.29 (m, 2H), 2.43-2.34 (m, 2H), 2.28-2.16 (m, 2H), 2.08-1.95 (m, 3H), 1.79-1.65 (m, 2H), 1.56 (d, J=8.0 Hz, 1H), 1.23 (s, 1H).

### Example 51: Synthesis of Compound 51

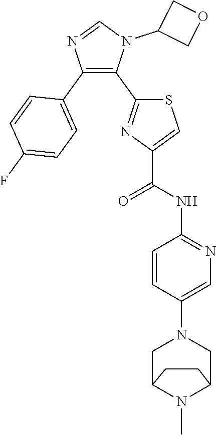 [SMILES: CN1C2CCC1CN(c1ccc(NC(=O)c3csc(-c4c(-c5ccc(F)cc5)ncn4C4COC4)n3)nc1)C2]

2-(4-(4-fluorophenyl)-1-(oxetan-3-yl)-1H-imidazol-5-yl)-N-(5-((1R,5S)-8-methyl-3,8-diazabicyclo[3.2.1]octan-3-yl)pyridin-2-yl)thiazole-4-carboxamide (Compound 51) was synthesized under the same synthetic route as for Compound 14 as light yellow solid, which was determined by ¹HNMR, LCMS and HPLC. LCMS: Retention time: 0.829 min, (M+H)=546.3. HPLC: Retention time: 2.202 min. HNMR: (400 MHz, DMSO-d₆), δ=9.81 (s, 1H), 8.48 (d, J=8.0 Hz, 2H), 8.05-7.95 (m, 2H), 7.56-7.47 (m, 2H), 7.38-7.32 (m, 1H), 7.28-7.20 (m, 2H), 5.84-5.74 (m, 1H), 4.96 (d, J=6.8 Hz, 4H), 3.27-3.20 (m, 4H), 2.90-2.82 (m, 2H), 2.24 (s, 3H), 2.03-1.88 (m, 2H), 1.72-1.59 (m, 2H).

### Example 52: Synthesis of Compound 52

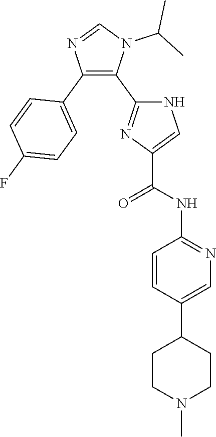 [SMILES: CC(C)n1cnc(-c2ccc(F)cc2)c1-c1nc(C(=O)Nc2ccc(C3CCN(C)CC3)cn2)c[nH]1]

5′-(4-fluorophenyl)-3′-isopropyl-N-(5-(1-methylpiperidin-4-yl)pyridin-2-yl)-1H,3′H-[2,4′-biimidazole]-4-carboxamide (Compound 52) was synthesized under the same synthetic route as for Compound 14 as white solid, which was determined by ¹HNMR, LCMS and HPLC. LCMS: Retention time: 1.831 min, (M+H)=488.0. HPLC: Retention time: 3.16 min. HNMR: (400 MHz, DMSO-d₆), δ=13.19 (s, 1H), 9.61 (s, 1H), 8.25 (s, 1H), 8.19 (d, J=8.4 Hz, 1H), 8.15 (s, 1H), 8.10 (s, 1H), 7.78-7.71 (m, 1H), 7.40-7.31 (m, 2H), 7.17-7.08 (m, 2H), 4.34-4.22 (m, 1H), 3.53 (d, J=12.0 Hz, 2H), 3.14-3.01 (m, 2H), 2.93-2.75 (m, 4H), 2.05 (d, J=13.6 Hz, 2H), 1.92-1.75 (m, 2H), 1.40 (d, J=6.8 Hz, 6H).

### Example 53: Synthesis of Compound 53

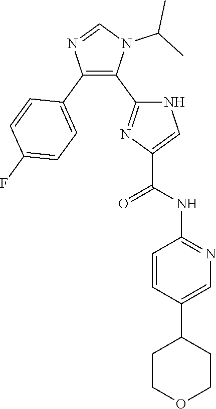 [SMILES: CC(C)n1cnc(-c2ccc(F)cc2)c1-c1nc(C(=O)Nc2ccc(C3CCOCC3)cn2)c[nH]1]

5′-(4-fluorophenyl)-3′-isopropyl-N-(5-(tetrahydro-2H-pyran-4-yl)pyridin-2-yl)-1H,3′H-[2,4′-biimidazole]-4-carboxamide (Compound 53) was synthesized under the same synthetic route as for Compound 14 as white solid, which was determined by ¹HNMR, LCMS and HPLC. LCMS: Retention time: 1.488 min, (M+H)=475.0. HPLC: Retention time: 1.565 min. HNMR: (400 MHz, DMSO-d₆), δ=13.38-13.02 (m, 1H), 9.56 (s, 1H), 8.24 (d, J=2.0 Hz, 1H), 8.18-8.11 (m, 2H), 8.09 (s, 1H), 7.78 (dd, J=2.0, 8.6 Hz, 1H), 7.41-7.33 (m, 2H), 7.18-7.10 (m, 2H), 4.32-4.24 (m, 1H), 3.99-3.91 (m, 2H), 3.47-3.41 (m, 2H), 2.86-2.76 (m, 1H), 1.74-1.65 (m, 4H), 1.40 (d, J=6.8 Hz, 6H).

### Example 54: Synthesis of Compound 54

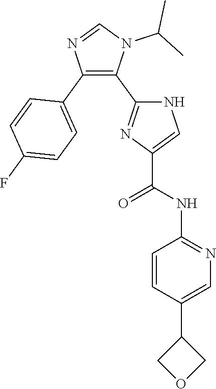 [SMILES: CC(C)n1cnc(-c2ccc(F)cc2)c1-c1nc(C(=O)Nc2ccc(C3COC3)cn2)c[nH]1]

5′-(4-fluorophenyl)-3′-isopropyl-N-(5-(oxetan-3-yl)pyridin-2-yl)-1H,3′H-[2,4′-biimidazole]-4-carboxamide (Compound 54) was synthesized under the same synthetic route as for Compound 14 as white solid, which was determined by ¹HNMR, LCMS and HPLC. LCMS: Retention time: 0.676 min, (M+H)=447.2. HPLC: Retention time: 1.984 min. HNMR: (400 MHz, DMSO-d₆), δ=13.16 (br s, 1H), 9.62 (s, 1H), 8.31 (s, 1H), 8.24 (d, J=8.0 Hz, 1H), 8.14 (s, 1H), 8.08 (s, 1H), 8.03-7.97 (m, 1H), 7.44-7.30 (m, 2H), 7.20-7.06 (m, 2H), 5.01-4.88 (m, 2H), 4.66-4.60 (m, 2H), 4.37-4.20 (m, 2H), 1.40 (d, J=8.0 Hz, 6H).

### Example 55: Synthesis of Compound 55

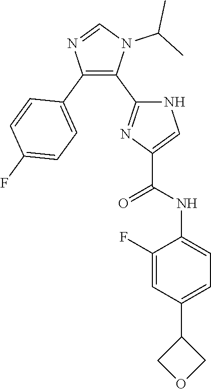 [SMILES: CC(C)n1cnc(-c2ccc(F)cc2)c1-c1nc(C(=O)Nc2ccc(C3COC3)cc2F)c[nH]1]

N-(2-fluoro-4-(oxetan-3-yl)phenyl)-5′-(4-fluorophenyl)-3′-isopropyl-1H,3′H-[2,4′-biimidazole]-4-carboxamide (Compound 55) was synthesized under the same synthetic route as for Compound 14 as white solid, which was determined by ¹HNMR, LCMS and HPLC. LCMS: Retention time: 1.711 min, (M+H)=463.9. HPLC: Retention time: 8.83 min. HNMR: (400 MHz, DMSO-d₆), δ=13.13 (s, 1H), 9.57 (s, 1H), 8.12-8.04 (m, 2H), 8.03-7.86 (m, 1H), 7.44-7.32 (m, 3H), 7.25 (d, J=8.8 Hz, 1H), 7.14 (t, J=8.8 Hz, 2H), 4.92 (d, J=6.0 Hz, 2H), 4.62 (t, J=6.4 Hz, 2H), 4.32-4.21 (m, 2H), 1.41 (d, J=6.4 Hz, 6H).

### Example 56: Synthesis of Compound 56

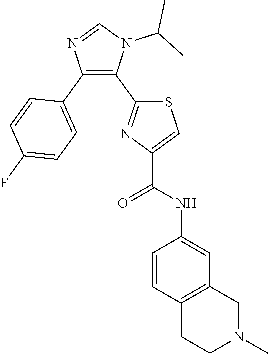 [SMILES: CC(C)n1cnc(-c2ccc(F)cc2)c1-c1nc(C(=O)Nc2ccc3c(c2)CN(C)CC3)cs1]

2-(4-(4-fluorophenyl)-1-isopropyl-1H-imidazol-5-yl)-N-(2-methyl-1,2,3,4-tetrahydroisoquinolin-7-yl)thiazole-4-carboxamide (Compound 56) was synthesized under the same synthetic route as for Compound 2 as white solid, which was determined by ¹HNMR, LCMS and HPLC. LCMS: Retention time: 0.643 min, (M+H)=476.1. HPLC: Retention time: 1.449 min. HNMR: (400 MHz, DMSO-d₆), δ=10.13 (s, 1H), 8.58 (s, 1H), 8.17 (s, 1H), 7.58-7.49 (m, 2H), 7.48-7.40 (m, 2H), 7.22-7.13 (m, 2H), 7.08 (d, J=8.4 Hz, 1H), 4.64 (quin, J=6.4 Hz, 1H), 3.47 (br s, 2H), 2.82-2.75 (m, 2H), 2.62-2.55 (m, 2H), 2.34 (s, 3H), 1.45 (d, J=6.8 Hz, 6H).

### Example 57: Synthesis of Compound 57

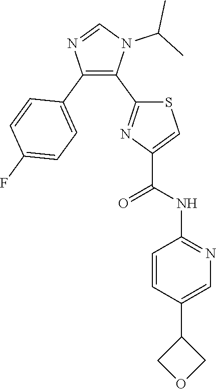 [SMILES: CC(C)n1cnc(-c2ccc(F)cc2)c1-c1nc(C(=O)Nc2ccc(C3COC3)cn2)cs1]

2-(4-(4-fluorophenyl)-1-isopropyl-1H-imidazol-5-yl)-N-(5-(oxetan-3-yl)pyridin-2-yl)thiazole-4-carboxamide (Compound 57) was synthesized under the same synthetic route as for Compound 2 as white solid, which was determined by ¹HNMR, LCMS and HPLC. LCMS: Retention time: 1.858 min, (M+H)=464.0. HPLC: Retention time: 2.700 min. HNMR (400 MHz, DMSO-d₆), δ=10.02 (s, 1H), 8.69 (s, 1H), 8.39-8.32 (m, 2H), 8.24 (d, J=8.4 Hz, 1H), 8.08-8.01 (m, 1H), 7.51-7.43 (m, 2H), 7.21 (t, J=8.8 Hz, 2H), 5.03-4.89 (m, 2H), 4.82-4.72 (m, 1H), 4.64 (t, J=6.4 Hz, 2H), 4.33-4.25 (m, 1H), 1.50 (d, J=6.8 Hz, 6H).

### Example 58: Synthesis of Compound 58

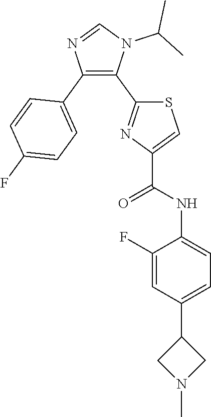 [SMILES: CC(C)n1cnc(-c2ccc(F)cc2)c1-c1nc(C(=O)Nc2ccc(C3CN(C)C3)cc2F)cs1]

N-(2-fluoro-4-(1-methylazetidin-3-yl)phenyl)-2-(4-(4-fluorophenyl)-1-isopropyl-1H-imidazol-5-yl)thiazole-4-carboxamide (Compound 58) was synthesized under the same synthetic route as for Compound 30 as white solid, which was determined by ¹HNMR, LCMS and HPLC. LCMS: Retention time: 1.030 min, (M+H)=494.3. HPLC: Retention time: 2.061 min. HNMR: (400 MHz, DMSO-d₆), δ=9.88 (s, 1H), 8.60 (s, 1H), 8.20 (s, 1H), 7.93-7.84 (m, 1H), 7.53-7.44 (m, 2H), 7.35 (d, J=11.2 Hz, 1H), 7.25-7.17 (m, 3H), 4.80-4.70 (m, 1H), 3.68-3.65 (m, 3H), 3.23-3.17 (m, 2H), 2.33 (s, 3H), 1.50 (d, J=6.8 Hz, 6H).

### Example 59: Synthesis of Compound 59

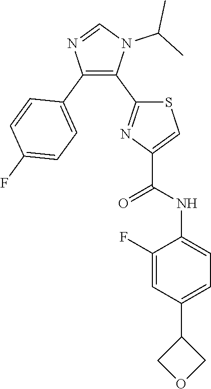 [SMILES: CC(C)n1cnc(-c2ccc(F)cc2)c1-c1nc(C(=O)Nc2ccc(C3COC3)cc2F)cs1]

N-(2-fluoro-4-(1-methylazetidin-3-yl)phenyl)-2-(4-(4-fluorophenyl)-1-isopropyl-1H-imidazol-5-yl)thiazole-4-carboxamide (Compound 59) was synthesized under the same synthetic route as for Compound 2 as white solid, which was determined by ¹HNMR, LCMS and HPLC. LCMS: Retention time: 0.717 min, (M+H)=481.1. HPLC: Retention time: 3.66 min. HNMR: (400 MHz, DMSO-d₆), δ=9.91 (s, 1H), 8.60 (s, 1H), 8.20 (s, 1H), 7.98-7.87 (m, 1H), 7.52-7.44 (m, 2H), 7.42-7.35 (m, 1H), 7.31-7.25 (m, 1H), 7.24-7.15 (m, 2H), 4.97-4.88 (m, 2H), 4.80-4.68 (m, 1H), 4.62 (t, J=6.4 Hz, 2H), 4.33-4.22 (m, 1H), 1.49 (d, J=6.8 Hz, 6H).

### Example 60: Synthesis of Compound 60

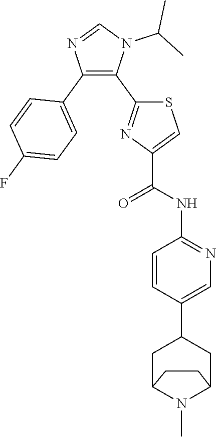 [SMILES: CC(C)n1cnc(-c2ccc(F)cc2)c1-c1nc(C(=O)Nc2ccc(C3CC4CCC(C3)N4C)cn2)cs1]

2-(4-(4-fluorophenyl)-1-isopropyl-1H-imidazol-5-yl)-N-(5-((1R,5S)-8-methyl-8-azabicyclo[3.2.1]octan-3-yl)pyridin-2-yl)thiazole-4-carboxamide (Compound 60) was synthesized under the same synthetic route as for Compound 2 as off-white solid, which was determined by ¹H NMR, LCMS and HPLC. LCMS: Retention time: 0.658 min, (M+H)=531.2. HPLC: Retention time: 2.79 min. HNMR: (400 MHz, DMSO-d₆), δ=9.92 (s, 1H), 8.66 (s, 1H), 8.47-8.37 (m, 1H), 8.20 (s, 1H), 8.14 (d, J=8.4 Hz, 1H), 7.98-7.87 (m, 1H), 7.50-7.40 (m, 2H), 7.22-7.14 (m, 2H), 4.78-4.66 (m, 1H), 3.21-3.00 (m, 1H), 2.55-2.52 (m, 2H), 2.45-2.28 (m, 5H), 2.08-1.81 (m, 4H), 1.56-1.37 (m, 8H).

### Example 61: Synthesis of Compound 61

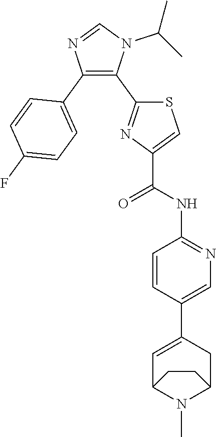 [SMILES: CC(C)n1cnc(-c2ccc(F)cc2)c1-c1nc(C(=O)Nc2ccc(C3=CC4CCC(C3)N4C)cn2)cs1]

2-(4-(4-fluorophenyl)-1-isopropyl-1H-imidazol-5-yl)-N-(5-((1R,5S)-8-methyl-8-azabicyclo[3.2.1]oct-2-en-3-yl)pyridin-2-yl)thiazole-4-carboxamide (Compound 61) was synthesized under the same synthetic route as for Compound 2 as white solid, which was determined by ¹HNMR, LCMS and HPLC. LCMS: Retention time: 0.667 min, (M+H)=529.1. HPLC: Retention time: 2.95 min. HNMR: (400 MHz, DMSO-d₆) (t=75° C.), δ=9.89 (s, 1H), 8.64 (s, 1H), 8.51 (d, J=2.4 Hz, 1H), 8.23 (d, J=8.8 Hz, 1H), 8.13 (s, 1H), 8.00 (dd, J=2.4, 8.8 Hz, 1H), 7.52-7.46 (m, 2H), 7.19-7.11 (m, 2H), 6.54-6.43 (m, 1H), 4.80-4.66 (m, 1H), 4.28-4.21 (m, 1H), 4.17-4.06 (m, 1H), 3.16-3.13 (m, 1H), 2.80 (s, 3H), 2.75-2.67 (m, 1H), 2.38-2.29 (m, 2H), 2.25-2.19 (m, 1H), 2.05-1.91 (m, 1H), 1.52 (d, J=6.8 Hz, 6H).

### Example 62: Synthesis of Compound 62

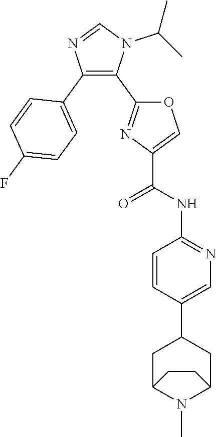 [SMILES: CC(C)n1cnc(-c2ccc(F)cc2)c1-c1nc(C(=O)Nc2ccc(C3CC4CCC(C3)N4C)cn2)co1]

2-(4-(4-fluorophenyl)-1-isopropyl-1H-imidazol-5-yl)-N-(5-((1R,5S)-8-methyl-8-azabicyclo[3.2.1]octan-3-yl)pyridin-2-yl)oxazole-4-carboxamide (Compound 62) was synthesized under the same synthetic route as for Compound 2 as off-white solid, which was determined by ¹HNMR, LCMS and HPLC. LCMS: Retention time: 0.652 min, (M+H)=515.1. HPLC: Retention time: 2.65 min. HNMR: (400 MHz, DMSO-d₆), δ=9.99 (s, 1H), 8.95 (s, 1H), 8.41 (d, J=2.0 Hz, 1H), 8.24 (s, 1H), 8.09 (d, J=8.8 Hz, 1H), 7.90 (dd, J=2.2, 8.8 Hz, 1H), 7.59-7.51 (m, 2H), 7.24-7.17 (m, 2H), 5.05-4.88 (m, 1H), 3.12-3.05 (m, 1H), 2.53-2.51 (m, 2H), 2.39-2.27 (m, 5H), 2.07-1.95 (m, 2H), 1.92-1.78 (m, 2H), 1.53-1.41 (m, 8H).

### Example 63: Synthesis of Compound 63

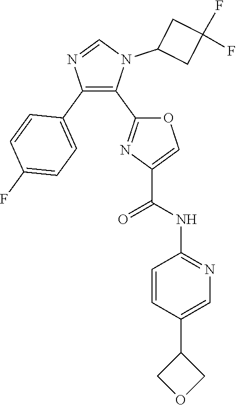 [SMILES: O=C(Nc1ccc(C2COC2)cn1)c1coc(-c2c(-c3ccc(F)cc3)ncn2C2CC(F)(F)C2)n1]

2-(1-(3,3-difluorocyclobutyl)-4-(4-fluorophenyl)-1H-imidazol-5-yl)-N-(5-(oxetan-3-yl)pyridin-2-yl)oxazole-4-carboxamide (Compound 63) was synthesized under the same synthetic route as for Compound 2 as white solid, which was determined by ¹HNMR, LCMS and HPLC. LCMS: Retention time: 1.863 min, (M+H)=496.1. HPLC: Retention time: 3.749 min. HNMR: (400 MHz, DMSO-d₆), δ=10.13 (s, 1H), 8.93 (s, 1H), 8.42-8.35 (m, 2H), 8.21 (d, J=8.6 Hz, 1H), 8.03 (dd, J=2.4, 8.4 Hz, 1H), 7.65-7.55 (m, 2H), 7.28-7.18 (m, 2H), 5.29-5.18 (m, 1H), 4.97-4.92 (m, 2H), 4.67-4.60 (m, 2H), 4.36-4.25 (m, 1H), 3.30-3.22 (m, 4H).

### Example 64: Synthesis of Compound 64

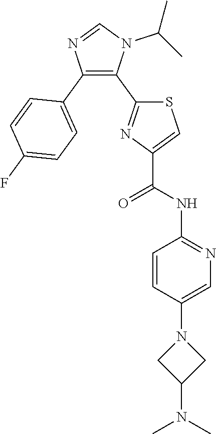 [SMILES: CC(C)n1cnc(-c2ccc(F)cc2)c1-c1nc(C(=O)Nc2ccc(N3CC(N(C)C)C3)cn2)cs1]

N-(5-(3-(dimethylamino)azetidin-1-yl)pyridin-2-yl)-2-(4-(4-fluorophenyl)-1-isopropyl-1H-imidazol-5-yl)thiazole-4-carboxamide (Compound 64) was synthesized under the same synthetic route as for Compound 2 as white solid, which was determined by ¹HNMR, LCMS and HPLC. LCMS: Retention time: 0.673 min, (M+Na)=528.1. HPLC: Retention time: 2.48 min. HNMR: (400 MHz, CDCl₃), δ=9.49 (s, 1H), 8.34-8.14 (m, 2H), 7.81 (s, 1H), 7.63-7.59 (m, 1H), 7.48-7.42 (m, 2H), 7.07-6.98 (m, 2H), 6.93-6.87 (m, 1H), 4.90-4.79 (m, 1H), 4.13-4.03 (m, 2H), 3.90-3.77 (m, 2H), 3.48 (s, 1H), 2.45-2.34 (m, 6H), 1.62-1.54 (m, 6H).

### Example 65: Synthesis of Compound 65

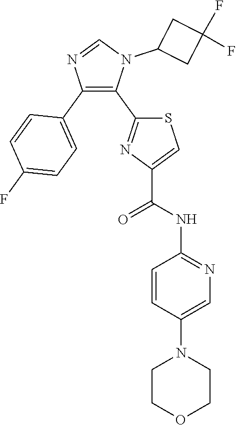 [SMILES: O=C(Nc1ccc(N2CCOCC2)cn1)c1csc(-c2c(-c3ccc(F)cc3)ncn2C2CC(F)(F)C2)n1]

2-(1-(3,3-difluorocyclobutyl)-4-(4-fluorophenyl)-1H-imidazol-5-yl)-N-(5-morpholinopyridin-2-yl)thiazole-4-carboxamide (Compound 65) was synthesized under the same synthetic route as for Compound 2 as white solid, which was determined by ¹HNMR, LCMS and HPLC. LCMS: Retention time: 1.513 min, (M+H)=541.1. HPLC: Retention time: 3.65 min. HNMR: (400 MHz, CDCl3), δ=9.98 (s, 1H), 8.61 (s, 1H), 8.48 (s, 1H), 8.14-8.03 (m, 2H), 7.59-7.43 (m, 3H), 7.29-7.17 (m, 2H), 5.19-5.03 (m, 1H), 3.85-3.64 (m, 4H), 3.31-3.20 (m, 2H), 3.19-3.06 (m, 6H).

### Example 66: Synthesis of Compound 66

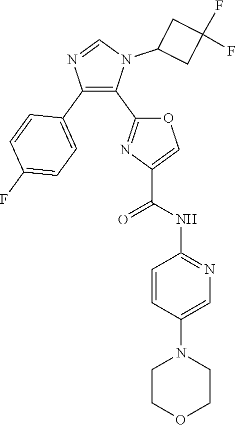 [SMILES: O=C(Nc1ccc(N2CCOCC2)cn1)c1coc(-c2c(-c3ccc(F)cc3)ncn2C2CC(F)(F)C2)n1]

2-(1-(3,3-difluorocyclobutyl)-4-(4-fluorophenyl)-1H-imidazol-5-yl)-N-(5-morpholinopyridin-2-yl)oxazole-4-carboxamide (Compound 66) was synthesized under the same synthetic route as for Compound 2 as white powder, which was determined by ¹HNMR, LCMS and HPLC. LCMS: Retention time: 3.073 min, (M+H)=525.1. HPLC: Retention time: 3.77 min. HNMR: (400 MHz, CDCl3), δ=9.18 (s, 1H), 8.32-8.23 (m, 2H), 8.01 (d, J=2.8 Hz, 1H), 7.89 (s, 1H), 7.62-7.55 (m, 2H), 7.39-7.31 (m, 1H), 7.15-7.05 (m, 2H), 5.39-5.24 (m, 1H), 3.94-3.85 (m, 4H), 3.44-3.28 (m, 2H), 3.22-3.14 (m, 4H), 3.11-2.96 (m, 2H).

### Example 67: Synthesis of Compound 67

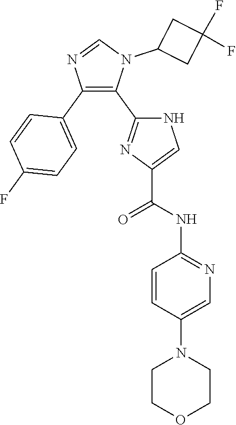 [SMILES: O=C(Nc1ccc(N2CCOCC2)cn1)c1c[nH]c(-c2c(-c3ccc(F)cc3)ncn2C2CC(F)(F)C2)n1]

3′-(3,3-difluorocyclobutyl)-5′-(4-fluorophenyl)-N-(5-morpholinopyridin-2-yl)-1H,3′H-[2,4′-biimidazole]-4-carboxamide (Compound 67) was synthesized under the same synthetic route as for Compound 14 as white solid, which was determined by ¹HNMR, LCMS and HPLC. LCMS: Retention time: 2.940 min, (M+H)=524.1. HPLC: Retention time: 5.70 min. HNMR: (400 MHz, DMSO-d₆), δ=13.08 (s, 1H), 9.47 (s, 1H), 8.23 (s, 1H), 8.13-8.04 (m, 3H), 7.52-7.46 (m, 1H), 7.44-7.39 (m, 2H), 7.21-7.12 (m, 2H), 4.74-4.65 (m, 1H), 3.79-3.71 (m, 4H), 3.18-2.98 (m, 8H).

### Example 68: Synthesis of Compound 68

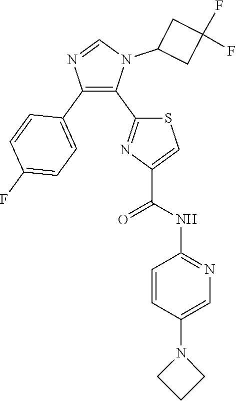 [SMILES: O=C(Nc1ccc(N2CCC2)cn1)c1csc(-c2c(-c3ccc(F)cc3)ncn2C2CC(F)(F)C2)n1]

N-(5-(azetidin-1-yl)pyridin-2-yl)-2-(1-(3,3-difluorocyclobutyl)-4-(4-fluorophenyl)-1H-imidazol-5-yl)thiazole-4-carboxamide (Compound 68) was synthesized under the same synthetic route as for Compound 2 as white solid, which was determined by ¹HNMR, LCMS and HPLC. LCMS: Retention time: 2.129 min, (M+H)=511.1. HPLC: Retention time: 4.273 min. HNMR: (400 MHz, DMSO-d₆), δ=9.81 (s, 1H), 8.55 (s, 1H), 8.33 (s, 1H), 8.01 (d, J=8.8 Hz, 1H), 7.61 (d, J=2.4 Hz, 1H), 7.54-7.44 (m, 2H), 7.29-7.15 (m, 2H), 6.97 (dd, J=2.8, 8.8 Hz, 1H), 5.14-5.02 (m, 1H), 3.93-3.79 (m, 4H), 3.28-3.06 (m, 4H), 2.42-2.29 (m, 2H).

### Example 69: Synthesis of Compound 69

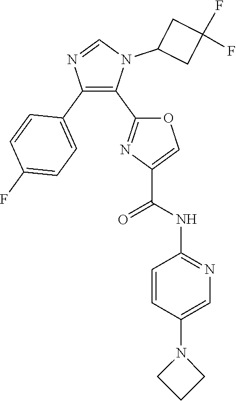 [SMILES: O=C(Nc1ccc(N2CCC2)cn1)c1coc(-c2c(-c3ccc(F)cc3)ncn2C2CC(F)(F)C2)n1]

N-(5-(azetidin-1-yl)pyridin-2-yl)-2-(1-(3,3-difluorocyclobutyl)-4-(4-fluorophenyl)-1H-imidazol-5-yl)oxazole-4-carboxamide (Compound 69) was synthesized under the same synthetic route as for Compound 2 as light gray powder, which was determined by ¹HNMR, LCMS and HPLC. LCMS: Retention time: 3.151 min, (M+H)=495.0. HPLC: Retention time: 3.88 min. HNMR: (400 MHz, CDCl₃), δ=9.20 (s, 1H), 8.26 (s, 1H), 8.22 (d, J=9.2 Hz, 1H), 7.89 (s, 1H), 7.62-7.53 (m, 3H), 7.15-7.06 (m, 2H), 6.93-6.85 (m, 1H), 5.44-5.25 (m, 1H), 4.01-3.83 (m, 4H), 3.44-3.27 (m, 2H), 3.11-2.93 (m, 2H), 2.51-2.38 (m, 2H).

### Example 70: Synthesis of Compound 70

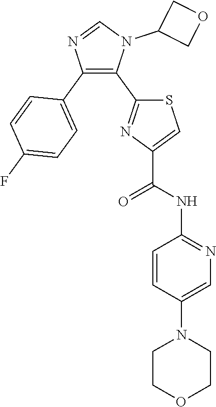 [SMILES: O=C(Nc1ccc(N2CCOCC2)cn1)c1csc(-c2c(-c3ccc(F)cc3)ncn2C2COC2)n1]

2-(4-(4-fluorophenyl)-1-(oxetan-3-yl)-1H-imidazol-5-yl)-N-(5-morpholinopyridin-2-yl)thiazole-4-carboxamide (Compound 70) was synthesized under the same synthetic route as for Compound 2 as white solid, which was determined by ¹HNMR, LCMS and HPLC. LCMS: Retention time: 2.500 min, (M+H)=507.0. HPLC: Retention time: 8.38 min. HNMR: (400 MHz, DMSO-d₆), δ=9.88 (s, 1H), 8.50 (s, 1H), 8.46 (s, 1H), 8.12 (d, J=2.8 Hz, 1H), 8.07 (d, J=8.8 Hz, 1H), 7.56-7.47 (m, 3H), 7.29-7.20 (m, 2H), 5.85-5.75 (m, 1H), 5.02-4.92 (m, 4H), 3.80-3.71 (m, 4H), 3.20-3.10 (m, 4H).

### Example 71: Synthesis of Compound 71

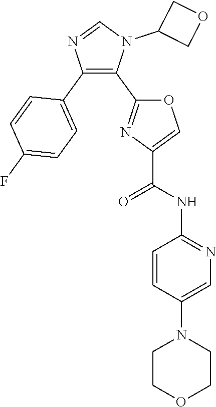 [SMILES: O=C(Nc1ccc(N2CCOCC2)cn1)c1coc(-c2c(-c3ccc(F)cc3)ncn2C2COC2)n1]

2-(4-(4-fluorophenyl)-1-(oxetan-3-yl)-1H-imidazol-5-yl)-N-(5-morpholinopyridin-2-yl)oxazole-4-carboxamide (Compound 71) was synthesized under the same synthetic route as for Compound 2 as white solid, which was determined by ¹HNMR, LCMS and HPLC. LCMS: Retention time: 0.700 min, (M+H)=491.1. HPLC: Retention time: 3.19 min. HNMR: (400 MHz, DMSO-d₆), δ=9.93 (s, 1H), 8.83 (s, 1H), 8.52 (s, 1H), 8.12 (d, J=2.8 Hz, 1H), 8.03 (d, J=9.2 Hz, 1H), 7.67-7.59 (m, 2H), 7.50 (dd, J=2.8, 9.2 Hz, 1H), 7.29-7.20 (m, 2H), 6.03-5.90 (m, 1H), 5.06-5.00 (m, 2H), 4.98-4.92 (m, 2H), 3.79-3.72 (m, 4H), 3.19-3.12 (m, 4H).

### Example 72: Synthesis of Compound 72

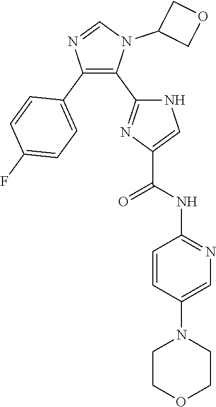 [SMILES: O=C(Nc1ccc(N2CCOCC2)cn1)c1c[nH]c(-c2c(-c3ccc(F)cc3)ncn2C2COC2)n1]

5′-(4-fluorophenyl)-N-(5-morpholinopyridin-2-yl)-3′-(oxetan-3-yl)-1H,3′H-[2,4′-biimidazole]-4-carboxamide (Compound 72) was synthesized under the same synthetic route as for Compound 14 as white solid, which was determined by ¹HNMR, LCMS and HPLC. LCMS: Retention time: 2.269 min, (M+H)=490.1. HPLC: Retention time: 2.296 min. HNMR: (400 MHz, DMSO-d₆), δ=13.01 (s., 1H), 9.46 (s, 1H), 8.39 (s, 1H), 8.16-8.00 (m, 3H), 7.53-7.47 (m, 1H), 7.47-7.40 (m, 2H), 7.17 (t, J=8.8 Hz, 2H), 5.43-5.31 (m, 1H), 4.90-4.77 (m, 4H), 3.81-3.71 (m, 4H), 3.17-3.08 (m, 4H).

### Example 73: Synthesis of Compound 93

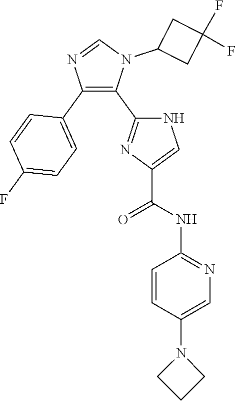 [SMILES: O=C(Nc1ccc(N2CCC2)cn1)c1c[nH]c(-c2c(-c3ccc(F)cc3)ncn2C2CC(F)(F)C2)n1]

N-(5-(azetidin-1-yl)pyridin-2-yl)-3′-(3,3-difluorocyclobutyl)-5′-(4-fluorophenyl)-1H,3′H-[2,4′-biimidazole]-4-carboxamide (Compound 93) was synthesized under the same synthetic route as for Compound 14 as white solid, which was determined by ¹HNMR, LCMS and HPLC. LCMS: Retention time: 0.720 min, (M+H)=494.1. HPLC: Retention time: 5.83 min. HNMR: (400 MHz, DMSO-d₆), δ=13.04 (br. s., 1H), 9.39 (s, 1H), 8.21 (s, 1H), 8.10-7.98 (m, 2H), 7.57 (d, J=2.4 Hz, 1H), 7.46-7.35 (m, 2H), 7.24-7.10 (m, 2H), 7.00-6.90 (m, 1H), 4.66 (d, J=8.0 Hz, 1H), 3.89-3.76 (m, 4H), 3.16-2.76 (m, 4H), 2.41-2.25 (m, 2H).

### Example 74: Synthesis of Compound 74

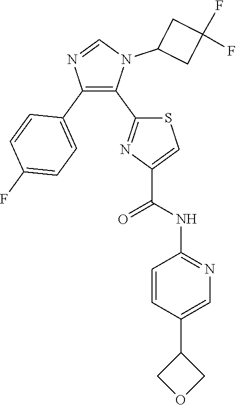 [SMILES: O=C(Nc1ccc(C2COC2)cn1)c1csc(-c2c(-c3ccc(F)cc3)ncn2C2CC(F)(F)C2)n1]

2-(1-(3,3-difluorocyclobutyl)-4-(4-fluorophenyl)-1H-imidazol-5-yl)-N-(5-(oxetan-3-yl)pyridin-2-yl)thiazole-4-carboxamide (Compound 74) was synthesized under the same synthetic route as for Compound 2 as white solid, which was determined by ¹HNMR, LCMS and HPLC. LCMS: Retention time: 1.009 min, (M+H)=512.3. HPLC: Retention time: 3.873 min. HNMR: (400 MHz, DMSO-d₆), δ=10.07 (s, 1H), 8.64 (s, 1H), 8.37 (s, 1H), 8.33 (s, 1H), 8.25 (d, J=8.4 Hz, 1H), 8.04 (d, J=8.4 Hz, 1H), 7.54-7.45 (m, 2H), 7.27-7.17 (m, 2H), 5.09 (s, 1H), 4.98-4.91 (m, 2H), 4.64 (t, J=6.4 Hz, 2H), 4.35-4.24 (m, 1H), 3.22-3.07 (m, 4H).

### Example 75: Synthesis of Compound 75

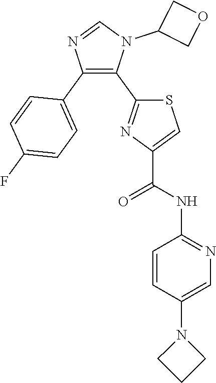 [SMILES: O=C(Nc1ccc(N2CCC2)cn1)c1csc(-c2c(-c3ccc(F)cc3)ncn2C2COC2)n1]

N-(5-(azetidin-1-yl)pyridin-2-yl)-2-(4-(4-fluorophenyl)-1-(oxetan-3-yl)-1H-imidazol-5-yl)thiazole-4-carboxamide (Compound 75) was synthesized under the same synthetic route as for Compound 2 as white solid, which was determined by ¹HNMR, LCMS and HPLC. LCMS: Retention time: 0.753 min, (M+H)=477.0. HPLC: Retention time: 1.832 min. HNMR: (400 MHz, DMSO-d₆), δ=9.95 (s, 1H), 8.64 (s, 1H), 8.51 (s, 1H), 8.00 (d, J=8.8 Hz, 1H), 7.64 (d, J=2.8 Hz, 1H), 7.56-7.50 (m, 2H), 7.32-7.23 (m, 2H), 7.05-7.00 (m, 1H), 5.87-5.79 (m, 1H), 4.98-4.94 (m, 4H), 3.89-3.85 (m, 4H), 2.38-2.31 (m, 2H).

### Example 76: Synthesis of Compound 76

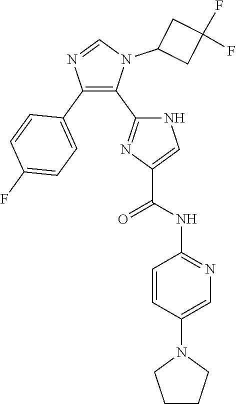 [SMILES: O=C(Nc1ccc(N2CCCC2)cn1)c1c[nH]c(-c2c(-c3ccc(F)cc3)ncn2C2CC(F)(F)C2)n1]

3′-(3,3-difluorocyclobutyl)-5′-(4-fluorophenyl)-N-(5-(pyrrolidin-1-yl)pyridin-2-yl)-1H,3′H-[2,4′-biimidazole]-4-carboxamide (Compound 76) was synthesized under the same synthetic route as for Compound 14 as off-white solid, which was determined by ¹HNMR, LCMS and HPLC. LCMS: Retention time: 0.735 min, (M+H)=508.2. HPLC: Retention time: 6.31 min. HNMR: (400 MHz, DMSO-d₆), δ=13.03 (br. s., 1H), 9.33 (s, 1H), 8.22 (s, 1H), 8.06-8.01 (m, 2H), 7.71-7.66 (m, 1H), 7.45-7.38 (m, 2H), 7.20-7.12 (m, 2H), 7.08-7.03 (m, 1H), 4.73-4.62 (m, 1H), 3.25-2.88 (m, 8H), 2.00-1.90 (m, 4H).

### Example 77: Synthesis of Compound 77

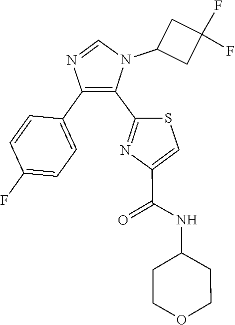 [SMILES: O=C(NC1CCOCC1)c1csc(-c2c(-c3ccc(F)cc3)ncn2C2CC(F)(F)C2)n1]

2-(1-(3,3-difluorocyclobutyl)-4-(4-fluorophenyl)-1H-imidazol-5-yl)-N-(tetrahydro-2H-pyran-4-yl)thiazole-4-carboxamide (Compound 77) was synthesized under the same synthetic route as for Compound 2 as white solid, which was determined by ¹HNMR, LCMS and HPLC. LCMS: Retention time: 1.767 min, (M+H)=463.1. HPLC: Retention time: 3.291 min. HNMR: (400 MHz, DMSO-d₆), δ=8.39 (s, 1H), 8.29 (s, 1H), 8.24 (d, J=8.0 Hz, 1H), 7.50-7.43 (m, 2H), 7.26-7.17 (m, 2H), 4.98 (d, J=2.4 Hz, 1H), 4.10-3.96 (m, 1H), 3.87 (d, J=11.2 Hz, 2H), 3.43-3.38 (m, 2H), 3.27-3.13 (m, 2H), 3.11-2.98 (m, 2H), 1.79-1.59 (m, 4H).

### Example 78: Synthesis of Compound 78

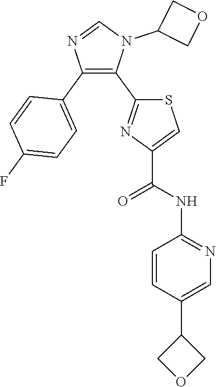 [SMILES: O=C(Nc1ccc(C2COC2)cn1)c1csc(-c2c(-c3ccc(F)cc3)ncn2C2COC2)n1]

2-(4-(4-fluorophenyl)-1-(oxetan-3-yl)-1H-imidazol-5-yl)-N-(5-(oxetan-3-yl)pyridin-2-yl)thiazole-4-carboxamide (Compound 78) was synthesized under the same synthetic route as for Compound 2 as white solid, which was determined by ¹HNMR, LCMS and HPLC. LCMS: Retention time: 1.710 min, (M+H)=478.1. HPLC: Retention time: 3.234 min. HNMR: (400 MHz, DMSO-d₆), δ=10.07 (s, 1H), 8.57 (s, 1H), 8.47 (s, 1H), 8.39 (d, J=2.0 Hz, 1H), 8.24 (d, J=8.8 Hz, 1H), 8.04 (dd, J=2.0, 8.8 Hz, 1H), 7.57-7.49 (m, 2H), 7.29-7.22 (m, 2H), 5.85-5.76 (m, 1H), 5.04-4.92 (m, 6H), 4.70-4.62 (m, J=6.4, 6.4 Hz, 2H), 4.36-4.25 (m, 1H).

### Example 79: Synthesis of Compound 79

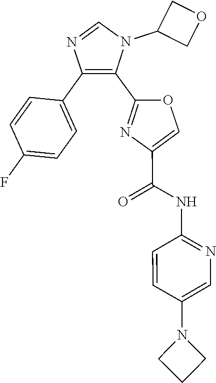 [SMILES: O=C(Nc1ccc(N2CCC2)cn1)c1coc(-c2c(-c3ccc(F)cc3)ncn2C2COC2)n1]

N-(5-(azetidin-1-yl)pyridin-2-yl)-2-(4-(4-fluorophenyl)-1-(oxetan-3-yl)-1H-imidazol-5-yl)oxazole-4-carboxamide (Compound 79) was synthesized under the same synthetic route as for Compound 2 as white solid, which was determined by ¹HNMR, LCMS and HPLC. LCMS: Retention time: 0.721 min, (M+H)=461.0. HPLC: Retention time: 2.882 min. HNMR: (400 MHz, DMSO-d₆), δ=9.85 (s, 1H), 8.80 (s, 1H), 8.52 (s, 1H), 7.95 (d, J=8.8 Hz, 1H), 7.69-7.59 (m, 3H), 7.28-7.20 (m, 2H), 6.96 (dd, J=2.8, 8.8 Hz, 1H), 6.02-5.91 (m, 1H), 5.07-4.99 (m, 2H), 4.98-4.90 (m, 2H), 3.86 (t, J=7.2 Hz, 4H), 2.38-2.32 (m, 2H).

### Example 80: Synthesis of Compound 80

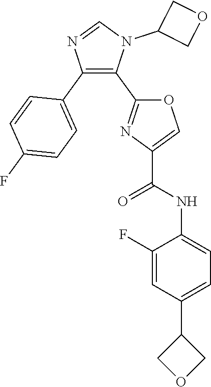 [SMILES: O=C(Nc1ccc(C2COC2)cc1F)c1coc(-c2c(-c3ccc(F)cc3)ncn2C2COC2)n1]

N-(2-fluoro-4-(oxetan-3-yl)phenyl)-2-(4-(4-fluorophenyl)-1-(oxetan-3-yl)-1H-imidazol-5-yl)oxazole-4-carboxamide (Compound 80) was synthesized under the same synthetic route as for Compound 2 as white solid, which was determined by ¹HNMR, LCMS and HPLC. LCMS: Retention time: 0.796 min, (M+H)=479.0. HPLC: Retention time: 2.042 min. HNMR: (400 MHz, DMSO-d₆), δ=9.78 (s, 1H), 8.81 (s, 1H), 8.57 (s, 1H), 7.80-7.72 (m, 1H), 7.68-7.59 (m, 2H), 7.45-7.37 (m, 1H), 7.29-7.22 (m, 3H), 5.98-5.86 (m, 1H), 5.07-5.00 (m, 2H), 4.98-4.92 (m, 4H), 4.67-4.61 (m, 2H), 4.34-4.25 (m, 1H).

### Example 81: Synthesis of Compound 81

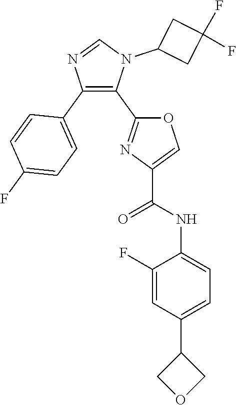 [SMILES: O=C(Nc1ccc(C2COC2)cc1F)c1coc(-c2c(-c3ccc(F)cc3)ncn2C2CC(F)(F)C2)n1]

2-(1-(3,3-difluorocyclobutyl)-4-(4-fluorophenyl)-1H-imidazol-5-yl)-N-(2-fluoro-4-(oxetan-3-yl)phenyl)oxazole-4-carboxamide (Compound 81) was synthesized under the same synthetic route as for Compound 2 as white powder, which was determined by ¹HNMR, LCMS and HPLC. LCMS: Retention time: 3.573 min, (M+H)=513.0. HPLC: Retention time: 4.46 min. HNMR: (400 MHz, CDCl₃), δ=8.94 (s, 1H), 8.51-8.43 (m, 1H), 8.29 (s, 1H), 7.99 (s, 1H), 7.68-7.60 (m, 2H), 7.30-7.27 (m, 1H), 7.24-7.19 (m, 1H), 7.17-7.09 (m, 2H), 5.33-5.15 (m, 1H), 5.14-5.06 (m, 2H), 4.82-4.70 (m, 2H), 4.27-4.17 (m, 1H), 3.42-3.27 (m, 2H), 3.20-3.01 (m, 2H).

### Example 82: Synthesis of Compound 82

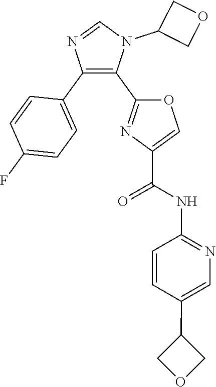 [SMILES: O=C(Nc1ccc(C2COC2)cn1)c1coc(-c2c(-c3ccc(F)cc3)ncn2C2COC2)n1]

2-(4-(4-fluorophenyl)-1-(oxetan-3-yl)-1H-imidazol-5-yl)-N-(5-(oxetan-3-yl)pyridin-2-yl)oxazole-4-carboxamide (Compound 82) was synthesized under the same synthetic route as for Compound 2 as white solid, which was determined by ¹HNMR, LCMS and HPLC. LCMS: Retention time: 1.653 min, (M+H)=462.1. HPLC: Retention time: 3.113 min. HNMR: (400 MHz, CDCl₃), δ=9.37-9.18 (m, 1H), 8.41 (d, J=8.0 Hz, 1H), 8.33 (d, J=2.0 Hz, 1H), 8.27 (s, 1H), 8.15 (s, 1H), 7.98 (d, J=8.0 Hz, 1H), 7.66-7.55 (m, 2H), 7.18-7.07 (m, 2H), 6.07-5.96 (m, 1H), 5.32-5.23 (m, 2H), 5.18-5.10 (m, 2H), 5.02-4.96 (m, 2H), 4.83-4.73 (m, 2H), 4.33-4.20 (m, 1H).

### Example 83: Synthesis of Compound 83

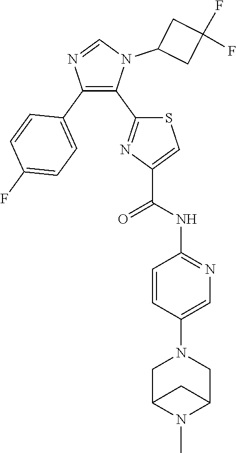 [SMILES: CN1C2CC1CN(c1ccc(NC(=O)c3csc(-c4c(-c5ccc(F)cc5)ncn4C4CC(F)(F)C4)n3)nc1)C2]

2-(1-(3,3-difluorocyclobutyl)-4-(4-fluorophenyl)-1H-imidazol-5-yl)-N-(5-(6-methyl-3,6-diazabicyclo[3.1.1]heptan-3-yl)pyridin-2-yl)thiazole-4-carboxamide (Compound 83) was synthesized under the same synthetic route as for Compound 2 as white solid, which was determined by ¹HNMR, LCMS and HPLC. LCMS: Retention time: 1.948 min, (M+H)=566.2. HPLC: Retention time: 4.292 min. HNMR: (400 MHz, DMSO-d₆), δ=9.84 (s, 1H), 8.57 (s, 1H), 8.34 (s, 1H), 8.14-8.08 (m, 1H), 7.96 (d, J=2.4 Hz, 1H), 7.53-7.46 (m, 2H), 7.37-7.28 (m, 1H), 7.27-7.19 (m, 2H), 5.14-5.04 (m, 1H), 4.42-3.95 (m, 2H), 3.80-3.54 (m, 2H), 3.24-3.21 (m, 2H), 3.17-3.07 (m, 4H), 2.47-2.39 (m, 3H), 2.30-2.23 (m, 1H), 1.94-1.74 (m, 1H).

### Example 84: Synthesis of Compound 84

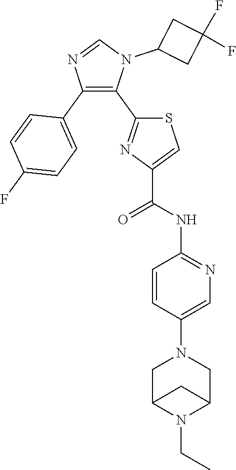 [SMILES: CCN1C2CC1CN(c1ccc(NC(=O)c3csc(-c4c(-c5ccc(F)cc5)ncn4C4CC(F)(F)C4)n3)nc1)C2]

2-(1-(3,3-difluorocyclobutyl)-4-(4-fluorophenyl)-1H-imidazol-5-yl)-N-(5-(6-ethyl-3,6-diazabicyclo[3.1.1]heptan-3-yl)pyridin-2-yl)thiazole-4-carboxamide (Compound 84) was synthesized under the same synthetic route as for Compound 2 as white solid, which was determined by ¹HNMR, LCMS and HPLC. LCMS: Retention time: 1.978 min, (M+H)=580.2. HPLC: Retention time: 4.436 min. HNMR: (400 MHz, DMSO-d₆), δ=9.80 (s, 1H), 8.56 (s, 1H), 8.33 (s, 1H), 8.06 (d, J=9.2 Hz, 1H), 7.90 (d, J=2.8 Hz, 1H), 7.53-7.47 (m, 2H), 7.27-7.19 (m, 3H), 5.13-5.04 (m, 1H), 3.67 (d, J=5.6 Hz, 2H), 3.48-3.41 (m, 2H), 3.31-3.27 (m, 2H), 3.26-3.09 (m, 4H), 2.46-2.38 (m, 1H), 2.34-2.25 (m, 2H), 1.55 (d, J=8.4 Hz, 1H), 0.95-0.87 (m, 3H).

### Example 85: Synthesis of Compound 85

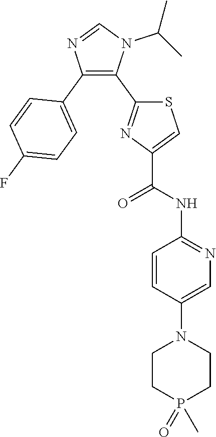 [SMILES: CC(C)n1cnc(-c2ccc(F)cc2)c1-c1nc(C(=O)Nc2ccc(N3CCP(C)(=O)CC3)cn2)cs1]

2-(4-(4-fluorophenyl)-1-isopropyl-1H-imidazol-5-yl)-N-(5-(4-methyl-4-oxido-1,4-azaphosphinan-1-yl)pyridin-2-yl)thiazole-4-carboxamide (Compound 85) was synthesized under the same synthetic route as for Compound 2 as white solid, which was determined by ¹HNMR, LCMS and HPLC. LCMS: Retention time: 1.613 min, (M+H)=539.1. HPLC: Retention time: 3.100 min. HNMR: (400 MHz, DMSO-d₆), δ=9.78 (s, 1H), 8.61 (s, 1H), 8.19 (s, 1H), 8.13 (d, J=2.8 Hz, 1H), 8.06 (d, J=9.2 Hz, 1H), 7.53 (dd, J=3.2, 9.2 Hz, 1H), 7.50-7.43 (m, 2H), 7.22-7.15 (m, 2H), 4.77-4.69 (m, 1H), 3.97-3.81 (m, 2H), 3.57-3.44 (m, 2H), 1.94-1.83 (m, 2H), 1.81-1.68 (m, 2H), 1.56-1.46 (m, 9H).

### Example 86: Synthesis of Compound 86

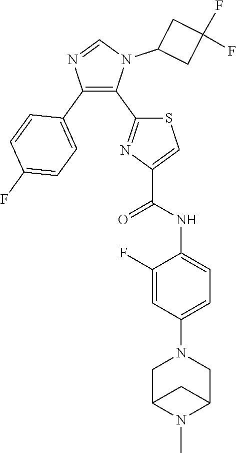 [SMILES: CN1C2CC1CN(c1ccc(NC(=O)c3csc(-c4c(-c5ccc(F)cc5)ncn4C4CC(F)(F)C4)n3)c(F)c1)C2]

2-(1-(3,3-difluorocyclobutyl)-4-(4-fluorophenyl)-1H-imidazol-5-yl)-N-(2-fluoro-4-(6-methyl-3,6-diazabicyclo[3.1.1]heptan-3-yl)phenyl)thiazole-4-carboxamide (Compound 86) was synthesized under the same synthetic route as for Compound 2 as white solid, which was determined by ¹HNMR, LCMS and HPLC. LCMS: Retention time: 0.738 min, (M+H)=583.0. HPLC: Retention time: 3.26 min. HNMR: (400 MHz, DMSO-d₆), δ=9.70 (s, 1H), 8.48 (s, 1H), 8.33 (s, 1H), 7.61-7.53 (m, 1H), 7.53-7.44 (m, 2H), 7.28-7.20 (m, 2H), 6.68-6.55 (m, 2H), 5.22-5.03 (m, 1H), 3.57 (d, J=5.6 Hz, 2H), 3.45-3.42 (m, 2H), 3.28-3.11 (m, 6H), 2.47-2.39 (m, 1H), 2.00 (s, 3H), 1.51 (d, J=8.4 Hz, 1H).

### Example 87: Synthesis of Compound 87

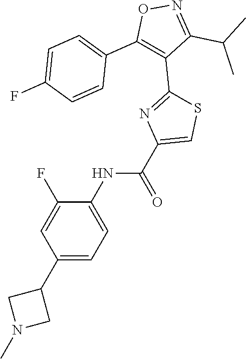 [SMILES: CC(C)c1noc(-c2ccc(F)cc2)c1-c1nc(C(=O)Nc2ccc(C3CN(C)C3)cc2F)cs1]

N-(2-fluoro-4-(1-methylazetidin-3-yl)phenyl)-2-(5-(4-fluorophenyl)-3-isopropylisoxazol-4-yl)thiazole-4-carboxamide (Compound 87) was synthesized under the same synthetic route as for Compound 30 as white solid, which was determined by ¹HNMR, LCMS and HPLC. LCMS: Retention time: 0.904 min, (M+H)=495.1. HPLC: Retention time: 2.184 min. HNMR: (400 MHz, DMSO-d₆), δ=9.85 (s, 1H), 8.61 (s, 1H), 8.04-7.86 (m, 1H), 7.83-7.71 (m, 2H), 7.59-7.52 (m, 1H), 7.44-7.36 (m, 2H), 7.33-7.26 (m, 1H), 4.47-4.16 (m, 3H), 4.15-3.98 (m, 2H), 3.45 (s, 1H), 2.96-2.82 (m, 3H), 1.31 (d, J=6.8 Hz, 6H).

### Example 88: Synthesis of Compound 88

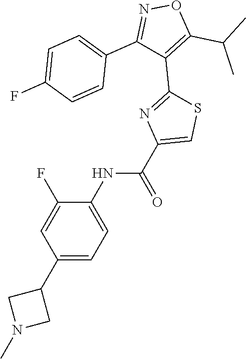 [SMILES: CC(C)c1onc(-c2ccc(F)cc2)c1-c1nc(C(=O)Nc2ccc(C3CN(C)C3)cc2F)cs1]

N-(2-fluoro-4-(1-methylazetidin-3-yl)phenyl)-2-(3-(4-fluorophenyl)-5-isopropylisoxazol-4-yl)thiazole-4-carboxamide (Compound 88) was synthesized under the same synthetic route as for Compound 30 as white solid, which was determined by ¹HNMR, LCMS and HPLC. LCMS: Retention time: 0.828 min, (M+H)=495.0. HPLC: Retention time: 4.58 min. HNMR: (400 MHz, DMSO-d₆), δ=9.64 (s, 1H), 8.51 (s, 1H), 8.02-7.96 (m, 1H), 7.68-7.60 (m, 2H), 7.45-7.33 (m, 3H), 7.23 (dd, J=1.6, 8.4 Hz, 1H), 3.97-3.91 (m, 2H), 3.85-3.73 (m, 2H), 3.61-3.53 (m, 2H), 2.55 (s, 3H), 1.40 (d, J=6.8 Hz, 6H).

### Example 89: Synthesis of Compound 89

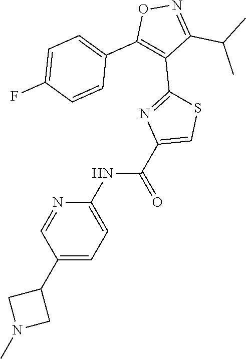 [SMILES: CC(C)c1noc(-c2ccc(F)cc2)c1-c1nc(C(=O)Nc2ccc(C3CN(C)C3)cn2)cs1]

2-(5-(4-fluorophenyl)-3-isopropylisoxazol-4-yl)-N-(5-(1-methylazetidin-3-yl)pyridin-2-yl)thiazole-4-carboxamide (Compound 89) was synthesized under the same synthetic route as for Compound 30 as white solid, which was determined by ¹HNMR, LCMS and HPLC. LCMS: Retention time: 0.752 min, (M+H)=478.2. HPLC: Retention time: 2.078 min. HNMR: (400 MHz, DMSO-d₆), δ=9.89 (s, 1H), 8.66 (s, 1H), 8.39 (s, 1H), 8.24-8.18 (m, 1H), 8.00 (d, J=8.0 Hz, 1H), 7.79-7.71 (m, 2H), 7.44-7.35 (m, 2H), 4.19-4.01 (m, 2H), 3.93-3.87 (m, 1H), 3.84-3.77 (m, 2H), 3.38-3.36 (m, 1H), 2.67 (s, 3H), 1.32 (d, J=6.8 Hz, 6H).

### Example 90: Synthesis of Compound 90

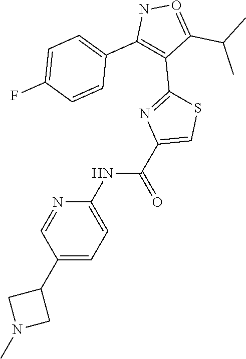

2-(3-(4-fluorophenyl)-5-isopropylisoxazol-4-yl)-N-(5-(1-methylazetidin-3-yl)pyridin-2-yl)thiazole-4-carboxamide (Compound 90) was synthesized under the same synthetic route as for Compound 30 as white solid, which was determined by ¹HNMR, LCMS and HPLC. LCMS: Retention time: 3.149 min, (M+H)=478.2. HPLC: Retention time: 4.23 min. HNMR: (400 MHz, CDCl₃), δ=9.57 (s, 1H), 8.38 (d, J=8.8 Hz, 1H), 8.26 (d, J=2.4 Hz, 1H), 8.23 (s, 1H), 7.78 (dd, J=2.4, 8.8 Hz, 1H), 7.59-7.51 (m, 2H), 7.21-7.12 (m, 2H), 4.34-4.22 (m, 2H), 4.16-4.03 (m, 1H), 3.78-3.59 (m, 3H), 2.75 (s, 3H), 1.49 (d, J=7.2 Hz, 6H).

### Example 91: Synthesis of Compound 91

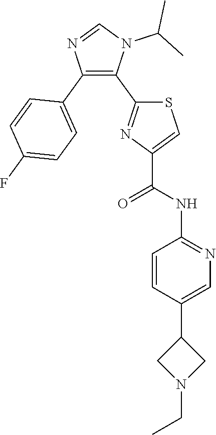 [SMILES: CCN1CC(c2ccc(NC(=O)c3csc(-c4c(-c5ccc(F)cc5)ncn4C(C)C)n3)nc2)C1]

N-(5-(1-ethylazetidin-3-yl)pyridin-2-yl)-2-(4-(4-fluorophenyl)-1-isopropyl-1H-imidazol-5-yl)thiazole-4-carboxamide (Compound 91) was synthesized under the same synthetic route as for Compound 30 as white solid, which was determined by ¹HNMR, LCMS and HPLC. LCMS: Retention time: 1.800 min, (M+H)=491.2. HPLC: Retention time: 2.63 min. HNMR: (400 MHz, CDCl₃), δ=9.67 (s, 1H), 8.39 (d, J=8.4 Hz, 1H), 8.28 (s, 1H), 8.26 (d, J=2.4 Hz, 1H), 7.82 (s, 1H), 7.80 (dd, J=2.0, 8.8 Hz, 1H), 7.49-7.38 (m, 2H), 7.09-6.94 (m, 2H), 4.98-4.79 (m, 1H), 4.15-4.00 (m, 2H), 4.00-3.86 (m, 1H), 3.54-3.32 (m, 2H), 2.79 (q, J=7.2 Hz, 2H), 1.59 (d, J=6.8 Hz, 6H), 1.16 (t, J=7.2 Hz, 3H).

### Example 92: Synthesis of Compound 92

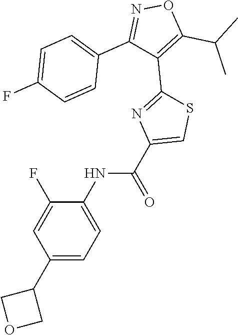 [SMILES: CC(C)c1onc(-c2ccc(F)cc2)c1-c1nc(C(=O)Nc2ccc(C3COC3)cc2F)cs1]

N-(2-fluoro-4-(oxetan-3-yl)phenyl)-2-(3-(4-fluorophenyl)-5-isopropylisoxazol-4-yl)thiazole-4-carboxamide (Compound 92) was synthesized under the same synthetic route as for Compound 31 as white solid, which was determined by ¹HNMR, LCMS and HPLC. LCMS: Retention time: 1.299 min, (M+H)=482.2. HPLC: Retention time: 2.887 min. HNMR: (400 MHz, DMSO-d₆), δ=9.65 (s, 1H), 8.51 (s, 1H), 8.07-7.99 (m, 1H), 7.69-7.61 (m, 2H), 7.44-7.33 (m, 3H), 7.28 (d, J=8.4 Hz, 1H), 4.96-4.89 (m, 2H), 4.65-4.58 (m, 2H), 4.32-4.22 (m, 1H), 3.85-3.74 (m, 1H), 1.40 (d, J=7.2 Hz, 6H).

### Example 93: Synthesis of Compound 93

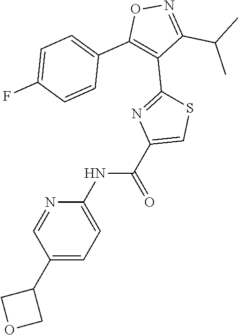 [SMILES: CC(C)c1noc(-c2ccc(F)cc2)c1-c1nc(C(=O)Nc2ccc(C3COC3)cn2)cs1]

2-(5-(4-fluorophenyl)-3-isopropylisoxazol-4-yl)-N-(5-(oxetan-3-yl)pyridin-2-yl)thiazole-4-carboxamide (Compound 93) was synthesized under the same synthetic route as for Compound 31 as white solid, which was determined by ¹HNMR, LCMS and HPLC. LCMS: Retention time: 1.223 min, (M+H)=465.2. HPLC: Retention time: 4.560 min. HNMR: (400 MHz, DMSO-d₆), δ=9.89 (s, 1H), 8.67 (s, 1H), 8.36 (s, 1H), 8.23 (d, J=8.8 Hz, 1H), 8.04 (d, J=8.4 Hz, 1H), 7.79-7.70 (m, 2H), 7.44-7.34 (m, 2H), 5.02-4.87 (m, 2H), 4.67-4.59 (m, 2H), 4.35-4.23 (m, 1H), 3.44-3.41 (m, 1H), 1.32 (d, J=6.8 Hz, 6H).

### Example 94: Synthesis of Compound 94

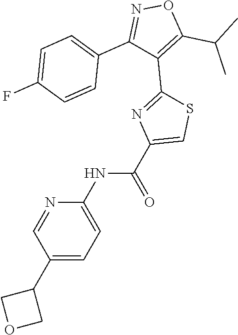 [SMILES: CC(C)c1onc(-c2ccc(F)cc2)c1-c1nc(C(=O)Nc2ccc(C3COC3)cn2)cs1]

2-(3-(4-fluorophenyl)-5-isopropylisoxazol-4-yl)-N-(5-(oxetan-3-yl)pyridin-2-yl)thiazole-4-carboxamide (Compound 94) was synthesized under the same synthetic route as for Compound 31 as white solid, which was determined by ¹HNMR, LCMS and HPLC. LCMS: Retention time: 1.222 min, (M+H)=465.2. HPLC: Retention time: 5.42 min. HNMR: (400 MHz, DMSO-d₆), δ=9.74 (s, 1H), 8.58 (s, 1H), 8.36 (d, J=2.4 Hz, 1H), 8.22 (d, J=8.8 Hz, 1H), 8.03 (dd, J=2.4, 8.8 Hz, 1H), 7.67-7.60 (m, 2H), 7.42-7.33 (m, 2H), 5.00-4.89 (m, 2H), 4.70-4.57 (m, 2H), 4.40-4.23 (m, 1H), 3.82-3.69 (m, 1H), 1.41 (d, J=6.8 Hz, 6H).

### Example 95: Synthesis of Compound 95

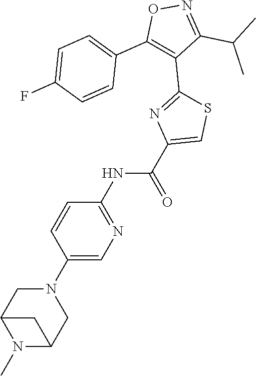 [SMILES: CC(C)c1noc(-c2ccc(F)cc2)c1-c1nc(C(=O)Nc2ccc(N3CC4CC(C3)N4C)cn2)cs1]

2-(5-(4-fluorophenyl)-3-isopropylisoxazol-4-yl)-N-(5-(6-methyl-3,6-diazabicyclo[3.1.1]heptan-3-yl)pyridin-2-yl)thiazole-4-carboxamide (Compound 95) was synthesized under the same synthetic route as for Compound 31 as yellow solid, which was determined by ¹HNMR, LCMS and HPLC. LCMS: Retention time: 1.018 min, (M+H)=519.3. HPLC: Retention time: 3.313 min. HNMR: (400 MHz, DMSO-d₆), δ=9.79 (d, J=12.4 Hz, 1H), 8.62 (d, J=2.4 Hz, 1H), 8.16-8.09 (m, 1H), 7.97 (d, J=2.8 Hz, 1H), 7.78-7.71 (m, 2H), 7.49-7.30 (m, 3H), 4.54-4.47 (m, 1H), 4.36 (d, J=6.4 Hz, 1H), 3.95-3.82 (m, 2H), 3.81-3.67 (m, 2H), 3.35-3.33 (m, 1H), 3.04 (d, J=5.2 Hz, 1.5H), 2.91-2.76 (m, 1H), 2.52-2.52 (m, 1.5H), 2.04-1.94 (m, 1H), 1.33 (d, J=6.8 Hz, 6H).

### Example 96: Synthesis of Compound 96

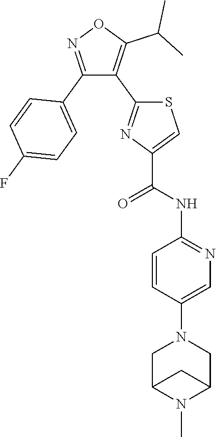 [SMILES: CC(C)c1onc(-c2ccc(F)cc2)c1-c1nc(C(=O)Nc2ccc(N3CC4CC(C3)N4C)cn2)cs1]

2-(3-(4-fluorophenyl)-5-isopropylisoxazol-4-yl)-N-(5-(6-methyl-3,6 diazabicyclo[3.1.1]heptan-3-yl)pyridin-2-yl)thiazole-4-carboxamide (Compound 96) was synthesized under the same synthetic route as for Compound 31 as white solid, which was determined by ¹HNMR, LCMS and HPLC. LCMS: Retention time: 1.016 min, (M+H)=519.2. HPLC: Retention time: 4.03 min. HNMR: (400 MHz, DMSO-d₆)_{6=9.58} (s, 1H), 8.52 (d, J=2.0 Hz, 1H), 8.11 (d, J=9.2 Hz, 1H), 7.99-7.95 (m, 1H), 7.67-7.60 (m, 2H), 7.42-7.32 (m, 3H), 4.54-4.47 (m, 1H), 4.40-4.31 (m, 1H), 3.94-3.83 (m, 2H), 3.79-3.68 (m, 3H), 3.04 (d, J=5.2 Hz, 1.5H), 2.91-2.79 (m, 1H), 2.52-2.51 (m, 1.5H), 2.08-1.93 (m, 1H), 1.41 (d, J=6.8 Hz, 6H).

### Example 97: Synthesis of Compound 97

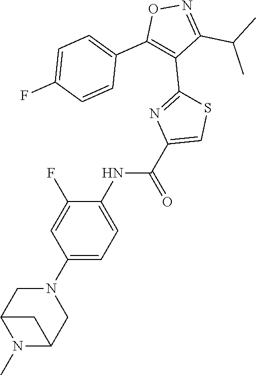 [SMILES: CC(C)c1noc(-c2ccc(F)cc2)c1-c1nc(C(=O)Nc2ccc(N3CC4CC(C3)N4C)cc2F)cs1]

N-(2-fluoro-4-(6-methyl-3,6-diazabicyclo[3.1.1]heptan-3-yl)phenyl)-2-(5-(4-fluorophenyl)-3-isopropylisoxazol-4-yl)thiazole-4-carboxamide (Compound 97) was synthesized under the same synthetic route as for Compound 31 as white solid, which was determined by ¹HNMR, LCMS and HPLC. LCMS: Retention time: 1.068 min, (M+H)=536.3. HPLC: Retention time: 4.64 min. H NMR: (400 MHz, DMSO-d₆), δ=9.62 (s, 1H), 8.54 (s, 1H), 7.79-7.71 (m, 2H), 7.64 (t, J=9.2 Hz, 1H), 7.40 (t, J=8.8 Hz, 2H), 6.67-6.53 (m, 2H), 3.58 (d, J=5.6 Hz, 2H), 3.49-3.41 (m, 3H), 3.27 (d, J=11.2 Hz, 2H), 2.44 (d, J=5.6 Hz, 1H), 2.00 (s, 3H), 1.51 (d, J=8.4 Hz, 1H), 1.30 (d, J=6.8 Hz, 6H).

### Example 98: Synthesis of Compound 98

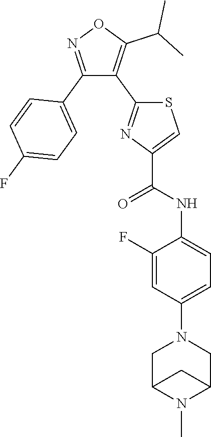 [SMILES: CC(C)c1onc(-c2ccc(F)cc2)c1-c1nc(C(=O)Nc2ccc(N3CC4CC(C3)N4C)cc2F)cs1]

N-(2-fluoro-4-(6-methyl-3,6-diazabicyclo[3.1.1]heptan-3-yl)phenyl)-2-(3-(4-fluorophenyl)-5-isopropylisoxazol-4-yl)thiazole-4-carboxamide (Compound 98) was synthesized under the same synthetic route as for Compound 31 as white solid, which was determined by ¹HNMR, LCMS and HPLC. LCMS: Retention time: 0.875 min, (M+Na)=558.2. HPLC: Retention time: 4.62 min. HNMR: (400 MHz, DMSO-d₆), δ=9.46 (s, 1H), 8.43 (s, 1H), 7.73-7.61 (m, 3H), 7.40-7.32 (m, 2H), 6.69-6.52 (m, 2H), 3.87-3.69 (m, 1H), 3.61-3.54 (m, 2H), 3.44-3.41 (m, 2H), 3.26 (d, J=12.0 Hz, 2H), 2.47-2.37 (m, 1H), 1.99 (s, 3H), 1.53-1.47 (m, 1H), 1.39 (d, J=6.8 Hz, 6H).

### Example 99: Synthesis of Compound 99

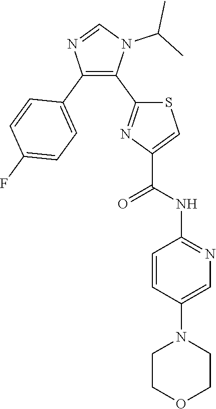 [SMILES: CC(C)n1cnc(-c2ccc(F)cc2)c1-c1nc(C(=O)Nc2ccc(N3CCOCC3)cn2)cs1]

2-(4-(4-fluorophenyl)-1-isopropyl-1H-imidazol-5-yl)-N-(5-morpholinopyridin-2-yl)thiazole-4-carboxamide (Compound 99) was synthesized under the same synthetic route as for Compound 2 as off-white solid, which was determined by ¹HNMR, LCMS and HPLC. LCMS: Retention time: 3.835 min, (M+H)=493.1. HPLC: Retention time: 6.37 min. HNMR: (400 MHz, DMSO-d₆), δ=9.82 (s, 1H), 8.61 (s, 1H), 8.19 (s, 1H), 8.10-8.06 (m, 2H), 7.53-7.42 (m, 3H), 7.22-7.15 (m, 2H), 4.76-4.68 (m, 1H), 3.78-3.71 (m, 4H), 3.17-3.11 (m, 4H), 1.48 (d, J=6.4 Hz, 6H).

### Example 100: Synthesis of Compound 100

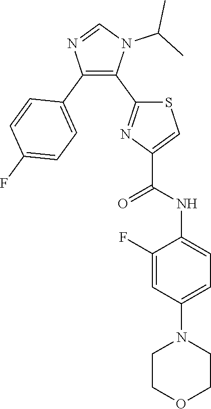 [SMILES: CC(C)n1cnc(-c2ccc(F)cc2)c1-c1nc(C(=O)Nc2ccc(N3CCOCC3)cc2F)cs1]

N-(2-fluoro-4-morpholinophenyl)-2-(4-(4-fluorophenyl)-1-isopropyl-1H-imidazol-5-yl) thiazole-4-carboxamide (Compound 100) was synthesized under the same synthetic route as for Compound 2 as white solid, which was determined by ¹HNMR, LCMS and HPLC. LCMS: Retention time: 2.081 min, (M+H)=510.1. HPLC: Retention time: 4.189 min. HNMR: (400 MHz, DMSO-d₆), δ=9.76 (s, 1H), 8.55 (s, 1H), 8.19 (s, 1H), 7.69-7.61 (m, 1H), 7.50-7.42 (m, 2H), 7.23-7.15 (m, 2H), 6.95-6.87 (m, 1H), 6.83-6.77 (m, 1H), 4.79-4.69 (m, 1H), 3.78-3.70 (m, 4H), 3.17-3.09 (m, 4H), 1.47 (d, J=6.8 Hz, 6H).

### Example 101: Synthesis of Compound 101

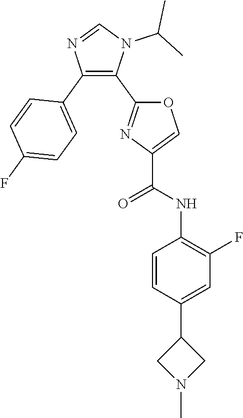 [SMILES: CC(C)n1cnc(-c2ccc(F)cc2)c1-c1nc(C(=O)Nc2ccc(C3CN(C)C3)cc2F)co1]

N-(2-fluoro-4-(1-methylazetidin-3-yl)phenyl)-2-(4-(4-fluorophenyl)-1-isopropyl-1H-imidazol-5-yl)oxazole-4-carboxamide (Compound 101) was synthesized under the same synthetic route as for Compound 30 as white solid, which was determined by ¹HNMR, LCMS and HPLC. LCMS: Retention time: 0.624 min, (M+H)=478.2. HPLC: Retention time: 1.579 min. HNMR: (400 MHz, DMSO-d₆), δ=9.83 (s, 1H), 8.88 (s, 1H), 8.23 (s, 1H), 7.75-7.66 (m, 1H), 7.59-7.51 (m, 2H), 7.42-7.34 (m, 1H), 7.25-7.18 (m, 3H), 4.98-4.86 (m, 1H), 3.87-3.80 (m, 2H), 3.78-3.72 (m, 1H), 3.44-3.43 (m, 2H), 2.46 (s, 3H), 1.49 (d, J=6.8 Hz, 6H).

### Example 102: Synthesis of Compound 102

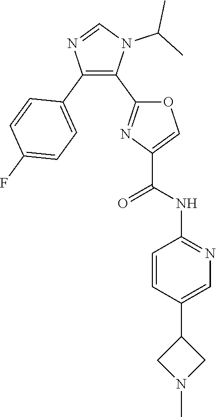 [SMILES: CC(C)n1cnc(-c2ccc(F)cc2)c1-c1nc(C(=O)Nc2ccc(C3CN(C)C3)cn2)co1]

2-(4-(4-fluorophenyl)-1-isopropyl-1H-imidazol-5-yl)-N-(5-(1-methylazetidin-3-yl)pyridin-2-yl)oxazole-4-carboxamide (Compound 102) was synthesized under the same synthetic route as for Compound 30 as white solid, which was determined by ¹HNMR, LCMS and HPLC. LCMS: Retention time: 0.603 min, (M+H)=461.2. HPLC: Retention time: 1.422 min. HNMR: (400 MHz, DMSO-d₆), δ=10.07 (s, 1H), 8.99-8.92 (m, 1H), 8.34 (d, J=2.4 Hz, 1H), 8.23 (s, 1H), 8.15 (d, J=8.4 Hz, 1H), 7.95 (dd, J=2.4, 8.8 Hz, 1H), 7.59-7.51 (m, 2H), 7.25-7.16 (m, 2H), 5.03-4.91 (m, 1H), 3.74-3.62 (m, 3H), 3.30-3.22 (m, 2H), 2.37 (s, 3H), 1.50 (d, J=6.8 Hz, 6H).

### Example 103: Synthesis of Compound 103

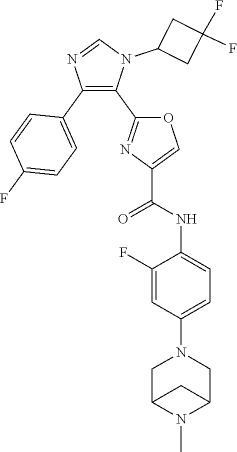 [SMILES: CN1C2CC1CN(c1ccc(NC(=O)c3coc(-c4c(-c5ccc(F)cc5)ncn4C4CC(F)(F)C4)n3)c(F)c1)C2]

2-(1-(3,3-difluorocyclobutyl)-4-(4-fluorophenyl)-1H-imidazol-5-yl)-N-(2-fluoro-4-(6-methyl-3,6-diazabicyclo[3.1.1]heptan-3-yl)phenyl)oxazole-4-carboxamide (Compound 103) was synthesized under the same synthetic route as for Compound 2 as white solid, which was determined by ¹HNMR, LCMS and HPLC. LCMS: Retention time: 2.009 min, (M+H)=567.2. HPLC: Retention time: 4.592 min. HNMR: (400 MHz, DMSO-d₆), δ=9.58 (s, 1H), 8.76 (s, 1H), 8.38 (s, 1H), 7.66-7.59 (m, 2H), 7.49-7.43 (m, 1H), 7.27-7.20 (m, 2H), 6.68-6.54 (m, 2H), 5.24-5.15 (m, 1H), 3.58 (d, J=5.6 Hz, 2H), 3.50-3.44 (m, 2H), 3.31-3.18 (m, 6H), 2.48-2.39 (m, 1H), 2.00 (s, 3H), 1.51 (d, J=8.4 Hz, 1H).

### Example 104: Synthesis of Compound 104

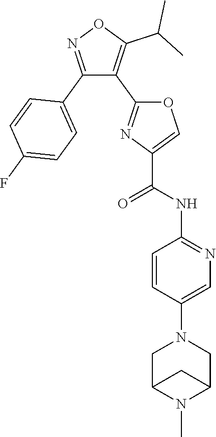 [SMILES: CC(C)c1onc(-c2ccc(F)cc2)c1-c1nc(C(=O)Nc2ccc(N3CC4CC(C3)N4C)cn2)co1]

2-(3-(4-fluorophenyl)-5-isopropylisoxazol-4-yl)-N-(5-(6-methyl-3,6-diazabicyclo[3.1.1]heptan-3-yl)pyridin-2-yl)oxazole-4-carboxamide (Compound 104) was synthesized under the same synthetic route as for Compound 31 as white solid, which was determined by ¹HNMR, LCMS and HPLC. LCMS: Retention time: 2.286 min, (M+H)=503.1. HPLC: Retention time: 1.261 min. HNMR: (400 MHz, DMSO-d₆), δ=9.55 (s, 1H), 8.83 (s, 1H), 8.00 (d, J=9.2 Hz, 1H), 7.89 (d, J=2.8 Hz, 1H), 7.73-7.65 (m, 2H), 7.39-7.32 (m, 2H), 7.23 (dd, J=2.8, 9.2 Hz, 1H), 3.90-3.82 (m, 1H), 3.58 (d, J=5.6 Hz, 2H), 3.47-3.44 (m, 2H), 3.32-3.28 (m, 2H), 2.47-2.41 (m, 1H), 2.00 (s, 3H), 1.54 (d, J=8.4 Hz, 1H), 1.39 (d, J=8.4 Hz, 6H).

### Example 105: Synthesis of Compound 105

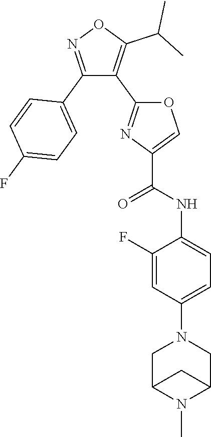 [SMILES: CC(C)c1onc(-c2ccc(F)cc2)c1-c1nc(C(=O)Nc2ccc(N3CC4CC(C3)N4C)cc2F)co1]

N-(2-fluoro-4-(6-methyl-3,6-diazabicyclo[3.1.1]heptan-3-yl)phenyl)-2-(3-(4-fluorophenyl)-5-isopropylisoxazol-4-yl)oxazole-4-carboxamide (Compound 105) was synthesized under the same synthetic route as for Compound 31 as white solid, which was determined by ¹HNMR, LCMS and HPLC. LCMS: Retention time: 2.411 min, (M+H)=520.2. HPLC: Retention time: 5.691 min. H NMR: (400 MHz, DMSO-d₆), δ=9.45 (s, 1H), 8.77 (s, 1H), 7.76-7.64 (m, 2H), 7.53-7.43 (m, 1H), 7.40-7.29 (m, 2H), 6.71-6.48 (m, 2H), 3.95-3.79 (m, 1H), 3.56 (d, J=5.6 Hz, 2H), 3.47-3.42 (m, 2H), 3.29-3.23 (m, 2H), 2.45-2.38 (m, 1H), 1.99 (s, 3H), 1.50 (d, J=8.0 Hz, 1H), 1.38 (d, J=6.8 Hz, 6H)

### Example 106: Synthesis of Compound 106

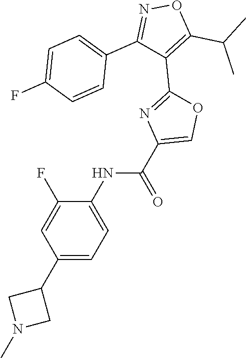 [SMILES: CC(C)c1onc(-c2ccc(F)cc2)c1-c1nc(C(=O)Nc2ccc(C3CN(C)C3)cc2F)co1]

N-(2-fluoro-4-(1-methylazetidin-3-yl)phenyl)-2-(3-(4-fluorophenyl)-5-isopropylisoxazol-4-yl)oxazole-4-carboxamide (Compound 106) was synthesized under the same synthetic route as for Compound 30 as white solid, which was determined by ¹HNMR, LCMS and HPLC. LCMS: Retention time: 0.816 min, (M+H)=479.0. HPLC: Retention time: 4.48 min. HNMR: (400 MHz, DMSO-d₆), δ=9.61 (s, 1H), 8.85 (s, 1H), 7.84-7.75 (m, 1H), 7.74-7.68 (m, 2H), 7.46-7.40 (m, 1H), 7.39-7.31 (m, 2H), 7.26-7.20 (m, 1H), 4.05-3.96 (m, 2H), 3.89-3.80 (m, 2H), 3.72-3.60 (m, 2H), 2.61 (s, 3H), 1.38 (d, J=6.8 Hz, 6H).

### Example 107: Synthesis of Compound 107

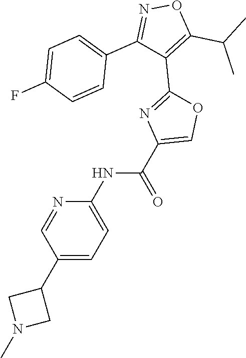 [SMILES: CC(C)c1onc(-c2ccc(F)cc2)c1-c1nc(C(=O)Nc2ccc(C3CN(C)C3)cn2)co1]

2-(3-(4-fluorophenyl)-5-isopropylisoxazol-4-yl)-N-(5-(1-methylazetidin-3-yl)pyridin-2-yl)oxazole-4-carboxamide (Compound 107) was synthesized under the same synthetic route as for Compound 30 as white solid, which was determined by ¹HNMR, LCMS and HPLC. LCMS: Retention time: 1.220 min, (M+H)=462.3. HPLC: Retention time: 2.631 min. HNMR: (400 MHz, DMSO-d₆), δ=9.91 (s, 1H), 8.92 (s, 1H), 8.37 (d, J=2.0 Hz, 1H), 8.19-8.11 (m, 1H), 8.00-7.94 (m, 1H), 7.74-7.66 (m, 2H), 7.40-7.32 (m, 2H), 4.04-3.96 (m, 2H), 3.90-3.80 (m, 2H), 3.72-3.63 (m, 2H), 2.60 (s, 3H), 1.39 (d, J=6.8 Hz, 6H).

### Example 108: Synthesis of Compound 108

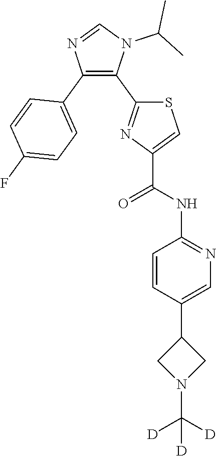 [SMILES: [2H]C([2H])([2H])N1CC(c2ccc(NC(=O)c3csc(-c4c(-c5ccc(F)cc5)ncn4C(C)C)n3)nc2)C1]

2-[5-(4-fluorophenyl)-3-isopropyl-imidazol-4-yl]-N-[5-[1-(trideuteriomethyl)azetidin-3-yl]-2-pyridyl]thiazole-4-carboxamide (Compound 108) was synthesized under the same synthetic route as for Compound 30 as white solid, which was determined by ¹HNMR, LCMS and HPLC. LCMS: Retention time: 1.258 min, (M+H)=480.3. HPLC: Retention time: 2.856 min. HNMR: (400 MHz, DMSO-d₆), δ=9.96 (s, 1H), 8.66 (s, 1H), 8.33 (d, J=2.0 Hz, 1H), 8.22-8.15 (m, 2H), 7.95 (dd, J=2.0, 8.4 Hz, 1H), 7.50-7.41 (m, 2H), 7.23-7.13 (m, 2H), 4.78-4.68 (m, 1H), 3.69-3.59 (m, 3H), 3.22-3.13 (m, 2H), 1.49 (d, J=6.8 Hz, 6H).

### Example 109: Synthesis of Compound 109

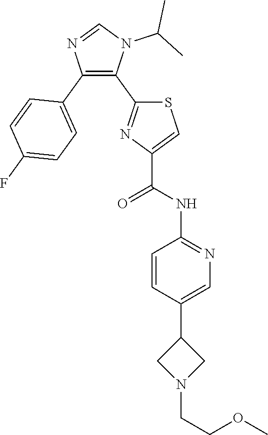 [SMILES: COCCN1CC(c2ccc(NC(=O)c3csc(-c4c(-c5ccc(F)cc5)ncn4C(C)C)n3)nc2)C1]

2-(4-(4-fluorophenyl)-1-isopropyl-1H-imidazol-5-yl)-N-(5-(1-(2-methoxyethyl)azetidin-3-yl)pyridin-2-yl)thiazole-4-carboxamide (Compound 109) was synthesized under the same synthetic route as for Compound 30 as white solid, which was determined by ¹HNMR, LCMS and HPLC. LCMS: Retention time: 0.663 min, (M+H)=521.1. HPLC: Retention time: 2.042 min. HNMR: (400 MHz, DMSO-d₆), δ=9.96 (s, 1H), 8.66 (s, 1H), 8.32 (d, J=2.0 Hz, 1H), 8.22-8.15 (m, 2H), 7.93 (dd, J=2.0, 8.8 Hz, 1H), 7.52-7.42 (m, 2H), 7.23-7.14 (m, 2H), 4.79-4.65 (m, 1H), 3.68-3.58 (m, 3H), 3.32 (t, J=5.6 Hz, 2H), 3.23 (s, 3H), 3.18-3.14 (m, 2H), 2.62 (t, J=5.6 Hz, 2H), 1.49 (d, J=6.8 Hz, 6H).

### Example 110: Synthesis of Compound 110

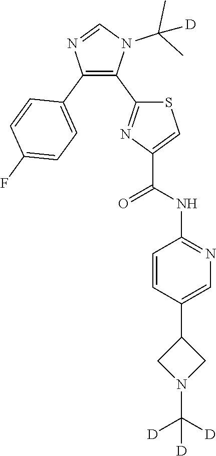 [SMILES: [2H]C([2H])([2H])N1CC(c2ccc(NC(=O)c3csc(-c4c(-c5ccc(F)cc5)ncn4C([2H])(C)C)n3)nc2)C1]

2-[3-(1-deuterio-1-methyl-ethyl)-5-(4-fluorophenyl)imidazol-4-yl]-N-[5-[1-(trideuteriomethyl)azetidin-3-yl]-2-pyridyl]thiazole-4-carboxamide (Compound 110) was synthesized under the same synthetic route as for Compound 30 as white solid, which was determined by ¹HNMR, LCMS and HPLC. LCMS: Retention time: 2.023 min, (M+H)=481.2. HPLC: Retention time: 5.201 min. HNMR: (400 MHz, DMSO-d₆), δ=9.96 (s, 1H), 8.66 (s, 1H), 8.35-8.30 (m, 1H), 8.22-8.15 (m, 2H), 7.95 (dd, J=2.4, 8.8 Hz, 1H), 7.50-7.44 (m, 2H), 7.23-7.14 (m, 2H), 3.62-3.53 (m, 3H), 3.09 (s, 2H), 1.52-1.43 (m, 6H).

### Example 111: Synthesis of Compound 111

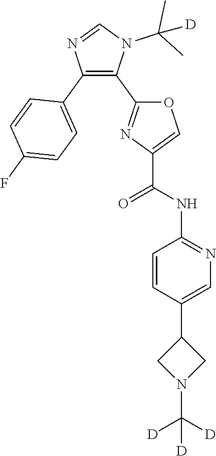 [SMILES: [2H]C([2H])([2H])N1CC(c2ccc(NC(=O)c3coc(-c4c(-c5ccc(F)cc5)ncn4C([2H])(C)C)n3)nc2)C1]

Compound 111 was synthesized under the same synthetic route as for Compound 30 as white solid, which was determined by ¹HNMR, LCMS and HPLC. LCMS: Retention time: 5.060 min, (M+H)=465.2. HPLC: Retention time: 4.867 min. HNMR: (400 MHz, CDCl₃), δ=9.20 (s, 1H), 8.36-8.32 (m, 1H), 8.29 (s, 1H), 8.25 (d, J=2.0 Hz, 1H), 7.83 (s, 1H), 7.80 (dd, J=2.4, 8.8 Hz, 1H), 7.60-7.51 (m, 2H), 7.13-7.04 (m, 2H), 3.88-3.80 (m, 2H), 3.79-3.68 (m, 1H), 3.34-3.22 (m, 2H), 1.60 (s, 6H).

### Example 112: Synthesis of Compound 112

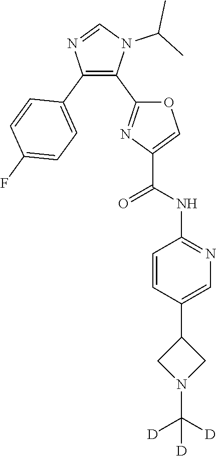 [SMILES: [2H]C([2H])([2H])N1CC(c2ccc(NC(=O)c3coc(-c4c(-c5ccc(F)cc5)ncn4C(C)C)n3)nc2)C1]

2-[5-(4-fluorophenyl)-3-isopropyl-imidazol-4-yl]-N-[5-[1 (trideuteriomethyl)azetidin-3-yl]-2-pyridyl]oxazole-4-carboxamide (Compound 112) was synthesized under the same synthetic route as for Compound 30 as white solid, which was determined by ¹HNMR, LCMS and HPLC. LCMS: Retention time: 0.772 min, (M+H)=464.3. HPLC: Retention time: 2.085 min. HNMR: (400 MHz, DMSO-d₆), δ=10.09 (s, 1H), 8.96 (s, 1H), 8.35 (d, J=2.4 Hz, 1H), 8.24 (s, 1H), 8.16 (d, J=8.4 Hz, 1H), 7.99-7.94 (m, 1H), 7.58-7.51 (m, 2H), 7.25-7.17 (m, 2H), 5.07-4.91 (m, 1H), 3.80-3.75 (m, 2H), 3.74-3.68 (m, 1H), 3.38-3.37 (m, 2H), 1.50 (d, J=6.8 Hz, 6H).

### Example 113: Synthesis of Compound 113

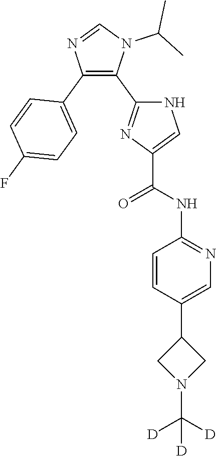 [SMILES: [2H]C([2H])([2H])N1CC(c2ccc(NC(=O)c3c[nH]c(-c4c(-c5ccc(F)cc5)ncn4C(C)C)n3)nc2)C1]

Compound 113 was synthesized under the same synthetic route as for Compound 30 as white powder, which was determined by ¹HNMR, LCMS and HPLC.

LCMS: Retention time: 2.781 min, (M+H)=463.2, 10-80CD_7 min_220&254_Agilent.M

HPLC: Retention time: 1.845 min. HNMR: (400 MHz, CDCl₃), δ=10.99 (br. s., 1H), 9.43 (s, 1H), 8.26 (d, J=8.8 Hz, 1H), 8.12 (d, J=2.4 Hz, 1H), 7.68-7.64 (m, 2H), 7.54 (s, 1H), 7.31-7.24 (m, 2H), 7.00-6.85 (m, 2H), 4.91-4.76 (m, 1H), 3.69-3.60 (m, 2H), 3.59-3.48 (m, 1H), 3.15-3.04 (m, 2H), 1.44 (s, 3H), 1.43 (s, 3H).

### Example 114: Synthesis of Compound 114

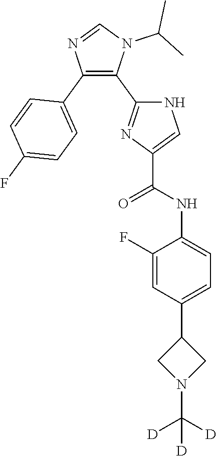 [SMILES: [2H]C([2H])([2H])N1CC(c2ccc(NC(=O)c3c[nH]c(-c4c(-c5ccc(F)cc5)ncn4C(C)C)n3)c(F)c2)C1]

2-[5-(4-fluorophenyl)-3-isopropyl-imidazol-4-yl]-N-[2-fluoro-4-[1-(trideuteriomethyl)azetidin-3-yl]phenyl]-1H-imidazole-4-carboxamide (Compound 114) was synthesized under the same synthetic route as for Compound 30 as white solid, which was determined by ¹HNMR, LCMS and HPLC. LCMS: Retention time: 1.134 min, (M+H)=480.3. HPLC: Retention time: 2.527 min. HNMR: (400 MHz, DMSO-d₆), δ=9.57 (s, 1H), 8.21 (s, 1H), 8.07 (s, 1H), 8.06 (s, 1H), 7.97-7.85 (m, 1H), 7.45-7.29 (m, 3H), 7.21-7.07 (m, 3H), 4.30-4.20 (m, 1H), 3.76-3.72 (m, 2H), 3.69-3.67 (m, 1H), 3.33-3.26 (m, 2H), 1.40 (d, J=6.8 Hz, 6H).

### Example 115: Synthesis of Compound 115

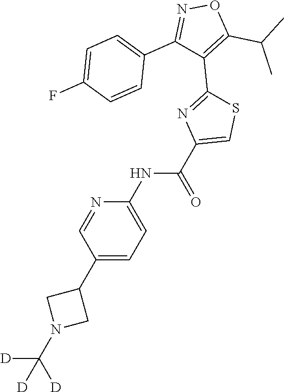 [SMILES: [2H]C([2H])([2H])N1CC(c2ccc(NC(=O)c3csc(-c4c(-c5ccc(F)cc5)noc4C(C)C)n3)nc2)C1]

2-[3-(4-fluorophenyl)-5-isopropyl-isoxazol-4-yl]-N-[5-[1-(trideuteriomethyl)azetidin-3-yl]-2-pyridyl]thiazole-4-carboxamide (Compound 115) was synthesized under the same synthetic route as for Compound 30 as white solid, which was determined by ¹HNMR, LCMS and HPLC. LCMS: Retention time: 1.009 min, (M+H)=481.3. HPLC: Retention time: 3.525 min. HNMR: (400 MHz, DMSO-d₆), δ=9.74 (s, 1H), 8.58 (s, 1H), 8.37 (d, J=2.0 Hz, 1H), 8.20 (d, J=5.6 Hz, 1H), 8.01-7.94 (m, 1H), 7.67-7.60 (m, 2H), 7.41-7.33 (m, 2H), 3.95-3.87 (m, 2H), 3.83-3.77 (m, 1H), 3.76-3.70 (m, 1H), 3.60-3.58 (m, 2H), 1.41 (d, J=7.2 Hz, 6H).

### Example 116: Synthesis of Compound 116

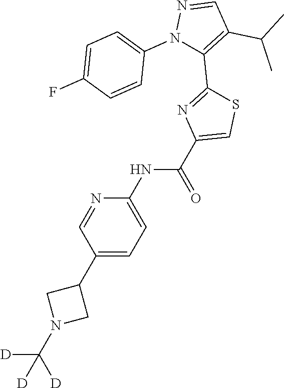 [SMILES: [2H]C([2H])([2H])N1CC(c2ccc(NC(=O)c3csc(-c4c(C(C)C)cnn4-c4ccc(F)cc4)n3)nc2)C1]

2-(1-(4-fluorophenyl)-4-isopropyl-1H-pyrazol-5-yl)-N-(5-(1-(trideuteriomethyl)azetidin-3-yl)pyridin-2-yl)thiazole-4-carboxamide (Compound 116) was synthesized under the same synthetic route as for Compound 30 as white solid, which was determined by ¹HNMR, LCMS and HPLC. LCMS: Retention time: 5.837 min, (M+H)=480.1. HPLC: Retention time: 3.863 min. HNMR: (400 MHz, CDCl₃), δ=9.43 (s, 1H), 8.30 (d, J=8.8 Hz, 1H), 8.27-8.23 (m, 2H), 7.76 (dd, J=2.4, 8.8 Hz, 1H), 7.72 (s, 1H), 7.41-7.35 (m, 2H), 7.18-7.10 (m, 2H), 3.84-3.77 (m, 2H), 3.76-3.66 (m, 1H), 3.33-3.19 (m, 3H), 1.36 (d, J=7.2 Hz, 6H).

### Example 117: Synthesis of Compound 117

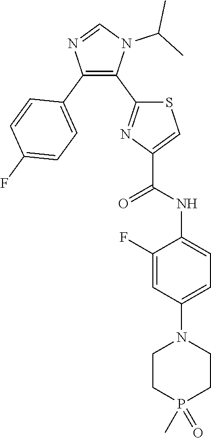 [SMILES: CC(C)n1cnc(-c2ccc(F)cc2)c1-c1nc(C(=O)Nc2ccc(N3CCP(C)(=O)CC3)cc2F)cs1]

N-(2-fluoro-4-(4-methyl-4-oxido-1,4-azaphosphinan-1-yl)phenyl)-2-(4-(4-fluorophenyl)-1-isopropyl-1H-imidazol-5-yl)thiazole-4-carboxamide (Compound 117) was synthesized under the same synthetic route as for Compound 2 as white solid, which was determined by ¹HNMR, LCMS and HPLC. LCMS: Retention time: 2.164 min, (M+H)=556.2. HPLC: Retention time: 3.661 min. HNMR: (400 MHz, DMSO-d₆), δ=9.72 (s, 1H), 8.54 (s, 1H), 8.18 (s, 1H), 7.65-7.58 (m, 1H), 7.50-7.44 (m, 2H), 7.24-7.16 (m, 2H), 6.99-6.91 (m, 1H), 6.84-6.78 (m, 1H), 4.80-4.71 (m, 1H), 3.97-3.83 (m, 2H), 3.56-3.45 (m, 2H), 1.93-1.65 (m, 4H), 1.56-1.43 (m, 9H).

### Example 118: Synthesis of Compound 118

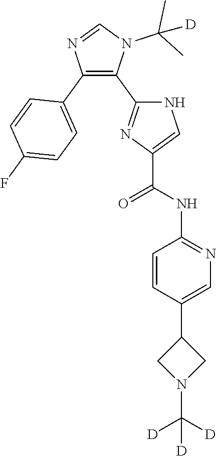 [SMILES: [2H]C([2H])([2H])N1CC(c2ccc(NC(=O)c3c[nH]c(-c4c(-c5ccc(F)cc5)ncn4C([2H])(C)C)n3)nc2)C1]

Compound 118 was synthesized under the same synthetic route as for Compound 30 as white solid, which was determined by ¹HNMR, LCMS and HPLC.

LCMS: Retention time: 1.135 min, (M+H)=464.0. HPLC: Retention time: 4.324 min. HNMR: (400 MHz, DMSO-d₆), δ=9.67 (br. s., 1H), 8.34-8.24 (m, 2H), 8.19 (d, J=8.4 Hz, 1H), 8.14 (s, 1H), 8.08 (s, 1H), 7.95-7.88 (m, 1H), 7.43-7.32 (m, 2H), 7.20-7.05 (m, 8.7 Hz, 2H), 3.67-3.60 (m, 3H), 3.26-3.12 (m, 2H), 1.39 (s, 6H).

### Example 119: Synthesis of Compound 119

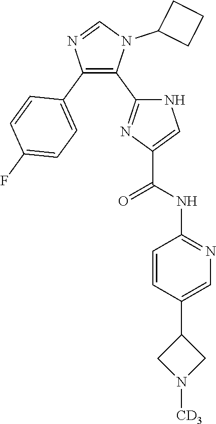 [SMILES: [2H]C([2H])([2H])N1CC(c2ccc(NC(=O)c3c[nH]c(-c4c(-c5ccc(F)cc5)ncn4C4CCC4)n3)nc2)C1]

2-[3-cyclobutyl-5-(4-fluorophenyl)imidazol-4-yl]-N-[5-[1-(trideuteriomethyl)azetidin-3-yl]-2-pyridyl]-1H-imidazole-4-carboxamide (Compound 119) was synthesized under the same synthetic route as for Compound 30 as white solid, which was determined by ¹HNMR, LCMS and HPLC. LCMS: Retention time: 0.854 min, (M+H)=475.3. HPLC: Retention time: 3.051 min. HNMR: (400 MHz, DMSO-d₆), δ=13.28-12.90 (m, 1H), 9.58 (s, 1H), 8.28 (s, 1H), 8.21-8.16 (m, 1H), 8.15-8.09 (m, 2H), 7.91 (dd, J=2.0, 8.4 Hz, 1H), 7.51-7.38 (m, 2H), 7.19-7.09 (m, 2H), 4.61-4.50 (m, 1H), 3.63-3.52 (m, 3H), 3.14-3.02 (m, 2H), 2.42-2.35 (m, 2H), 2.25-2.18 (m, 2H), 1.77-1.68 (m, 2H).

### Example 120: Synthesis of Compound 120

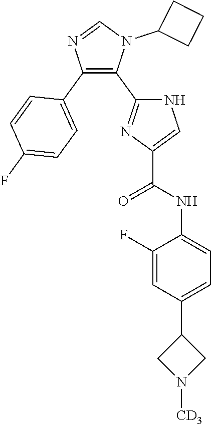 [SMILES: [2H]C([2H])([2H])N1CC(c2ccc(NC(=O)c3c[nH]c(-c4c(-c5ccc(F)cc5)ncn4C4CCC4)n3)c(F)c2)C1]

2-[3-cyclobutyl-5-(4-fluorophenyl)imidazol-4-yl]-N-[2-fluoro-4-[1-(trideuteriomethyl)azetidin-3-yl]phenyl]-1H-imidazole-4-carboxamide (Compound 120) was synthesized under the same synthetic route as for Compound 30 as white solid, which was determined by ¹HNMR, LCMS and HPLC. LCMS: Retention time: 1.892 min, (M+H)=492.0. HPLC: Retention time: 3.451 min. HNMR: (400 MHz, DMSO-d₆), δ=13.09 (s, 1H), 9.57 (s, 1H), 8.16 (s, 1H), 8.07-8.04 (m, 1H), 7.97 (s, 1H), 7.47-7.32 (m, 3H), 7.25-7.10 (m, 3H), 4.60-4.48 (m, 1H), 3.94-3.88 (m, 2H), 3.82-3.75 (m, 1H), 3.54-3.52 (m, 2H), 2.42-2.32 (m, 2H), 2.28-2.18 (m, 2H), 1.80-1.69 (m, 2H).

### Example 121: Synthesis of Compound 121

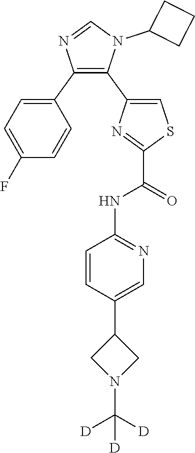 [SMILES: [2H]C([2H])([2H])N1CC(c2ccc(NC(=O)c3nc(-c4c(-c5ccc(F)cc5)ncn4C4CCC4)cs3)nc2)C1]

4-(1-cyclobutyl-4-(4-fluorophenyl)-1H-imidazol-5-yl)-N-(5-(1-(trideuteriomethyl) azetidin-3-yl)pyridin-2-yl)thiazole-2-carboxamide (Compound 121) was synthesized under the same synthetic route as for Compound 30 as white solid, which was determined by ¹HNMR, LCMS and HPLC. LCMS: Retention time: 0.714 min, (M+H)=492.3. HPLC: Retention time: 2.557 min. HNMR: (400 MHz, DMSO-d₆), δ=10.29 (s, 1H), 8.37 (s, 1H), 8.20 (s, 1H), 8.14-8.06 (m, 2H), 7.98 (dd, J=2.0, 6.4 Hz, 1H), 7.51-7.38 (m, 2H), 7.17-7.02 (m, 2H), 4.67-4.54 (m, 1H), 3.80-3.69 (m, 3H), 3.51-3.49 (m, 2H), 2.41-2.35 (m, 2H), 2.27-2.11 (m, 2H), 1.80-1.60 (m, 2H)

### Example 122: Synthesis of Compound 122

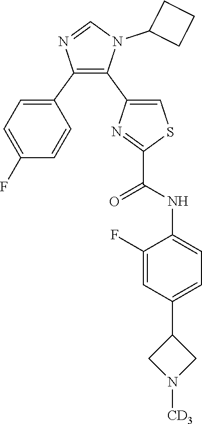 [SMILES: [2H]C([2H])([2H])N1CC(c2ccc(NC(=O)c3nc(-c4c(-c5ccc(F)cc5)ncn4C4CCC4)cs3)c(F)c2)C1]

4-[3-cyclobutyl-5-(4-fluorophenyl)imidazol-4-yl]-N-[2-fluoro-4-[1-(trideuteriomethyl)azetidin-3-yl]phenyl]thiazole-2-carboxamide (Compound 122) was synthesized under the same synthetic route as for Compound 30 as white solid, which was determined by ¹HNMR, LCMS and HPLC. LCMS: Retention time: 0.799 min, (M+H)=509.3. HPLC: Retention time: 2.763 min. HNMR: (400 MHz, DMSO-d₆), δ=10.39 (s, 1H), 8.21 (s, 1H), 8.12 (s, 1H), 7.64-7.58 (m, 1H), 7.49-7.42 (m, 2H), 7.35-7.28 (m, 1H), 7.20 (d, J=8.4 Hz, 1H), 7.16-7.07 (m, 2H), 4.59-4.49 (m, 1H), 3.61 (s, 3H), 3.13 (s, 2H), 2.43-2.33 (m, 2H), 2.25-2.18 (m, 2H), 1.77-1.68 (m, 2H).

### Example 123: Synthesis of Compound 123

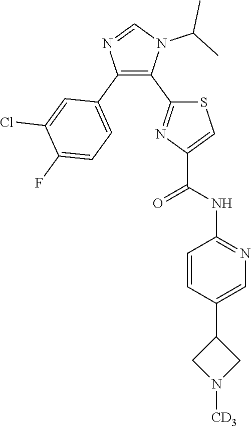 [SMILES: [2H]C([2H])([2H])N1CC(c2ccc(NC(=O)c3csc(-c4c(-c5ccc(F)c(Cl)c5)ncn4C(C)C)n3)nc2)C1]

2-[5-(3-chloro-4-fluoro-phenyl)-3-isopropyl-imidazol-4-yl]-N-[5-[1-(trideuteriomethyl)azetidin-3-yl]-2-pyridyl]thiazole-4-carboxamide (Compound 123) was synthesized under the same synthetic route as for Compound 30 as white solid, which was determined by ¹HNMR, LCMS and HPLC. LCMS: Retention time: 0.856 min, (M+H)=514.3. HPLC: Retention time: 2.545 min. HNMR: (400 MHz, DMSO-d₆), δ=9.98 (s, 1H), 8.71 (s, 1H), 8.33 (d, J=2.0 Hz, 1H), 8.24 (s, 1H), 8.21-8.17 (m, 1H), 7.98-7.93 (m, 1H), 7.68-7.64 (m, 1H), 7.42-7.33 (m, 2H), 4.76-4.60 (m, 1H), 3.68-3.55 (m, 3H), 3.19-3.09 (m, 2H), 1.49 (d, J=6.8 Hz, 6H).

### Example 124: Synthesis of Compound 124

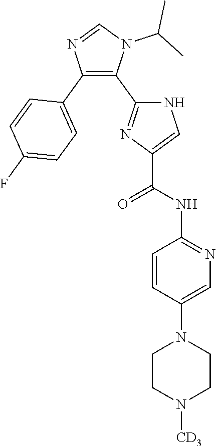 [SMILES: [2H]C([2H])([2H])N1CCN(c2ccc(NC(=O)c3c[nH]c(-c4c(-c5ccc(F)cc5)ncn4C(C)C)n3)nc2)CC1]

2-[5-(4-fluorophenyl)-3-isopropyl-imidazol-4-yl]-N-[5-[4 (trideuteriomethyl)piperazin-1-yl]-2-pyridyl]-1H-imidazole-4-carboxamide (Compound 124) was synthesized under the same synthetic route as for Compound 14 as white solid, which was determined by ¹HNMR, LCMS and HPLC. LCMS: Retention time: 1.490 min, (M+H)=492.3. HPLC: Retention time: 3.133 min. HNMR: (400 MHz, DMSO-d₆), δ=13.11 (s, 1H), 9.42 (s, 1H), 8.25-7.94 (m, 4H), 7.56-7.44 (m, 1H), 7.41-7.32 (m, 2H), 7.20-7.07 (m, 2H), 4.36-4.16 (m, 1H), 3.14 (s, 4H), 2.47-2.43 (m, 4H), 1.40 (d, J=6.4 Hz, 6H).

### Example 125: Synthesis of Compound 125

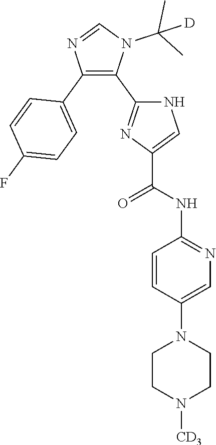 [SMILES: [2H]C([2H])([2H])N1CCN(c2ccc(NC(=O)c3c[nH]c(-c4c(-c5ccc(F)cc5)ncn4C([2H])(C)C)n3)nc2)CC1]

2-[3-(1-deuterio-1-methyl-ethyl)-5-(4-fluorophenyl)imidazol-4-yl]-N-[5-[4-(trideuteriomethyl)piperazin-1-yl]-2-pyridyl]-1H-imidazole-4-carboxamide (Compound 125) was synthesized under the same synthetic route as for Compound 14 as white solid, which was determined by ¹HNMR, LCMS and HPLC. LCMS: Retention time: 1.488 min, (M+H)=493.3. HPLC: Retention time: 3.508 min. HNMR: (400 MHz, DMSO-d₆), δ=13.11 (s, 1H), 9.43 (s, 1H), 8.16-7.95 (m, 4H), 7.57-7.44 (m, 1H), 7.41-7.34 (m, 2H), 7.20-7.09 (m, 2H), 3.22-3.09 (m, 4H), 2.48-2.44 (m, 4H), 1.40 (s, 6H).

### Example 126: Synthesis of Compound 126

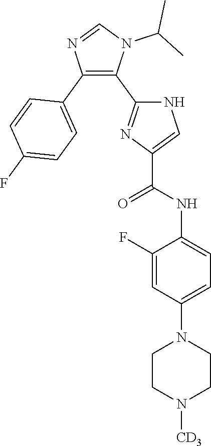 [SMILES: [2H]C([2H])([2H])N1CCN(c2ccc(NC(=O)c3c[nH]c(-c4c(-c5ccc(F)cc5)ncn4C(C)C)n3)c(F)c2)CC1]

2-[5-(4-fluorophenyl)-3-isopropyl-imidazol-4-yl]-N-[2-fluoro-4-[4-(trideuteriomethyl)piperazin-1-yl]phenyl]-1H-imidazole-4-carboxamide (Compound 126) was synthesized under the same synthetic route as for Compound 14 as white solid, which was determined by ¹HNMR, LCMS and HPLC. LCMS: Retention time: 1.672 min, (M+H)=509.3. HPLC: Retention time: 3.638 min. HNMR: (400 MHz, DMSO-d₆), δ=13.06 (s, 1H), 9.37 (s, 1H), 8.07 (s, 1H), 8.00 (s, 1H), 7.72-7.62 (m, 1H), 7.43-7.32 (m, 2H), 7.18-7.09 (m, 2H), 6.85 (dd, J=2.4, 14.8 Hz, 1H), 6.76 (d, J=9.2 Hz, 1H), 4.33-4.19 (m, 1H), 3.21-3.08 (m, 4H), 2.46-2.40 (m, 4H), 1.40 (d, J=6.4 Hz, 6H).

### Example 127: Synthesis of Compound 127

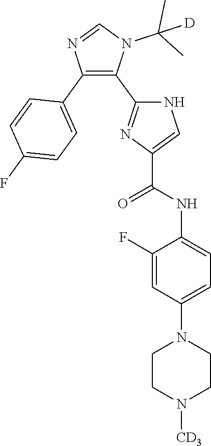 [SMILES: [2H]C([2H])([2H])N1CCN(c2ccc(NC(=O)c3c[nH]c(-c4c(-c5ccc(F)cc5)ncn4C([2H])(C)C)n3)c(F)c2)CC1]

2-[3-(1-deuterio-1-methyl-ethyl)-5-(4-fluorophenyl)imidazol-4-yl]-N-[2-fluoro-4-[4-(trideuteriomethyl)piperazin-1-yl]phenyl]-1H-imidazole-4-carboxamide (Compound 127) was synthesized under the same synthetic route as for Compound 14 as white solid, which was determined by ¹HNMR, LCMS and HPLC. LCMS: Retention time: 1.678 min, (M+H)=510.3. HPLC: Retention time: 2.231 min. HNMR: (400 MHz, DMSO-d₆), δ=13.06 (s, 1H), 9.37 (s, 1H), 8.07 (s, 1H), 8.00 (s, 1H), 7.71-7.61 (m, 1H), 7.42-7.33 (m, 2H), 7.19-7.09 (m, 2H), 6.89-6.81 (m, 1H), 6.78-6.72 (m, 1H), 3.24-3.05 (m, 4H), 2.45-2.42 (m, 4H), 1.39 (s, 6H).

### Example 128: Synthesis of Compound 128

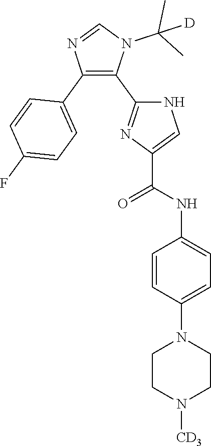 [SMILES: [2H]C([2H])([2H])N1CCN(c2ccc(NC(=O)c3c[nH]c(-c4c(-c5ccc(F)cc5)ncn4C([2H])(C)C)n3)cc2)CC1]

2-[3-(1-deuterio-1-methyl-ethyl)-5-(4-fluorophenyl)imidazol-4-yl]-N-[4-[4-(trideuteriomethyl)piperazin-1-yl]phenyl]-1H-imidazole-4-carboxamide (Compound 128) was synthesized under the same synthetic route as for Compound 14 as white solid, which was determined by ¹HNMR, LCMS and HPLC. LCMS: Retention time: 1.579 min, (M+H)=492.3. HPLC: Retention time: 1.498 min. HNMR: (400 MHz, DMSO-d₆), δ=13.04 (s, 1H), 9.72 (s, 1H), 8.06 (s, 1H), 7.98 (s, 1H), 7.66 (d, J=8.8 Hz, 2H), 7.43-7.33 (m, 2H), 7.18-7.08 (m, 2H), 6.89 (d, J=8.8 Hz, 2H), 3.17-3.02 (m, 4H), 2.48-2.39 (m, 4H), 1.38 (s, 6H).

### Example 129: Synthesis of Compound 129

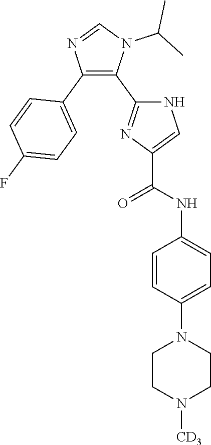 [SMILES: [2H]C([2H])([2H])N1CCN(c2ccc(NC(=O)c3c[nH]c(-c4c(-c5ccc(F)cc5)ncn4C(C)C)n3)cc2)CC1]

2-[5-(4-fluorophenyl)-3-isopropyl-imidazol-4-yl]-N-[4-[4 (trideuteriomethyl)piperazin-1-yl]phenyl]-1H-imidazole-4-carboxamide (Compound 129) was synthesized under the same synthetic route as for Compound 14 as white solid, which was determined by ¹HNMR, LCMS and HPLC. LCMS: Retention time: 1.577 min, (M+H)=491.3. HPLC: Retention time: 1.504 min. HNMR: (400 MHz, DMSO-d₆), δ=13.14 (s, 1H), 9.82 (s, 1H), 8.16 (s, 1H), 8.07 (s, 1H), 7.75 (d, J=8.8 Hz, 2H), 7.51-7.43 (m, 2H), 7.27-7.18 (m, 2H), 6.99 (d, J=8.8 Hz, 2H), 4.48-4.20 (m, 1H), 3.23-3.13 (m, 4H), 2.57-2.50 (m, 4H), 1.48 (d, J=6.8 Hz, 6H).

### Example 130: Synthesis of Compound 130

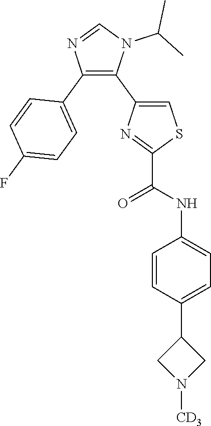 [SMILES: [2H]C([2H])([2H])N1CC(c2ccc(NC(=O)c3nc(-c4c(-c5ccc(F)cc5)ncn4C(C)C)cs3)cc2)C1]

4-[5-(4-fluorophenyl)-3-isopropyl-imidazol-4-yl]-N-[5-[1-(trideuteriomethyl)azetidin-3-yl]-2-pyridyl]thiazole-2-carboxamide (Compound 130) was synthesized under the same synthetic route as for Compound 30 as white solid, which was determined by ¹HNMR, LCMS and HPLC. LCMS: Retention time: 0.723 min, (M+H)=480.1. HPLC: Retention time: 2.508 min. HNMR: (400 MHz, DMSO-d₆), δ=9.65-9.51 (m, 1H), 8.33 (d, J=8.4 Hz, 1H), 8.27 (d, J=2.0 Hz, 1H), 7.83 (dd, J=2.0, 8.4 Hz, 1H), 7.77 (s, 1H), 7.51 (s, 1H), 7.44-7.36 (m, 2H), 7.01-6.93 (m, 2H), 4.57-4.37 (m, 1H), 3.83-3.74 (m, 2H), 3.73-3.66 (m, 1H), 3.27-3.18 (m, 2H), 1.52 (d, J=6.8 Hz, 6H).

### Example 131: Synthesis of Compound 131

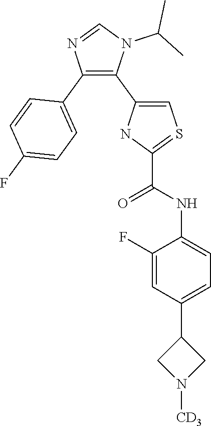

4-[5-(4-fluorophenyl)-3-isopropyl-imidazol-4-yl]-N-[2-fluoro-4-[1-(trideuteriomethyl)azetidin-3-yl]phenyl]thiazole-2-carboxamide (Compound 131) was synthesized under the same synthetic route as for Compound 30 as white solid, which was determined by ¹HNMR, LCMS and HPLC. LCMS: Retention time: 2.131 min, (M+H)=497.2. HPLC: Retention time: 2.670 min. HNMR: (400 MHz, DMSO-d₆), δ=10.43 (s, 1H), 8.27 (s, 1H), 8.06 (s, 1H), 7.64-7.55 (m, 1H), 7.47-7.40 (m, 2H), 7.35-7.29 (m, 1H), 7.20 (d, J=8.4 Hz, 1H), 7.15-7.06 (m, 2H), 4.27-4.17 (m, 1H), 3.58 (s, 3H), 3.07 (s, 2H), 1.41 (d, J=6.8 Hz, 6H).

### Example 132: Synthesis of Compound 132

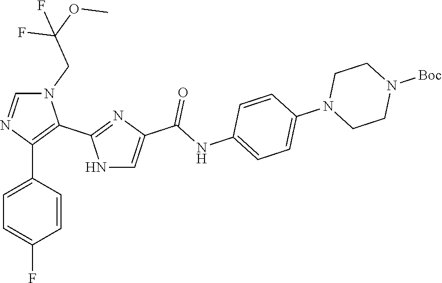 [SMILES: COC(F)(F)Cn1cnc(-c2ccc(F)cc2)c1-c1nc(C(=O)Nc2ccc(N3CCN(C(=O)OC(C)(C)C)CC3)cc2)c[nH]1]

tert-butyl 4-(4-(3′-(2,2-difluoro-2-methoxyethyl)-5′-(4-fluorophenyl)-1H,3′H-[2,4′-biimidazole]-4-carboxamido)phenyl)piperazine-1-carboxylate (Compound 132) was synthesized under the same synthetic route as for Compound 30 as white solid, which was determined by ¹HNMR, LCMS and HPLC. LCMS: Retention time: 0.798 min, (M+H)=626.3. HPLC: Retention time: 3.989 min. HNMR: (400 MHz, DMSO-d₆), δ=12.95 (s, 1H), 9.76 (s, 1H), 8.03-7.86 (m, 2H), 7.76-7.62 (m, 2H), 7.50-7.34 (m, 2H), 7.24-7.11 (m, 2H), 7.02-6.87 (m, 2H), 4.82 (t, J=8.8 Hz, 2H), 3.50-3.43 (m, 4H), 3.42 (s, 3H), 3.09-3.01 (m, 4H), 1.42 (s, 9H).

### Example 133: Synthesis of Compound 133

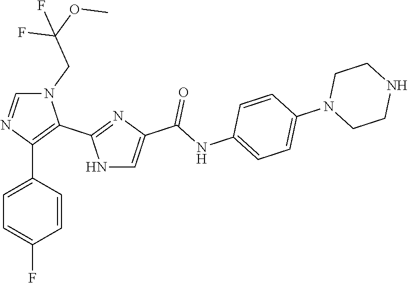 [SMILES: COC(F)(F)Cn1cnc(-c2ccc(F)cc2)c1-c1nc(C(=O)Nc2ccc(N3CCNCC3)cc2)c[nH]1]

3′-(2,2-difluoro-2-methoxyethyl)-5′-(4-fluorophenyl)-N-(4-(piperazin-1-yl)phenyl)-1H,3′H-[2,4′-biimidazole]-4-carboxamide (Compound 133) was synthesized under the same synthetic route as for Compound 30 as white solid, which was determined by ¹HNMR, LCMS and HPLC. LCMS: Retention time: 0.587 min, (M+H)=526.3. HPLC: Retention time: 2.370 min. HNMR: (400 MHz, DMSO-d₆), δ=9.84 (s, 1H), 8.91-8.63 (m, 2H), 8.01 (s, 1H), 7.93 (s, 1H), 7.71 (d, J=8.8 Hz, 2H), 7.47-7.37 (m, 2H), 7.23-7.12 (m, 2H), 6.98 (d, J=8.8 Hz, 2H), 4.82 (t, J=9.2 Hz, 2H), 4.52-4.33 (m, 4H), 3.42 (s, 3H), 3.34-3.28 (m, 4H).

### Example 134: Synthesis of Compound 134

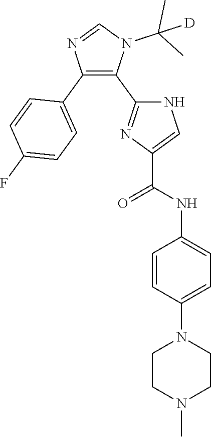 [SMILES: [2H]C(C)(C)n1cnc(-c2ccc(F)cc2)c1-c1nc(C(=O)Nc2ccc(N3CCN(C)CC3)cc2)c[nH]1]

2-[3-(1-deuterio-1-methyl-ethyl)-5-(4-fluorophenyl)imidazol-4-yl]-N-[4-(4-methylpiperazin-1-yl)phenyl]-1H-imidazole-4-carboxamide (Compound 134) was synthesized under the same synthetic route as for Compound 14 as white solid, which was determined by ¹HNMR, LCMS and HPLC. LCMS: Retention time: 1.728 min, (M+H)=489.2. HPLC: Retention time: 3.311 min. HNMR: (400 MHz, DMSO-d₆), δ=13.02 (s, 1H), 9.70 (s, 1H), 8.05 (s, 1H), 7.97 (s, 1H), 7.66 (d, J=8.8 Hz, 2H), 7.43-7.34 (m, 2H), 7.18-7.08 (m, 2H), 6.95-6.84 (m, 2H), 3.15-3.02 (m, 4H), 2.47-2.41 (m, 4H), 2.21 (s, 3H), 1.38 (s, 6H).

### Example 135: Synthesis of Compound 135

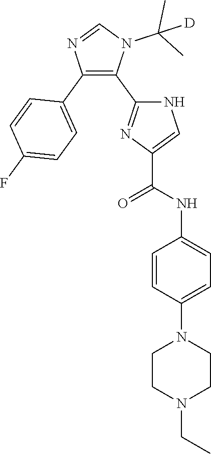 [SMILES: [2H]C(C)(C)n1cnc(-c2ccc(F)cc2)c1-c1nc(C(=O)Nc2ccc(N3CCN(CC)CC3)cc2)c[nH]1]

2-[3-(1-deuterio-1-methyl-ethyl)-5-(4-fluorophenyl)imidazol-4-yl]-N-[4-(4-ethylpiperazin-1-yl)phenyl]-1H-imidazole-4-carboxamide (Compound 135) was synthesized under the same synthetic route as for Compound 14 as white solid, which was determined by ¹HNMR, LCMS and HPLC. LCMS: Retention time: 1.809 min, (M+H)=503.3. HPLC: Retention time: 4.921 min. HNMR: (400 MHz, DMSO-d₆), δ=13.03 (s, 1H), 9.73 (s, 1H), 8.06 (s, 1H), 7.98 (s, 1H), 7.70-7.61 (m, 2H), 7.41-7.34 (m, 2H), 7.18-7.08 (m, 2H), 6.89 (d, J=8.8 Hz, 2H), 3.13-3.04 (m, 4H), 2.49-2.46 (m, 4H), 2.36 (q, J=7.2 Hz, 2H), 1.38 (s, 5H), 1.03 (t, J=7.2 Hz, 3H).

Certain compounds of Table 1 can be prepared employing alternative reagents in the examples above. Exemplary compounds may include, but are not limited to, a compound or salt thereof selected from Table 1 which may be prepared using the examples above and the accompanying procedures described herein.

TABLE 1
Compounds and Assay Data

| | | Human Liver | |
|---|---|---|---|
| | | Microsome | TNIK |
| Compound # | Compound Structure | (μM/min/mg) | (IC50, nM) |
| 1 | 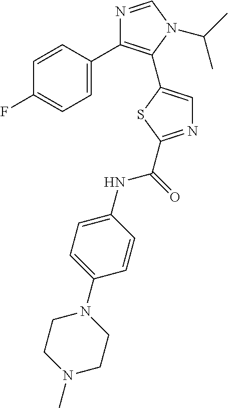 [SMILES: CC(C)n1cnc(-c2ccc(F)cc2)c1-c1cnc(C(=O)Nc2ccc(N3CCN(C)CC3)cc2)s1] | | + |
| 2 | 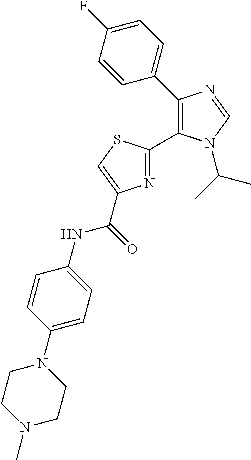 [SMILES: CC(C)n1cnc(-c2ccc(F)cc2)c1-c1nc(C(=O)Nc2ccc(N3CCN(C)CC3)cc2)cs1] | ++ | +++ |
| 3 | 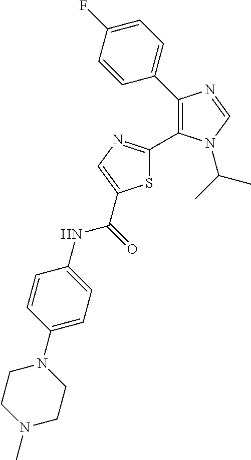 [SMILES: CC(C)n1cnc(-c2ccc(F)cc2)c1-c1ncc(C(=O)Nc2ccc(N3CCN(C)CC3)cc2)s1] | | + |
| 4 | 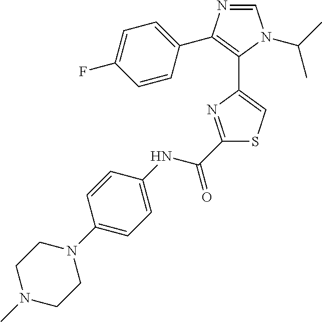 [SMILES: CC(C)n1cnc(-c2ccc(F)cc2)c1-c1csc(C(=O)Nc2ccc(N3CCN(C)CC3)cc2)n1] | | +++ |
| 5 | 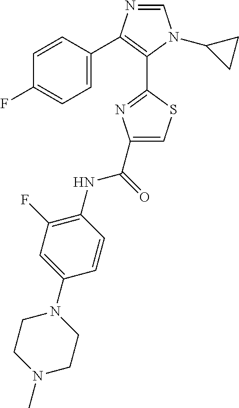 [SMILES: CN1CCN(c2ccc(NC(=O)c3csc(-c4c(-c5ccc(F)cc5)ncn4C4CC4)n3)c(F)c2)CC1] | | |
| 6 | 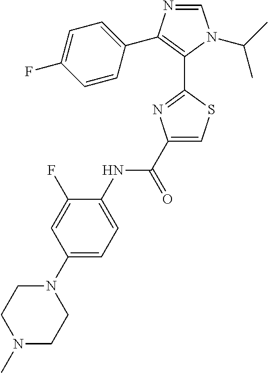 [SMILES: CC(C)n1cnc(-c2ccc(F)cc2)c1-c1nc(C(=O)Nc2ccc(N3CCN(C)CC3)cc2F)cs1] | + | +++ |
| 7 | 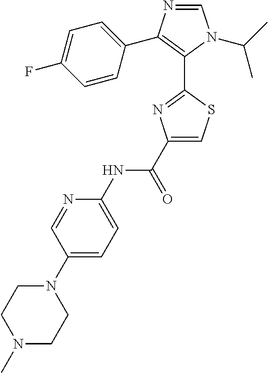 [SMILES: CC(C)n1cnc(-c2ccc(F)cc2)c1-c1nc(C(=O)Nc2ccc(N3CCN(C)CC3)cn2)cs1] | + | +++ |
| 8 | 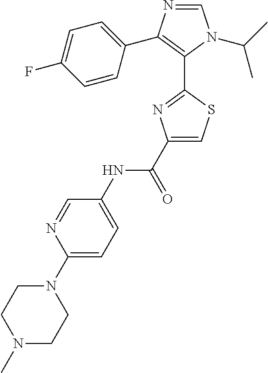 [SMILES: CC(C)n1cnc(-c2ccc(F)cc2)c1-c1nc(C(=O)Nc2ccc(N3CCN(C)CC3)nc2)cs1] | + | +++ |
| 9 | 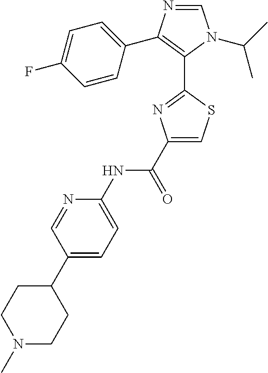 [SMILES: CC(C)n1cnc(-c2ccc(F)cc2)c1-c1nc(C(=O)Nc2ccc(C3CCN(C)CC3)cn2)cs1] | ++ | +++ |
| 10 | 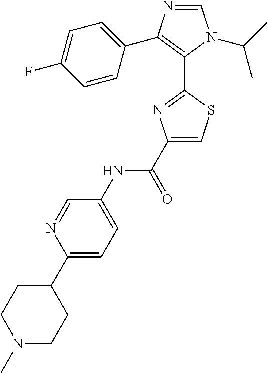 [SMILES: CC(C)n1cnc(-c2ccc(F)cc2)c1-c1nc(C(=O)Nc2ccc(C3CCN(C)CC3)nc2)cs1] | | |
| 11 | 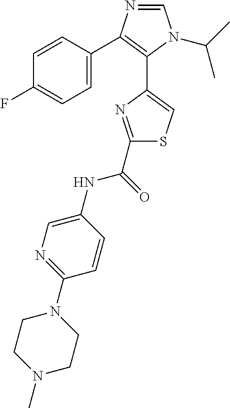 [SMILES: CC(C)n1cnc(-c2ccc(F)cc2)c1-c1csc(C(=O)Nc2ccc(N3CCN(C)CC3)nc2)n1] | | |
| 12 | 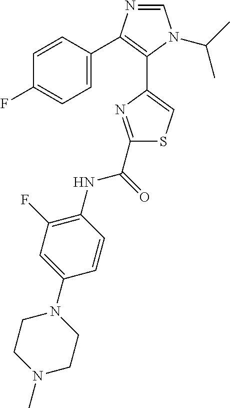 [SMILES: CC(C)n1cnc(-c2ccc(F)cc2)c1-c1csc(C(=O)Nc2ccc(N3CCN(C)CC3)cc2F)n1] | ++ | +++ |
| 13 | 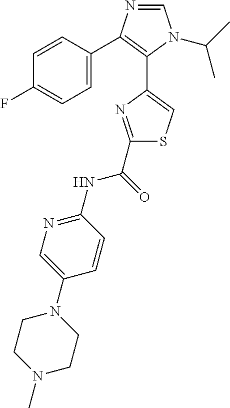 [SMILES: CC(C)n1cnc(-c2ccc(F)cc2)c1-c1csc(C(=O)Nc2ccc(N3CCN(C)CC3)cn2)n1] | + | +++ |
| 14 | 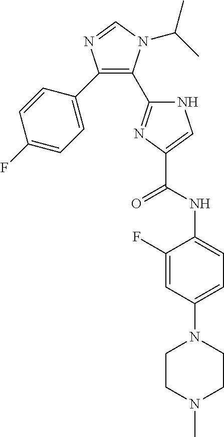 [SMILES: CC(C)n1cnc(-c2ccc(F)cc2)c1-c1nc(C(=O)Nc2ccc(N3CCN(C)CC3)cc2F)c[nH]1] | ++ | +++ |
| 15 | 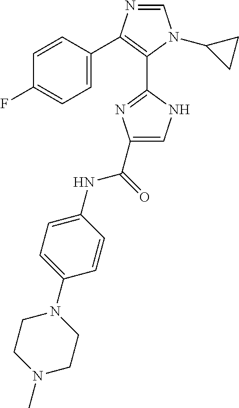 [SMILES: CN1CCN(c2ccc(NC(=O)c3c[nH]c(-c4c(-c5ccc(F)cc5)ncn4C4CC4)n3)cc2)CC1] | | ++ |
| 16 | 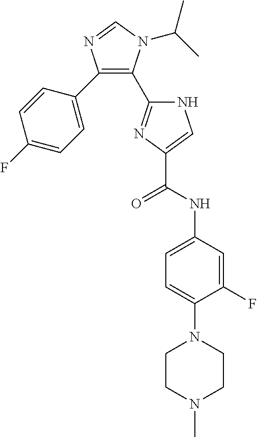 [SMILES: CC(C)n1cnc(-c2ccc(F)cc2)c1-c1nc(C(=O)Nc2ccc(N3CCN(C)CC3)c(F)c2)c[nH]1] | | +++ |
| 17 | 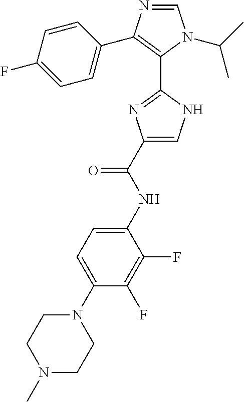 [SMILES: CC(C)n1cnc(-c2ccc(F)cc2)c1-c1nc(C(=O)Nc2ccc(N3CCN(C)CC3)c(F)c2F)c[nH]1] | | +++ |
| 18 | 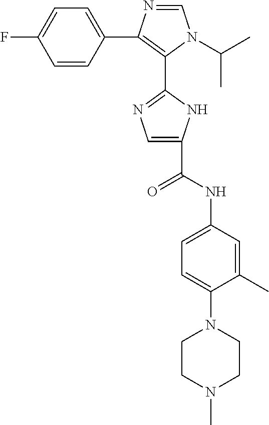 [SMILES: Cc1cc(NC(=O)c2cnc(-c3c(-c4ccc(F)cc4)ncn3C(C)C)[nH]2)ccc1N1CCN(C)CC1] | | ++ |
| 19 | 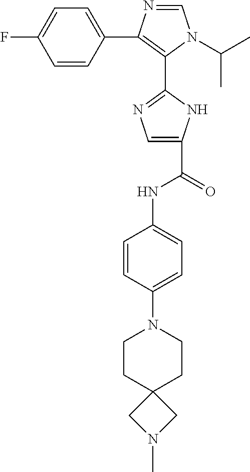 [SMILES: CC(C)n1cnc(-c2ccc(F)cc2)c1-c1ncc(C(=O)Nc2ccc(N3CCC4(CC3)CN(C)C4)cc2)[nH]1] | | +++ |
| 20 | 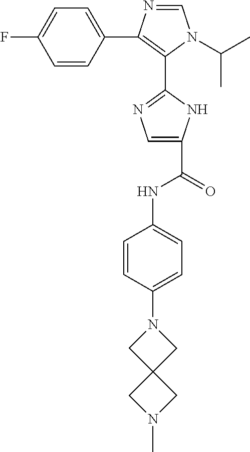 [SMILES: CC(C)n1cnc(-c2ccc(F)cc2)c1-c1ncc(C(=O)Nc2ccc(N3CC4(CN(C)C4)C3)cc2)[nH]1] | | +++ |
| 21 | 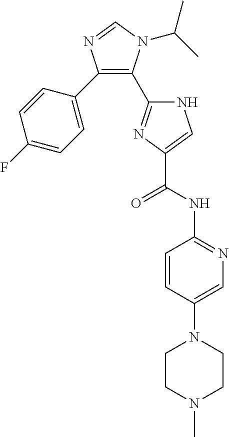 [SMILES: CC(C)n1cnc(-c2ccc(F)cc2)c1-c1nc(C(=O)Nc2ccc(N3CCN(C)CC3)cn2)c[nH]1] | ++ | +++ |
| 22 | 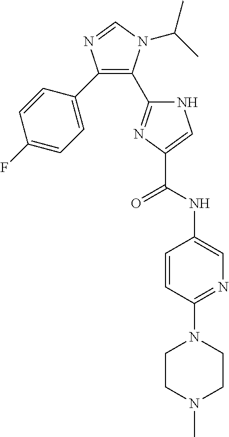 [SMILES: CC(C)n1cnc(-c2ccc(F)cc2)c1-c1nc(C(=O)Nc2ccc(N3CCN(C)CC3)nc2)c[nH]1] | | ++ |
| 23 | 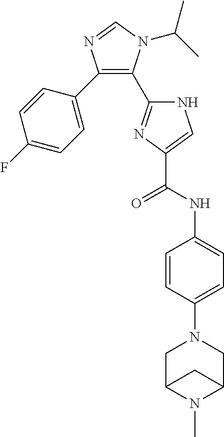 [SMILES: CC(C)n1cnc(-c2ccc(F)cc2)c1-c1nc(C(=O)Nc2ccc(N3CC4CC(C3)N4C)cc2)c[nH]1] | ++ | +++ |
| 24 | 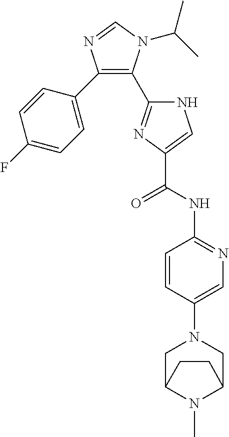 [SMILES: CC(C)n1cnc(-c2ccc(F)cc2)c1-c1nc(C(=O)Nc2ccc(N3CC4CCC(C3)N4C)cn2)c[nH]1] | ++ | +++ |
| 25 | 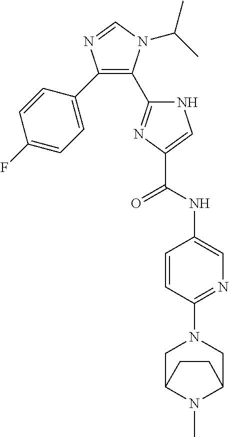 [SMILES: CC(C)n1cnc(-c2ccc(F)cc2)c1-c1nc(C(=O)Nc2ccc(N3CC4CCC(C3)N4C)nc2)c[nH]1] | | +++ |
| 26 | 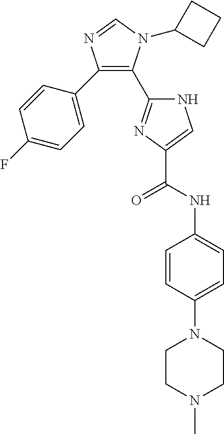 [SMILES: CN1CCN(c2ccc(NC(=O)c3c[nH]c(-c4c(-c5ccc(F)cc5)ncn4C4CCC4)n3)cc2)CC1] | ++ | ++ |
| 27 | 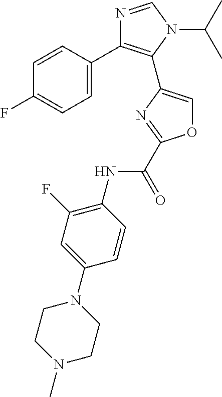 [SMILES: CC(C)n1cnc(-c2ccc(F)cc2)c1-c1coc(C(=O)Nc2ccc(N3CCN(C)CC3)cc2F)n1] | | ++ |
| 28 | 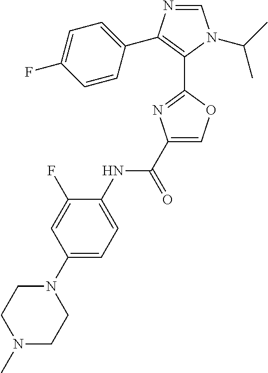 [SMILES: CC(C)n1cnc(-c2ccc(F)cc2)c1-c1nc(C(=O)Nc2ccc(N3CCN(C)CC3)cc2F)co1] | + | +++ |
| 29 | 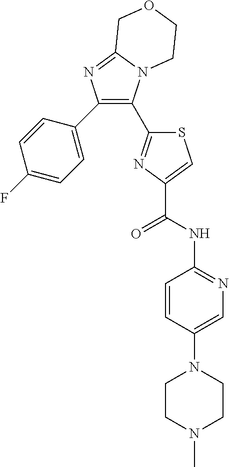 [SMILES: CN1CCN(c2ccc(NC(=O)c3csc(-c4c(-c5ccc(F)cc5)nc5n4CCOC5)n3)nc2)CC1] | | +++ |
| 30 | 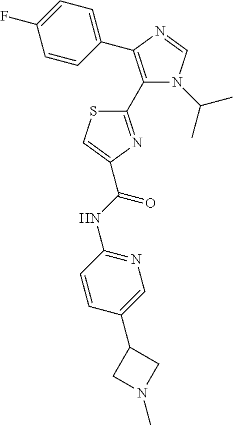 [SMILES: CC(C)n1cnc(-c2ccc(F)cc2)c1-c1nc(C(=O)Nc2ccc(C3CN(C)C3)cn2)cs1] | ++ | +++ |
| 31 | 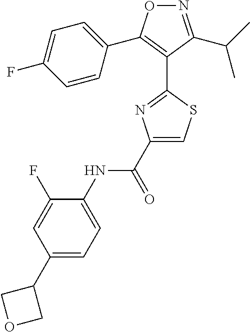 [SMILES: CC(C)c1noc(-c2ccc(F)cc2)c1-c1nc(C(=O)Nc2ccc(C3COC3)cc2F)cs1] | ++ | ++ |
| 32 | 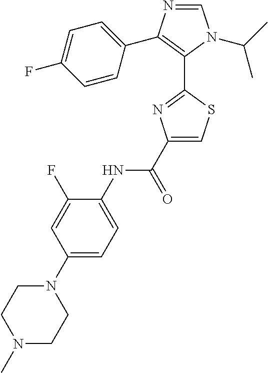 [SMILES: CC(C)n1cnc(-c2ccc(F)cc2)c1-c1nc(C(=O)Nc2ccc(N3CCN(C)CC3)cc2F)cs1] | + | +++ |
| 33 | 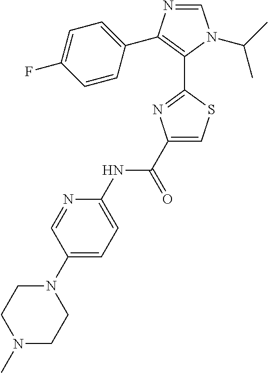 [SMILES: CC(C)n1cnc(-c2ccc(F)cc2)c1-c1nc(C(=O)Nc2ccc(N3CCN(C)CC3)cn2)cs1] | + | +++ |
| 34 | 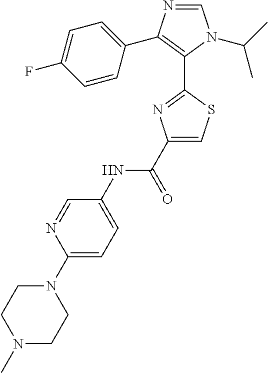 [SMILES: CC(C)n1cnc(-c2ccc(F)cc2)c1-c1nc(C(=O)Nc2ccc(N3CCN(C)CC3)nc2)cs1] | + | +++ |
| 35 | 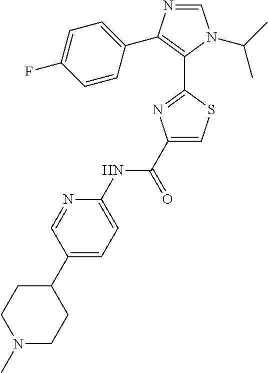 [SMILES: CC(C)n1cnc(-c2ccc(F)cc2)c1-c1nc(C(=O)Nc2ccc(C3CCN(C)CC3)cn2)cs1] | ++ | +++ |
| 36 | 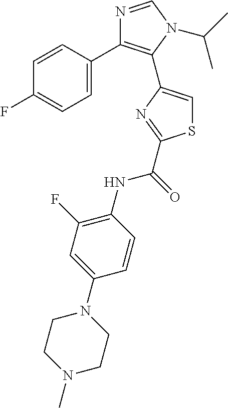 [SMILES: CC(C)n1cnc(-c2ccc(F)cc2)c1-c1csc(C(=O)Nc2ccc(N3CCN(C)CC3)cc2F)n1] | ++ | +++ |
| 37 | 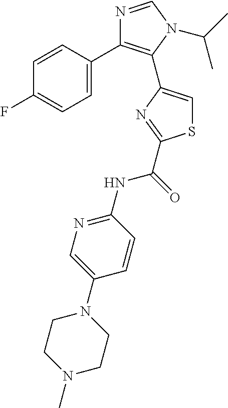 [SMILES: CC(C)n1cnc(-c2ccc(F)cc2)c1-c1csc(C(=O)Nc2ccc(N3CCN(C)CC3)cn2)n1] | + | +++ |
| 38 | 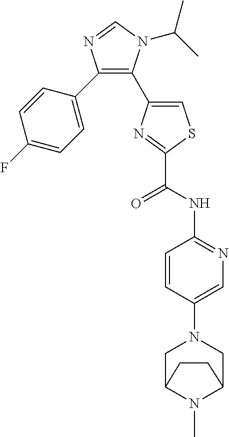 [SMILES: CC(C)n1cnc(-c2ccc(F)cc2)c1-c1csc(C(=O)Nc2ccc(N3CC4CCC(C3)N4C)cn2)n1] | | +++ |
| 39 | 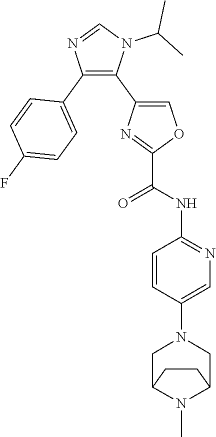 [SMILES: CC(C)n1cnc(-c2ccc(F)cc2)c1-c1coc(C(=O)Nc2ccc(N3CC4CCC(C3)N4C)cn2)n1] | | ++ |
| 40 | 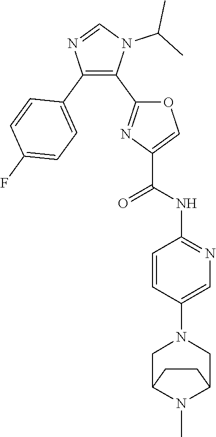 [SMILES: CC(C)n1cnc(-c2ccc(F)cc2)c1-c1nc(C(=O)Nc2ccc(N3CC4CCC(C3)N4C)cn2)co1] | + | +++ |
| 41 | 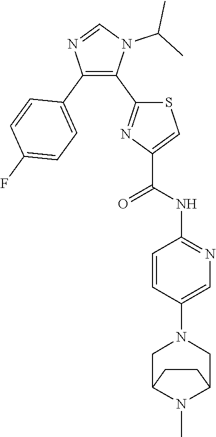 [SMILES: CC(C)n1cnc(-c2ccc(F)cc2)c1-c1nc(C(=O)Nc2ccc(N3CC4CCC(C3)N4C)cn2)cs1] | ++ | +++ |
| 42 | 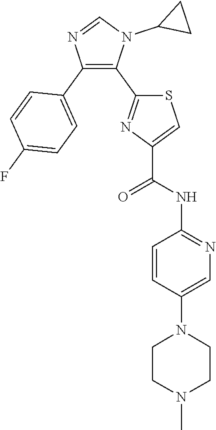 [SMILES: CN1CCN(c2ccc(NC(=O)c3csc(-c4c(-c5ccc(F)cc5)ncn4C4CC4)n3)nc2)CC1] | ++ | +++ |
| 43 | 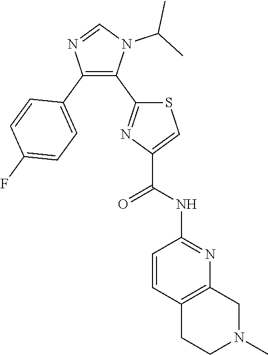 [SMILES: CC(C)n1cnc(-c2ccc(F)cc2)c1-c1nc(C(=O)Nc2ccc3c(n2)CN(C)CC3)cs1] | + | +++ |
| 44 | 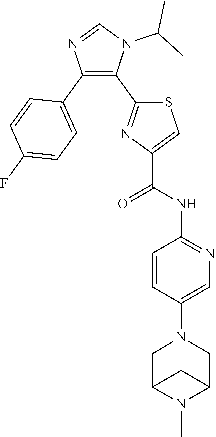 [SMILES: CC(C)n1cnc(-c2ccc(F)cc2)c1-c1nc(C(=O)Nc2ccc(N3CC4CC(C3)N4C)cn2)cs1] | ++ | +++ |
| 45 | 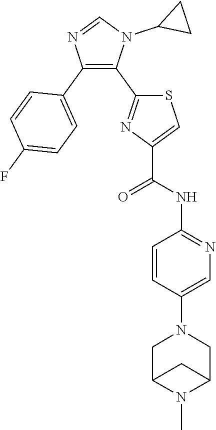 [SMILES: CN1C2CC1CN(c1ccc(NC(=O)c3csc(-c4c(-c5ccc(F)cc5)ncn4C4CC4)n3)nc1)C2] | ++ | +++ |
| 46 | 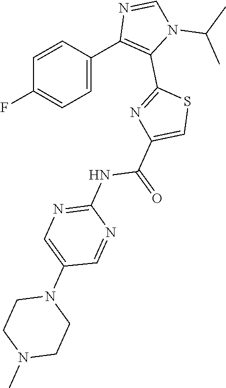 [SMILES: CC(C)n1cnc(-c2ccc(F)cc2)c1-c1nc(C(=O)Nc2ncc(N3CCN(C)CC3)cn2)cs1] | | + |
| 47 | 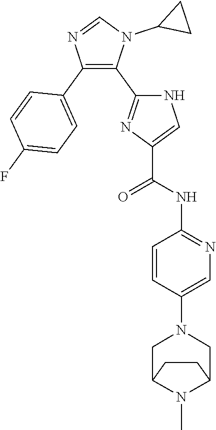 [SMILES: CN1C2CCC1CN(c1ccc(NC(=O)c3c[nH]c(-c4c(-c5ccc(F)cc5)ncn4C4CC4)n3)nc1)C2] | | +++ |
| 48 | 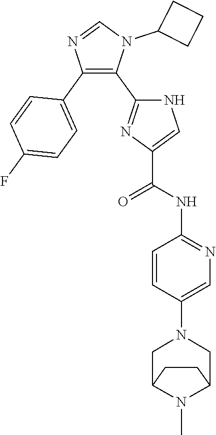 [SMILES: CN1C2CCC1CN(c1ccc(NC(=O)c3c[nH]c(-c4c(-c5ccc(F)cc5)ncn4C4CCC4)n3)nc1)C2] | | +++ |
| 49 | 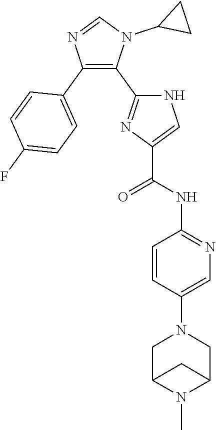 [SMILES: CN1C2CC1CN(c1ccc(NC(=O)c3c[nH]c(-c4c(-c5ccc(F)cc5)ncn4C4CC4)n3)nc1)C2] | | ++ |
| 50 | 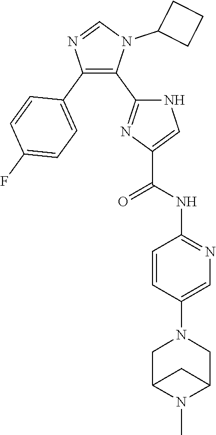 [SMILES: CN1C2CC1CN(c1ccc(NC(=O)c3c[nH]c(-c4c(-c5ccc(F)cc5)ncn4C4CCC4)n3)nc1)C2] | | +++ |
| 51 | 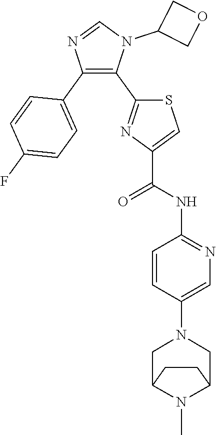 [SMILES: CN1C2CCC1CN(c1ccc(NC(=O)c3csc(-c4c(-c5ccc(F)cc5)ncn4C4COC4)n3)nc1)C2] | ++ | +++ |
| 52 | 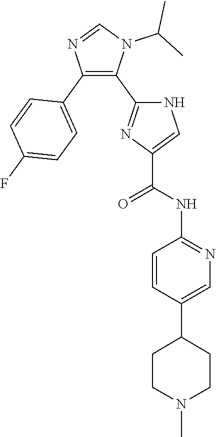 [SMILES: CC(C)n1cnc(-c2ccc(F)cc2)c1-c1nc(C(=O)Nc2ccc(C3CCN(C)CC3)cn2)c[nH]1] | | +++ |
| 53 | 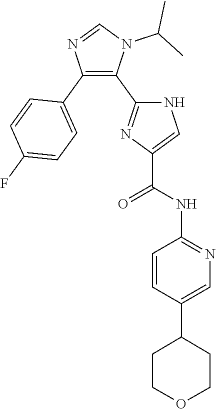 [SMILES: CC(C)n1cnc(-c2ccc(F)cc2)c1-c1nc(C(=O)Nc2ccc(C3CCOCC3)cn2)c[nH]1] | ++ | +++ |
| 54 | 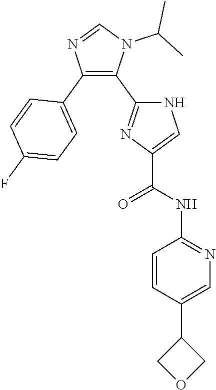 [SMILES: CC(C)n1cnc(-c2ccc(F)cc2)c1-c1nc(C(=O)Nc2ccc(C3COC3)cn2)c[nH]1] | ++ | +++ |
| 55 | 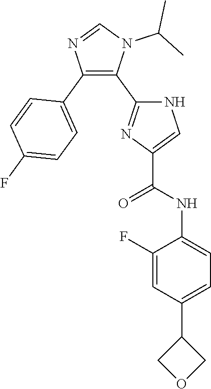 [SMILES: CC(C)n1cnc(-c2ccc(F)cc2)c1-c1nc(C(=O)Nc2ccc(C3COC3)cc2F)c[nH]1] | ++ | +++ |
| 56 | 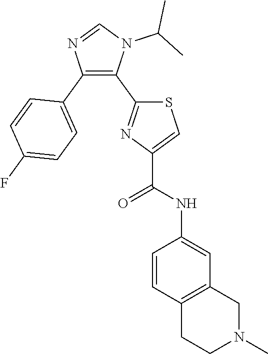 [SMILES: CC(C)n1cnc(-c2ccc(F)cc2)c1-c1nc(C(=O)Nc2ccc3c(c2)CN(C)CC3)cs1] | + | +++ |
| 57 | 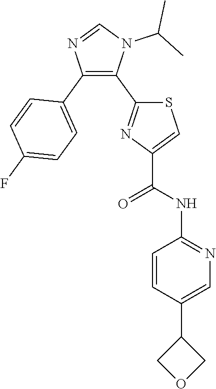 [SMILES: CC(C)n1cnc(-c2ccc(F)cc2)c1-c1nc(C(=O)Nc2ccc(C3COC3)cn2)cs1] | + | +++ |
| 58 | 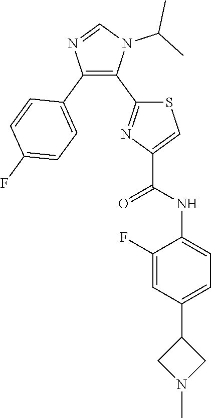 [SMILES: CC(C)n1cnc(-c2ccc(F)cc2)c1-c1nc(C(=O)Nc2ccc(C3CN(C)C3)cc2F)cs1] | ++ | +++ |
| 59 | 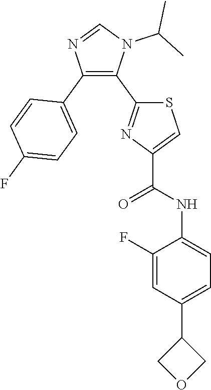 [SMILES: CC(C)n1cnc(-c2ccc(F)cc2)c1-c1nc(C(=O)Nc2ccc(C3COC3)cc2F)cs1] | + | +++ |
| 60 | 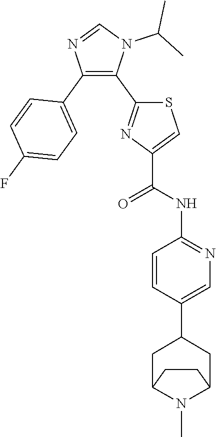 [SMILES: CC(C)n1cnc(-c2ccc(F)cc2)c1-c1nc(C(=O)Nc2ccc(C3CC4CCC(C3)N4C)cn2)cs1] | | +++ |
| 61 | 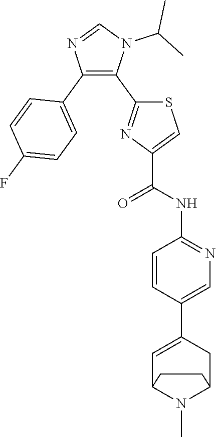 [SMILES: CC(C)n1cnc(-c2ccc(F)cc2)c1-c1nc(C(=O)Nc2ccc(C3=CC4CCC(C3)N4C)cn2)cs1] | ++ | +++ |
| 62 | 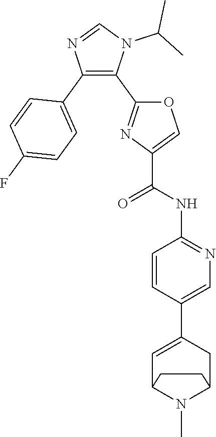 [SMILES: CC(C)n1cnc(-c2ccc(F)cc2)c1-c1nc(C(=O)Nc2ccc(C3=CC4CCC(C3)N4C)cn2)co1] | | ++ |
| 63 | 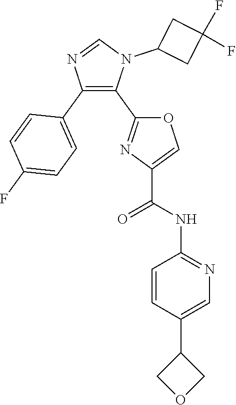 [SMILES: O=C(Nc1ccc(C2COC2)cn1)c1coc(-c2c(-c3ccc(F)cc3)ncn2C2CC(F)(F)C2)n1] | + | + |
| 64 | 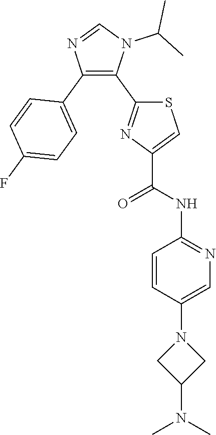 [SMILES: CC(C)n1cnc(-c2ccc(F)cc2)c1-c1nc(C(=O)Nc2ccc(N3CC(N(C)C)C3)cn2)cs1] | + | +++ |
| 65 | 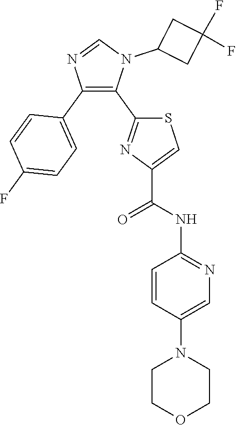 [SMILES: O=C(Nc1ccc(N2CCOCC2)cn1)c1csc(-c2c(-c3ccc(F)cc3)ncn2C2CC(F)(F)C2)n1] | ++ | +++ |
| 66 | 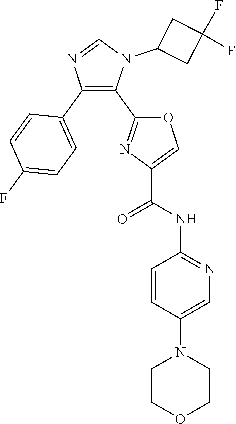 [SMILES: O=C(Nc1ccc(N2CCOCC2)cn1)c1coc(-c2c(-c3ccc(F)cc3)ncn2C2CC(F)(F)C2)n1] | ++ | ++ |
| 67 | 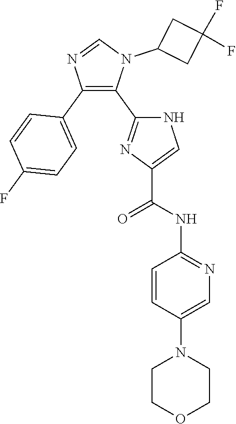 [SMILES: O=C(Nc1ccc(N2CCOCC2)cn1)c1c[nH]c(-c2c(-c3ccc(F)cc3)ncn2C2CC(F)(F)C2)n1] | | ++ |
| 68 | 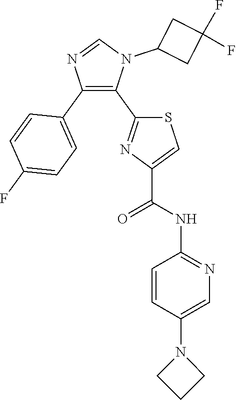 [SMILES: O=C(Nc1ccc(N2CCC2)cn1)c1csc(-c2c(-c3ccc(F)cc3)ncn2C2CC(F)(F)C2)n1] | ++ | +++ |
| 69 | 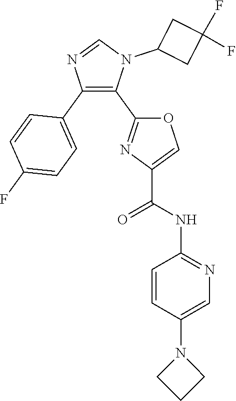 [SMILES: O=C(Nc1ccc(N2CCC2)cn1)c1coc(-c2c(-c3ccc(F)cc3)ncn2C2CC(F)(F)C2)n1] | | + |
| 70 | 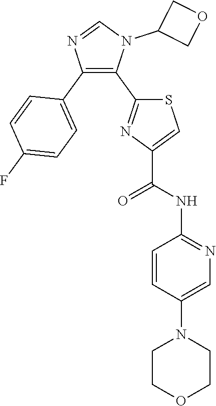 [SMILES: O=C(Nc1ccc(N2CCOCC2)cn1)c1csc(-c2c(-c3ccc(F)cc3)ncn2C2COC2)n1] | ++ | +++ |
| 71 | 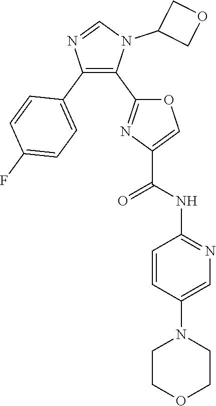 [SMILES: O=C(Nc1ccc(N2CCOCC2)cn1)c1coc(-c2c(-c3ccc(F)cc3)ncn2C2COC2)n1] | | + |
| 72 | 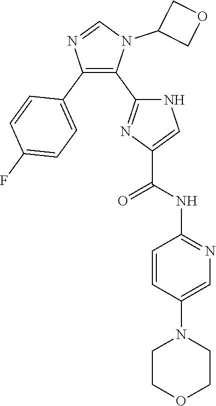 [SMILES: O=C(Nc1ccc(N2CCOCC2)cn1)c1c[nH]c(-c2c(-c3ccc(F)cc3)ncn2C2COC2)n1] | ++ | |
| 73 | 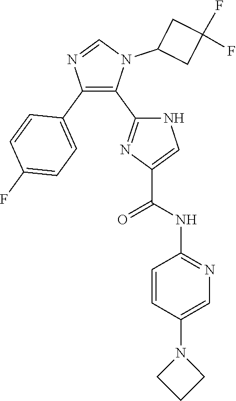 [SMILES: O=C(Nc1ccc(N2CCC2)cn1)c1c[nH]c(-c2c(-c3ccc(F)cc3)ncn2C2CC(F)(F)C2)n1] | | ++ |
| 74 | 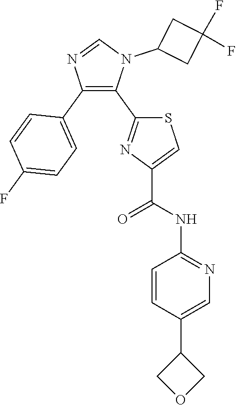 [SMILES: O=C(Nc1ccc(C2COC2)cn1)c1csc(-c2c(-c3ccc(F)cc3)ncn2C2CC(F)(F)C2)n1] | + | +++ |
| 75 | 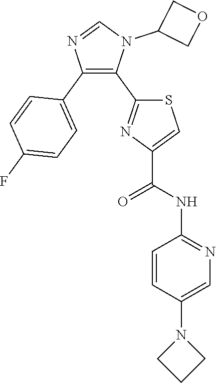 [SMILES: O=C(Nc1ccc(N2CCC2)cn1)c1csc(-c2c(-c3ccc(F)cc3)ncn2C2COC2)n1] | | +++ |
| 76 | 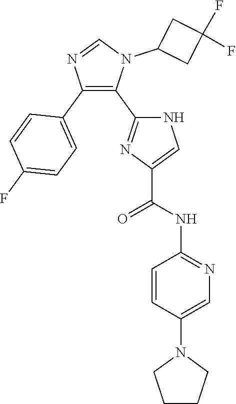 [SMILES: O=C(Nc1ccc(N2CCCC2)cn1)c1c[nH]c(-c2c(-c3ccc(F)cc3)ncn2C2CC(F)(F)C2)n1] | | ++ |
| 77 | 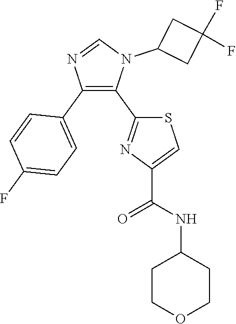 [SMILES: O=C(NC1CCOCC1)c1csc(-c2c(-c3ccc(F)cc3)ncn2C2CC(F)(F)C2)n1] | | + |
| 78 | 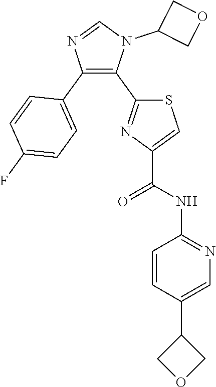 [SMILES: O=C(Nc1ccc(C2COC2)cn1)c1csc(-c2c(-c3ccc(F)cc3)ncn2C2COC2)n1] | | +++ |
| 79 | 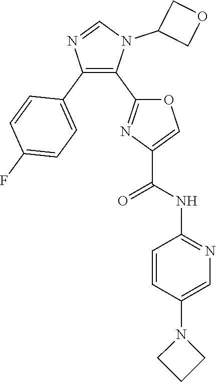 [SMILES: O=C(Nc1ccc(N2CCC2)cn1)c1coc(-c2c(-c3ccc(F)cc3)ncn2C2COC2)n1] | | ++ |
| 80 | 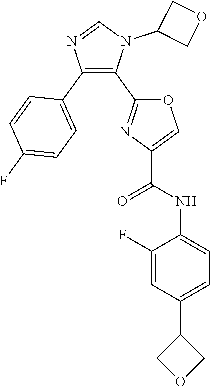 [SMILES: O=C(Nc1ccc(C2COC2)cc1F)c1coc(-c2c(-c3ccc(F)cc3)ncn2C2COC2)n1] | | ++ |
| 81 | 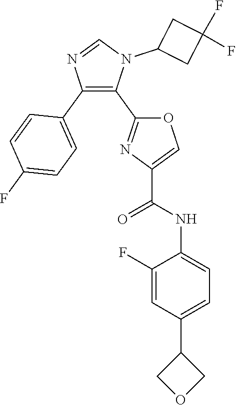 [SMILES: O=C(Nc1ccc(C2COC2)cc1F)c1coc(-c2c(-c3ccc(F)cc3)ncn2C2CC(F)(F)C2)n1] | + | ++ |
| 82 | 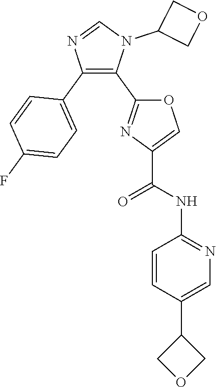 [SMILES: O=C(Nc1ccc(C2COC2)cn1)c1coc(-c2c(-c3ccc(F)cc3)ncn2C2COC2)n1] | | ++ |
| 83 | 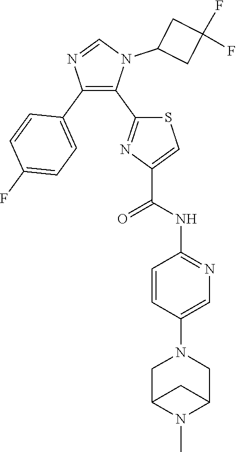 [SMILES: CN1C2CC1CN(c1ccc(NC(=O)c3csc(-c4c(-c5ccc(F)cc5)ncn4C4CC(F)(F)C4)n3)nc1)C2] | ++ | +++ |
| 84 | 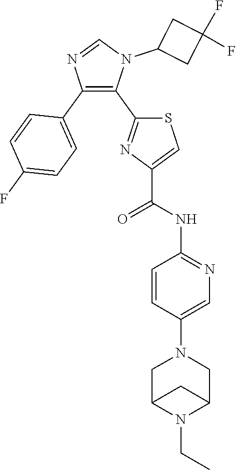 [SMILES: CCN1C2CC1CN(c1ccc(NC(=O)c3csc(-c4c(-c5ccc(F)cc5)ncn4C4CC(F)(F)C4)n3)nc1)C2] | ++ | +++ |
| 85 | 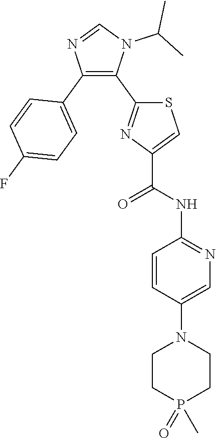 [SMILES: CC(C)n1cnc(-c2ccc(F)cc2)c1-c1nc(C(=O)Nc2ccc(N3CCP(C)(=O)CC3)cn2)cs1] | ++ | +++ |
| 86 | 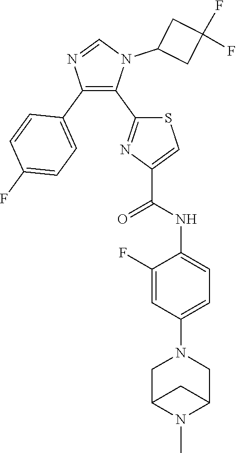 [SMILES: CN1C2CC1CN(c1ccc(NC(=O)c3csc(-c4c(-c5ccc(F)cc5)ncn4C4CC(F)(F)C4)n3)c(F)c1)C2] | ++ | +++ |
| 87 | 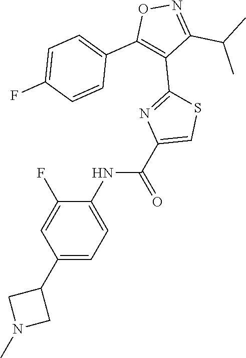 [SMILES: CC(C)c1noc(-c2ccc(F)cc2)c1-c1nc(C(=O)Nc2ccc(C3CN(C)C3)cc2F)cs1] | | +++ |
| 88 | 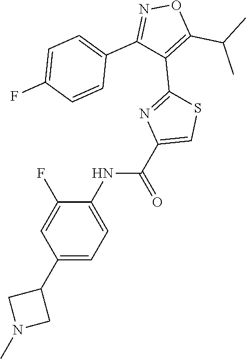 [SMILES: CC(C)c1onc(-c2ccc(F)cc2)c1-c1nc(C(=O)Nc2ccc(C3CN(C)C3)cc2F)cs1] | ++ | +++ |
| 89 | 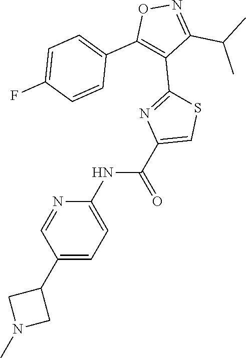 [SMILES: CC(C)c1noc(-c2ccc(F)cc2)c1-c1nc(C(=O)Nc2ccc(C3CN(C)C3)cn2)cs1] | | ++ |
| 90 | 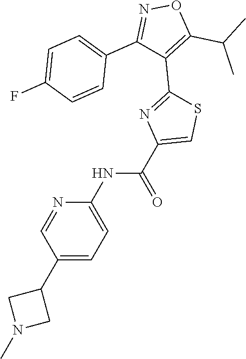 [SMILES: CC(C)c1onc(-c2ccc(F)cc2)c1-c1nc(C(=O)Nc2ccc(C3CN(C)C3)cn2)cs1] | ++ | +++ |
| 91 | 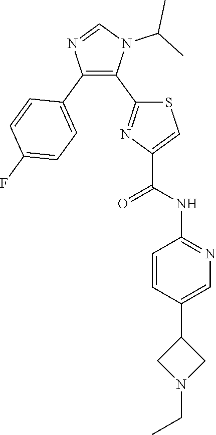 [SMILES: CCN1CC(c2ccc(NC(=O)c3csc(-c4c(-c5ccc(F)cc5)ncn4C(C)C)n3)nc2)C1] | ++ | +++ |
| 92 | 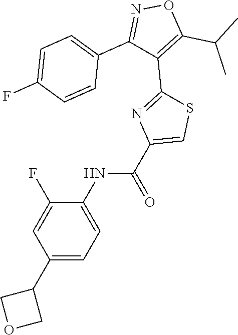 [SMILES: CC(C)c1onc(-c2ccc(F)cc2)c1-c1nc(C(=O)Nc2ccc(C3COC3)cc2F)cs1] | ++ | ++ |
| 93 | 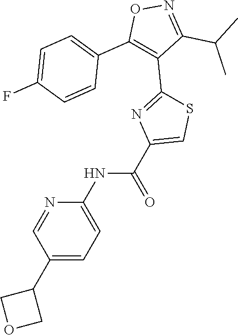 [SMILES: CC(C)c1noc(-c2ccc(F)cc2)c1-c1nc(C(=O)Nc2ccc(C3COC3)cn2)cs1] | ++ | +++ |
| 94 | 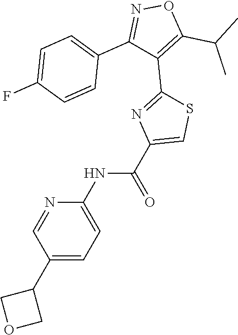 [SMILES: CC(C)c1onc(-c2ccc(F)cc2)c1-c1nc(C(=O)Nc2ccc(C3COC3)cn2)cs1] | ++ | +++ |
| 95 | 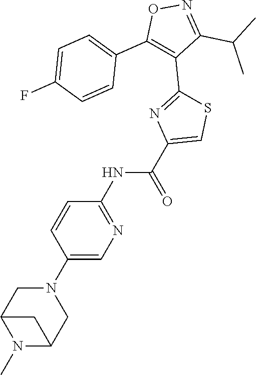 [SMILES: CC(C)c1noc(-c2ccc(F)cc2)c1-c1nc(C(=O)Nc2ccc(N3CC4CC(C3)N4C)cn2)cs1] | ++ | +++ |
| 96 | 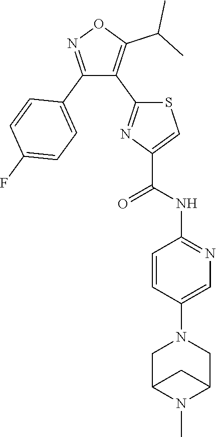 [SMILES: CC(C)c1onc(-c2ccc(F)cc2)c1-c1nc(C(=O)Nc2ccc(N3CC4CC(C3)N4C)cn2)cs1] | ++ | +++ |
| 97 | 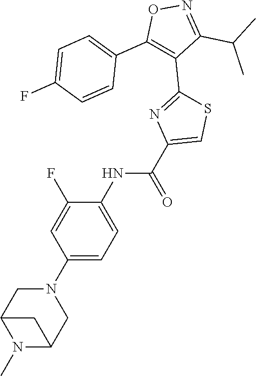 [SMILES: CC(C)c1noc(-c2ccc(F)cc2)c1-c1nc(C(=O)Nc2ccc(N3CC4CC(C3)N4C)cc2F)cs1] | ++ | +++ |
| 98 | 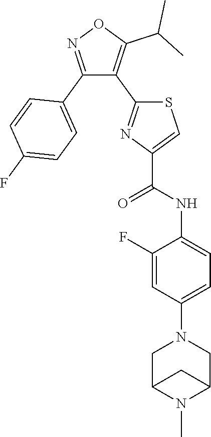 [SMILES: CC(C)c1onc(-c2ccc(F)cc2)c1-c1nc(C(=O)Nc2ccc(N3CC4CC(C3)N4C)cc2F)cs1] | ++ | +++ |
| 99 | 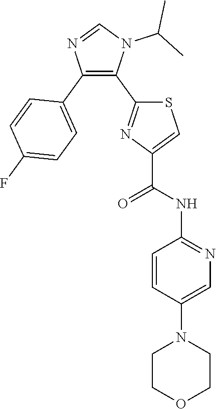 [SMILES: CC(C)n1cnc(-c2ccc(F)cc2)c1-c1nc(C(=O)Nc2ccc(N3CCOCC3)cn2)cs1] | ++ | +++ |
| 100 | 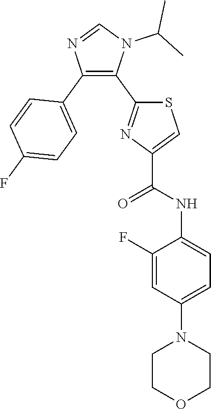 [SMILES: CC(C)n1cnc(-c2ccc(F)cc2)c1-c1nc(C(=O)Nc2ccc(N3CCOCC3)cc2F)cs1] | ++ | +++ |
| 101 | 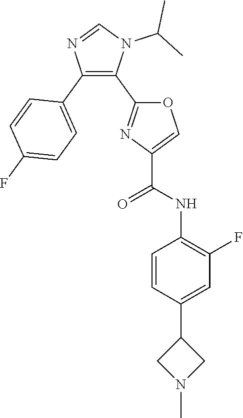 [SMILES: CC(C)n1cnc(-c2ccc(F)cc2)c1-c1nc(C(=O)Nc2ccc(C3CN(C)C3)cc2F)co1] | | +++ |
| 102 | 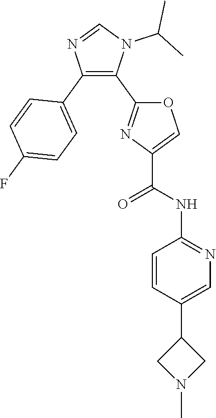 [SMILES: CC(C)n1cnc(-c2ccc(F)cc2)c1-c1nc(C(=O)Nc2ccc(C3CN(C)C3)cn2)co1] | ++ | ++ |
| 103 | 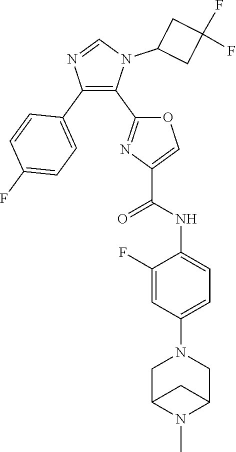 [SMILES: CN1C2CC1CN(c1ccc(NC(=O)c3coc(-c4c(-c5ccc(F)cc5)ncn4C4CC(F)(F)C4)n3)c(F)c1)C2] | | +++ |
| 104 | 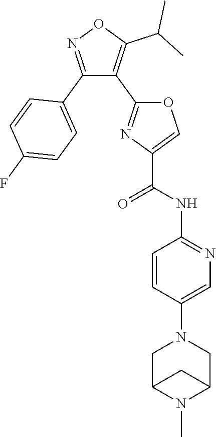 [SMILES: CC(C)c1onc(-c2ccc(F)cc2)c1-c1nc(C(=O)Nc2ccc(N3CC4CC(C3)N4C)cn2)co1] | | ++ |
| 105 | 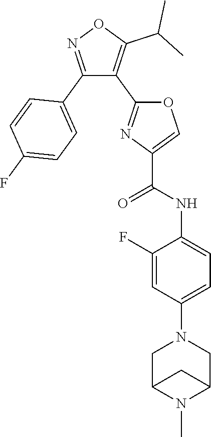 [SMILES: CC(C)c1onc(-c2ccc(F)cc2)c1-c1nc(C(=O)Nc2ccc(N3CC4CC(C3)N4C)cc2F)co1] | | ++ |
| 106 | 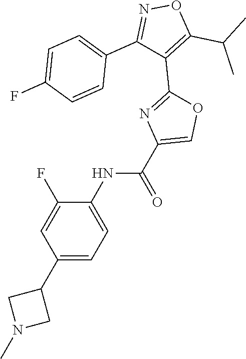 [SMILES: CC(C)c1onc(-c2ccc(F)cc2)c1-c1nc(C(=O)Nc2ccc(C3CN(C)C3)cc2F)co1] | | ++ |
| 107 | 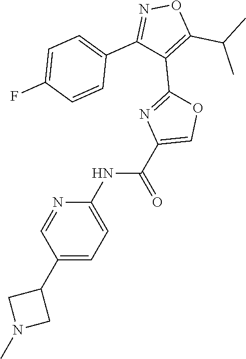 [SMILES: CC(C)c1onc(-c2ccc(F)cc2)c1-c1nc(C(=O)Nc2ccc(C3CN(C)C3)cn2)co1] | | ++ |
| 108 | 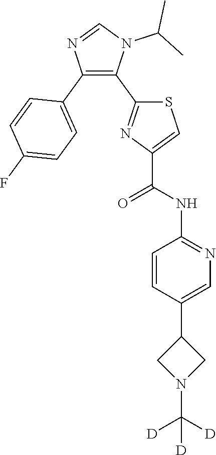 [SMILES: [2H]C([2H])([2H])N1CC(c2ccc(NC(=O)c3csc(-c4c(-c5ccc(F)cc5)ncn4C(C)C)n3)nc2)C1] | ++ | +++ |
| 109 | 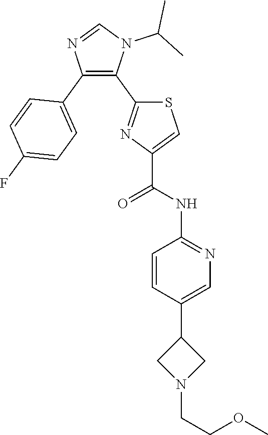 [SMILES: COCCN1CC(c2ccc(NC(=O)c3csc(-c4c(-c5ccc(F)cc5)ncn4C(C)C)n3)nc2)C1] | ++ | +++ |
| 110 | 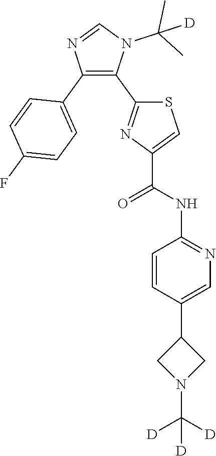 [SMILES: [2H]C([2H])([2H])N1CC(c2ccc(NC(=O)c3csc(-c4c(-c5ccc(F)cc5)ncn4C([2H])(C)C)n3)nc2)C1] | | +++ |
| 111 | 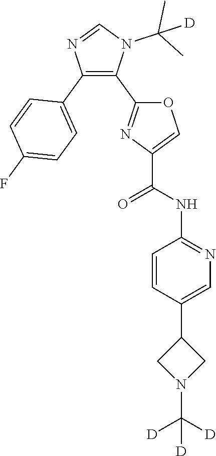 [SMILES: [2H]C([2H])([2H])N1CC(c2ccc(NC(=O)c3coc(-c4c(-c5ccc(F)cc5)ncn4C([2H])(C)C)n3)nc2)C1] | | ++ |
| 112 | 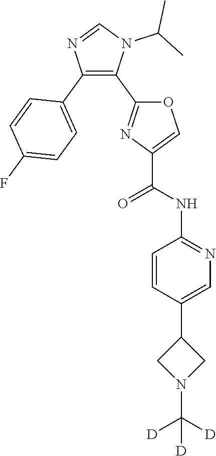 [SMILES: [2H]C([2H])([2H])N1CC(c2ccc(NC(=O)c3coc(-c4c(-c5ccc(F)cc5)ncn4C(C)C)n3)nc2)C1] | | ++ |
| 113 | 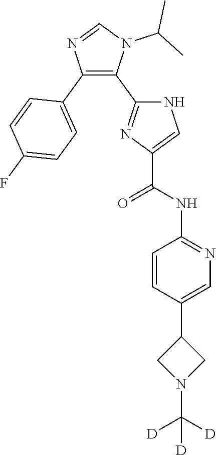 [SMILES: [2H]C([2H])([2H])N1CC(c2ccc(NC(=O)c3c[nH]c(-c4c(-c5ccc(F)cc5)ncn4C(C)C)n3)nc2)C1] | | ++ |
| 114 | 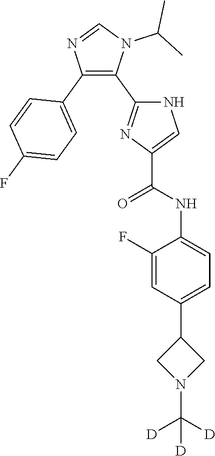 [SMILES: [2H]C([2H])([2H])N1CC(c2ccc(NC(=O)c3c[nH]c(-c4c(-c5ccc(F)cc5)ncn4C(C)C)n3)c(F)c2)C1] | +++ | +++ |
| 115 | 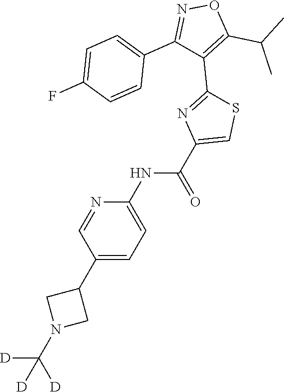 [SMILES: [2H]C([2H])([2H])N1CC(c2ccc(NC(=O)c3csc(-c4c(-c5ccc(F)cc5)noc4C(C)C)n3)nc2)C1] | ++ | +++ |
| 116 | 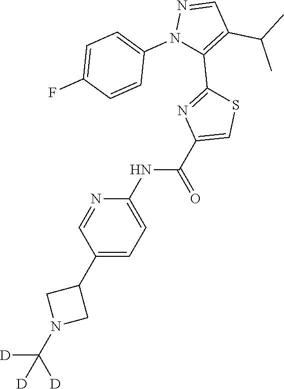 [SMILES: [2H]C([2H])([2H])N1CC(c2ccc(NC(=O)c3csc(-c4c(C(C)C)cnn4-c4ccc(F)cc4)n3)nc2)C1] | | +++ |
| 117 | 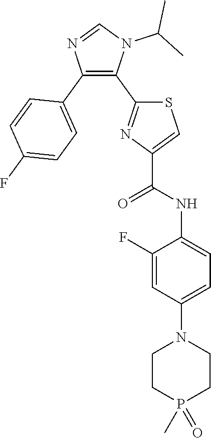 [SMILES: CC(C)n1cnc(-c2ccc(F)cc2)c1-c1nc(C(=O)Nc2ccc(N3CCP(C)(=O)CC3)cc2F)cs1] | ++ | +++ |
| 118 | 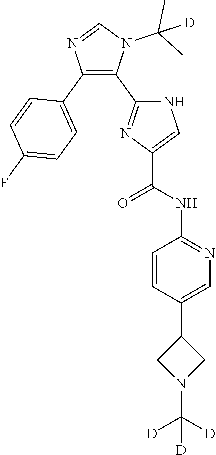 [SMILES: [2H]C([2H])([2H])N1CC(c2ccc(NC(=O)c3c[nH]c(-c4c(-c5ccc(F)cc5)ncn4C([2H])(C)C)n3)nc2)C1] | | ++ |
| 119 | 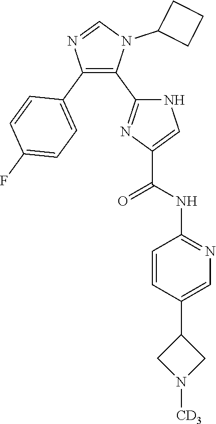 [SMILES: [2H]C([2H])([2H])N1CC(c2ccc(NC(=O)c3c[nH]c(-c4c(-c5ccc(F)cc5)ncn4C4CCC4)n3)nc2)C1] | | ++ |
| 120 | 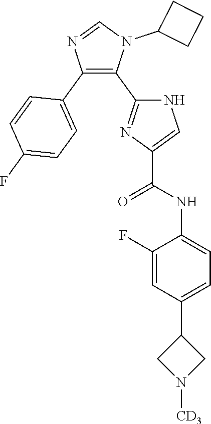 [SMILES: [2H]C([2H])([2H])N1CC(c2ccc(NC(=O)c3c[nH]c(-c4c(-c5ccc(F)cc5)ncn4C4CCC4)n3)c(F)c2)C1] | | +++ |
| 121 | 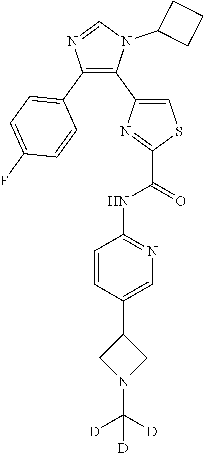 [SMILES: [2H]C([2H])([2H])N1CC(c2ccc(NC(=O)c3nc(-c4c(-c5ccc(F)cc5)ncn4C4CCC4)cs3)nc2)C1] | ++ | +++ |
| 122 | 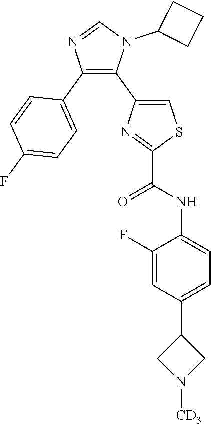 [SMILES: [2H]C([2H])([2H])N1CC(c2ccc(NC(=O)c3nc(-c4c(-c5ccc(F)cc5)ncn4C4CCC4)cs3)c(F)c2)C1] | ++ | +++ |
| 123 | 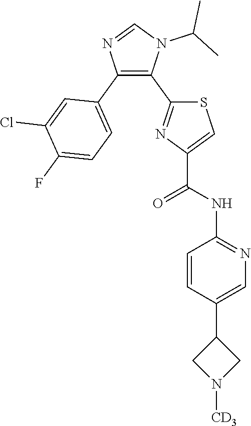 [SMILES: [2H]C([2H])([2H])N1CC(c2ccc(NC(=O)c3csc(-c4c(-c5ccc(F)c(Cl)c5)ncn4C(C)C)n3)nc2)C1] | ++ | +++ |
| 124 | 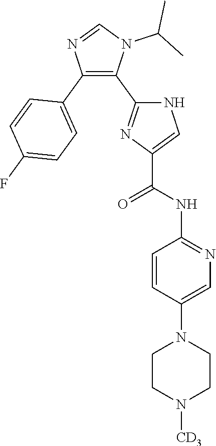 [SMILES: [2H]C([2H])([2H])N1CCN(c2ccc(NC(=O)c3c[nH]c(-c4c(-c5ccc(F)cc5)ncn4C(C)C)n3)nc2)CC1] | ++ | +++ |
| 125 | 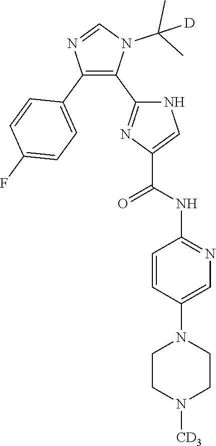 [SMILES: [2H]C([2H])([2H])N1CCN(c2ccc(NC(=O)c3c[nH]c(-c4c(-c5ccc(F)cc5)ncn4C([2H])(C)C)n3)nc2)CC1] | ++ | +++ |
| 126 | 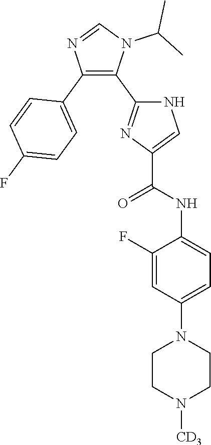 [SMILES: [2H]C([2H])([2H])N1CCN(c2ccc(NC(=O)c3c[nH]c(-c4c(-c5ccc(F)cc5)ncn4C(C)C)n3)c(F)c2)CC1] | ++ | +++ |
| 127 | 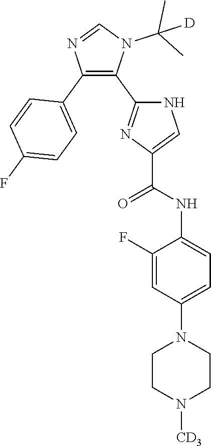 [SMILES: [2H]C([2H])([2H])N1CCN(c2ccc(NC(=O)c3c[nH]c(-c4c(-c5ccc(F)cc5)ncn4C([2H])(C)C)n3)c(F)c2)CC1] | ++ | +++ |
| 128 | 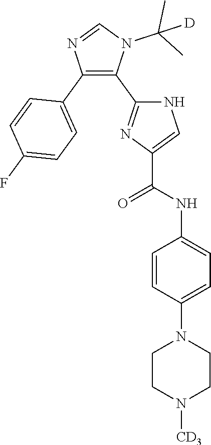 [SMILES: [2H]C([2H])([2H])N1CCN(c2ccc(NC(=O)c3c[nH]c(-c4c(-c5ccc(F)cc5)ncn4C([2H])(C)C)n3)cc2)CC1] | ++ | ++ |
| 129 | 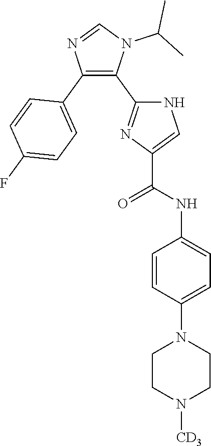 [SMILES: [2H]C([2H])([2H])N1CCN(c2ccc(NC(=O)c3c[nH]c(-c4c(-c5ccc(F)cc5)ncn4C(C)C)n3)cc2)CC1] | ++ | +++ |
| 130 | 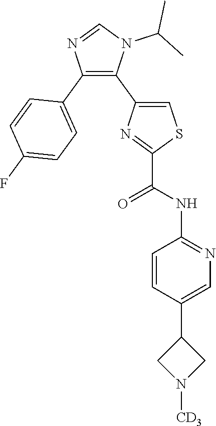 [SMILES: [2H]C([2H])([2H])N1CC(c2ccc(NC(=O)c3nc(-c4c(-c5ccc(F)cc5)ncn4C(C)C)cs3)nc2)C1] | ++ | |
| 131 | 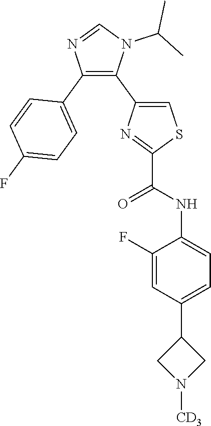 [SMILES: [2H]C([2H])([2H])N1CC(c2ccc(NC(=O)c3nc(-c4c(-c5ccc(F)cc5)ncn4C(C)C)cs3)c(F)c2)C1] | ++ | |
| 132 | 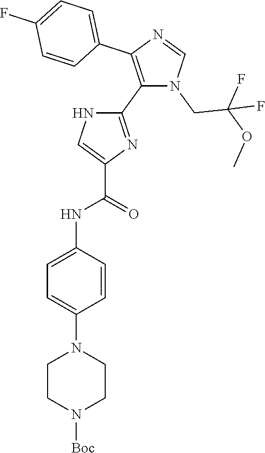 [SMILES: COC(F)(F)Cn1cnc(-c2ccc(F)cc2)c1-c1nc(C(=O)Nc2ccc(N3CCN(C(=O)OC(C)(C)C)CC3)cc2)c[nH]1] | | |
| 133 | 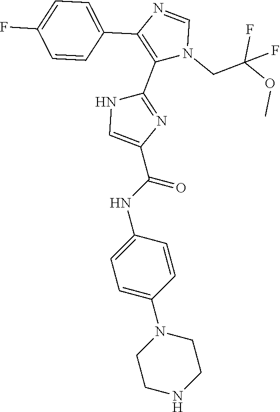 [SMILES: COC(F)(F)Cn1cnc(-c2ccc(F)cc2)c1-c1nc(C(=O)Nc2ccc(N3CCNCC3)cc2)c[nH]1] | | |
| 134 | 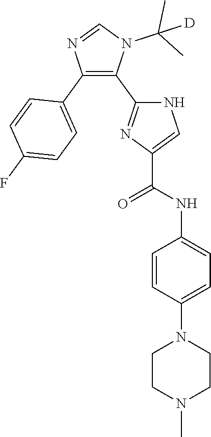 [SMILES: [2H]C(C)(C)n1cnc(-c2ccc(F)cc2)c1-c1nc(C(=O)Nc2ccc(N3CCN(C)CC3)cc2)c[nH]1] | | |
| 135 | 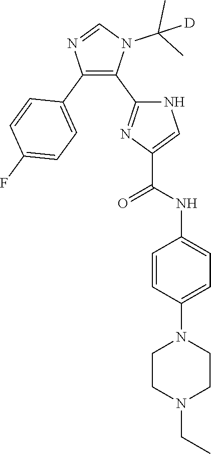 [SMILES: [2H]C(C)(C)n1cnc(-c2ccc(F)cc2)c1-c1nc(C(=O)Nc2ccc(N3CCN(CC)CC3)cc2)c[nH]1] | | ++ |

TABLE 1.1
Compound Information
Example 136: Metabolic Stability in Human and Mouse Liver Microsomes

| | | | | |
|---|---|---|---|---|
| | | | | Stock |
| Compound | Compound | Batch | Exact | Concentration |
| No. | ID | No. | Mass | (mM) |

| | | | |
|---|---|---|---|
| 1 | XXX | | 10 |
| Control | Testosterone | 288.42 | 10 |
| Control | Diclofenac | 295.14 | 10 |
| Control | Propafenone | 341.44 | 10 |

2.1. Test Compound and Control Working Solution Preparation: Working solution: 5 L of compound and control stock solution (10 mM in dimethyl sulfoxide (DMSO)) were diluted with 495 μL of acetonitrile (ACN) (intermediate solution concentration: 100 μM, 99% ACN).

### 2.2. NADPH Cofactor Preparation

2.2.1. Materials: NADPH powder: β-Nicotinamide adenine dinucleotide phosphate reduced form, tetrasodium salt; NADPH-4Na (Vendor: Chem-Impex International, Cat. No. 00616).
2.2.2. Preparation Procedure: The appropriate amount of NADPH powder were weighed and diluted into a 10 mM MgCl2 solution (working solution concentration: 10 unit/mL; final concentration in reaction system: 1 unit/mL).

### 2.3. Liver Microsomes Preparation:

### 2.3.1. Materials:

TABLE 2.1
Liver Microsomes Information

| | Product | | |
|---|---|---|---|
| Species | Information | Vendor | Abbrevation |
| Human | Cat No. | Corning | HLM |
| | 452117 | | |
| | Lot No. | | |
| | 38295 | | |
| CD-1 | Cat No. | BioIVT | MLM |
| Mouse | M00501 | | |
| | Lot | | |
| | No. WQP | | |

2.3.2. Preparation Procedure: The appropriate concentrations of microsome working solutions were prepared in 100 mM potassium phosphate buffer.
2.4. Stop Solution Preparation: Cold (4° C.) acetonitrile (ACN) containing 200 ng/mL tolbutamide and 200 ng/mL labetalol as internal standards (IS) were used as the stop solution.

### 2.5. Assay Procedure:

2.5.1. Pre-warm empty ‘Incubation’ plates T60 and NCF60 for 10 min minutes.
2.5.2. Liver microsomes were diluted to 0.56 mg/mL in 100 mM phosphate buffer.
2.5.3. 445 uL microsome working solutions (0.56 mg/mL) were transferred into pre-warmed ‘Incubation’ plates T60 and NCF60. Then ‘Incubation’ plates T60 and NCF60 were pre-incubated for 10 min at 37° C. with constant shaking. 54 μL liver microsomes were transferred to blank plate, then 6 μL NAPDH cofactor were added to the blank plate, and then 180 μL quenching solution can be added to blank plate.
2.5.4 5 μL compound working solution (100 μM) were added into ‘incubation’ plates (T60 and NCF60) containing microsomes and mix 3 times thoroughly.
2.5.5. For the NCF60 plate, 50 uL of buffer were added and were mixed 3 times thoroughly. Start timing; plate was incubated at 37° C. for 60 min while shaking.
2.5.6. In ‘Quenching’ plate TO, 180 μL quenching solution were added and 6 μL NAPDH cofactor were added. Ensured the plate was chilled to prevent evaporation.
2.5.7. For the T60 plate, were mixed 3 times thoroughly, and immediately removed 54 μL mixture for the 0-min time point to ‘Quenching’ plate. Then 44 μL NAPDH cofactor were added to incubation plate (T60). Start timing; plate were incubated at 37° C. for 60 min while shaking.

TABLE 2.2
Final Concentration of Each Component in Incubation Medium

| Component | Concentration |
|---|---|
| Microsome | 0.5 mg protein/mL |
| Test Compound | 1 μM |
| Control Compound | 1 μM |
| Acetonitrile | 0.99% |
| DMSO | 0.01% |

2.5.8. At 5, 10, 20, 30, and 60 min, 180 μL quenching solution were added to ‘Quenching’ plates, mix once, and were serially transferred 60 μL sample from T60 plate per time point to ‘Quenching’ plates.

TABLE 2.3
Reaction Plates Incubation

| Time Point | Start Time | End Time |
|---|---|---|
| Blank | 1:00:00 | 0:00:00 |
| T60 | 1:00:00 | 0:00:00 |
| T30 | 0:30:00 | 0:00:00 |
| T20 | 0:20:00 | 0:00:00 |
| T10 | 0:10:00 | 0:00:00 |
| T5 | 0:05:00 | 0:00:00 |

| | |
|---|---|
| T0 | mix 3 times and remove out |
| | to ‘Quenching’ plate |

2.5.9. For NCF60: were mixed once, and 60 μL sample were transferred from the NCF60 incubation to ‘Quenching’ plate containing quenching solution at the 60-min time point.

TABLE 2.4
NCF60 Incubation

| | | End |
|---|---|---|
| Time Point | Start Time | Time |
| NCF60 | 1:00:00 | 0:00:00 |

2.5.10. All sampling plates were shaken for 10 min, then centrifuged at 4000 rpm for 20 minutes at 4° C.
2.5.11. 80 μL supernatant were transferred into 240 μL HPLC water, and were mixed by plate shaker for 10 min.
2.5.12. Each bioanalysis plate were sealed and shaken for 10 minutes prior to LC-MS/MS analysis.

3.1. The equation of first order kinetics were used to calculate T1/2 and Intrinsic clearance (CLint mic) in (μL/min/mg).

Equation of first order kinetic.

C₁ = C₀ • e^{-k}^{ₑ}^{•t}
$when {C}_{t} = \frac{1}{2} {C}_{0} ,$
${T}_{1 / 2} = \frac{Ln 2}{{k}_{c}} = \frac{0.693}{{k}_{c}}$
${CL}_{i n t ( mic )} = \frac{0.693}{In vitro {T}_{1 / 2}} \cdot \frac{1}{mg / mL microsomal protein in reaction system}$
${CL}_{{int}_{( liver )}} = {CL}_{{int}_{( mic )}} \cdot \frac{mg microsomes}{g liver} \cdot \frac{g liver}{kg body weight}$

Table 1 includes μM/min/mg values of selected compounds; compounds having a LM Clint of μM/min/mg of 1-10 μM/min/mg as +++, 10-100 μM/min/mg as ++, and >100 μM/min/mg as +.

### Example 137: TNIK Human STE Kinase Enzymatic Radiometric Assay

Z-lyte assay kit was purchased from Invitrogen. A site-specific protease recognizes and cleaves non-phosphorylated FRET-peptides. Phosphorylation of FRET-peptides suppresses cleavage by protease and will maintain FRET from donor to acceptor.

Inhibition of Tnik activity was assessed by incubating 10 μl assay solution system containing 0.625 nM TNIK kinase [amino acid sequence (1-367), Invitrogen], 2 μM Ser/Thr 7 peptide, 64 μM ATP with assay buffer 50 mM Hepes (pH7.5), 10 mM MgCl₂, 1 mM EDTA, 0.01% Brij 35 in the presence or absence of inhibitor for one hour at room temperature. 5 μl of Development Reagent A solution was then added to cleave non-phosphorylated peptide with a dilution 1:65000 in Development Buffer B. The assay plate was incubated at room temperature for another one hour and then read in the Envision plate reader (PerkinElmer).

The test compounds were dispensed to 384 microplates by Echo (Labcyte) which performs 11 concentrations with 3-fold dilution as the final concentrations varied from 10 μM to 0.51 nM. The IC₅₀ data of test compound was generated by using four parameters curve fitting (Model 205 in XLFIT5, IDBS).

Table 1 includes IC50 values for TNIK of selected compounds; compounds having an IC50 value of 1-12 nM as +++, 12-150 nM as ++, and >150 nM as +.

### Example 138: MAP4K4 Human STE Kinase Enzymatic Radiometric Assay

ASSAY TYPE: Biochemical

ASSAY SUB TYPE: Enzymatic

FUNCTIONAL MODE: Antagonist

DETECTION METHOD: Radiometric

MEASURED RESPONSE: Scintillation

PROCEDURE SUMMARY: MAP4K4 (h) will be incubated with 8 mM MOPS pH 7.0, 0.2 mM EDTA, 250 μM RLGRDKYKTLRQIRQ, 10 mM Magnesium Acetate and [gamma-33P-ATP] (specific activity and concentration as required). The reaction will be initiated by the addition of the Mg/ATP mix. After incubation for 40 minutes at room temperature, the reaction will be stopped by the addition of phosphoric acid to a concentration of 0.5%. 10 ul of the reaction will then be spotted onto a P30 filtermat and washed four times for 4 minutes in 0.425% phosphoric acid and once in methanol prior to drying and scintillation counting.

SUBSTRATE: 250 uM RLGRDKYKTLRQI

TRACER: 33P

ATP CONCENTRATION: 200 μM

INCUBATION: 40 min at Room temperature

CONTROL INHIBITOR: Staurosporine

COMPOUND CONCENTRATION: 10 μM, 3 μM, 1 μM, 0.3 μM, 0.1 μM, 0.03 μM, 0.01 μM, 0.003 μM, 0.001 μM.

COMPOUND DILUTION SCHEME: All compounds supplied will be prepared to a working stock of 50× final assay concentration in 100% DMSO. Where appropriate, more concentrated stocks will be diluted manually to 50× using 100% DMSO. Compounds supplied as powders will be reconstituted to a 10 mM stock in 100% DMSO before further dilution to 50×.

ASSAY PROCEDURE: The required volume of the 50× stock of test compound is added to the assay well, before a reaction mix containing the enzyme and substrate is added. The reaction is initiated by the addition of ATP at the selected concentration. There is no pre-incubation of the compound with the enzyme/substrate mix prior to ATP addition. For further details of each individual assay, please refer to the website or the accompanying protocol document.

DATA ANALYSIS: Data are handled using a custom-built in-house analysis software. Results are expressed as kinase activity remaining, as a percentage of the DMSO control. This is calculated using the following formula:

$$\\ \frac{Mean of Sample Counts - Mean of Blank Counts}{Mean of Control Counts}$$

For IC50 determinations, data are analysed using XLFit version 5.3 (ID Business Solutions). Sigmoidal dose-response (variable slope) curves will be fit based on the mean result for each test concentration using non-linear regression analysis. Where the top and/or bottom of the curve fall >10% out with 100 and 0, respectively, either or both of these limits may be constrained at 100 and 0, provided that the QC criterion on R2 is met.

IC50 values for MAP4K4 of selected compounds; compounds having an IC50 value of 1-12 nM as +++, 12-120 nM as ++, and >120 nM as +.

1. A compound represented by Formula (A*): 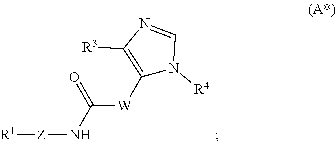
or a pharmaceutically acceptable salt thereof, wherein:
R¹ is selected from:
N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
optionally substituted 3 to 10-membered heterocycle; wherein the 3- to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
R³ is selected from optionally substituted C₁-C₆ alkyl, optionally substituted 3- to 10-membered heterocycle and optionally substituted C₃₋₁₀ carbocycle, wherein each is optionally substituted with one or more substituents independently selected at each occurrence from halogen, —OH, —CN, —NO₂, —NH₂, oxo, ═S, C₁₋₆ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
R⁴ is selected from:
hydrogen;
optionally substituted C₁-C₆ alkyl, wherein the C₁-C₆ alkyl is optionally substituted with one or more substituents are independently selected at each occurrence from halogen, —OH, —CN, —NO₂, —NH₂, oxo, ═S, —O—C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
optionally substituted C₃₋₁₀ carbocycle, wherein the C₃₋₁₀ carbocycle is optionally substituted with one or more substituents independently selected at each occurrence from halogen, —OH, —CN, —NO₂, —NH₂, oxo, ═S, C₁₋₁₀ alkyl, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
optionally substituted 3- to 8-membered heterocycle, wherein the 3- to 8-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
W is selected from optionally substituted thiazole, wherein the thiazole is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, and 3- to 12-membered heterocycle.

2. (canceled)

3. (canceled)

4. The compound or salt of claim 1, wherein Z is selected from optionally substituted phenyl and optionally substituted pyridine.

5. The compound or salt of claim 4, wherein the phenyl of Z is optionally substituted with one or more substituents independently selected from halogen, —OH, —CN, —NO₂, —NH₂, oxo, ═S, C₁₋₁₀ alkyl, —C₁₋₁₀ haloalkyl, and —O—C₁₋₁₀ alkyl.

6. (canceled)

7. (canceled)

8. (canceled)

9. (canceled)

10. (canceled)

11. (canceled)

12. The compound or salt of claim 1, wherein W is

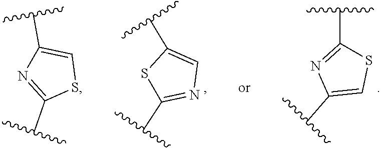 [SMILES: *c1csc(*)n1 ; *c1cnc(*)s1 ; *c1csc(*)n1]

13. (canceled)

14. (canceled)

15. The compound or salt of claim 1, wherein R⁴ is selected from optionally substituted C₁-C₆ alkyl, and optionally substituted C₃₋₆ carbocycle.

16. (canceled)

17. (canceled)

18. The compound or salt of claim 15, wherein for R⁴, the optional substituents of the optionally substituted C₁-C₆ alkyl are independently selected from halogen, —OH, —CN, —NO₂, —NH₂, oxo, —O—C₁₋₁₀ alkyl, —C₁₋₁₀ haloalkyl, and —O—C₁₋₁₀ alkyl.

19. (canceled)

20. (canceled)

21. (canceled)

22. (canceled)

23. (canceled)

24. (canceled)

25. The compound or salt of claim 1, wherein R⁴ is selected from unsubstituted C₁-C₆ alkyl and unsubstituted C₃₋₆ cycloalkyl.

26. (canceled)

27. The compound or salt of claim 1, wherein R³ is selected from optionally substituted C₃₋₆ carbocycle and optionally substituted phenyl.

28. (canceled)

29. The compound or salt of claim 27, wherein the optional substituents of phenyl of R³ are selected from halogen and —C₁₋₁₀ haloalkyl.

30. (canceled)

31. (canceled)

32. The compound or salt of claim 1, wherein R¹ is substituted C₁-C₆ alkyl.

33. (canceled)

34. The compound or salt of claim 1, wherein R¹ is an optionally substituted 3 to 10-membered heterocycle, wherein the heterocycle is optionally substituted with one or more substituents independently selected at each occurrence from halogen, —OH, —CN, —NO₂, —NH₂, oxo, —C₁₋₁₀ haloalkyl, —O—C₁₋₁₀ alkyl, and optionally substituted C₁₋₁₀ alkyl, wherein the optional substituents on the C₁₋₁₀ alkyl are independently selected at each occurrence from one or more hydroxy, halogen, oxo, —C₁₋₁₀ haloalkyl, —NH₂, —CN, and —NO₂.

35. The compound or salt of claim 34, wherein for R¹, the heterocycle has at least one nitrogen atom, phosphorous atom, or oxygen atom.

36-47. (canceled)

48. The compound or salt of claim 1, wherein R¹ is selected from

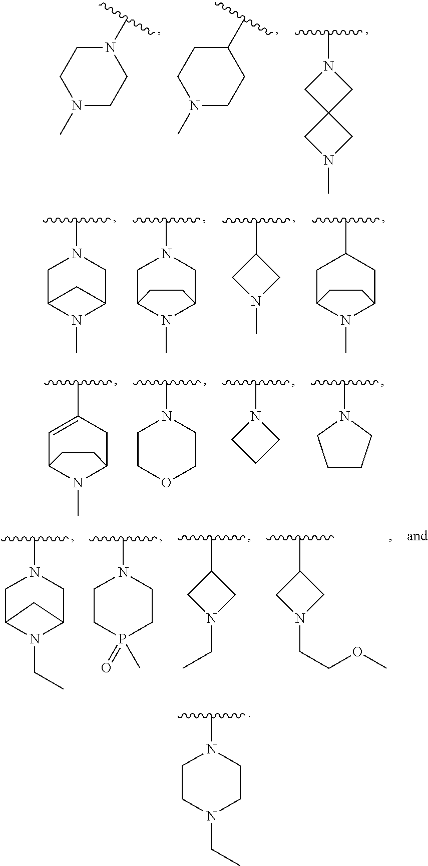

49. (canceled)

50. (canceled)

51. (canceled)

52. (canceled)

53. (canceled)

54. The compound or salt of claim 1, wherein the compound is selected from 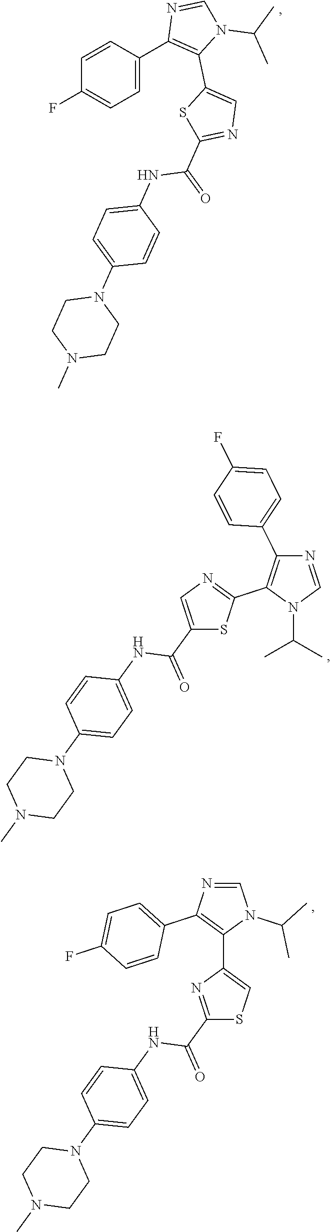 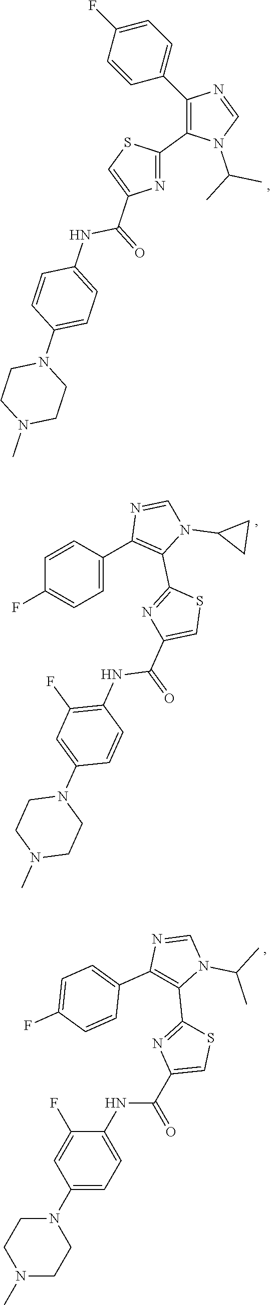 [SMILES: CC(C)n1cnc(-c2ccc(F)cc2)c1-c1nc(C(=O)Nc2ccc(N3CCN(C)CC3)cc2)cs1 ; CN1CCN(c2ccc(NC(=O)c3csc(-c4c(-c5ccc(F)cc5)ncn4C4CC4)n3)c(F)c2)CC1 ; CC(C)n1cnc(-c2ccc(F)cc2)c1-c1nc(C(=O)Nc2ccc(N3CCN(C)CC3)cc2F)cs1] 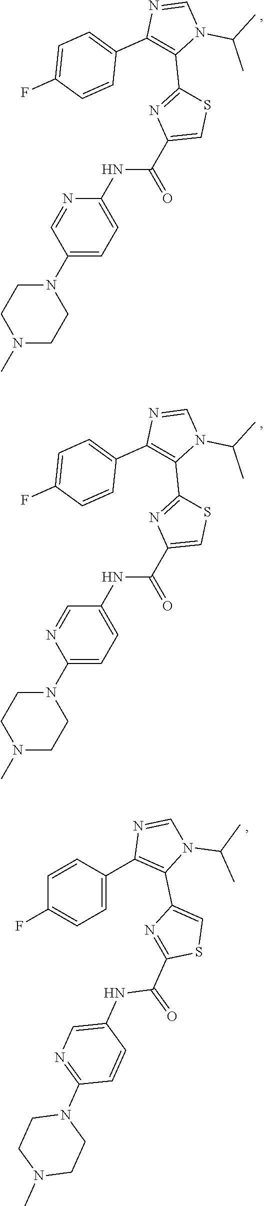 [SMILES: CC(C)n1cnc(-c2ccc(F)cc2)c1-c1nc(C(=O)Nc2ccc(N3CCN(C)CC3)cn2)cs1 ; CC(C)n1cnc(-c2ccc(F)cc2)c1-c1nc(C(=O)Nc2ccc(N3CCN(C)CC3)nc2)cs1 ; CC(C)n1cnc(-c2ccc(F)cc2)c1-c1csc(C(=O)Nc2ccc(N3CCN(C)CC3)nc2)n1] 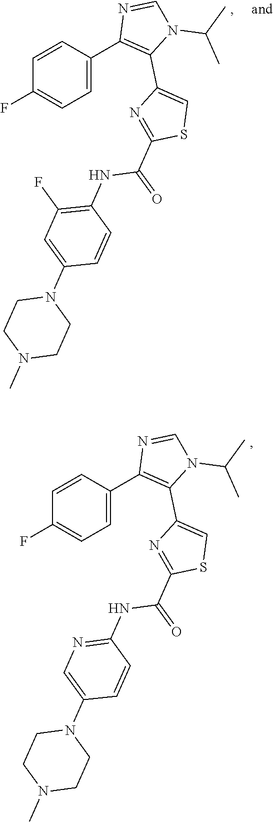 [SMILES: CC(C)n1cnc(-c2ccc(F)cc2)c1-c1csc(C(=O)Nc2ccc(N3CCN(C)CC3)cc2F)n1 ; CC(C)n1cnc(-c2ccc(F)cc2)c1-c1csc(C(=O)Nc2ccc(N3CCN(C)CC3)cn2)n1]
and/or a pharmaceutically acceptable salt of any one thereof.

55-134. (canceled)

135. A compound represented by Formula (B*): 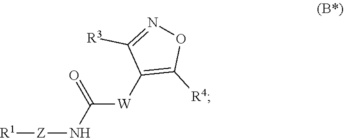
or a pharmaceutically acceptable salt thereof, wherein:
R¹ is selected from:
optionally substituted 3 to 10-membered heterocycle; wherein the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from hydroxy, halogen, oxo, —C₁₋₁₀ haloalkyl, —NH₂, —CN, —O—C₁₋₁₀ alkyl, and —NO₂;
—N(R⁵)₂, wherein R⁵ is selected from hydrogen, and optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein each is optionally substituted with one or more substituents independently selected at each occurrence from one or more halogen, —OH, —CN, —NO₂, —NH₂, oxo, ═S, —C₁₋₁₀ haloalkyl, —O—C₁₋₁₀ alkyl;
W is selected from optionally substituted 3- to 12-membered heterocycle, wherein the 3- to 12-membered heterocycle is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

136. The compound or salt of claim 135, wherein the compound is selected from 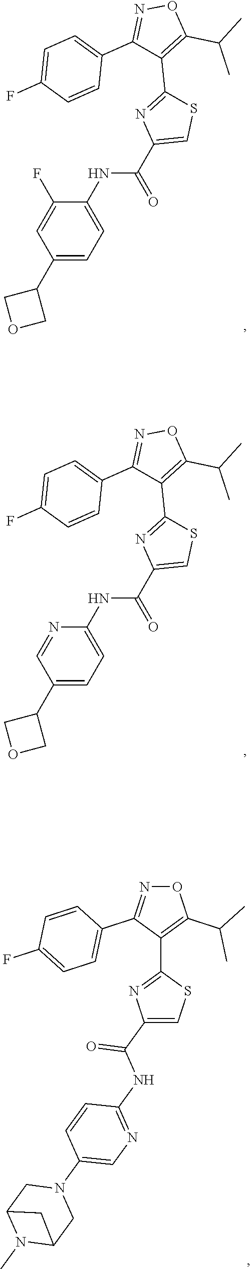 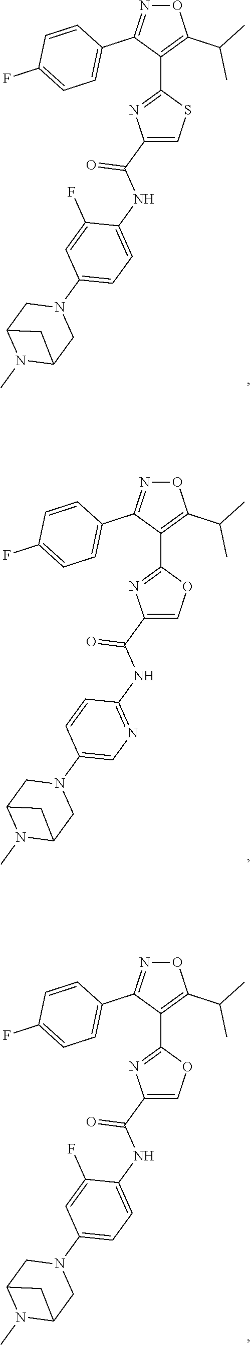 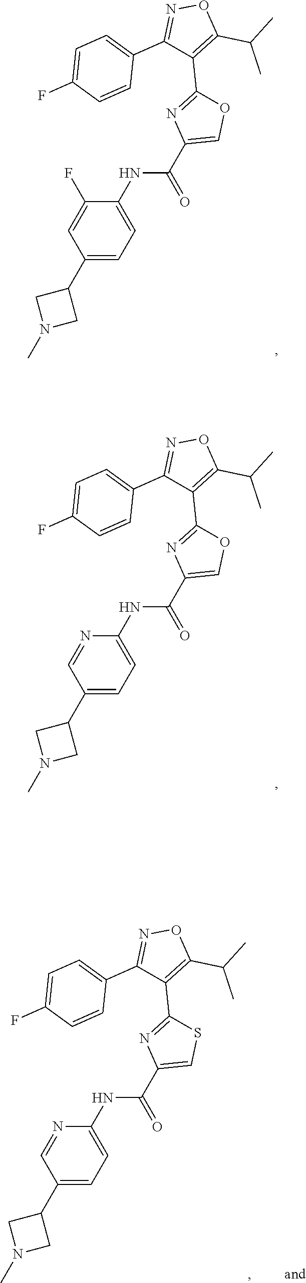 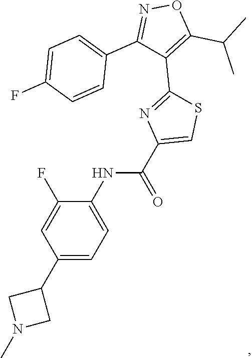 [SMILES: CC(C)c1onc(-c2ccc(F)cc2)c1-c1nc(C(=O)Nc2ccc(C3CN(C)C3)cc2F)cs1]
or a pharmaceutically acceptable salt of any one thereof.

137. A compound represented by Formula (C*). 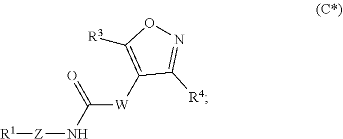
or a pharmaceutically acceptable salt thereof, wherein:
R³ is selected from a C₃₋₁₂ carbocycle optionally substituted with one or more substituents independently selected at each occurrence from halogen, —OH, —CN, —NO₂, —NH₂, oxo, ═S, C₁₋₁₀ alkyl, —C₁₋₁₀ haloalkyl, and —O—C₁₋₁₀ alkyl;
R⁴ is independently selected from halogen, —OH, —CN, —NO₂, —NH₂, —N(H)C₁-C₆ alkyl, —N(C₁-C₆ alkyl)₂, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle, wherein the C₁₋₁₀ alkyl, C₃₋₁₂ carbocycle and 3- to 12-membered heterocycle are each optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, C₁₋₁₀ alkyl, —C₁₋₁₀ haloalkyl, and —O—C₁₋₁₀ alkyl;
Z is selected from optionally substituted 3- to 12-membered heterocycle and optionally substituted C₃-C₁₂ carbocycle, wherein each is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl;
R¹ is selected from:
optionally substituted 3 to 10-membered heterocycle; wherein the 3 to 10-membered heterocycle is optionally substituted with one or more substituents independently selected at each occurrence from halogen, —OH, —CN, —NO₂, —NH₂, —N(H)C₁-C₆ alkyl, —N(C₁-C₆ alkyl)₂, oxo, ═S, —S(O₂)NH₂, —C₁₋₁₀ haloalkyl, —O—C₁₋₁₀ alkyl, C₂₋₁₀ alkenyl, C₂₋₁₀ alkynyl, C₃₋₁₂ carbocycle, 3- to 12-membered heterocycle, and optionally substituted C₁₋₁₀ alkyl, wherein the C₁₋₁₀ alkyl is optionally substituted with one or more substituents independently selected at each occurrence from one or more hydroxy, halogen, oxo, —C₁₋₁₀ haloalkyl, —NH₂, —CN, —O—C₁₋₁₀ alkyl, and —NO₂;
—N(R⁵)₂, wherein R⁵ is selected from hydrogen, and optionally substituted C₁-C₆ alkyl, wherein the C₁-C₆ alkyl is optionally substituted with one or more substituents independently selected at each occurrence from selected from halogen, —OH, —CN, —NO₂, —NH₂, oxo, ═S, —C₁₋₁₀ haloalkyl, —O—C₁₋₁₀ alkyl, C₂₋₁₀ alkenyl, C₂₋₁₀ alkynyl, C₃₋₁₂ carbocycle, and 3- to 12-membered heterocycle;
optionally substituted C₁-C₆ alkyl, wherein the C₁-C₆ alkyl is optionally substituted with one or more substituents independently selected at each occurrence from halogen, —OH, —CN, —NO₂, —NH₂, oxo, ═S, —C₁₋₁₀ haloalkyl, —O—C₁₋₁₀ alkyl, —O—C₁₋₆alkyl-O—C(O)(O—C₁₋₁₀ alkyl), C₂₋₁₀ alkenyl, C₂₋₁₀ alkynyl, C₃₋₁₂ carbocycle, and 3- to 12-membered heterocycle;
W is selected from optionally substituted 3- to 12-membered heterocycle, wherein the 3- to 12-membered heterocycle is optionally substituted with one or more substituents independently selected at each occurrence from one or more halogen, —OH, —CN, —NO₂, —NH₂, oxo, ═S, —C₁₋₁₀ haloalkyl, —O—C₁₋₁₀ alkyl, C₁₋₁₀ alkyl, C₂₋₁₀ alkenyl, C₂₋₁₀ alkynyl, C₃₋₁₂ carbocycle, 3- to 12-membered heterocycle.

138. The compound or salt of claim 137, wherein the compound is selected from 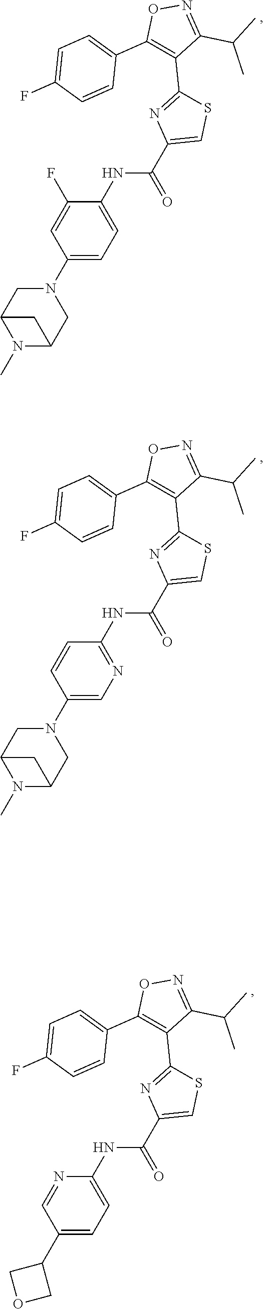 [SMILES: CC(C)c1noc(-c2ccc(F)cc2)c1-c1nc(C(=O)Nc2ccc(N3CC4CC(C3)N4C)cc2F)cs1 ; CC(C)c1noc(-c2ccc(F)cc2)c1-c1nc(C(=O)Nc2ccc(N3CC4CC(C3)N4C)cn2)cs1 ; CC(C)c1noc(-c2ccc(F)cc2)c1-c1nc(C(=O)Nc2ccc(C3COC3)cn2)cs1] 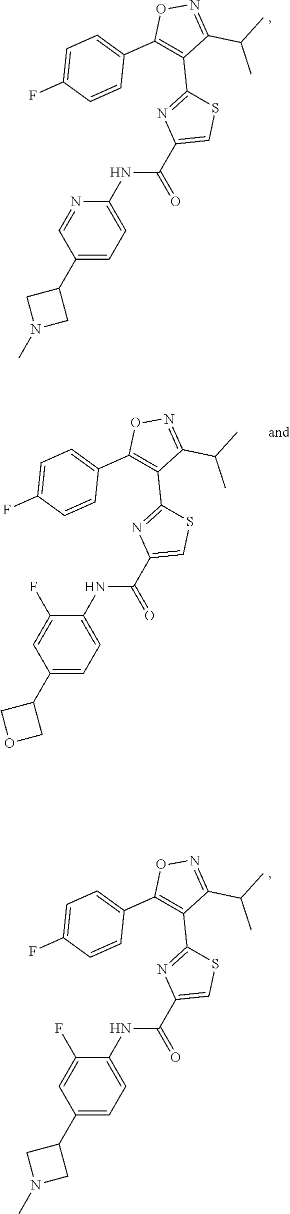
or a pharmaceutically acceptable salt of any one thereof.

139-140. (canceled)

141. A pharmaceutical composition comprising a compound or salt of claim 1 and a pharmaceutically acceptable excipient.

142. A method of treating or preventing disease, comprising administering a compound or salt of claim 1 to a subject in need thereof.

143-152. (canceled)
